(19) United States
(12) Patent Application Publication
(10) Pub. No.: US 2025/0115551 A1
(43) Pub. Date: Apr. 10, 2025

(54) SYNTHESIS OF RAS INHIBITORS

(71) Applicant: Revolution Medicines, Inc., Redwood City, CA (US)

(72) Inventors: Yi LI, San Jose, CA (US); Benjamin MENDOZA, Foster City, CA (US); Sriram NAGANATHAN, San Jose, CA (US); Ross WANG, Fremont, CA (US); Yi YI, Hayward, CA (US); Steven G. BALLMER, Burlingame, CA (US); James DAVIDSON, Mountain View, CA (US); Xiaojun HUANG, San Jose, CA (US)

(21) Appl. No.: 18/904,234

(22) Filed: Oct. 2, 2024

Related U.S. Application Data
(60) Provisional application No. 63/560,883, filed on Mar. 4, 2024.
(60) Provisional application No. 63/542,112, filed on Oct. 3, 2023.

(51) Int. Cl. C07D 203/24; C07D 203/08; C07D 209/00; C07D 401/04; C07D 498/22

(52) U.S. Cl. CPC C07D 203/24; C07D 203/08; C07D 209/00; C07D 401/04; C07D 498/22

(57) ABSTRACT

The present invention relates to Ras inhibitors, intermediates in the synthesis thereto, and methods for preparing the Ras inhibitors and the intermediates.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable to above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61 K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations, and there remains a need for convenient, scalable synthetic methods thereto.

### SUMMARY OF THE INVENTION

The invention features methods of preparing Ras inhibitors, intermediates useful in the synthesis thereto, and methods of preparing the intermediates.

In an aspect, the disclosure provides a method of separating enantiomers of a compound of Formula I

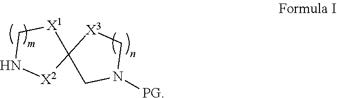

The method includes the steps of: a) contacting the compound of Formula I with a chiral acid to form a diastereomeric salt of the compound of Formula I; and b) separating each diastereomer of the diastereomeric salt, thereby separating enantiomers of a compound of Formula I, wherein
n and m are each, independently, 0, 1, 2, 3, 4, or 5;
PG is a nitrogen protecting group; and
X¹, X², and X³ are each, independently, —CH₂—, —CHF—, —CF₂—, —C(O)—, or —O—.

In some embodiments, the chiral acid is a carboxylic acid. In some embodiments, the chiral carboxylic acid is a chiral dicarboxylic acid. In some embodiments, the chiral dicarboxylic acid is L(+)-tartaric acid. In some embodiments, the separating step (b) includes recrystallization. In some embodiments, the nitrogen protecting group is Boc (tert-Butyloxycarbonyl).

In some embodiments, the compound of Formula I is further described by the compound of Formula Ia:

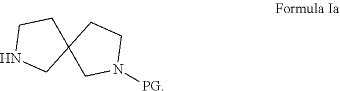

In some embodiments, the compound of Formula I or Formula Ia is Compound B:

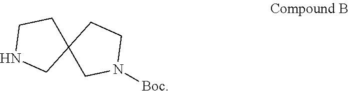 [SMILES: CC(C)(C)OC(=O)N1CCC2(CCNC2)C1]

In another aspect, the disclosure provides a barium salt of Compound A:

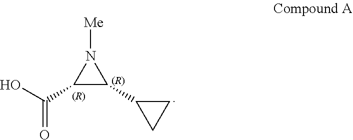

In some embodiments, the barium salt is a 2:1 (compound A: barium) salt.

In a further aspect, the disclosure provides a method of preparing the barium salt of Compound A. The method includes the steps of:
a) condensing a compound of Formula IIa and Compound C to form a compound of Formula IIb:

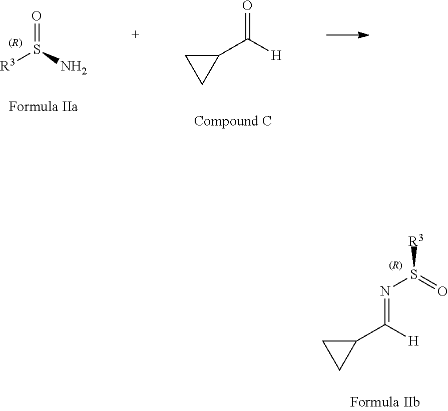

b) cyclizing the compound of Formula IIb and a compound of Formula IIc to form a compound of Formula IId:

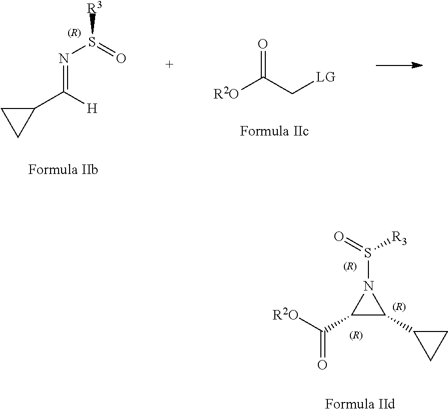

c) hydrolyzing the compound of Formula IId to form a compound of Formula IIe:

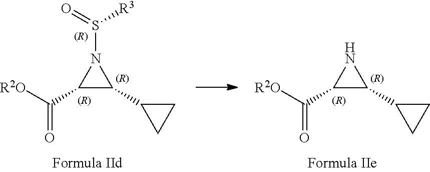

d) methylating the compound of Formula IIe to form a compound of Formula IIf:

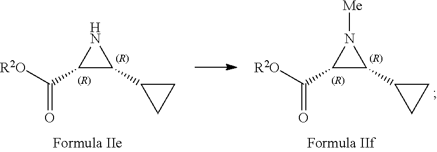

e) hydrolyzing the compound of Formula IIf to form the barium salt of Compound A:

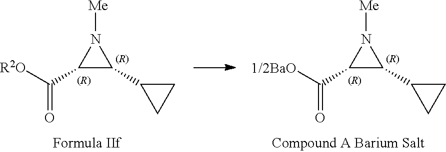

wherein R² and R³ are each, independently, optionally substituted C₁-C₆ alkyl, optionally substituted C₃₋₆ cycloalkyl, or optionally substituted C₆-C₁₀ aryl, and LG is a leaving group selected from the group consisting of halogen, triflate, mesylate, and tosylate.

In some embodiments of the method of preparing the barium salt of Compound A, the condensing step (a) includes contacting the compound of Formula IIa and Compound C with a base. In some embodiments, the cyclizing step (b) includes contacting the compound of Formula IIb and the Compound of Formula IIc with a base. In some embodiments, the hydrolyzing step (c) includes contacting the compound of Formula IId with an acid. In some embodiments, the methylating step (d) includes contacting the compound of Formula IIe with MeB(OH)₂. In some embodiments, the hydrolyzing step (e) includes contacting the compound of Formula IIf with barium hydroxide. In some embodiments, R³ is optionally substituted C₆-C₁₀ aryl. In some embodiments, R³ is

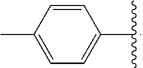 [SMILES: *c1ccc(C)cc1 ; "."]

In some embodiments, R³ is optionally substituted C₁-C₆ alkyl. In some embodiments, R³ is tert-butyl. In some embodiments, R² is optionally substituted C₁-C₆ alkyl, optionally substituted C₃₋₆ cycloalkyl, or optionally substituted C₆₋₁₀ aryl. In some embodiments, R² is benzyl, methyl, or t-Butyl. In some embodiments, LG is halogen. In some embodiments, LG is —Br.

In another aspect, the disclosure provides a method of preparing a compound, or a salt thereof, of Formula III:

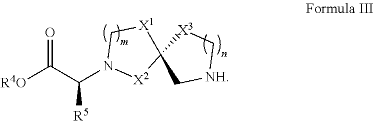

The method includes the steps of:
a) coupling a compound of Formula IIId with a compound of Formula I to form a compound of Formula IIIe:

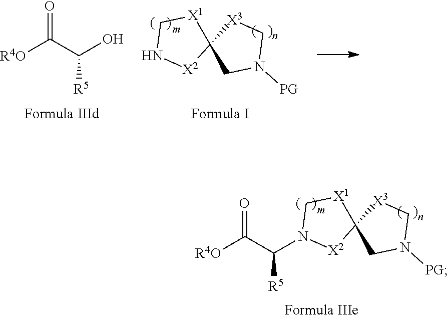

and
b) deprotecting the compound of Formula IIIe to form the compound of Formula III:

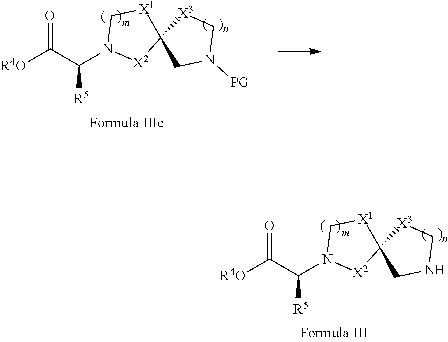

wherein
n and m are each, independently, 0, 1, 2, 3, 4, or 5;
PG is a nitrogen protecting group;
each R⁴ is independently, optionally substituted C₁-C₆ alkyl or optionally substituted C₆-C₁₀ aryl; and
R⁵ is optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and X¹, X², and X³ are each, independently, —CH₂—, —CHF—, —CF₂—, —C(O)—, or —O—.

In some embodiments, the nitrogen protecting group is Boc (tert-Butyloxycarbonyl). In some embodiments, the compound of Formula IIId is prepared by a method including the steps of:
A1) combining a compound of Formula IIIa and a compound of Formula IIIb to form a compound of Formula IIIc:

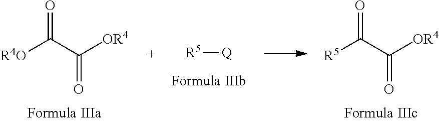

B1) reducing the compound of Formula IIIc to form the compound of Formula IIId:

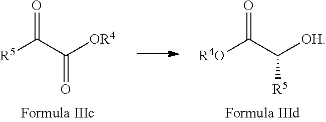

wherein Q is MgBr, MgCl, MgI, or Li.

In some embodiments, the reducing step (B1) includes contacting the compound of Formula IIIc with a ketoreductase enzyme.

In some embodiments, the compound of Formula IIId is prepared by a method including the steps of:
A2) converting a compound of Formula IIIf to form a compound of Formula IIIg:

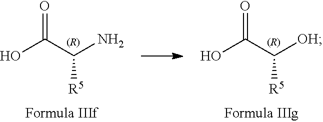

and
B2) reacting (e.g., esterifying) the compound of Formula IIIg to form the compound of Formula IIId:

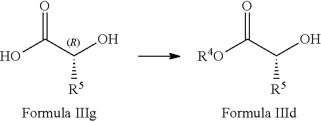

In some embodiments, the converting step (A2) includes forming a diazonium salt of the compound of Formula IIIf.

In some embodiments, step (b) includes contacting the compound of Formula IIIe with an acid.

In some embodiments, the compound of Formula III is Compound D:

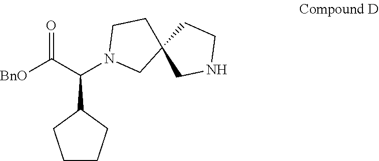 [SMILES: O=C(OCc1ccccc1)[C@H](C1CCCC1)N1CC[C@]2(CCNC2)C1]

which is prepared by:
a) coupling a compound of Formula IIId-1 with Compound B to form a compound of Formula IIIe-1 having the following structure:

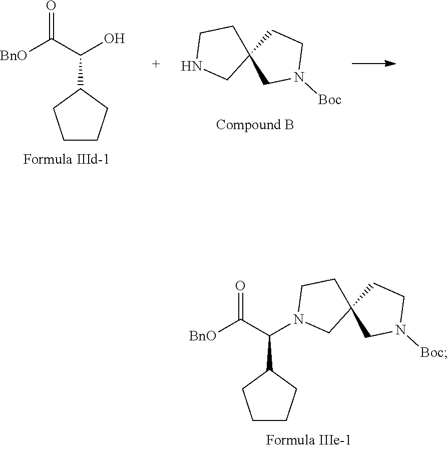 [SMILES: O=C(OCc1ccccc1)[C@H](O)C1CCCC1 ; CC(C)(C)OC(=O)N1CC[C@]2(CCNC2)C1 ; CC(C)(C)OC(=O)N1CC[C@]2(CCN([C@H](C(=O)OCc3ccccc3)C3CCCC3)C2)C1]

b) deprotecting the compound of Formula IIIe-1 to form Compound D having the following structure:

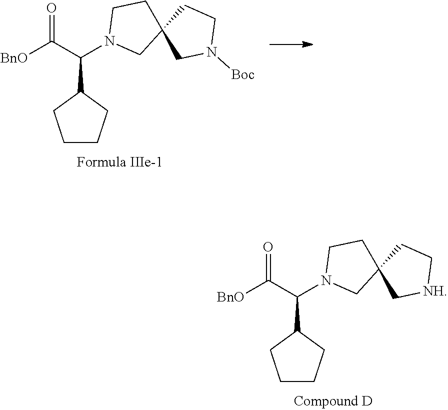 [SMILES: CC(C)(C)OC(=O)N1CC[C@]2(CCN([C@H](C(=O)OCc3ccccc3)C3CCCC3)C2)C1 ; O=C(OCc1ccccc1)[C@H](C1CCCC1)N1CC[C@]2(CCNC2)C1]

In yet another aspect, the disclosure provides a method of preparing a compound, or a salt thereof, of Formula IV:

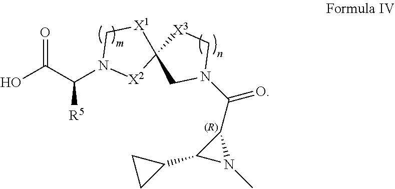

The method includes the steps of:
a) coupling a compound of Formula III and a barium salt of compound A to form a compound of Formula IVa:

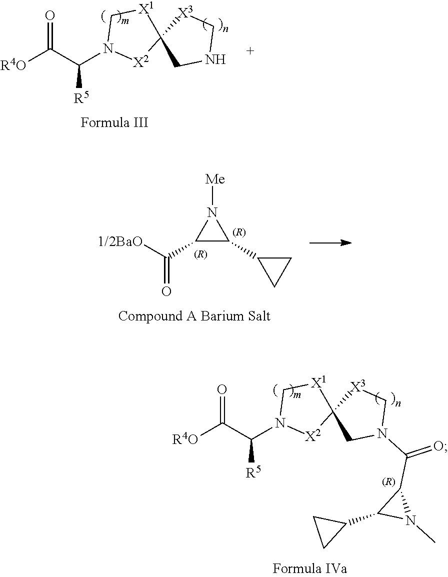

and
b) hydrolyzing the compound of Formula IVa to form the compound of Formula IV:

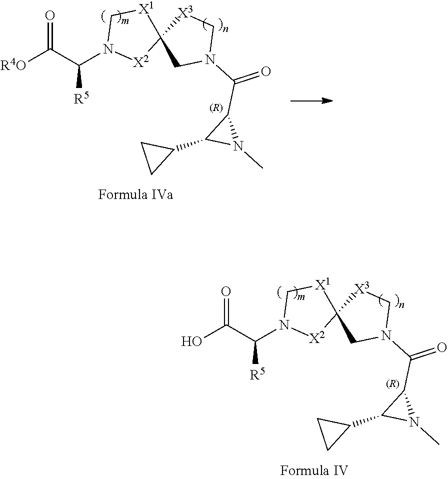

wherein
n and m are each, independently, 0, 1, 2, 3, 4, or 5;
R⁴ is optionally substituted C₁-C₆ alkyl or optionally substituted C₆-C₁₀ aryl;
R⁵ is optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

In some embodiments, the coupling step (a) includes contacting the compound of Formula III and the Compound A barium salt with chloro-N,N,N′,N′-tetramethylformamidinium hexafluorophosphate (TCFH). In some embodiments, the compound of Formula IVa is isolated as a maleate salt after the completion of step (a). In some embodiments, the compound of Formula IVa is isolated as a besylate salt after the completion of step (a). In some embodiments, the hydrolyzing step (b) includes contacting the compound of Formula IVa with an acid or base. In some embodiments, the hydrolyzing step (b) includes hydrogenating the compound of Formula IVa. In some embodiments, the hydrogenating further includes contacting the compound of Formula IVa with a palladium catalyst.

In some embodiments, the method includes preparing Compound E:

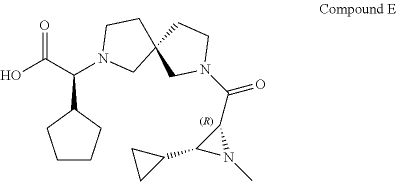

and the method including the steps of:
a) coupling Compound D and a barium salt of compound A to form a compound of Formula IVa having the following structure:

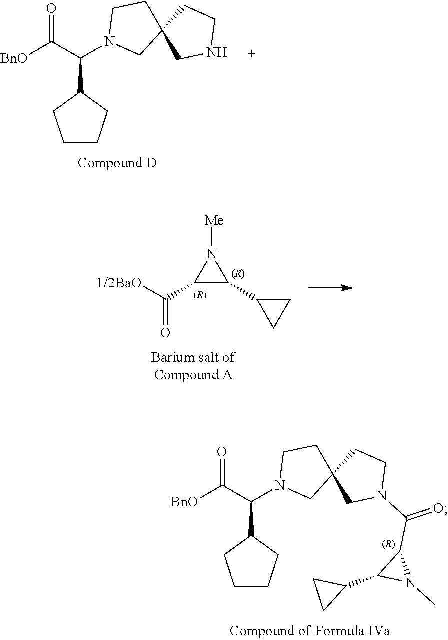

and
b) deprotecting the compound of Formula IVa to form Compound E having the following structure:

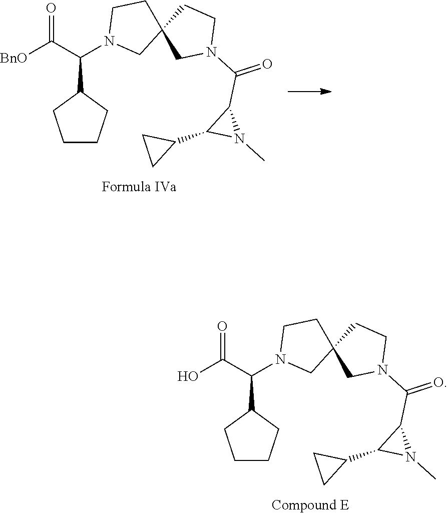

In some embodiments, the compound of Formula IVa is isolated as a maleate salt after the completion of step (a). In some embodiments, the compound of Formula IVa is isolated as a besylate salt after the completion of step (a).

In some embodiments of any of the methods and compounds described herein, m is 0. In some embodiments, m is 1. In some embodiments, m is 2. In some embodiments, n is 0. In some embodiments, n is 1. In some embodiments, n is 2.

In some embodiments of any of the methods and compounds described herein, each of X¹, X², and X³ is CH₂. In some embodiments, PG is

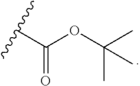 [SMILES: *C(=O)OC(C)(C)C ; "."]

In some embodiments, LG is halogen. In some embodiments, LG is Br.

In some embodiments, R³ is optionally substituted C₆-C₁₀ aryl. In some embodiments, R³ is

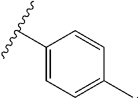 [SMILES: *c1ccc(C)cc1 ; "."]

In some embodiments, R³ is optionally substituted C₁-C₆ alkyl. In some embodiments, R³ is

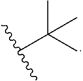 [SMILES: *C(C)(C)C ; "."]

In some embodiments, R² is optionally substituted C₁-C₆ alkyl, optionally substituted C₃₋₆ cycloalkyl, or optionally substituted C₆₋₁₀ aryl. In some embodiments, R² is benzyl, methyl, or t-Butyl. In some embodiments, R⁴ is optionally substituted C₁-C₆ alkyl or optionally substituted C₆-C₁₀ aryl. In some embodiments, R⁴ is benzyl, methyl, or t-Butyl. In some embodiments, R⁵ is optionally substituted C₁-C₆ alkyl or optionally substituted 3- to 6-membered cycloalkyl. In some embodiments, R⁵ is optionally substituted C₁-C₆ alkyl. In some embodiments, R⁵ is isopropyl. In some embodiments, R⁵ is optionally substituted 3- to 6-membered cycloalkyl. In some embodiments, R⁵ is cyclopentyl.

In yet another aspect, the disclosure provides a method of preparing a compound, or a salt thereof, of Formula VIII:

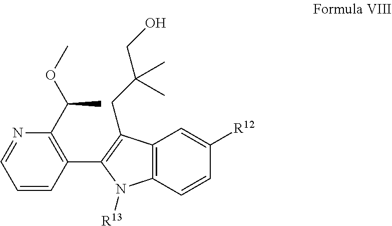

the method including the steps of:
a) esterifying and N-alkylating a compound of Formula VIIIa to form a compound of Formula VIIIb:

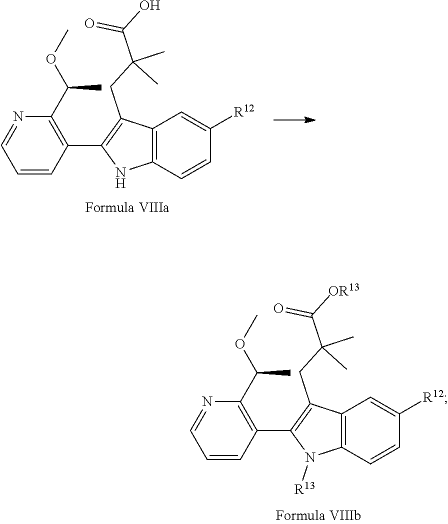

b) reducing the compound of Formula VIIIb to form a compound of Formula VIIIc as a mixture of atropisomers:

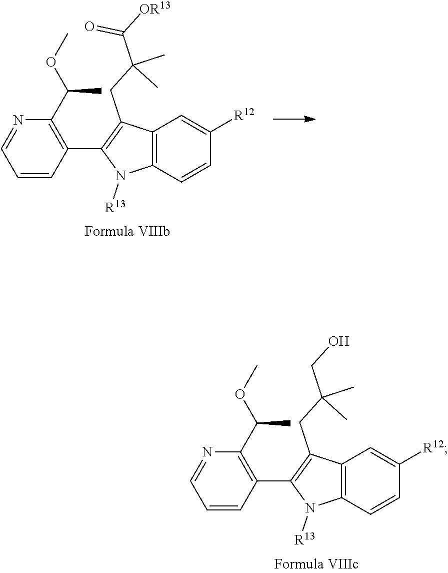

and
c) separating the mixture of atropisomers of Formula VIIIc to obtain a stereochemically pure compound of Formula VIII;
wherein R¹² is halogen; and
R¹³ is optionally substituted C₁-C₆ alkyl.

In yet another aspect, the disclosure provides a method of preparing a compound, or a salt thereof, of Formula VIII-1:

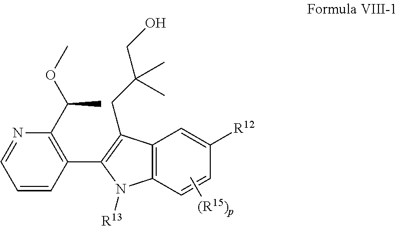

the method including the steps of:
a) esterifying and N-alkylating a compound of Formula VIIIa-1 to form a compound of Formula VIIIb-1:

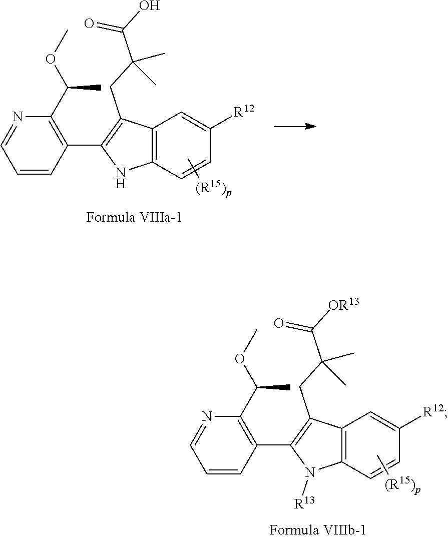

b) reducing the compound of Formula VIIIb-1 to form a compound of Formula VIIIc-1 as a mixture of atropisomers:

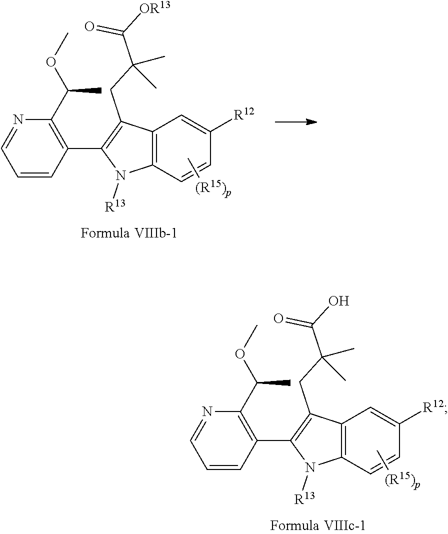

c) separating the mixture of atropisomers of Formula VIIIc-1 to obtain a stereochemically pure compound of Formula VIII-1;
wherein R¹² is halogen;
R¹³ is optionally substituted C₁-C₆ alkyl;
each R¹⁵ is, independently, hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
p is 0, 1, 2, or 3.

In some embodiments, the compound of Formula VIII is Compound F:

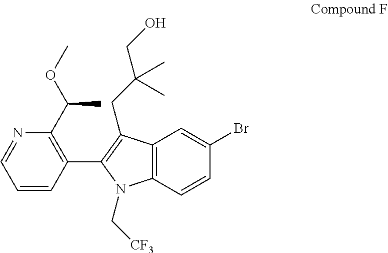 [SMILES: CO[C@@H](C)c1ncccc1-c1c(CC(C)(C)CO)c2cc(Br)ccc2n1CC(F)(F)F]

and the method includes the steps of:
a) esterifying and N-alkylating a compound of Formula VIIIa to form a compound of Formula VIIIb having the following structure:

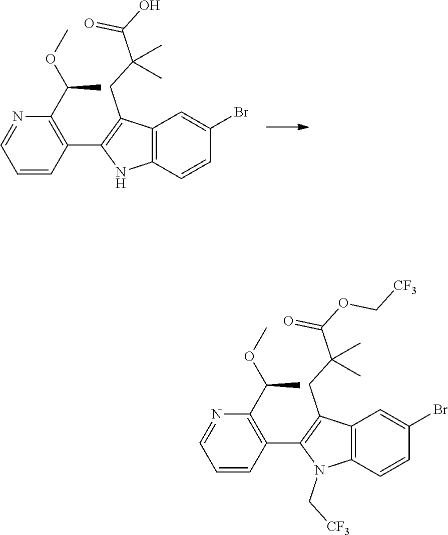 [SMILES: CO[C@@H](C)c1ncccc1-c1[nH]c2ccc(Br)cc2c1CC(C)(C)C(=O)O ; CO[C@@H](C)c1ncccc1-c1c(CC(C)(C)C(=O)OCC(F)(F)F)c2cc(Br)ccc2n1CC(F)(F)F]

b) reducing the compound of Formula VIIIb to form a compound of Formula VIIIc as a mixture of atropisomers having the following structure:

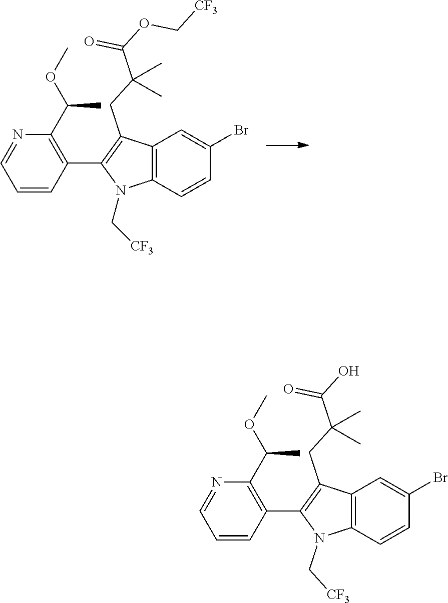 [SMILES: CO[C@@H](C)c1ncccc1-c1c(CC(C)(C)C(=O)OCC(F)(F)F)c2cc(Br)ccc2n1CC(F)(F)F ; CO[C@@H](C)c1ncccc1-c1c(CC(C)(C)C(=O)O)c2cc(Br)ccc2n1CC(F)(F)F]

c) separating the mixture of atropisomers of Formula VIIIc to obtain the stereochemically pure compound of Formula VIII.

In yet another aspect, the disclosure provides a method of preparing a compound, or a salt thereof, of Formula VIII:

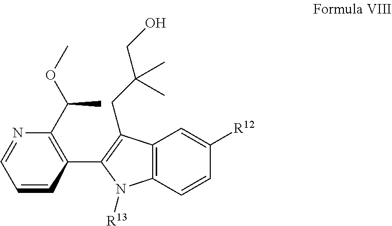

the method including the steps of:
a) reacting (e.g., esterifying) a compound of Formula VIIIa to form a compound of Formula VIIId:

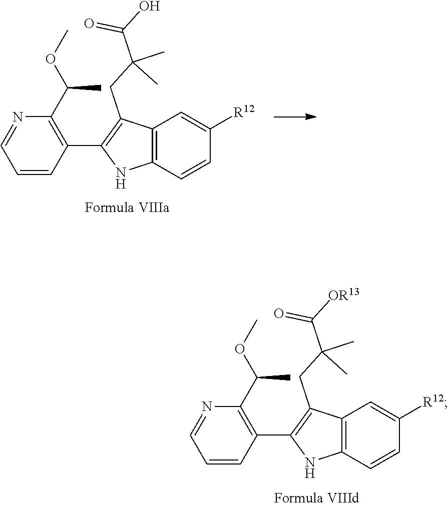

b) N-alkylating the compound of Formula VIIId to form the compound of Formula VIIIb:

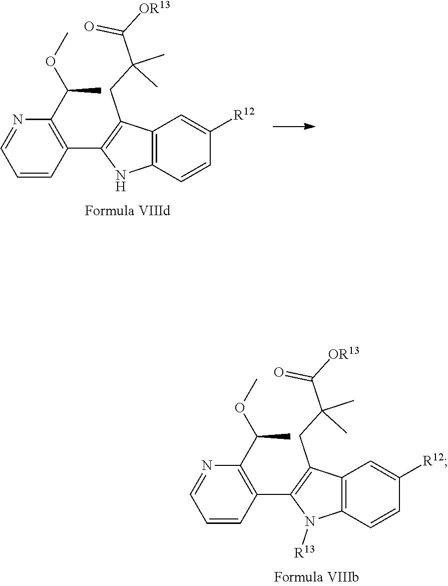

c) reducing the compound of Formula VIIIb to form a compound of Formula VIIIc as a mixture of atropisomers:

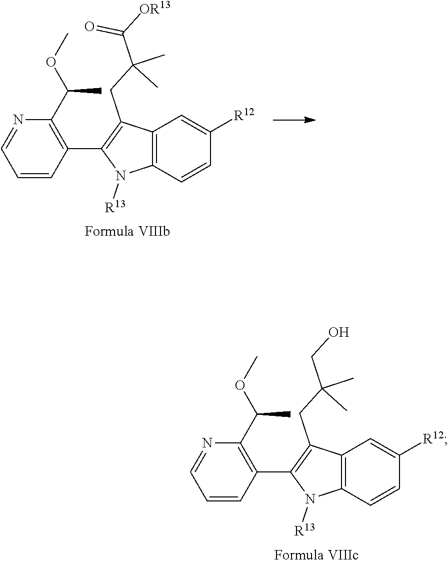

and
d) separating the mixture of atropisomers of Formula VIIIc to obtain the stereochemically pure compound of Formula VIII;
wherein R¹² is halogen; and
R¹³ is optionally substituted C₁-C₆ alkyl.

In yet another aspect, the disclosure provides a method of preparing a compound, or a salt thereof, of Formula VIII-1:

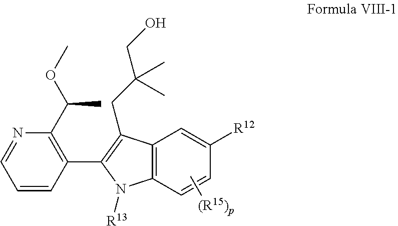

the method including the steps of:
a) reacting (e.g., esterifying) a compound of Formula VIIIa-1 to form a compound of Formula VIIId-1:

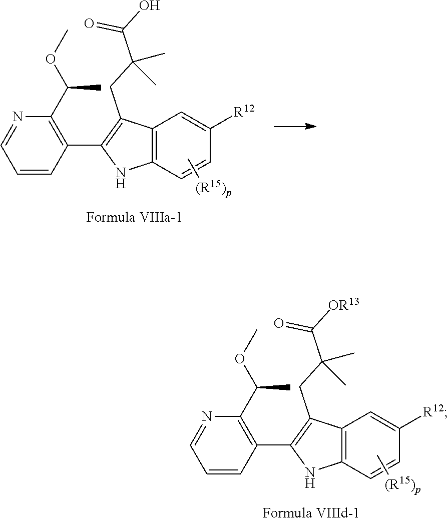

b) N-alkylating the compound of Formula VIIId-1 to form the compound of Formula VIIIb-1:

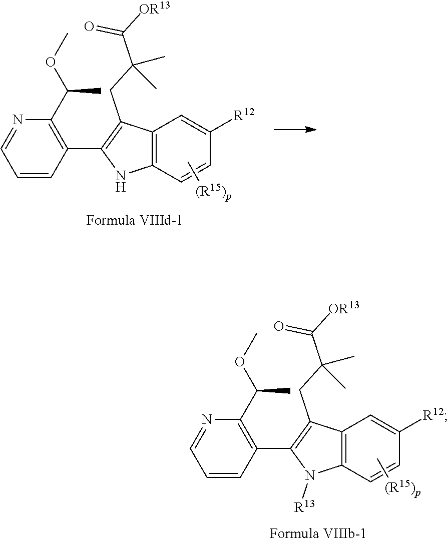

c) reducing the compound of Formula VIIIb-1 to form a compound of Formula VIIIc-1 as a mixture of atropisomers:

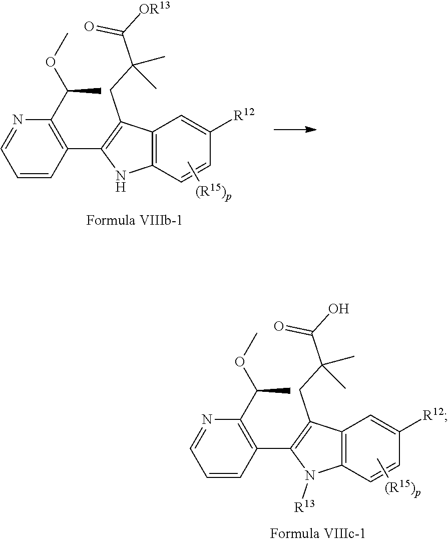

d) separating the mixture of atropisomers of Formula VIIIc-1 to obtain the stereochemically pure compound of Formula VIII-1;
wherein R¹² is halogen;
R¹³ is optionally substituted C₁-C₆ alkyl;
each R¹⁵ is, independently, hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
p is 0, 1, 2, or 3.

In some embodiments, the compound of Formula VIII is Compound G:

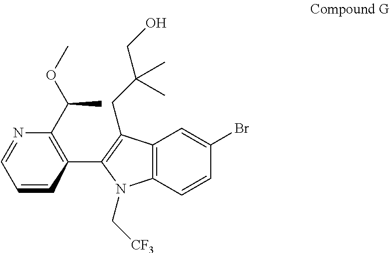

and the method includes the steps of:
a) reacting (e.g., esterifying) a compound of Formula VIIIa to form a compound of Formula VIIId having the following structure:

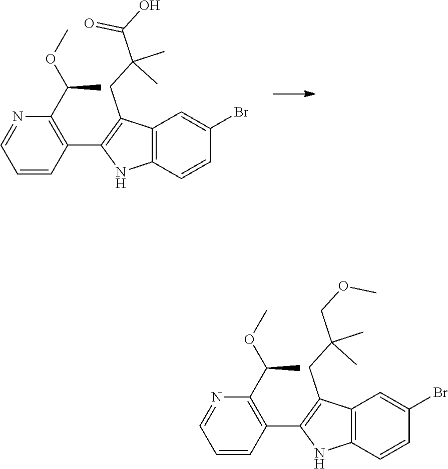 [SMILES: CO[C@@H](C)c1ncccc1-c1[nH]c2ccc(Br)cc2c1CC(C)(C)C(=O)O ; COCC(C)(C)Cc1c(-c2cccnc2[C@H](C)OC)[nH]c2ccc(Br)cc12]

b) N-alkylating the compound of Formula VIIId to form the compound of Formula VIIIb having the following structure:

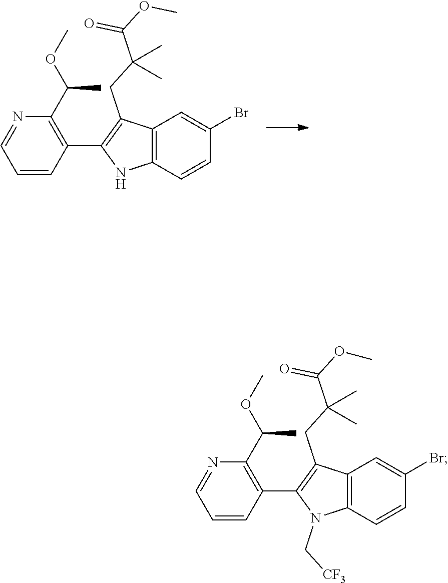 [SMILES: COC(=O)C(C)(C)Cc1c(-c2cccnc2[C@H](C)OC)[nH]c2ccc(Br)cc12 ; COC(=O)C(C)(C)Cc1c(-c2cccnc2[C@H](C)OC)n(CC(F)(F)F)c2ccc(Br)cc12]

c) reducing the compound of Formula VIIIb to form Compound G as a mixture of atropisomers having the following structure:

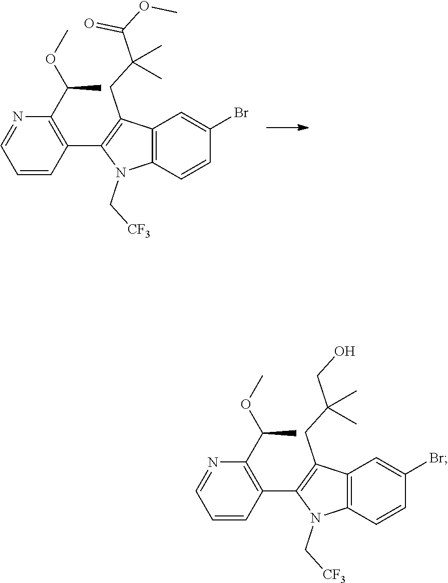 [SMILES: COC(=O)C(C)(C)Cc1c(-c2cccnc2[C@H](C)OC)n(CC(F)(F)F)c2ccc(Br)cc12 ; CO[C@@H](C)c1ncccc1-c1c(CC(C)(C)CO)c2cc(Br)ccc2n1CC(F)(F)F]

d) separating the mixture of atropisomers of Compound G to obtain the stereochemically pure Compound G.

In some embodiments, the mixture of atropisomers is separated by column chromatography or chemical resolution. In some embodiments, the chemical resolution includes contacting the compound of Formula VIIIc with an acid. In some embodiments, the acid is methanesulfonic acid. In some embodiments, the acid is camphor sulfonic acid (e.g., D-camphor-10-sulfonic acid). In some embodiments, step (b) includes contacting the compound of Formula VIIIa or the compound of Formula VIIId with 2,2,2-trifluoroethyl trifluoromethanesulfonate:

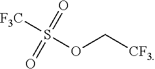 [SMILES: O=S(=O)(OCC(F)(F)F)C(F)(F)F]

In some embodiments, step (c) includes contacting the compound of Formula VIIIb with lithium borohydride (LiBH₄).

In yet another aspect, the disclosure provides a method of preparing a compound, or a salt thereof, of Formula IX:

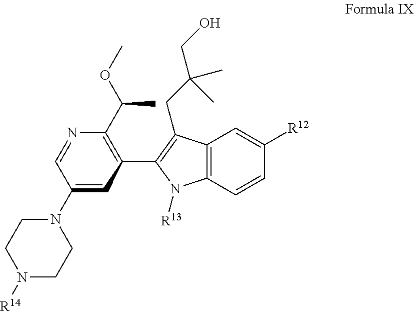

the method including the steps of:
a) borylating a compound of Formula VIII to form a compound Formula X:

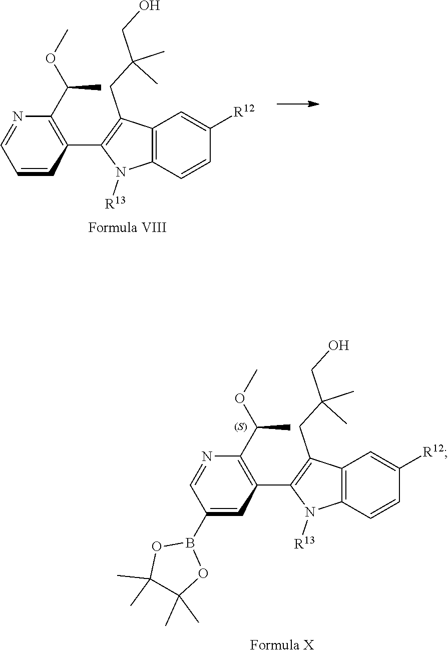

b) coupling the compound of Formula X with the compound of Formula XI to form a compound of Formula XII:

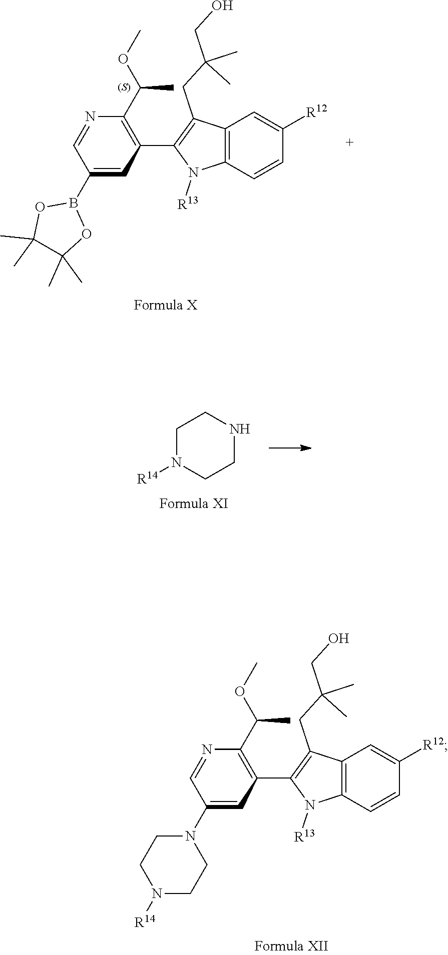

wherein R¹² is halogen;
R¹³ is optionally substituted C₁-C₆ alkyl; and
R¹⁴ is optionally substituted 3- to 6-membered cycloalkyl.

In yet another aspect, the disclosure provides a method of preparing a compound, or a salt thereof, of Formula IX-1:

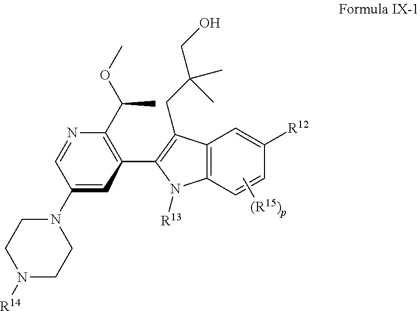

the method including the steps of:
a) borylating a compound of Formula VIII-1 to form a compound Formula X-1:

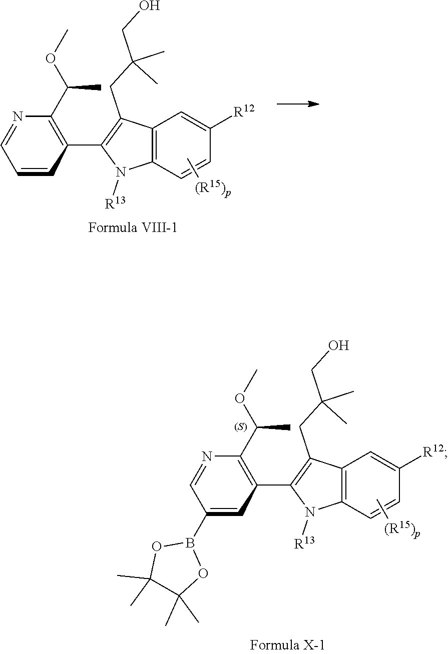

and
b) coupling the compound of Formula X-1 with the compound of Formula XI to form a compound of Formula XII-1:

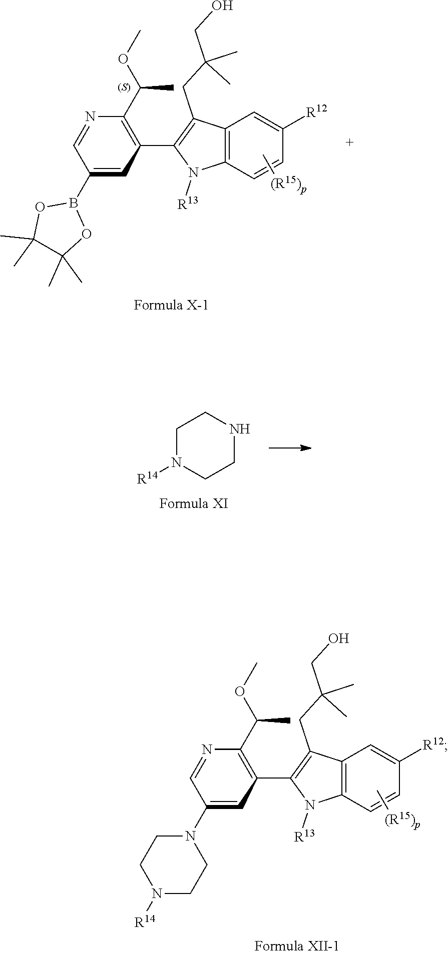

wherein R¹² is halogen;
R¹³ is optionally substituted C₁-C₆ alkyl;
R¹⁴ is optionally substituted 3- to 6-membered cycloalkyl;
each R¹⁵ is, independently, hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
p is 0, 1, 2, or 3.

In some embodiments, the compound of Formula IX is Compound H:

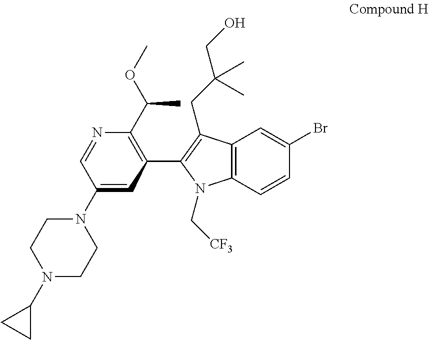

the method includes the steps of:
a) borylating a compound of Formula VIII to form a compound Formula X having the following structure:

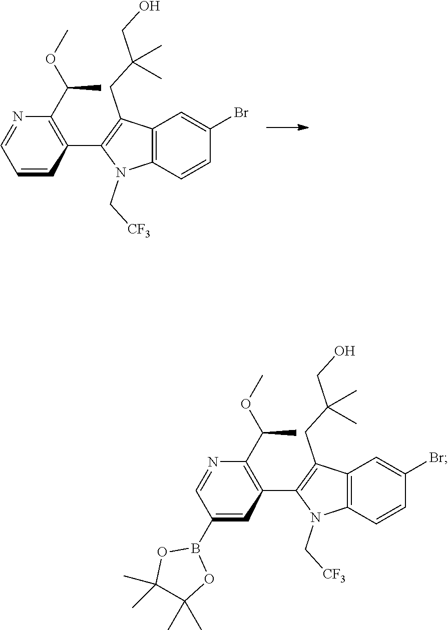

and
b) coupling the compound of Formula X with the compound of Formula XI to form Compound H having the following structure:

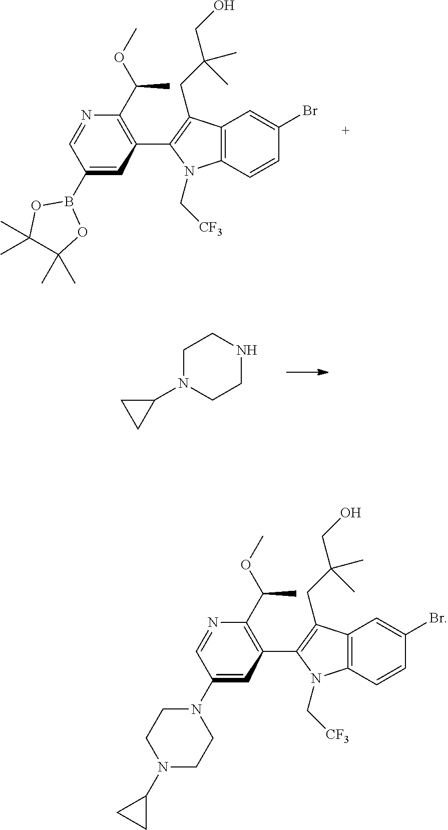

In some embodiments, the borylating step (a) includes contacting the compound of Formula VIII with an iridium catalyst. In some embodiments, the coupling step (b) includes contacting the compound of Formula X and the compound of Formula XI with a copper source and a base. In some embodiments, the copper source is Cu(OAc)₂. In some embodiments, the base is triethylamine or tetramethylpiperidine.

In some embodiments, the compound, or a salt thereof, of Formula X:

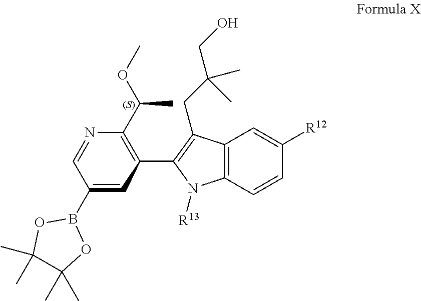

is further prepared by:
a) borylating the compound of Formula VIII to form a compound of Formula XIII:

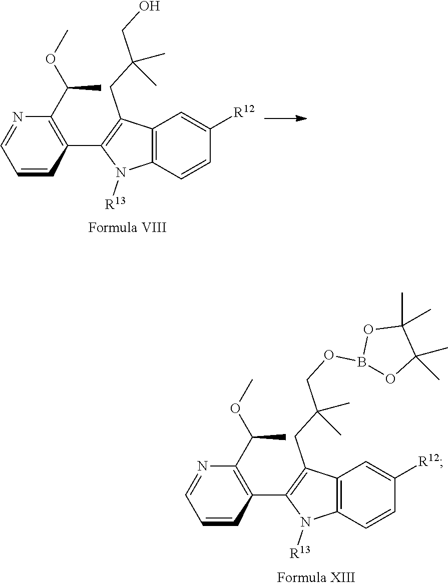

and
b) hydrolyzing and borylating the compound of Formula XIII in one step to form Formula X:

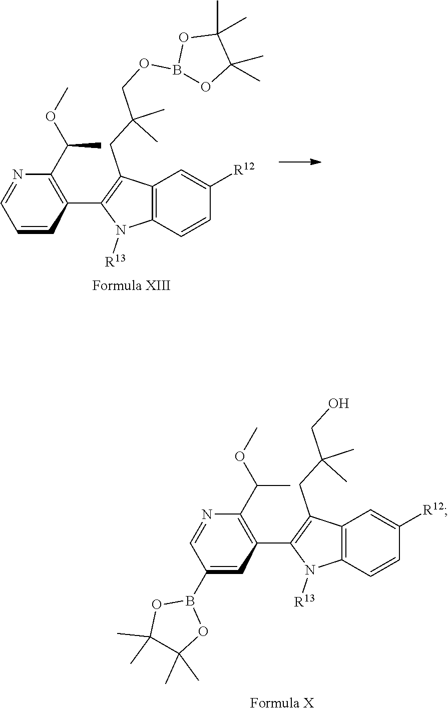

wherein R¹² is halogen; and
R¹³ is optionally substituted C₁-C₆ alkyl.

In some embodiments, the compound, or a salt thereof, of Formula X-1:

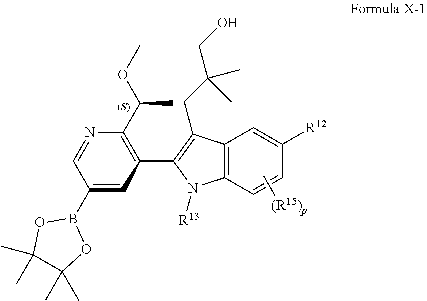

is further prepared by:
a) borylating the compound of Formula VIII-1 to form a compound of Formula XIII-1:

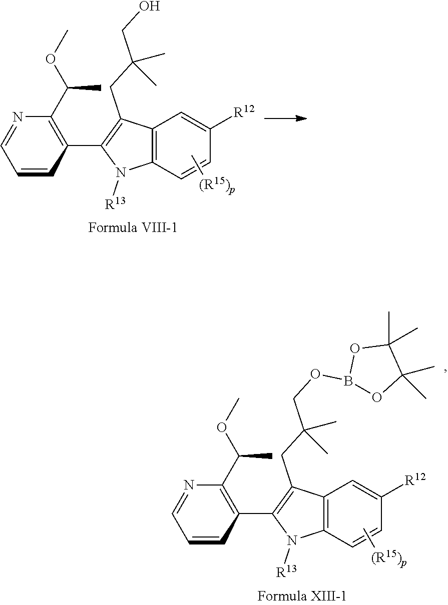

b) hydrolyzing and borylating the compound of Formula XIII-1 in one step to form Formula X-1:

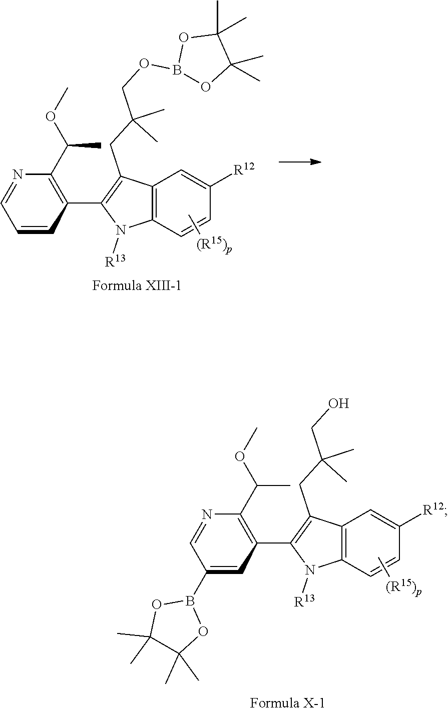

wherein R¹² is halogen;
R¹³ is optionally substituted C₁-C₆ alkyl;
each R¹⁵ is, independently, hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
p is 0, 1, 2, or 3.

In another aspect, the disclosure provides a method of preparing a compound, or a salt thereof, of Formula XIV:

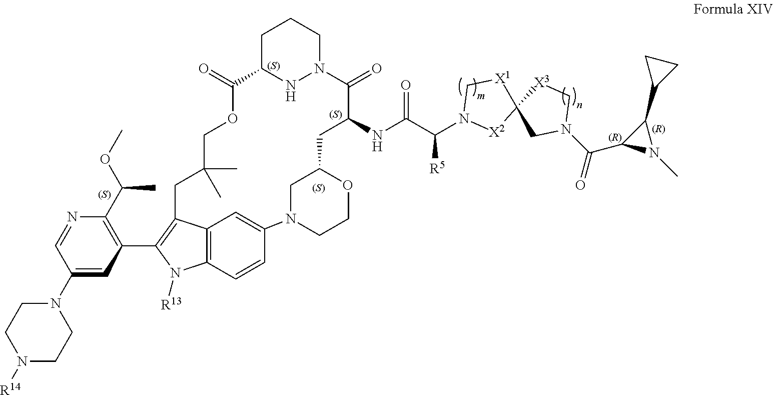

the method including the steps of:
a) reacting (e.g., esterifying) a compound of Formula XV with a compound of Formula XII to form a compound of Formula XVI:

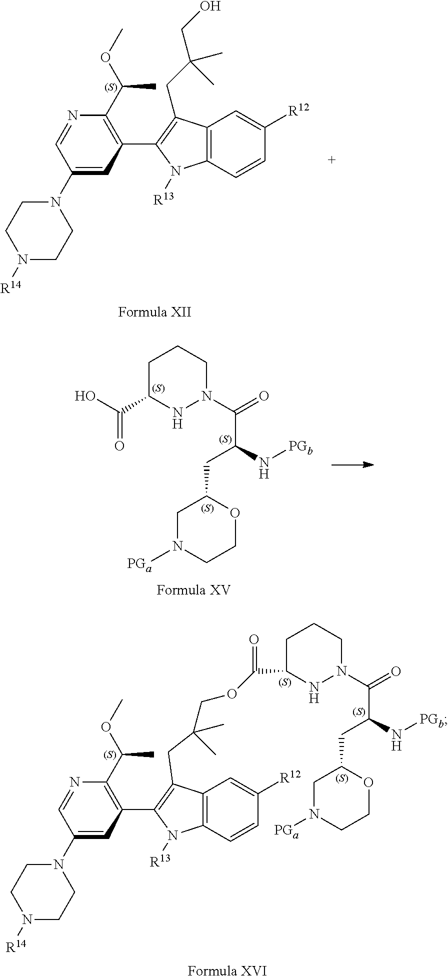

b) removing (e.g., deprotecting) PGₐ of the compound of Formula XVI to form a compound of Formula XVII:

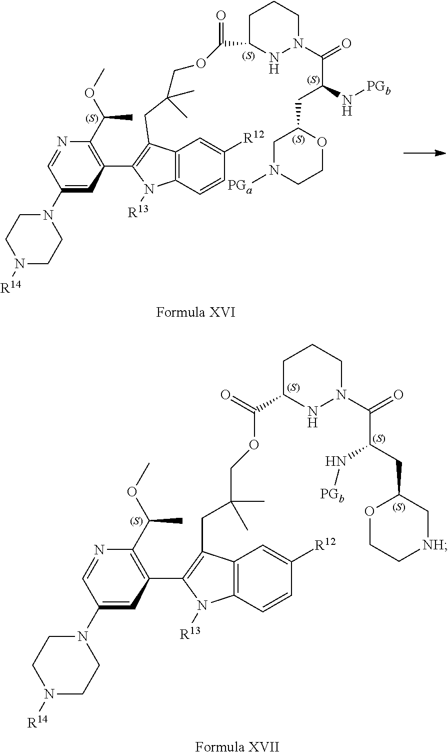

c) cyclizing the compound of Formula XVII with a transition metal catalyst to form a compound of Formula XVIII:

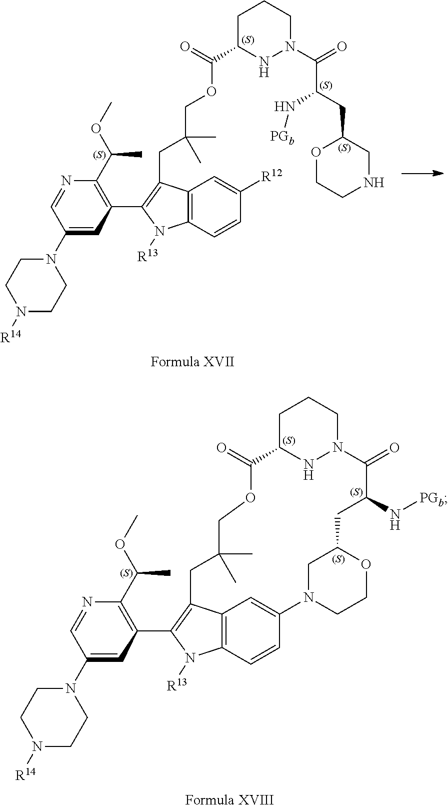

d) removing (e.g., deprotecting) PG_{b} of the compound of Formula XVIII to form a compound of Formula XIX:

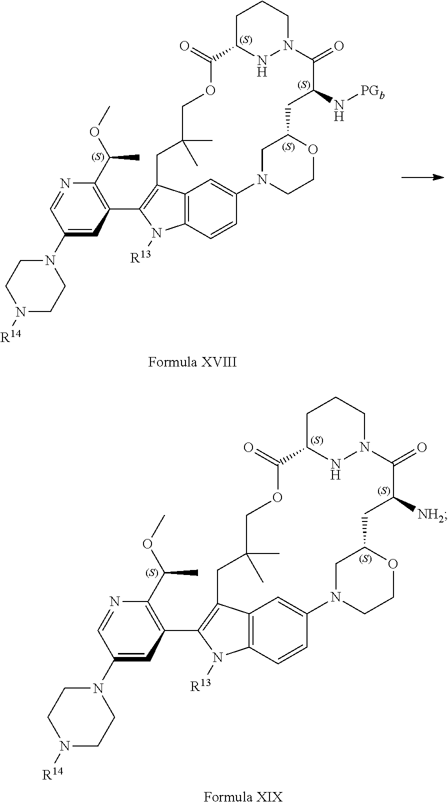

e) coupling the compound of Formula XIX with the compound of Formula IV to form a compound of Formula XIV:

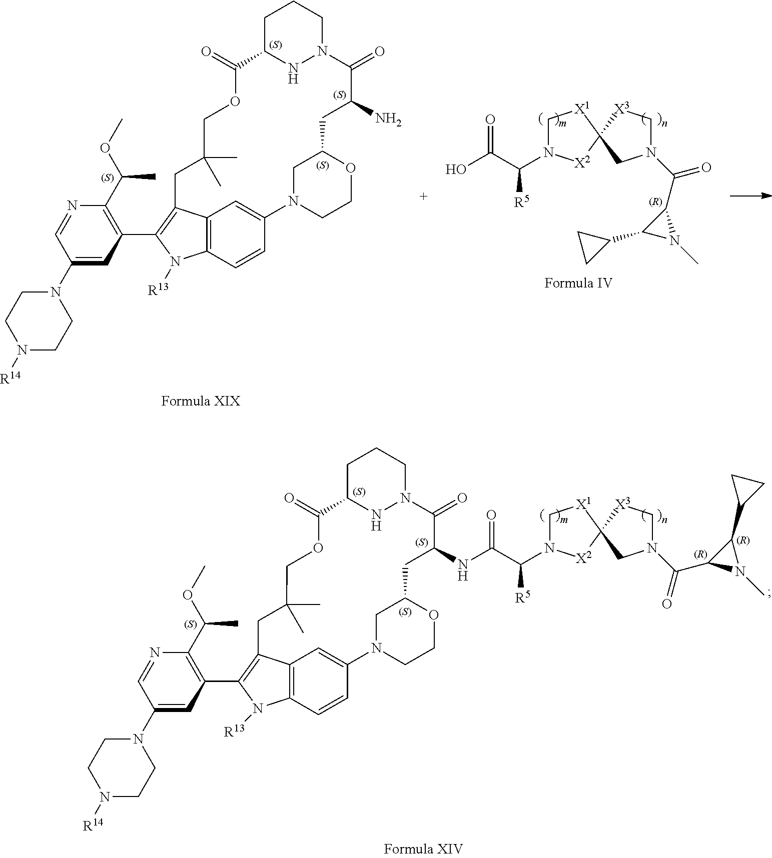

wherein n and m are each, independently, 0, 1, 2, 3, 4, or 5;
X¹, X², and X³ are each, independently, —CH₂—, —CHF—, —CF₂—, —C(O)—, or —O—;
R¹² is halogen;
R¹³ is optionally substituted C₁-C₆ alkyl;
R¹⁴ is optionally substituted 3- to 6-membered cycloalkyl;
PGₐ is an acid-labile protecting group; and PG_{b} is an acid-stable protecting group.

In some embodiments of step (c), the transition metal catalyst includes palladium, platinum, nickel, copper, and iron catalysts. In some embodiments, the transition metal catalyst is a palladium catalyst.

In some embodiments, the disclosure provides a method of preparing a compound of Formula XVI. The method includes reacting a compound of Formula XV with a compound of Formula XII under conditions sufficient to form a compound of Formula XVI. The compound of Formula XV can be prepared in accordance with the processes described herein.

In some embodiments, the disclosure provides a method of preparing a compound of Formula XVIII. The method includes cyclizing a compound of Formula XVII in the presence of transition metal catalyst, such as a palladium catalyst, to form a compound of Formula XVIII. The compound of Formula XVII can be prepared in accordance with the processes described herein.

In some embodiments, the disclosure provides a method of preparing a compound of Formula XIV. The method includes reacting a compound of Formula XIX with a compound of Formula IV under conditions sufficient to form a compound of Formula XIV. The compound of Formula XIX and the compound of Formula IV can be prepared in accordance with the processes described herein.

In another aspect, the disclosure provides a method of preparing a compound, or a salt thereof, of Formula XIV-1:

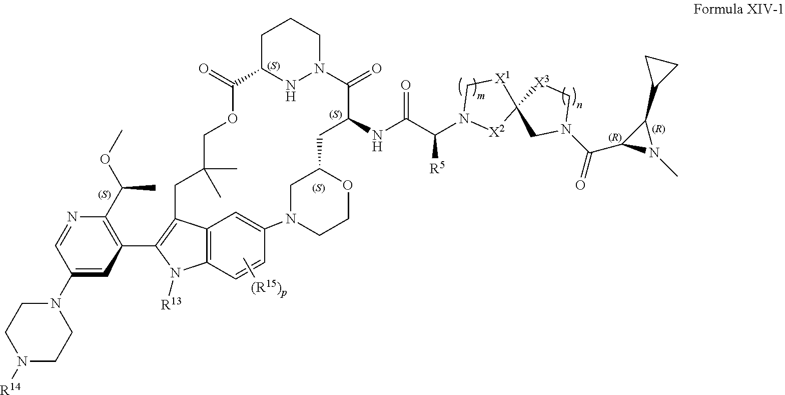

the method including the steps of:
a) reacting (e.g., esterifying) a compound of Formula XV with a compound of Formula XII-1 to form a compound of Formula XVI-1:

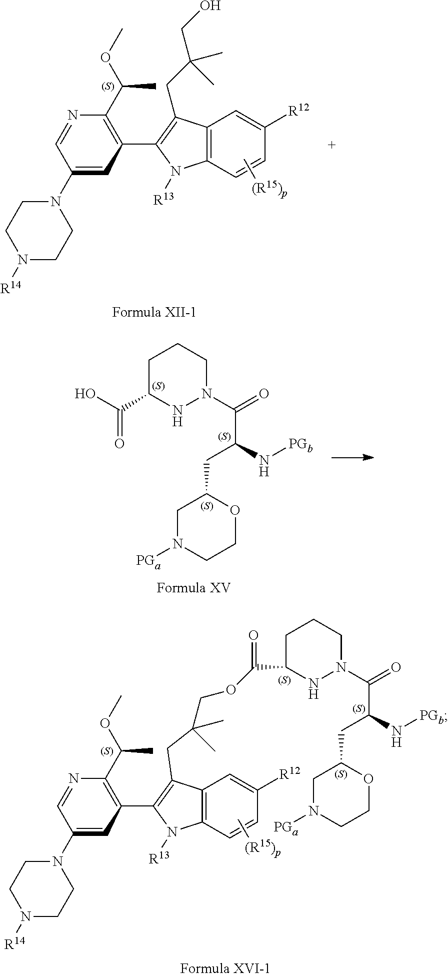

b) removing (e.g., deprotecting) PGₐ of the compound of Formula XVI-1 to form a compound of Formula XVII-1:

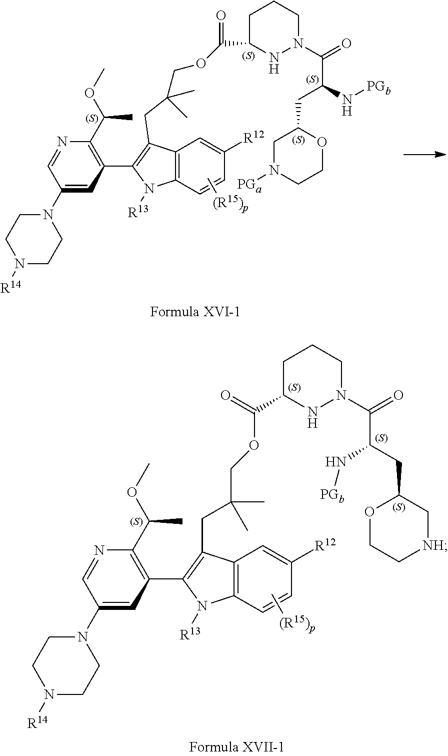

c) cyclizing the compound of Formula XVII-1 with a transition metal catalyst to form a compound of Formula XVII-1:

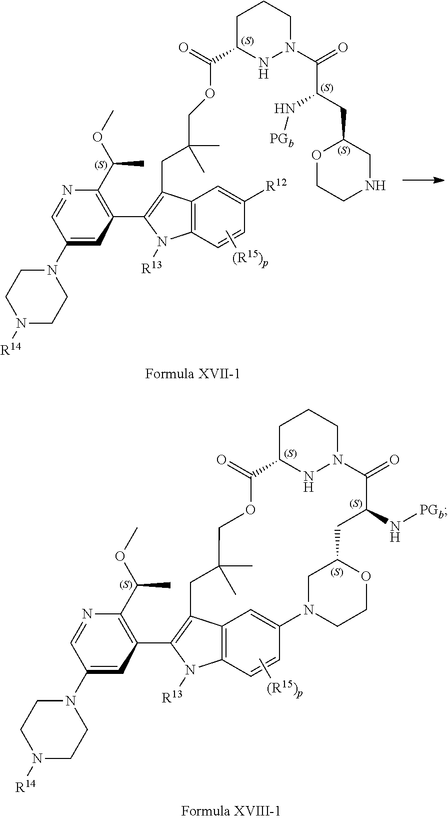

d) removing (e.g., deprotecting) PG_{b} of the compound of Formula XVIII-1 to form a compound of Formula XIX-1:

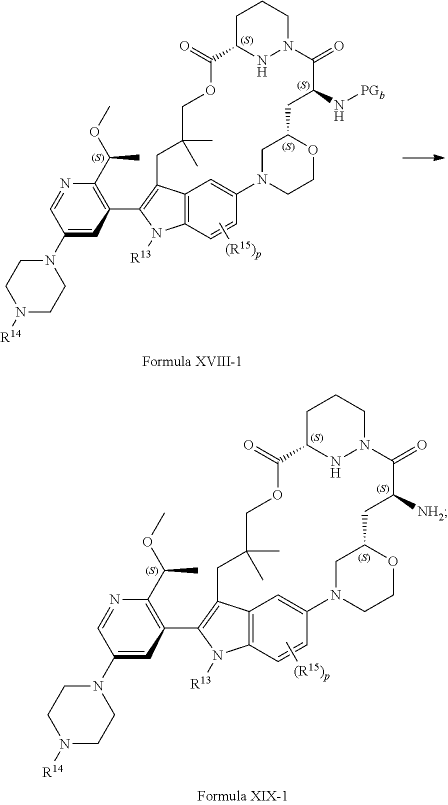

and
e) coupling the compound of Formula XIX-1 with the compound of Formula IV to form a compound of Formula XIV-1:

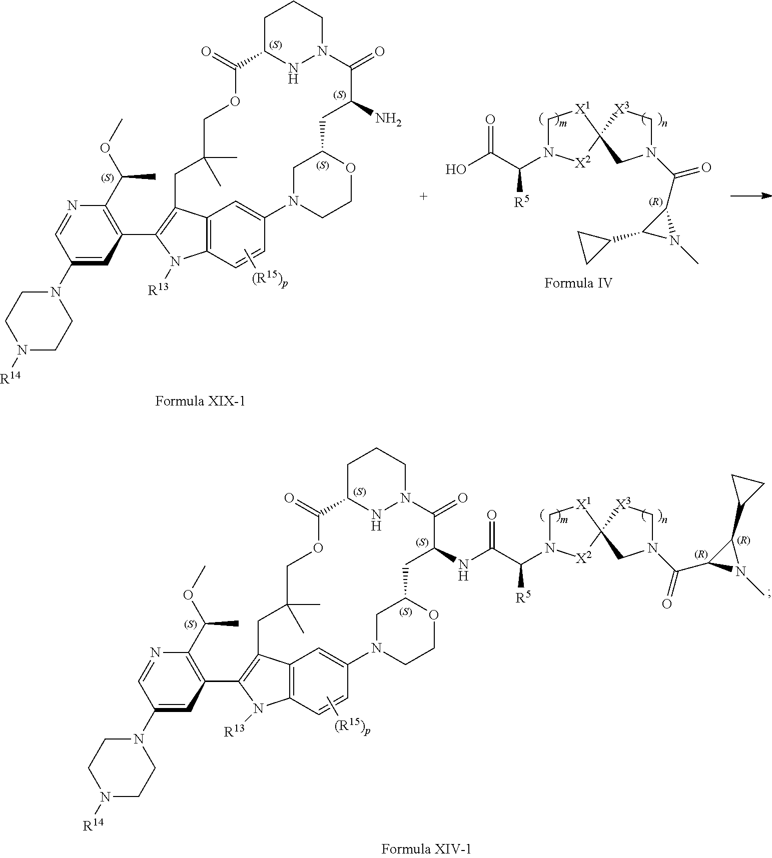

wherein n and m are each, independently, 0, 1, 2, 3, 4, or 5;
X¹, X², and X³ are each, independently, —CH₂—, —CHF—, —CF₂—, —C(O)—, or —O—;
R¹² is halogen;
R¹³ is optionally substituted C₁-C₆ alkyl;
R¹⁴ is optionally substituted 3- to 6-membered cycloalkyl;
each R¹⁵ is, independently, hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
p is 0, 1, 2, or 3;
PGₐ is an acid-labile protecting group; and
PG_{b} is an acid-stable protecting group.

In some embodiments of step (c), the transition metal catalyst includes palladium, platinum, nickel, copper, and iron catalysts. In some embodiments, the transition metal catalyst is a palladium catalyst.

In some embodiments, the compound of Formula XIV is Compound 1:

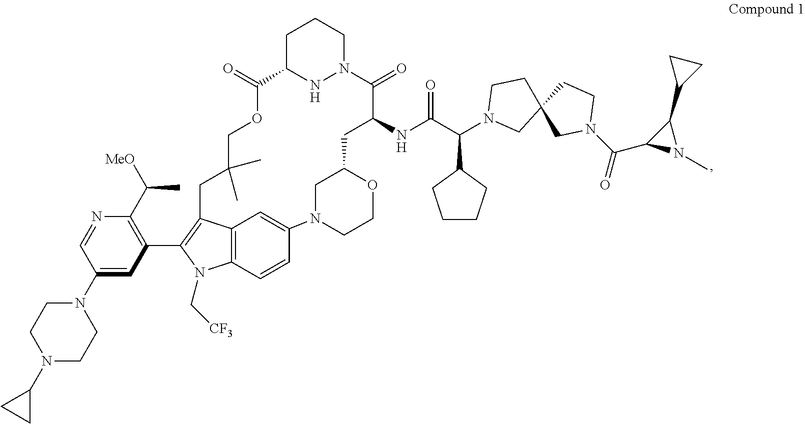

and the method includes the steps of:
a) reacting (e.g., esterifying) a compound of Formula XV with a compound of Formula XII to form a compound of Formula XVI having the following structure:

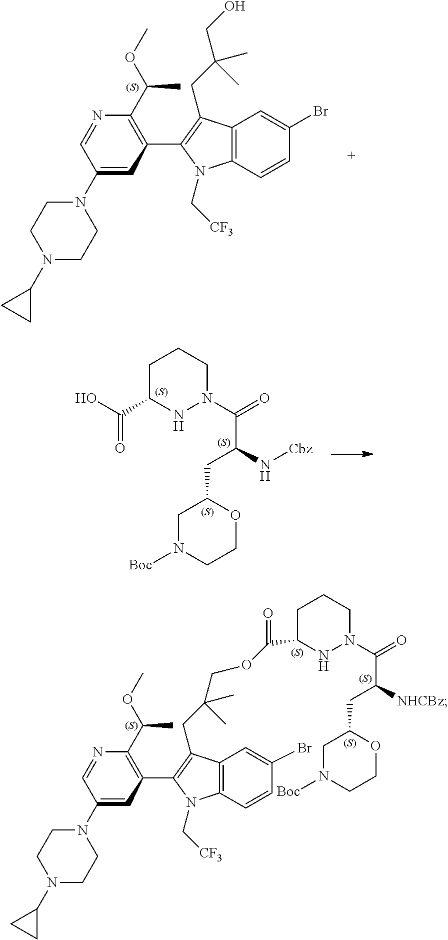

b) deprotecting the Boc of the compound of Formula XVI to form a compound of Formula XVII having the following structure:

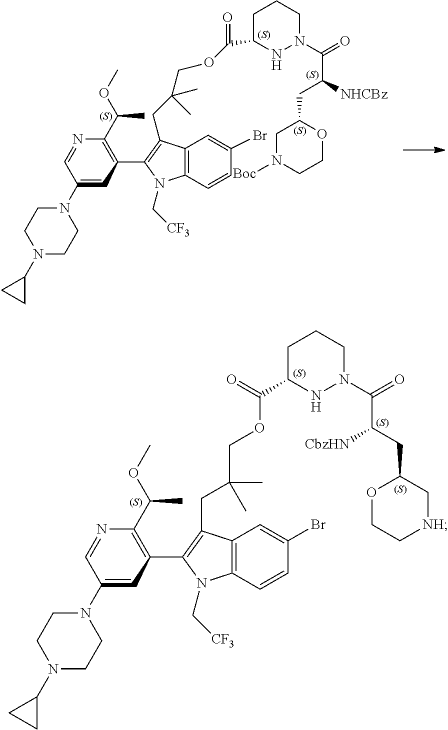

c) cyclizing the compound of Formula XVII with a transition metal catalyst to form a compound of Formula XVII having the following structure:

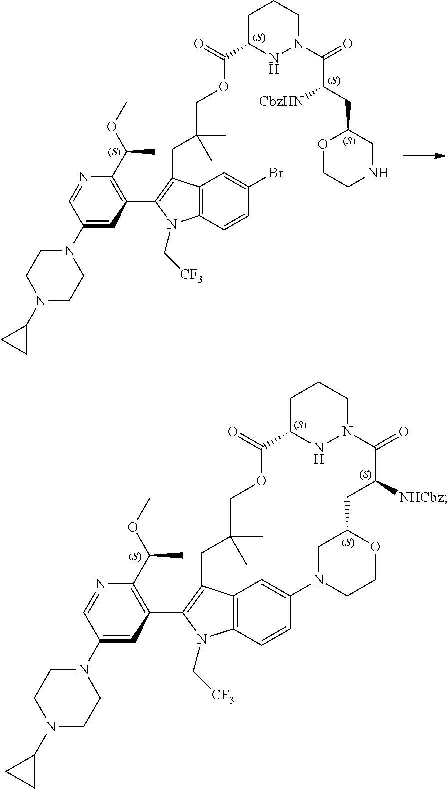

d) removing (e.g., deprotecting) PG_{b} of the compound of Formula XVIII to form a compound of Formula XIX having the following structure:

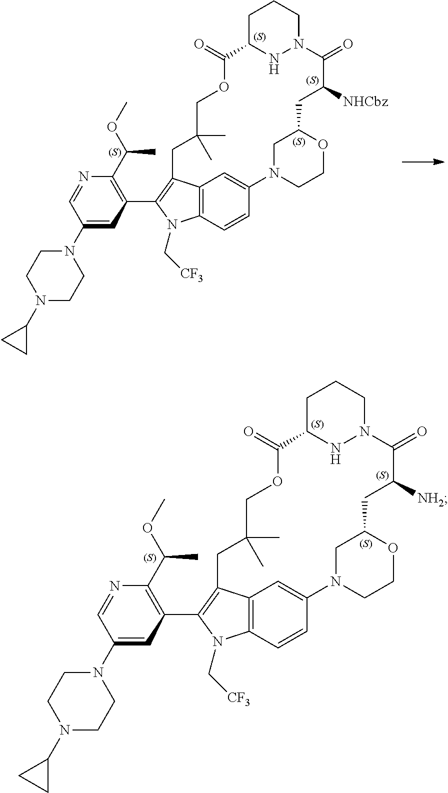

and
e) coupling the compound of Formula XIX with the compound of Formula IV to form Compound 1 having the following structure:

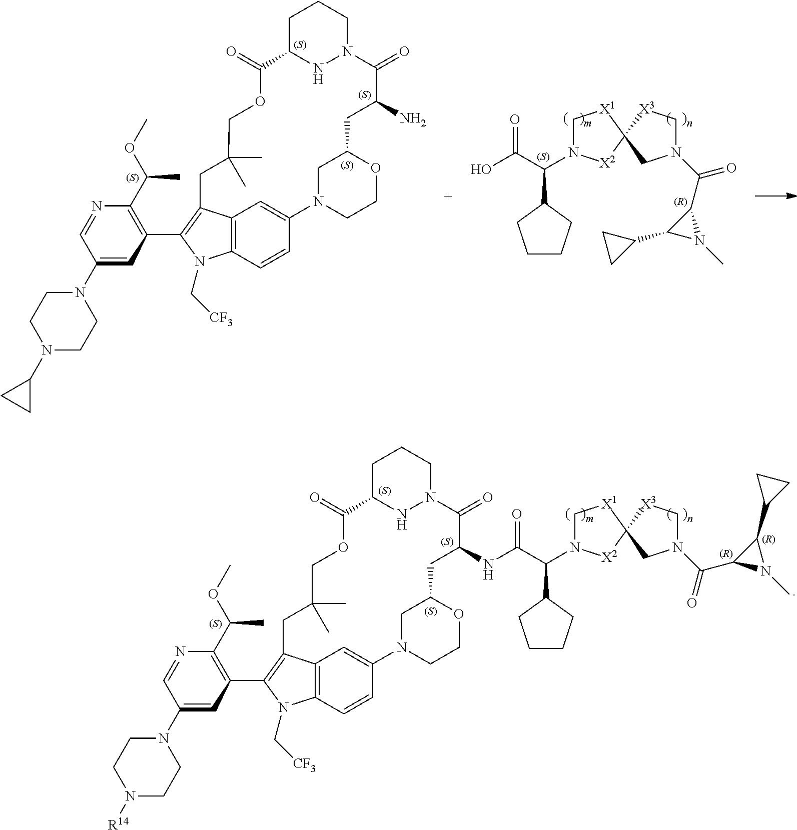

In some embodiments, step (a) includes contacting the compound of Formula XII and the compound of Formula XV in the presence of a carbodiimide coupling reagent, an anti-racemization agent, and a base. In some embodiments, the carbodiimide coupling reagent is EDCl, the anti-racemization agent is HOBt, and the base is N,N-diisopropylethylamine. In some embodiments, the deprotecting step (b) further includes contacting the compound of Formula XVI with an acid. In some embodiments, the acid is trifluoroacetic acid. In some embodiments, the transition metal catalyst of step (c) includes palladium, platinum, nickel, copper, and iron catalysts. In some embodiments, the transition metal catalyst is a palladium catalyst. In some embodiments, the cyclizing step (c) further includes contacting the compound of Formula XVIII with hydrochloric acid to form a hydrochloride salt of the compound of Formula XVIII. In some embodiments, the deprotecting step (d) further includes washing the compound of Formula XIX with N-acetyl cysteine. In some embodiments, step (e) includes coupling the compound of Formula XIX and the compound of Formula IV in the presence of a coupling reagent, an anti-racemization reagent, and a base. In some embodiments, the coupling reagent is PyBOP or EDCl. In some embodiments, the anti-racemization agent is selected from the group consisting of Oxyma, HOBt, or HOPO. In some embodiments, the base is N,N-diisopropylethylamine. In some embodiments, the method further includes the step of purifying the compound of Formula XIV. In some embodiments, the purifying includes recrystallizing the compound of Formula XIV. In some embodiments, the recrystallizing includes adding a first solvent, followed by adding a second solvent. In some embodiments, the first solvent is dioxane or 2-methyltetrahydrofuran. In some embodiments, the second solvent is diisopropyl ether or isopropyl alcohol. In some embodiments, the recrystallizing further includes adding a third solvent. In some embodiments, the third solvent is heptane. In some embodiments, R¹² is —Br. In some embodiments, R¹³ is —CH₂CF₃. In some embodiments, PGₐ is Boc. In some embodiments, PG_{b} is CBz.

In yet another aspect, the disclosure provides a compound of Formula IIf-1:

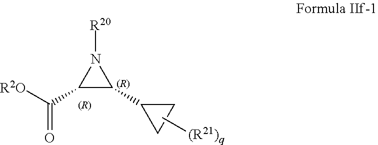

or a salt thereof, wherein
R² is optionally substituted C₁₋₆ alkyl, optionally substituted C₃₋₆ cycloalkyl, or optionally substituted C₆₋₁₀ aryl;
R²⁰ is hydrogen or C₁₋₆ alkyl optionally substituted with 1 or 2 substituents independently selected from CN, OH, C₁₋₆ alkoxy, and C₁₋₆ haloalkyl;
R²¹ is OH, halogen, CN, C₁₋₆ alkyl, C₁₋₆ alkoxy or C₁₋₆ haloalkyl; and
q is 0, 1, or 2.

In some embodiments, R² is benzyl, methyl, or t-Butyl. In some embodiments, R² is benzyl.

In some embodiments, the compound of Formula IIf-1 has the structure of Formula IIe or a salt thereof:

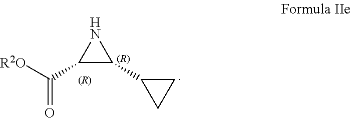

In some embodiments, R² is benzyl, methyl, or t-Butyl. In some embodiments, R² is benzyl.

In some embodiments, the compound of Formula IIf-1 has the structure of Formula IIf or a salt thereof:

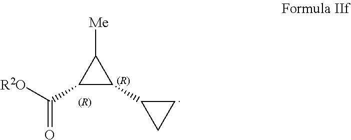

In some embodiments, R² is benzyl, methyl, or t-Butyl. In some embodiments, R² is benzyl.

In yet another aspect, the disclosure provides a method of preparing a compound of Formula IIf:

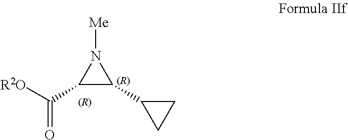

the method including alkylating a compound of Formula IIe with a methylating agent:

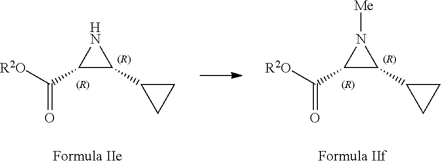

wherein R² is optionally substituted C₁₋₆ alkyl, optionally substituted C₃₋₆ cycloalkyl, or optionally substituted C₆₋₁₀ aryl.

In some embodiments, the methylating agent includes trimethylboroxine. In some embodiments, the methylating agent includes trimethylboroxine and Cu(OAc)₂. In some embodiments, the methylating agent includes trimethylboroxine and CuOAc.

In yet another aspect, the disclosure provides a method of preparing a compound of Formula IVa:

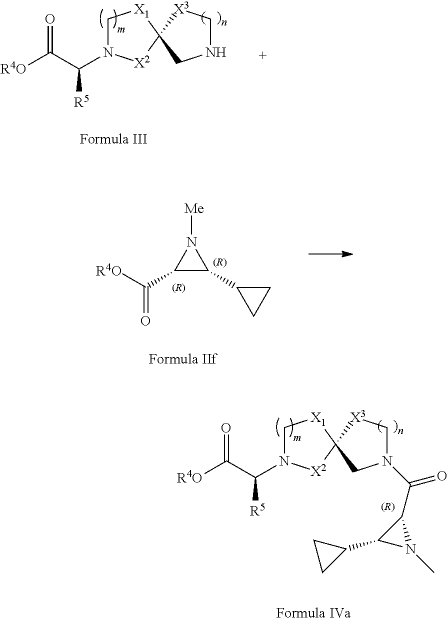

the method including coupling a compound of Formula III with a compound of Formula IIf
wherein n and m are each, independently, 0, 1, 2, 3, 4, or 5;
each R⁴ is independently optionally substituted C₁-C₆ alkyl or optionally substituted C₆-C₁₀ aryl;
each R⁵ is independently optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

In some embodiments, R⁴ is benzyl, methyl, ort-Butyl.

In some embodiments, R⁵ is isopropyl, cyclopropyl, cyclobutyl, cyclopentyl, or cyclohexyl. In some embodiments, R⁵ is cyclopentyl.

In some embodiments, the coupling includes contacting the compound of Formula III and the compound of Formula IIf with a base. In some embodiments, the base is 1,5,7-triazobicyclo[4,4,0]dec-5-ene (TBD).

In yet another aspect, the disclosure provides a method of preparing a compound of Formula VIIIc:

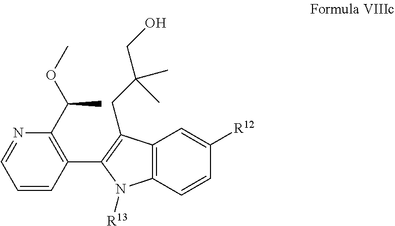

the method including N-alkylating a compound of Formula VIIIe with a compound having the structure of R¹³-LG¹:

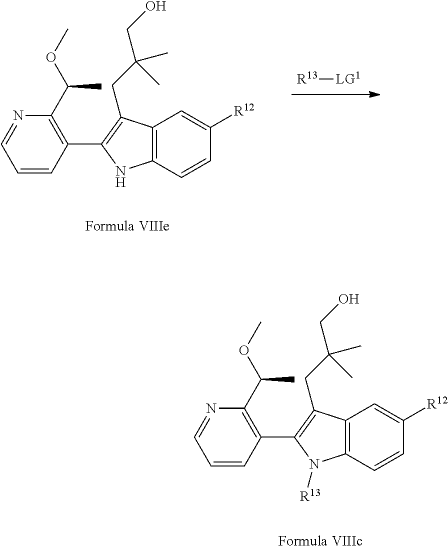

wherein each R¹² is independently halogen;
each R¹³ is independently optionally substituted C₁-C₆ alkyl; and
LG¹ is a leaving group.

In some embodiments, each R¹² is Br.

In some embodiments, each R¹³ is —CH₂CF₃.

In some embodiments, LG¹ is halogen, triflate, mesylate, or tosylate.

In some embodiments, R¹³-LG is 2,2,2-trifluoroethyl trifluoromethanesulfonate.

In yet another aspect, the disclosure provides a method of preparing a compound of Formula VIIIe:

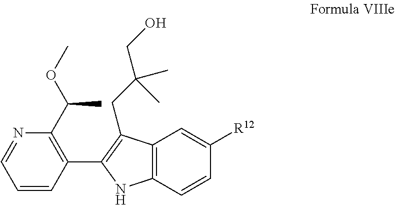

the method including reducing a compound of Formula VIIIf with a reducing agent:

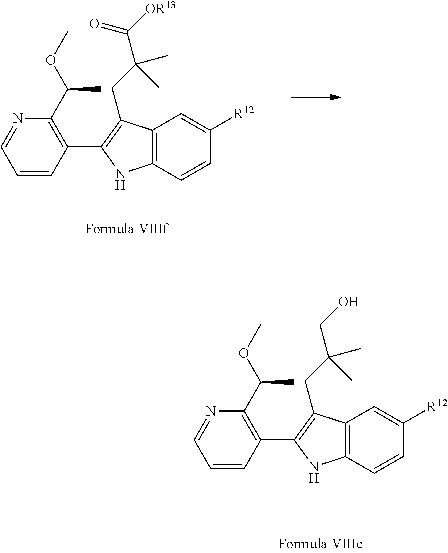

wherein R¹² is halogen; and
R¹³ is optionally substituted C₁-C₆ alkyl.

In some embodiments, each R¹² is Br.

In some embodiments, each R¹³ is —CH₂CF₃.

In some embodiments, the reducing agent is NaBH₄.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein (e.g., Compound A) and intermediates in the synthesis thereto, as well as salts (e.g., pharmaceutically acceptable salts), solvates, hydrates, stereoisomers (including atropisomers), and tautomers thereof.

As used herein, the term “nitrogen protecting group” refers to a removable chemical moiety on a nitrogen atom in a compound described herein that protects the nitrogen from undesirable side reactions. Non-limiting examples of nitrogen protecting groups include Boc (tert-Butyloxycarbonyl), Fmoc (fluorenylmethyloxycarbonyl), and Cbz (benzyl carbamate).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄₀(CH₂)₀₋₁Ph which may be substituted with R^{∘}; —CH═CHPh, which may be substituted with R^{∘}; —(CH₂)₀₋₄₀(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R⁰)—S(O)₂—R⁰; —C(NCN)NR^{∘}₂; —(CH₂)₀₋₄C(O)SR^{∘}; —(CH₂)₀₋₄C(O)OsiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, -OsiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted -Oph, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cr-Cy alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N. or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, recrystallizing the compound as a chiral salt complex, or recrystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

Provided herein are synthetic methods and intermediates for making Ras inhibitors, or salts thereof. The methods and intermediates can be useful for achieving a higher yield, a higher chemical purity, and/or a higher stereoisomeric purity, and a lower cost for the preparation of the Ras inhibitors or the intermediates. Further synthetic details are provided in the Examples.

The compounds described herein may be prepared using the methods described herein and/or using known organic, inorganic, or enzymatic processes. The synthetic methods may employ the use of commercially available starting materials or starting materials prepared by processes known to those skilled in the art of organic synthesis. These methods include but are not limited to those methods described in the Schemes below and in WO 2021/091967, WO 2022/235870, and WO 2023/060253 the disclosure of each of which is incorporated herein by reference.

### Synthetic Methods

In one aspect, the disclosure provides a method of separating enantiomers of a compound of Formula I

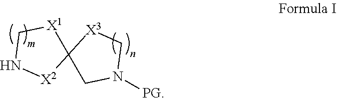

The method includes the steps of:
a) contacting the compound of Formula I with a chiral acid to form a diastereomeric salt of the compound of Formula I; and
b) separating each diastereomer of the diastereomeric salt, thereby separating enantiomers of a compound of Formula I
   wherein
n and m are each, independently, 0, 1, 2, 3, 4, or 5;
PG is a nitrogen protecting group;
and
X¹, X², and X³ are each, independently, —CH₂—, —CHF—, —CF₂—, —C(O)—, or —O—.

In some embodiments, the nitrogen protecting group is Boc (tert-Butyloxycarbonyl). In some embodiments, the chiral acid is a chiral carboxylic acid. In some embodiments, the chiral carboxylic acid is a chiral dicarboxylic acid. In some embodiments, the chiral dicarboxylic acid is tartaric acid. In some embodiments, the chiral carboxylic acid is L(+)-tartaric acid. In some embodiments, the separating step (b) includes recrystallization.

In some embodiments, the compound of Formula I is further described by the compound of Formula Ia:

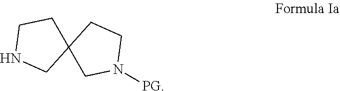

In some embodiments, the compound of Formula I or Formula Ia is Compound B:

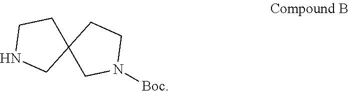 [SMILES: CC(C)(C)OC(=O)N1CCC2(CCNC2)C1]

In another aspect, the disclosure provides a barium salt of Compound A:

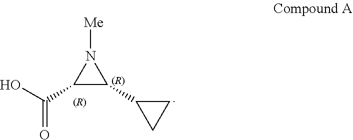

In some embodiments, the barium salt is a 2:1 (compound A: barium) salt.

In a further aspect, the disclosure provides a method of preparing the barium salt of Compound A. The method includes the steps of:
a) condensing a compound of Formula IIa and Compound C to form a compound of Formula IIb:

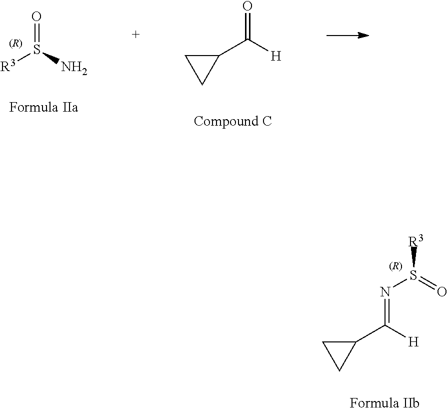

b) cyclizing the compound of Formula IIb and a compound of Formula IIc to form a compound of Formula IIId:

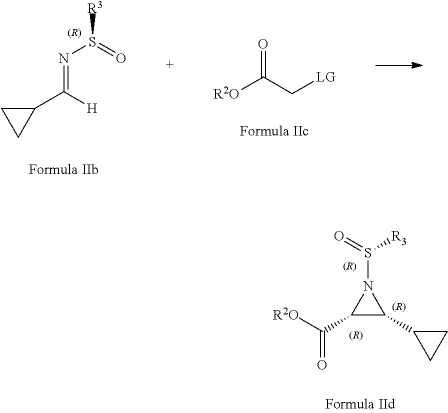

c) hydrolyzing the compound of Formula IIId to form a compound of Formula IIe:

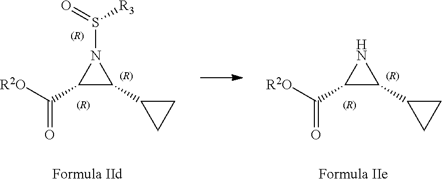

d) methylating the compound of formula IIe to form a compound of formula IIf:

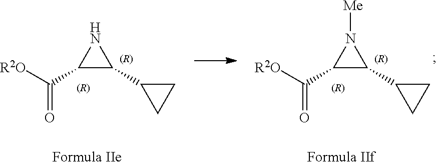

and
e) hydrolyzing the compound of Formula IIf to form the barium salt of Compound A:

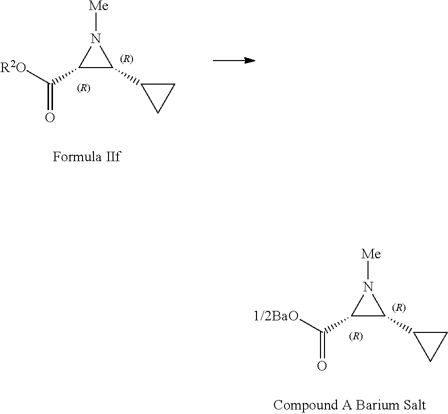

wherein R² and R³ are each, independently, optionally substituted C₁-C₆ alkyl, optionally substituted C₃₋₆ cycloalkyl, or optionally substituted C₆-C₁₀ aryl, and LG is a leaving group selected from the group consisting of halogen, triflate, mesylate, and tosylate. In some embodiments, R³ is optionally substituted C₆-C₁₀ aryl. In some embodiments, R³ is

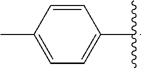 [SMILES: *c1ccc(C)cc1 ; "."]

In some embodiments, R³ is optionally substituted C₁-C₆ alkyl. In some embodiments, R³ is tert-butyl. In some embodiments, R² is optionally substituted C₁-C₆ alkyl, optionally substituted C₃₋₆ cycloalkyl, or optionally substituted C₆₋₁₀ aryl. In some embodiments, R² is benzyl, methyl, or t-Butyl. In some embodiments, LG is halogen. In some embodiments, LG is —Br.

In some embodiments of the method of preparing the barium salt of Compound A, the condensing step (a) includes contacting the compound of Formula IIa and Compound C with a base. In some embodiments, the base is an amine base. In some embodiments, the base is pyrrolidine. In some embodiments, the condensing step (a) is carried out according to the following scheme:

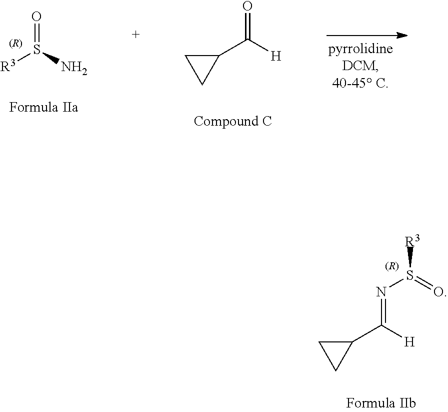

In some embodiments, the condensing step (a) is carried out according to the following scheme:

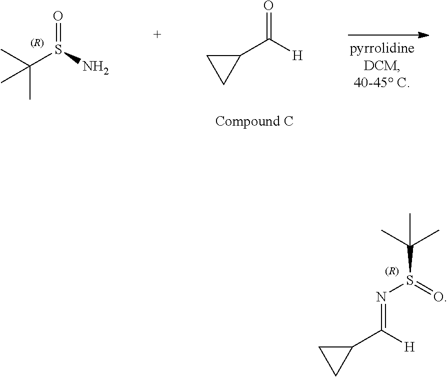

In some embodiments, the condensing step (a) is carried out according to the following scheme:

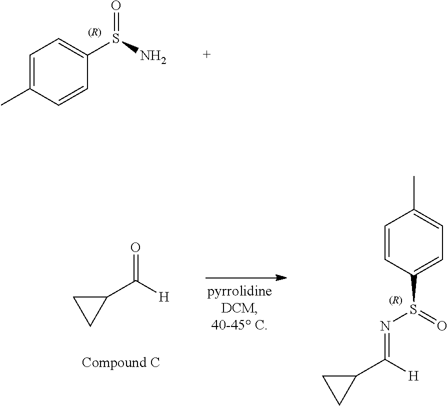

In some embodiments, the cyclizing step (b) includes contacting the compound of Formula IIb and the compound of Formula IIc with a base. In some embodiments, the base is a lithium base. In some embodiments, the base is LiHMDS.

In some embodiments, the cyclizing step (b) is carried out according to the following scheme:

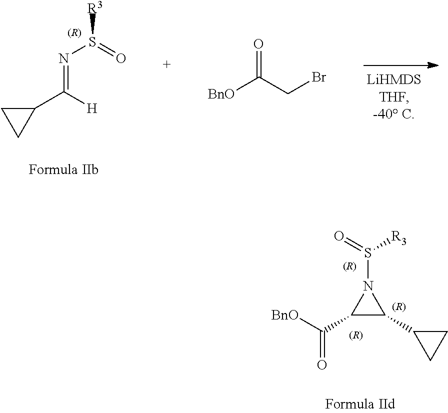

In some embodiments, the hydrolyzing step (c) includes contacting the compound of formula Ild with an acid. In some embodiments, the acid is a carboxylic acid. In some embodiments, the carboxylic acid is trifluoroacetic acid. In some embodiments, the acid is hydrochloric acid.

In some embodiments, the hydrolyzing step (c) is carried out according to the following scheme:

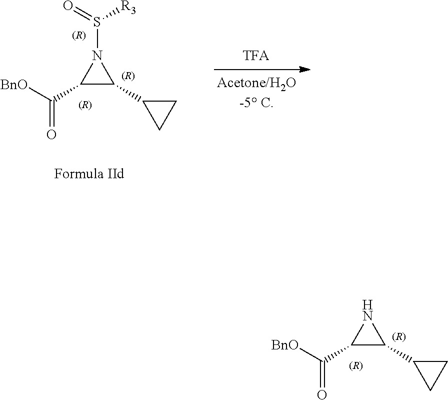

In some embodiments, the hydrolyzing step (c) is carried out according to the following scheme:

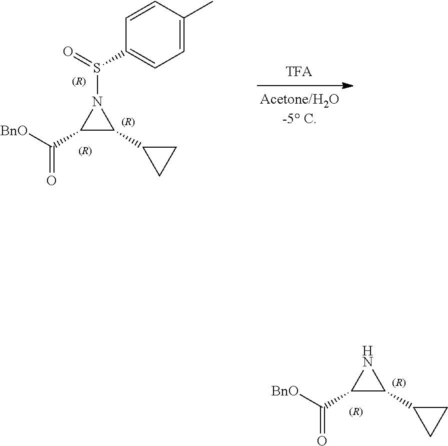

In some embodiments, the hydrolyzing step (c) is carried out according to the following scheme:

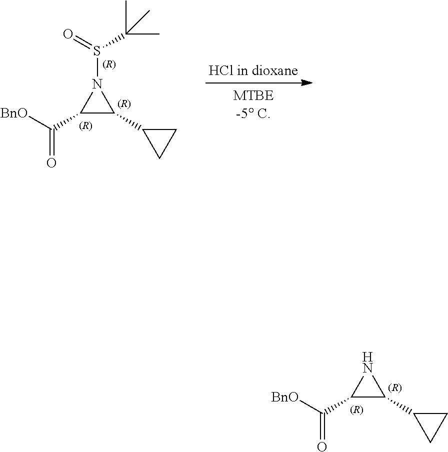

In some embodiments, the methylating step (d) includes contacting the compound of Formula IIe with MeB(OH)₂. In some embodiments, the methylating step (d) further includes contacting the compound of Formula IIe with a copper catalyst. In some embodiments, the methylating step (d) is carried out according to the following scheme:

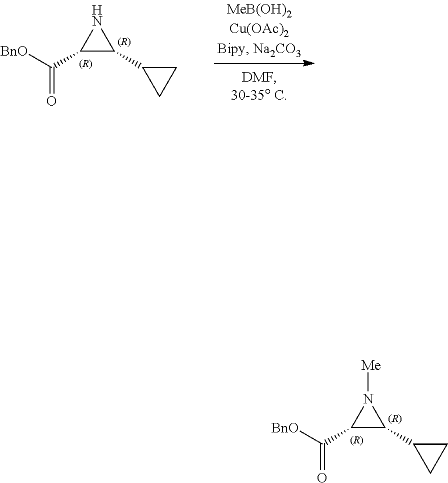

In some embodiments, the hydrolyzing step (e) includes contacting the compound of Formula IIf with barium hydroxide.

In some embodiments, the hydrolyzing step (e) is carried out according to the following scheme:

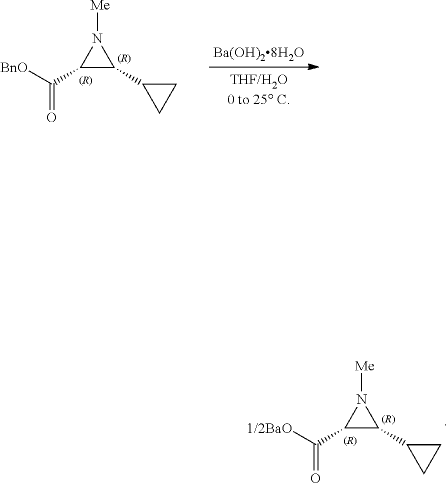

In yet another aspect, the disclosure provides a method of preparing a compound of Formula IIf:

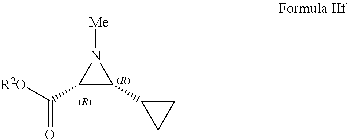

the method including alkylating a compound of Formula IIe with a methylating agent:

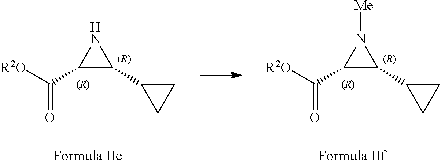

wherein R² is optionally substituted C₁₋₆ alkyl, optionally substituted C₃₋₆ cycloalkyl, or optionally substituted C₆₋₁₀ aryl.

In some embodiments, the methylating agent includes trimethylboroxine. In some embodiments, the methylating agent includes trimethylboroxine and Cu(OAc)₂. In some embodiments, the methylating agent includes trimethylboroxine and CuOAc.

In some embodiments of the method of preparing the compound of Formula IIf, the alkylating step includes contacting the compound of Formula IIe with an alkaline salt. In some embodiments, the alkaline salt is potassium carbonate (K₂CO₃) or sodium carbonate (Na₂CO₃). In some embodiments, the alkylating step includes 2,2′-bipyridine (bipy). In some embodiments, the alkylating step includes di-tert-butyl peroxide (DTBP). In some embodiments, the alkylating step is carried out a temperature of at least 25° C. (e.g., 25, 30, 35, or 40° C.). In some embodiments, the alkylating step is carried out in an organic solvent (e.g., acetonitrile (ACN)). In some embodiments of the method of preparing the compound of Formula IIf, the alkylating step is carried out according to the following scheme:

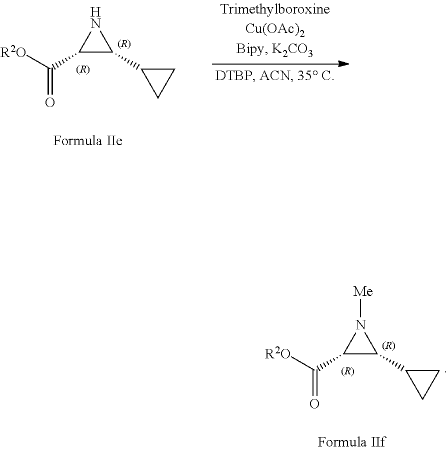

In some embodiments of the method of preparing the compound of Formula IIf, the alkylating step is carried out according to the following scheme:

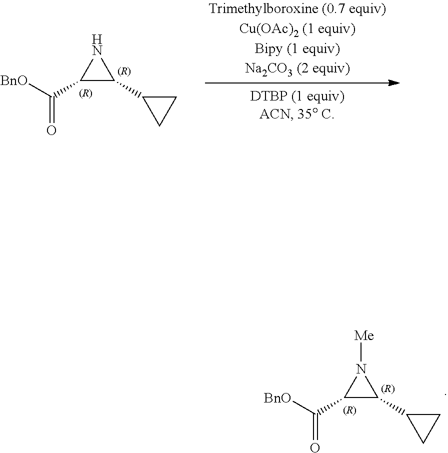

In another aspect, the disclosure provides a method of preparing a compound, or a salt thereof, of Formula III:

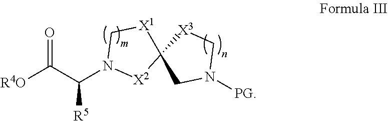

The method includes the steps of:
a) coupling a compound of Formula IIId with a compound of Formula I to form a compound of Formula IIIe:

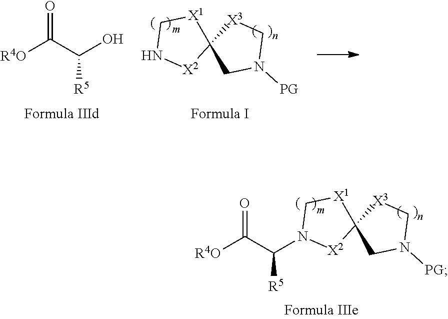

and
b) deprotecting the compound of Formula IIIe to form the compound of Formula III:

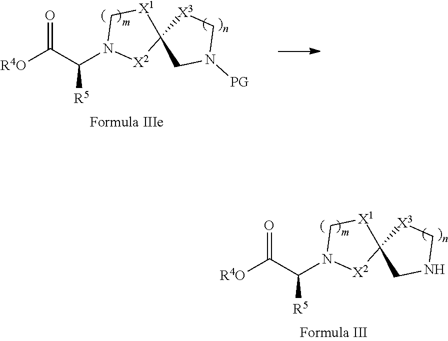

wherein
n and m are each, independently, 0, 1, 2, 3, 4, or 5;
PG is a nitrogen protecting group;
each R⁴ is independently, optionally substituted C₁-C₆ alkyl or optionally substituted C₆-C₁₀ aryl; and
R⁵ is optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and X¹, X², and X³ are each, independently, —CH₂—, —CHF—, —CF₂—, —C(O)—, or —O—.

In some embodiments, the nitrogen protecting group is Boc (tert-Butyloxycarbonyl). In some embodiments, the compound of Formula IIId is prepared by a method including the steps of:
A1) combining a compound of Formula IIIa and a compound of Formula IIIb to form a compound of Formula IIIc:

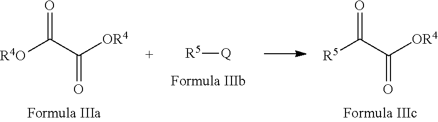

B1) reducing the compound of Formula IIIc to form the compound of Formula IIId:

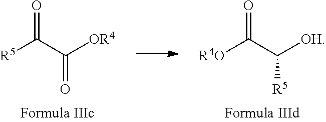

wherein Q is MgBr, MgCl, MgI, or Li.

In some embodiments, the combining step (A1) includes contacting the Compound of Formula IIIa with a compound of Formula IIIb, wherein Q is MgBr. In some embodiments, the contacting is carried out in tetrahydrofuran at −78° C.

In some embodiments, the reducing step (B1) includes contacting the compound of Formula IIIc with a ketoreductase enzyme.

In some embodiments, the compound of Formula IIId is prepared by a method including the steps of:
A2) converting a compound of Formula IIIf to form a compound of Formula IIIg:

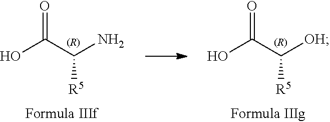

B2) reacting (e.g., esterifying) the compound of Formula IIIg to form the compound of Formula IIId:

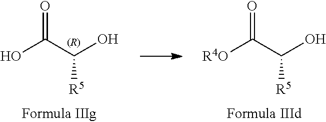

In some embodiments, the converting step (A2) includes forming a diazonium salt of the compound of Formula IIIf. In some embodiments, the converting step (A2) and reacting (e.g., esterifying) step (B2) is carried out according to the following scheme

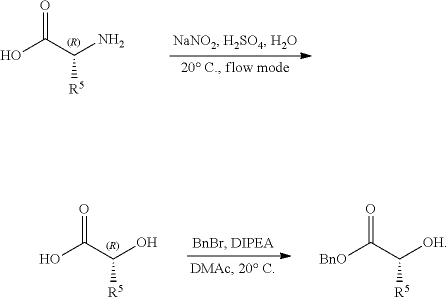

In some embodiments, the combining step (a) of Formula IIId with a compound of Formula I to form a compound of Formula IIIe includes contacting the compound of Formula IIId with triflic anhydride followed by contacting the compound of Formula IIId with the compound of Formula I. In some embodiments, the combining step (a) of Formula IIId with a compound of Formula I to form a compound of Formula IIIe is carried out according to the following scheme:

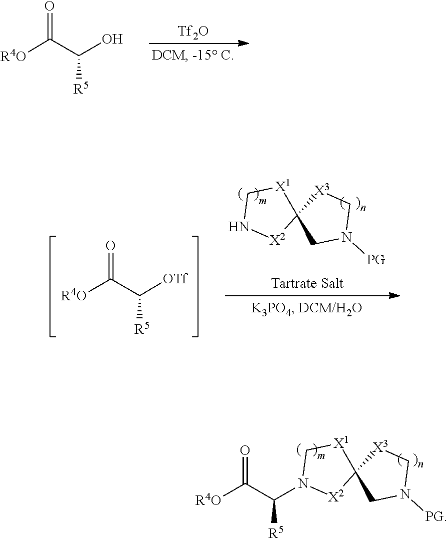

In some embodiments, the deprotecting step (b) includes contacting the compound of Formula Ille with an acid. In some embodiments, the acid is hydrochloric acid. In some embodiments, the hydrochloric acid is delivered an ethyl acetate solution of hydrochloric acid.

In yet another aspect, the disclosure provides a method of preparing a compound of Formula IVa:

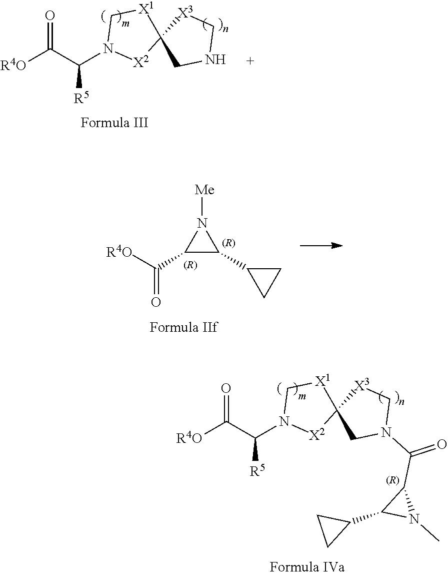

the method including coupling a compound of Formula III with a compound of Formula IIf
wherein n and m are each, independently, 0, 1, 2, 3, 4, or 5;
each R⁴ is independently optionally substituted C₁-C₆ alkyl or optionally substituted C₆-C₁₀ aryl;
each R⁵ is independently optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

In some embodiments, R⁴ is benzyl, methyl, or t-Butyl. In some embodiments, R⁵ is isopropyl, cyclopropyl, cyclobutyl, cyclopentyl, or cyclohexyl. In some embodiments, R⁵ is cyclopentyl.

In some embodiments, the coupling includes contacting the compound of Formula III and the compound of Formula IIf with a base. In some embodiments, the base is 1,5,7-triazobicyclo[4,4,0]dec-5-ene (TBD). In some embodiments, the coupling is carried out in an organic solvent (e.g., methyl tert-butyl ether (MTBE)). In some embodiments of the method of preparing the compound of Formula IVa, the coupling is carried out at a temperature of at least −10° C. (e.g., −10, −5, 0, or 5° C.). In some embodiments of the method of preparing the compound of Formula IVa, the coupling is carried out according to the following scheme:

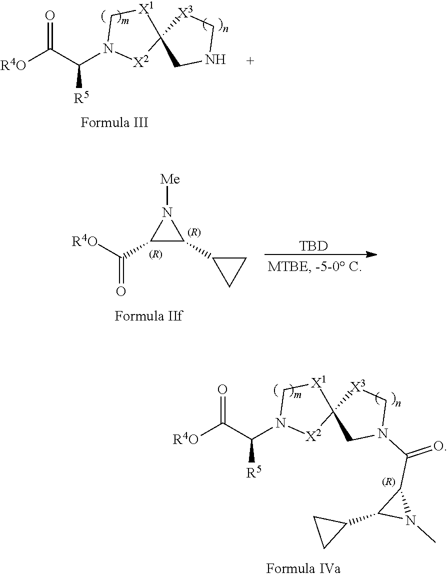

In some embodiments of the method of preparing the compound of Formula IVa, the compound of Formula III is a compound of Formula III-1.

In some embodiments of the method of preparing the compound of Formula IVa, the coupling is carried out according to the following scheme:

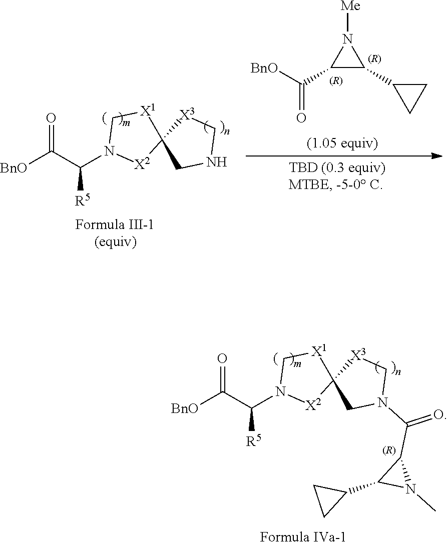

In some embodiments, the compound of Formula III is Compound D:

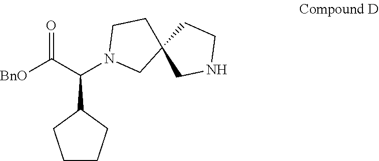 [SMILES: O=C(OCc1ccccc1)[C@H](C1CCCC1)N1CC[C@]2(CCNC2)C1]

which is prepared by:
a) coupling a compound of Formula IIId-1 with Compound B to form a compound of Formula IIIe-1 having the following structure:

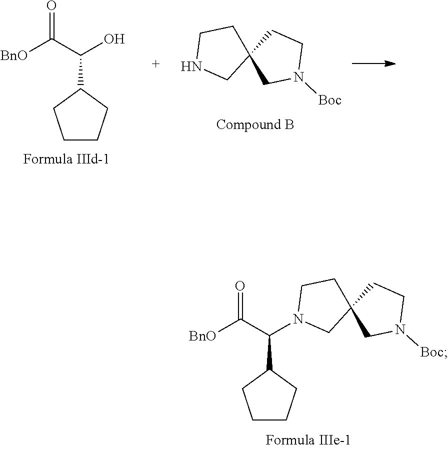 [SMILES: O=C(OCc1ccccc1)[C@H](O)C1CCCC1 ; CC(C)(C)OC(=O)N1CC[C@]2(CCNC2)C1 ; CC(C)(C)OC(=O)N1CC[C@]2(CCN([C@H](C(=O)OCc3ccccc3)C3CCCC3)C2)C1]

and
b) deprotecting the compound of Formula IIIe-1 to form Compound D having the following structure:

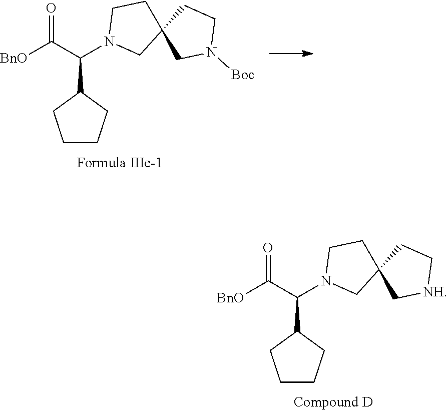 [SMILES: CC(C)(C)OC(=O)N1CC[C@]2(CCN([C@H](C(=O)OCc3ccccc3)C3CCCC3)C2)C1 ; O=C(OCc1ccccc1)[C@H](C1CCCC1)N1CC[C@]2(CCNC2)C1]

In some embodiments, the combining step (a) of Formula IIId with Compound B to form a compound of Formula IIIe includes contacting the compound of Formula IIId with triflic anhydride followed by contacting the compound of Formula IIId with Compound B.

In some embodiments, the combining step (a) of Formula IIId with Compound B to form a compound of Formula IIIe is carried out according to the following scheme:

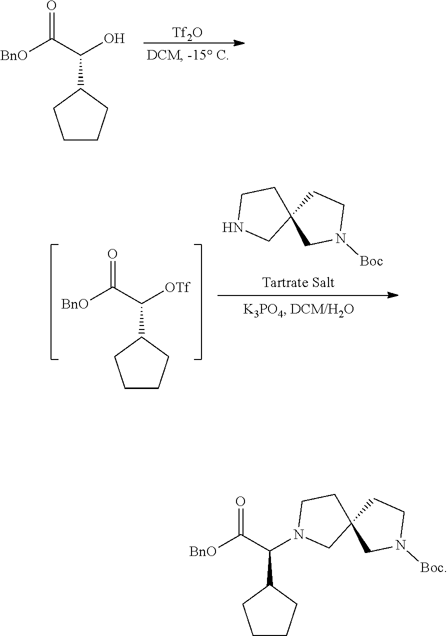

In some embodiments, the deprotecting step (b) includes contacting the compound of Formula Ille with an acid. In some embodiments, the acid is hydrochloric acid. In some embodiments, the hydrochloric acid is delivered an ethyl acetate solution of hydrochloric acid.

In yet another aspect, the disclosure provides a method of preparing a compound, or a salt thereof, of Formula IV:

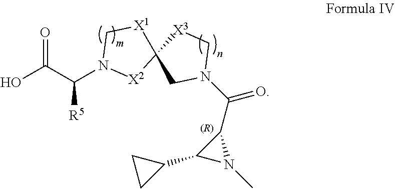

The method includes the steps of:
a) coupling a compound of Formula III and a barium salt of compound A to form a compound of Formula IVa:

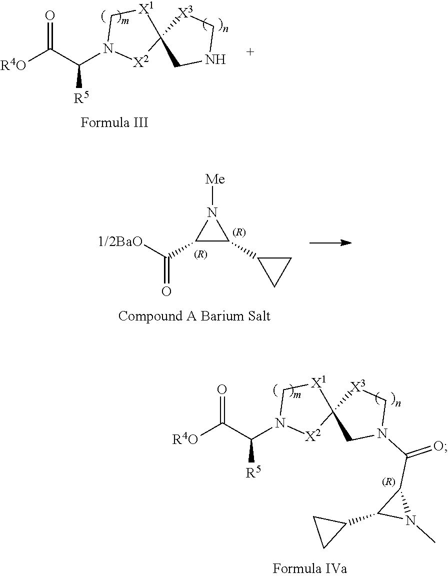

and
b) hydrolyzing the compound of Formula IVa to form the compound of Formula IV:

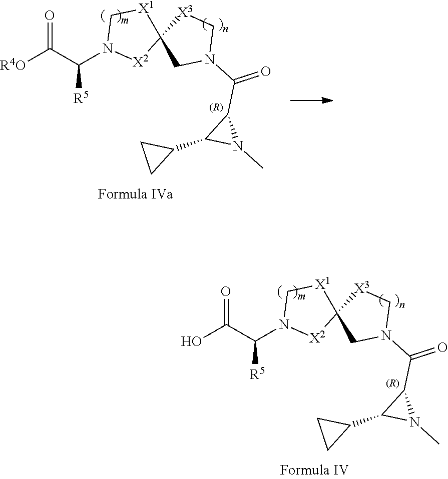

wherein
n and m are each, independently, 0, 1, 2, 3, 4, or 5;
R⁴ is optionally substituted C₁-C₆ alkyl or optionally substituted C₆-C₁₀ aryl;
R⁵ is optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

In some embodiments, the coupling step (a) includes contacting the compound of Formula III and the Compound A barium salt with chloro-N,N,N′,N′-tetramethylformamidinium hexafluorophosphate (TCFH). In some embodiments, the coupling step (a) further includes contacting the compound of Formula III and the Compound A barium salt with NMI. In some embodiments, the coupling step (a) is carried out according to the following scheme:

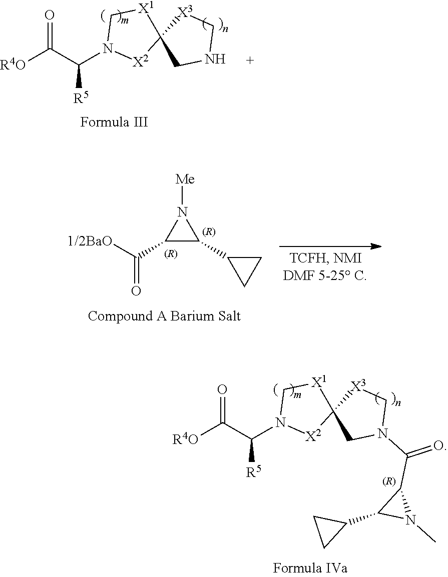

In some embodiments, the hydrolyzing step (b) includes contacting the compound of Formula IVa with an acid or base. In some embodiments, the hydrolyzing step (b) includes hydrogenating the compound of Formula IVa. In some embodiments, the hydrogenating further includes contacting the compound of Formula IVa with a palladium catalyst.

In some embodiments, the hydrolyzing step (b) is carried out according to the following scheme:

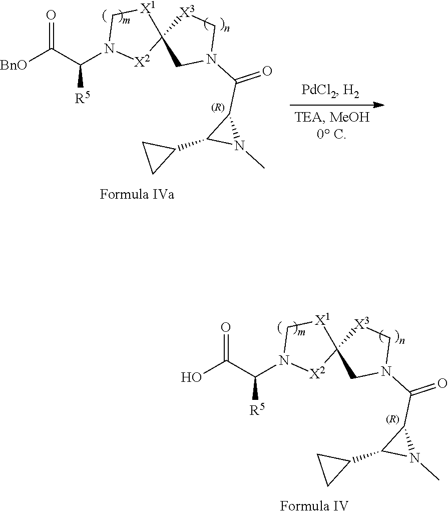

In some embodiments of any of the methods and compounds described herein, m is 0. In some embodiments, m is 1. In some embodiments, m is 2. In some embodiments, n is 0. In some embodiments, n is 1. In some embodiments, n is 2.

In some embodiments of any of the methods and compounds described herein, each of X¹, X², and X³ is CH₂. In some embodiments, PG is

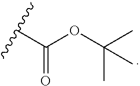 [SMILES: *C(=O)OC(C)(C)C ; "."]

In some embodiments, LG is halogen. In some embodiments, LG is Br.

In some embodiments of any of the methods and compounds described herein, R² is optionally substituted C₁-C₆ alkyl, optionally substituted C₃₋₆ cycloalkyl, or optionally substituted C₆₋₁₀ aryl. In some embodiments, R² is benzyl, methyl, or t-Butyl. In some embodiments, R³ is optionally substituted C₆-C₁₀ aryl. In some embodiments, R³ is

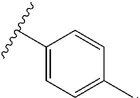 [SMILES: *c1ccc(C)cc1 ; "."]

In some embodiments, R³ is optionally substituted C₁-C₆ alkyl. In some embodiments, R³ is

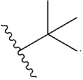 [SMILES: *C(C)(C)C ; "."]

In some embodiments, R⁴ is optionally substituted C₁-C₆ alkyl or optionally substituted C₆-C₁₀ aryl. In some embodiments, R⁴ is benzyl, methyl, or t-Butyl. In some embodiments, R⁵ is optionally substituted C₁-C₆ alkyl or optionally substituted 3- to 6-membered cycloalkyl. In some embodiments, R⁵ is optionally substituted C₁-C₆ alkyl. In some embodiments, R⁵ is isopropyl. In some embodiments, R⁵ is optionally substituted 3- to 6-membered cycloalkyl. In some embodiments, R⁵ is cyclopentyl.

In some embodiments, the method includes preparing Compound E:

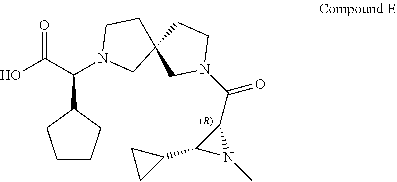

and the method including the steps of:
a) coupling Compound D and a barium salt of compound A to form a compound of Formula IVa having the following structure:

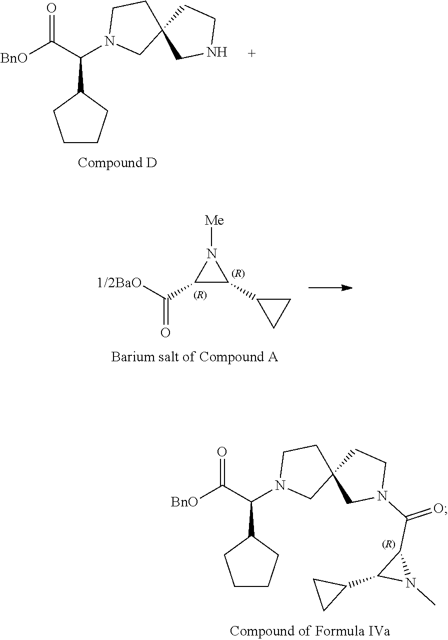

and
b) hydrolyzing the compound of Formula IVa to form Compound E having the following structure:

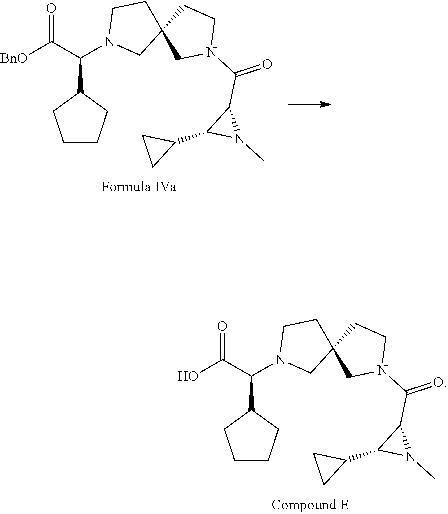

In some embodiments, the coupling step (a) includes contacting Compound D and the Compound A barium salt with chloro-N,N,N′,N′-tetramethylformamidinium hexafluorophosphate (TCFH). In some embodiments, the coupling step (a) further includes contacting Compound D and the barium salt of Compound A with NMI. In some embodiments, the coupling step (a) is carried out according to the following scheme:

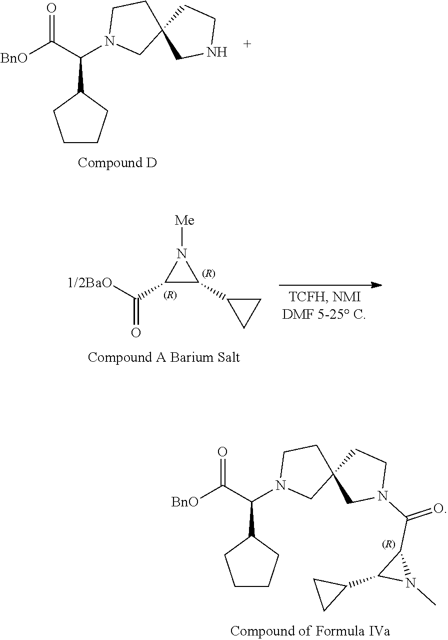

In some embodiments, the hydrolyzing step (b) includes contacting the compound of Formula IVa with an acid or base. In some embodiments, the hydrolyzing step (b) includes hydrogenating the compound of Formula IVa. In some embodiments, the hydrogenating further includes contacting the compound of Formula IVa with a palladium catalyst. In some embodiments, the hydrolyzing step (b) is carried out according to the following scheme:

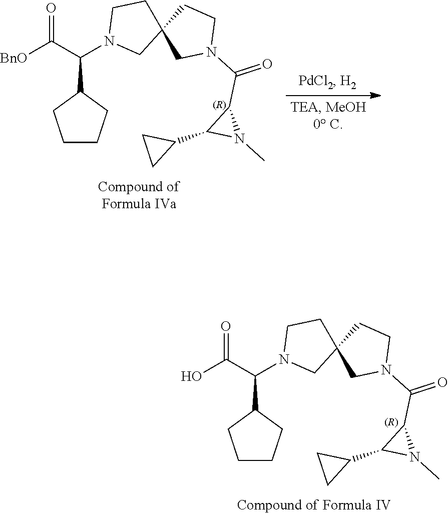

In yet another aspect, the disclosure provides a method of preparing a compound, or a salt thereof, of Formula VIII:

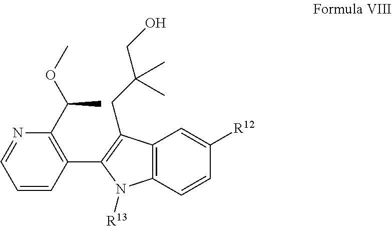

the method including the steps of:
a) esterifying and N-alkylating a compound of Formula VIIIa to form a compound of Formula VIIIb:

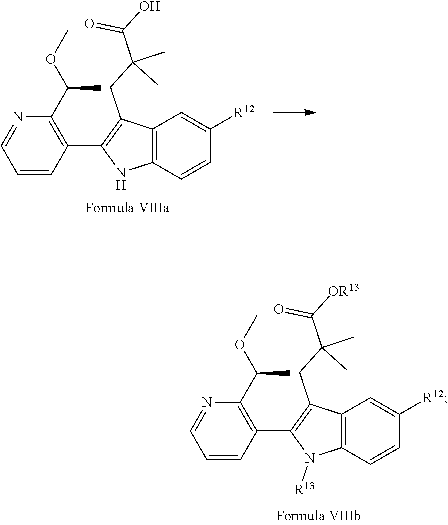

b) reducing the compound of Formula VIIIb to form a compound of Formula VIIIc as a mixture of atropisomers:

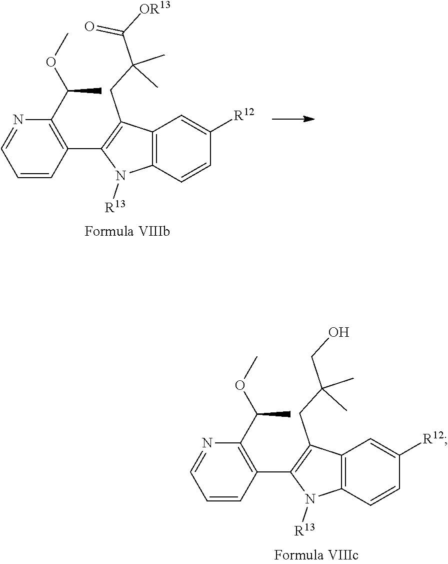

and
c) separating the mixture of atropisomers of Formula VIIIc to obtain a stereochemically pure compound of Formula VIII;
wherein R¹² is halogen; and
R¹³ is optionally substituted C₁-C₆ alkyl.

In some embodiments, the esterifying and N-alkylating step (a) includes contacting the compound of Formula VIIIa with an alkylating agent. In some embodiments, the alkylating agent is R¹³-LG, wherein R¹³ is optionally substituted C₁-C₆ alkyl and LG is a leaving group. In some embodiments, the alkylating agent is 2,2,2-trifluoroethyl trifluoromethanesulfonate.

In some embodiments, the esterifying and N-alkylating step (a) is carried out according to the following scheme:

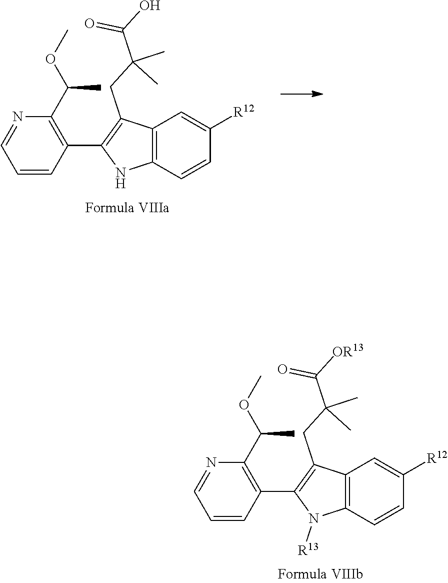

In some embodiments, the reducing step (b) includes contacting the compound of Formula VIIIb with a reducing agent. In some embodiments, the reducing agent is a borohydride. In some embodiments, the borohydride is lithium borohydride. In some embodiments, the reducing step (b) is carried out according to the following scheme:

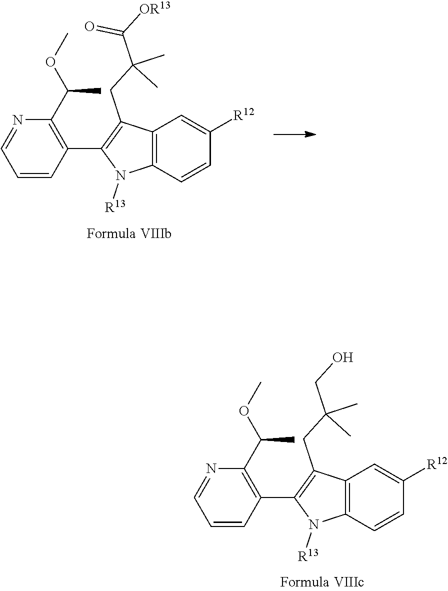

In some embodiments, the compound of Formula VIII is Compound F:

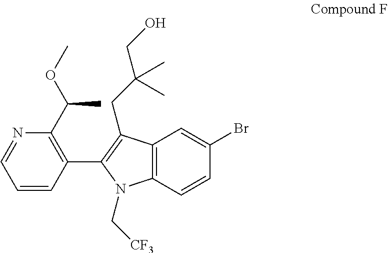 [SMILES: CO[C@@H](C)c1ncccc1-c1c(CC(C)(C)CO)c2cc(Br)ccc2n1CC(F)(F)F]

and the method includes the steps of:
a) esterifying and N-alkylating a compound of Formula VIIIa to form a compound of Formula VIIIb having the following structure:

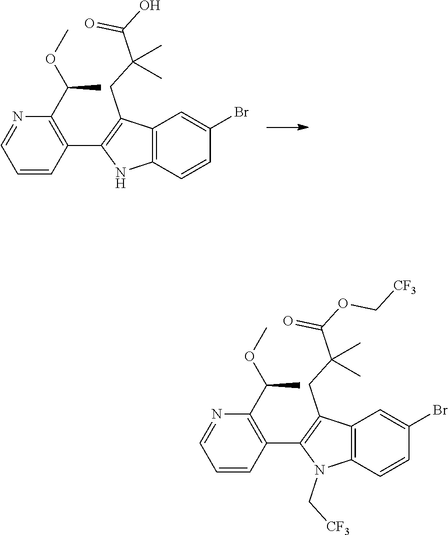 [SMILES: CO[C@@H](C)c1ncccc1-c1[nH]c2ccc(Br)cc2c1CC(C)(C)C(=O)O ; CO[C@@H](C)c1ncccc1-c1c(CC(C)(C)C(=O)OCC(F)(F)F)c2cc(Br)ccc2n1CC(F)(F)F]

b) reducing the compound of Formula VIIIb to form a compound of Formula VIIIc as a mixture of atropisomers having the following structure:

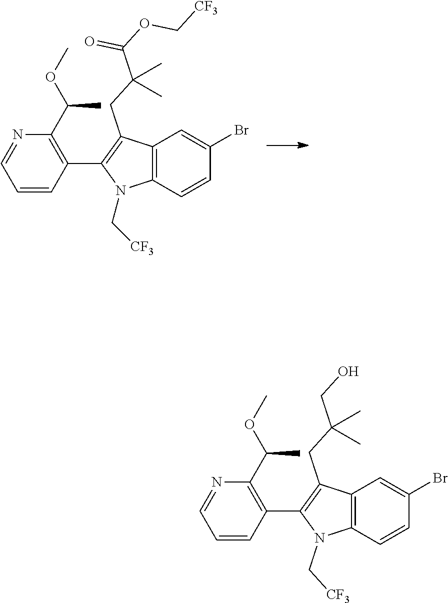 [SMILES: CO[C@@H](C)c1ncccc1-c1c(CC(C)(C)C(=O)OCC(F)(F)F)c2cc(Br)ccc2n1CC(F)(F)F ; CO[C@@H](C)c1ncccc1-c1c(CC(C)(C)CO)c2cc(Br)ccc2n1CC(F)(F)F]

and
c) separating the mixture of atropisomers of Formula VIIIc to obtain the stereochemically pure compound of Formula VIII.

In some embodiments, the esterifying and N-alkylating step (a) includes contacting the compound of Formula VIIIa with an alkylating agent. In some embodiments, the alkylating agent is R¹³-LG, wherein R¹³ is optionally substituted C₁-C₆ alkyl and LG is a leaving group. In some embodiments, the alkylating agent is 2,2,2-trifluoroethyl trifluoromethanesulfonate. In some embodiments, the esterifying and N-alkylating step (a) is carried out according to the following scheme:

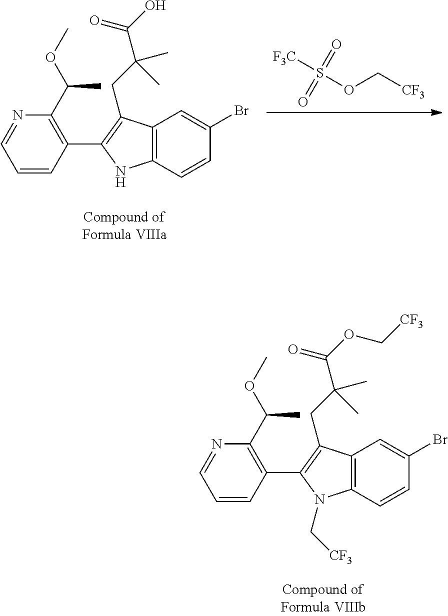 [SMILES: CO[C@@H](C)c1ncccc1-c1[nH]c2ccc(Br)cc2c1CC(C)(C)C(=O)O ; O=S(=O)(OCC(F)(F)F)C(F)(F)F ; CO[C@@H](C)c1ncccc1-c1c(CC(C)(C)C(=O)OCC(F)(F)F)c2cc(Br)ccc2n1CC(F)(F)F]

In some embodiments, the reducing step (b) includes contacting the compound of Formula VIIIb with a reducing agent. In some embodiments, the reducing agent is a borohydride. In some embodiments, the borohydride is lithium borohydride.

In some embodiments, the reducing step (b) is carried out according to the following scheme:

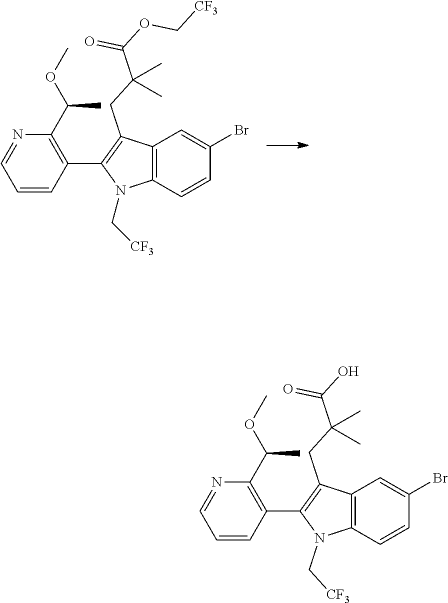 [SMILES: CO[C@@H](C)c1ncccc1-c1c(CC(C)(C)C(=O)OCC(F)(F)F)c2cc(Br)ccc2n1CC(F)(F)F ; CO[C@@H](C)c1ncccc1-c1c(CC(C)(C)C(=O)O)c2cc(Br)ccc2n1CC(F)(F)F]

In yet another aspect, the disclosure provides a method of preparing a compound, or a salt thereof, of Formula VIII:

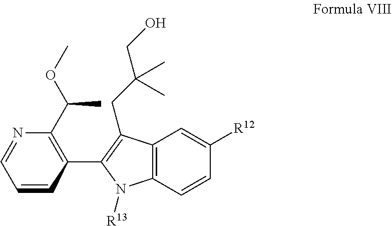

the method including the steps of:
a) reacting (e.g., esterifying) a compound of Formula VIIIa to form a compound of Formula VIIId:

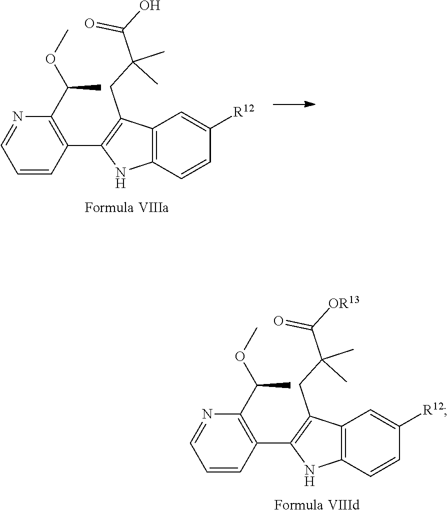

b) N-alkylating the compound of Formula VIIId to form the compound of Formula VIIIb:

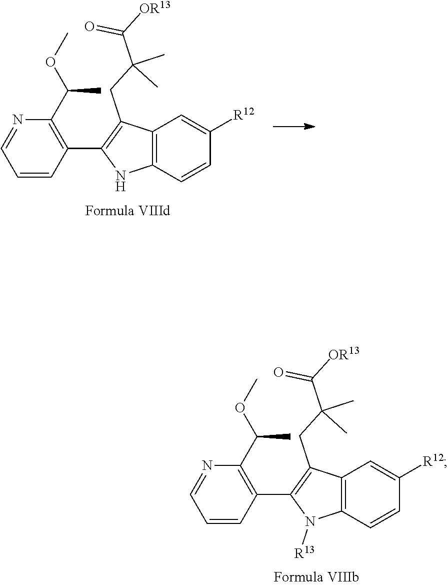

c) reducing the compound of Formula VIIIb to form a compound of Formula VIIIc as a mixture of atropisomers:

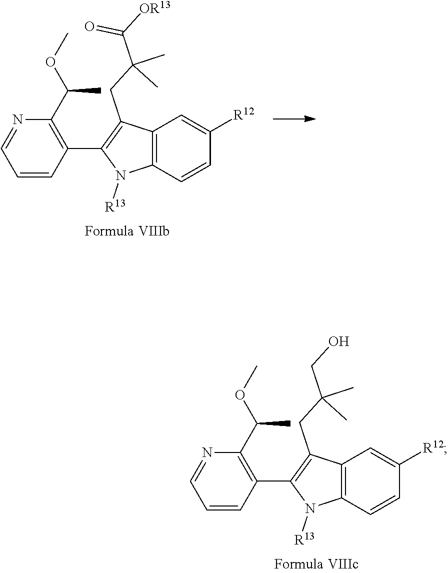

and
d) separating the mixture of atropisomers of Formula VIIIc to obtain the stereochemically pure compound of Formula VIII;
wherein R¹² is halogen; and
R¹³ is optionally substituted C₁-C₆ alkyl.

In some embodiments, step (a) includes contacting the compound of Formula VIIIa with R¹³OH, wherein R¹³ is optionally substituted C₁-C₆ alkyl. In some embodiments, the alcohol is methanol. In some embodiments, step (a) further includes contacting the compound of Formula VIIIa with an acid. In some embodiments, the acid is sulfuric acid. In some embodiments, step (a) is carried out according to the following scheme:

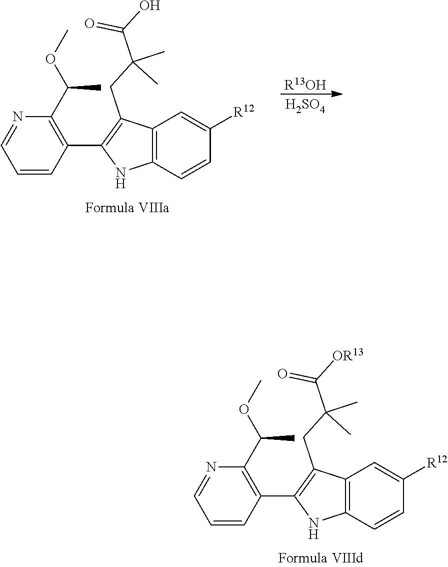

In some embodiments, the N-alkylating step (b) includes contacting the compound of Formula VIIId with an alkylating agent. In some embodiments, the alkylating agent is R¹³-LG, wherein R¹³ is optionally substituted C₁-C₆ alkyl and LG is a leaving group. In some embodiments, the alkylating agent is 2,2,2-trifluoroethyl trifluoromethanesulfonate. In some embodiments, the N-alkylating step (b) is carried out according to the following scheme:

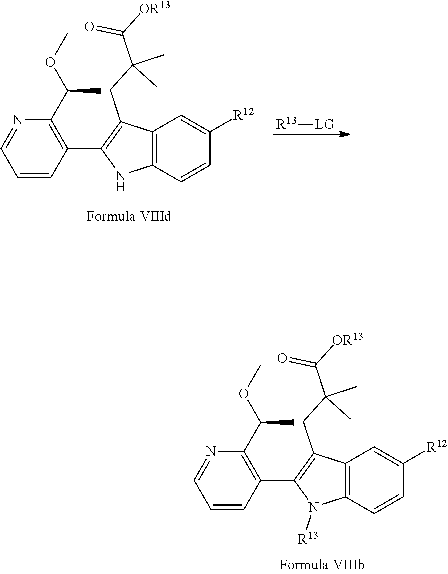

In some embodiments, the reducing step (c) includes contacting the compound of Formula VIIIb with a reducing agent. In some embodiments, the reducing agent is a borohydride. In some embodiments, the borohydride is lithium borohydride.

In some embodiments, the reducing step (c) is carried out according to the following scheme:

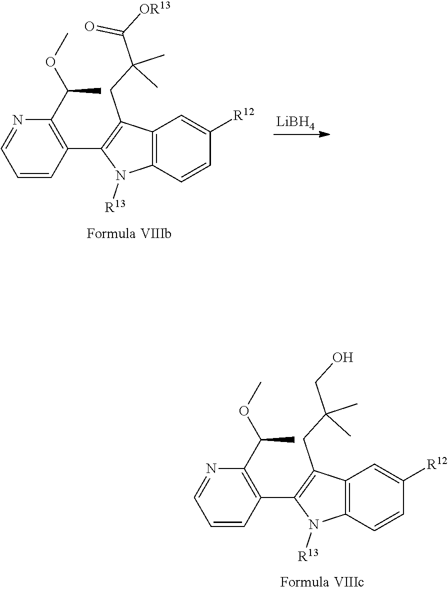

In some embodiments, the compound of Formula VIII is Compound G:

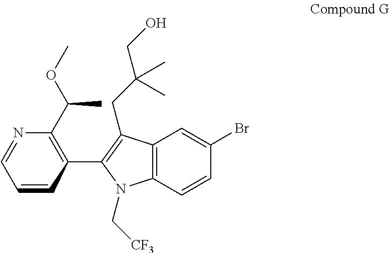

and the method includes the steps of:
a) reacting (e.g., esterifying) a compound of Formula VIIIa to form a compound of Formula VIIId having the following structure:

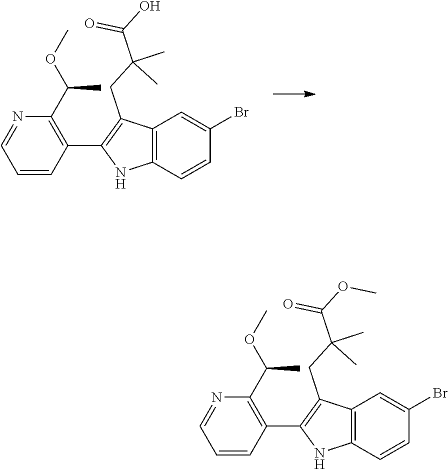 [SMILES: CO[C@@H](C)c1ncccc1-c1[nH]c2ccc(Br)cc2c1CC(C)(C)C(=O)O ; COC(=O)C(C)(C)Cc1c(-c2cccnc2[C@H](C)OC)[nH]c2ccc(Br)cc12]

b) N-alkylating the compound of Formula VIIId to form the compound of Formula VIIIb having the following structure:

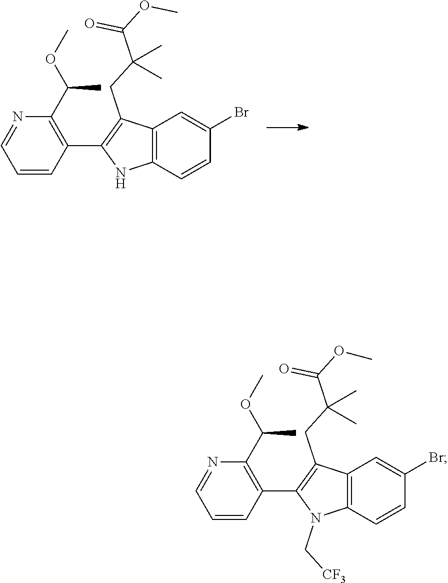 [SMILES: COC(=O)C(C)(C)Cc1c(-c2cccnc2[C@H](C)OC)[nH]c2ccc(Br)cc12 ; COC(=O)C(C)(C)Cc1c(-c2cccnc2[C@H](C)OC)n(CC(F)(F)F)c2ccc(Br)cc12]

c) reducing the compound of Formula VIIIb to form Compound G as a mixture of atropisomers having the following structure:

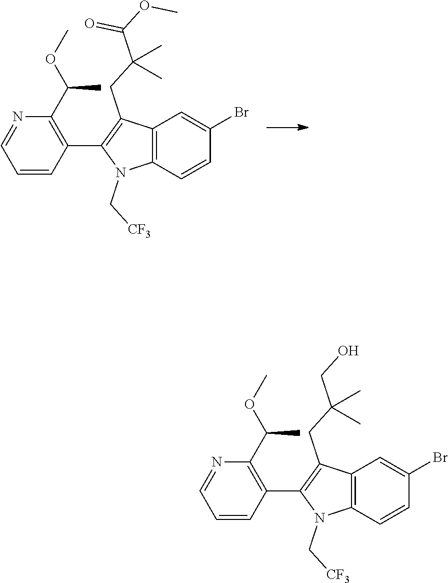 [SMILES: COC(=O)C(C)(C)Cc1c(-c2cccnc2[C@H](C)OC)n(CC(F)(F)F)c2ccc(Br)cc12 ; CO[C@@H](C)c1ncccc1-c1c(CC(C)(C)CO)c2cc(Br)ccc2n1CC(F)(F)F]

In some embodiments, step (a) includes contacting the compound of Formula VIIIa with R¹³OH, wherein R¹³ is optionally substituted C₁-C₆ alkyl. In some embodiments, the alcohol is methanol. In some embodiments, step (a) further includes contacting the compound of Formula VIIIa with an acid. In some embodiments, the acid is sulfuric acid. In some embodiments, step (a) is carried out according to the following scheme:

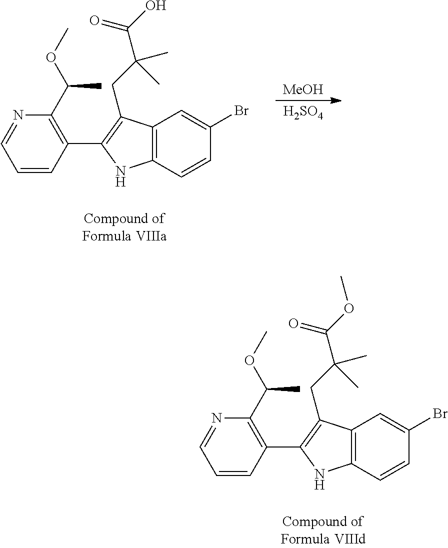

In some embodiments, the N-alkylating step (b) includes contacting the compound of Formula VIIId with an alkylating agent. In some embodiments, the alkylating agent is R¹³-LG, wherein R¹³ is optionally substituted C₁-C₆ alkyl and LG is a leaving group. In some embodiments, the alkylating agent is 2,2,2-trifluoroethyl trifluoromethanesulfonate. In some embodiments, the N-alkylating step (b) is carried out according to the following scheme:

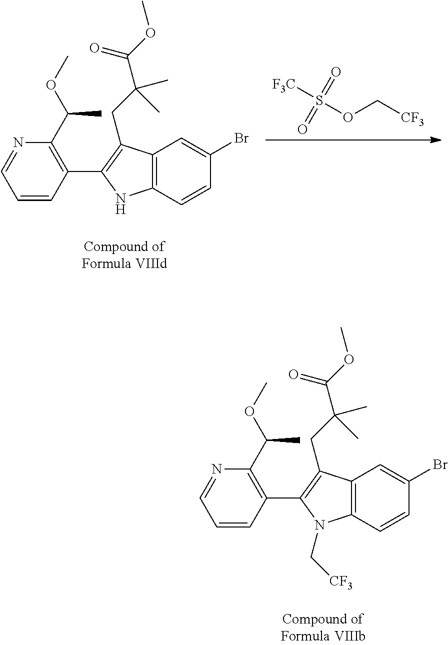 [SMILES: COC(=O)C(C)(C)Cc1c(-c2cccnc2[C@H](C)OC)[nH]c2ccc(Br)cc12 ; O=S(=O)(OCC(F)(F)F)C(F)(F)F ; COC(=O)C(C)(C)Cc1c(-c2cccnc2[C@H](C)OC)n(CC(F)(F)F)c2ccc(Br)cc12]

In some embodiments, the reducing step (c) includes contacting the compound of Formula VIIIb with a reducing agent. In some embodiments, the reducing agent is a borohydride. In some embodiments, the borohydride is lithium borohydride. In some embodiments, the reducing step (c) is carried out according to the following scheme:

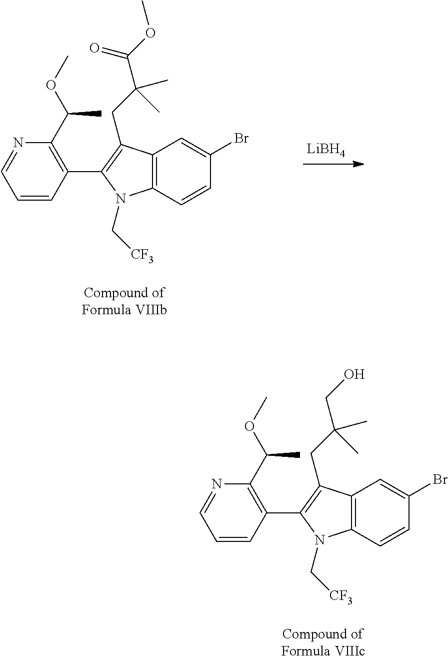 [SMILES: COC(=O)C(C)(C)Cc1c(-c2cccnc2[C@H](C)OC)n(CC(F)(F)F)c2ccc(Br)cc12 ; "LiBH4" ; CO[C@@H](C)c1ncccc1-c1c(CC(C)(C)CO)c2cc(Br)ccc2n1CC(F)(F)F]

In some embodiments, the mixture of atropisomers is separated by column chromatography or chemical resolution. In some embodiments, the chemical resolution includes contacting the compound of Formula VIIIc with an acid. In some embodiments, the acid is methanesulfonic acid. In some embodiments, the acid is camphor sulfonic acid (e.g., D-camphor-10-sulfonic acid).

In yet another aspect, the disclosure provides a method of preparing a compound of Formula VIIIc:

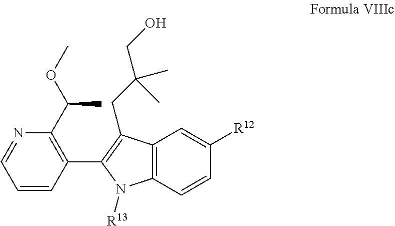

the method including N-alkylating a compound of Formula VIIIe with a compound having the structure of R¹³-LG¹:

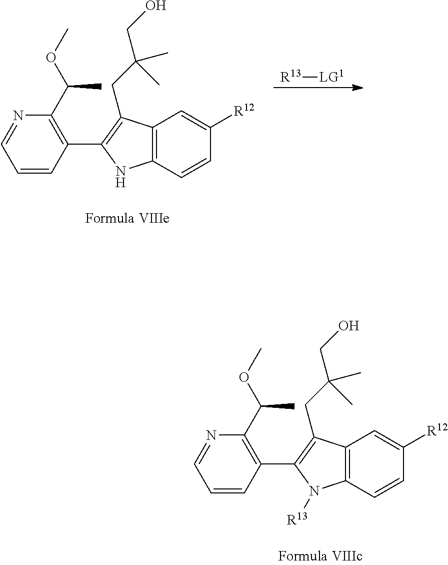

wherein each R¹² is independently halogen;
each R¹³ is independently optionally substituted C₁-C₆ alkyl; and
LG¹ is a leaving group.

In some embodiments, each R¹² is Br. In some embodiments, each R¹³ is —CH₂CF₃. In some embodiments, LG¹ is halogen, triflate, mesylate, or tosylate. In some embodiments, R¹³-LG is 2,2,2-trifluoroethyl trifluoromethanesulfonate. In some embodiments, the method includes contacting the compound of Formula VIIIe and R¹³-LG¹ with a base. In some embodiments, the base is tripotassium phosphate (K₃PO₄). In some embodiments, the In some embodiments, the method is carried out at a temperature of at least 10° C. (e.g., 10, 15, 20, or 25° C.). In some embodiments, the method produces a mixture of atropisomers of the compound of Formula VIIIc. In some embodiments, the N-alkylating is carried out according to the following scheme:

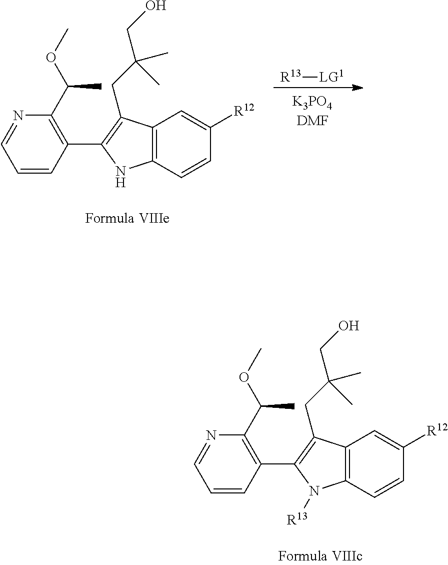

In some embodiments of the method of preparing the compound of Formula VIIIc, the compound of Formula Ville is Compound K. In some embodiments, the compound of Formula VIIIc is Compound G. In some embodiments, the N-alkylating is carried out according to the following scheme:

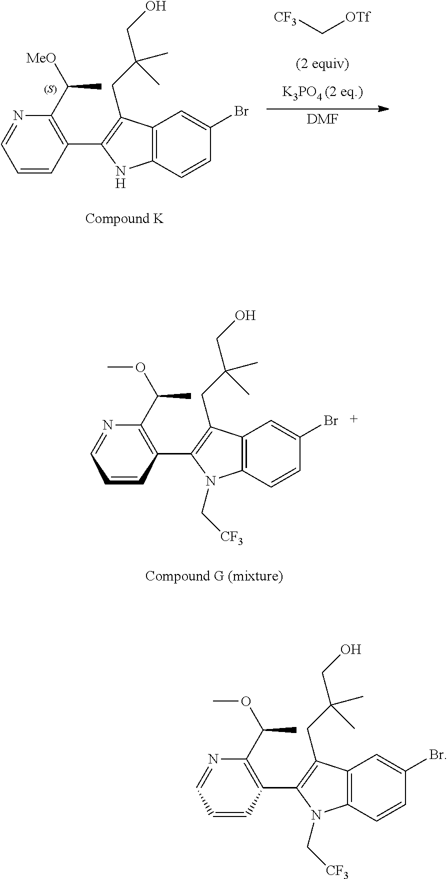

In yet another aspect, the disclosure provides a method of preparing a compound of Formula VIIIe:

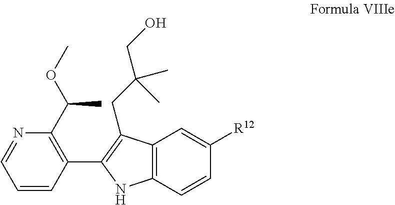

the method including reducing a compound of Formula VIIIf with a reducing agent:

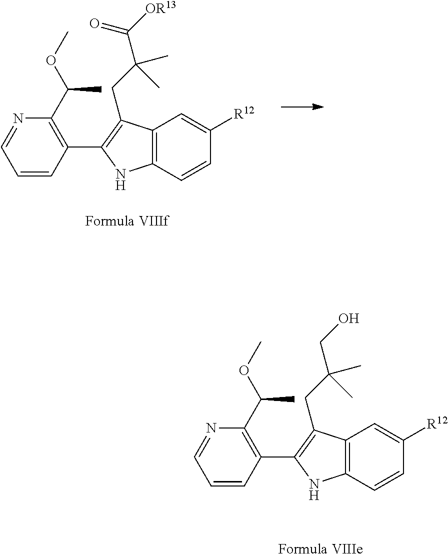

wherein R¹² is halogen; and
R¹³ is optionally substituted C₁-C₆ alkyl.

In some embodiments, each R¹² is Br. In some embodiments, each R¹³ is —CH₂CF₃. In some embodiments, the reducing agent is NaBH₄. In some embodiments, the method includes contacting the compound of Formula VIIIf with calcium chloride (CaCl₂). In some embodiments, the reducing is carried out at a temperature of at least 20° C. (e.g., 20, 25, or 30° C.). In some embodiments, the reducing is carried out according to the following scheme:

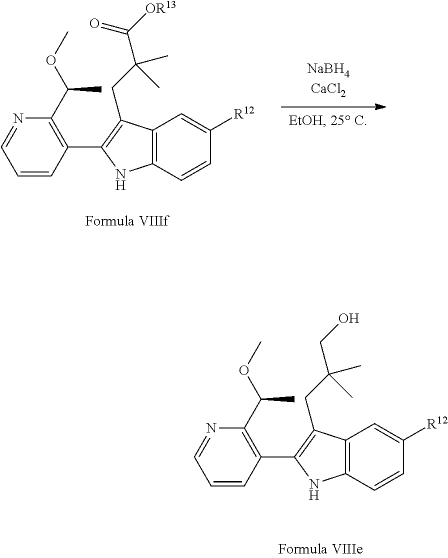

In some embodiments, the reducing is carried out according to the following scheme:

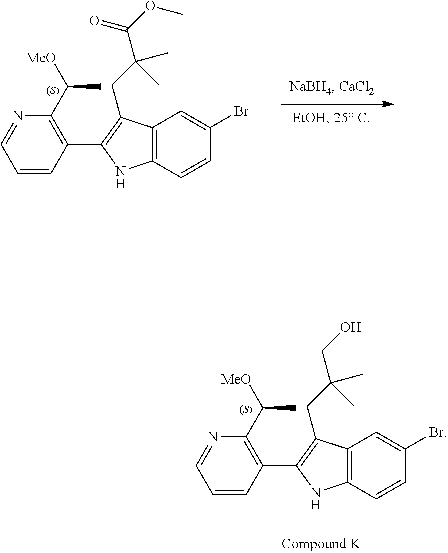

In yet another aspect, the disclosure provides a method of preparing a compound, or a salt thereof, of Formula IX:

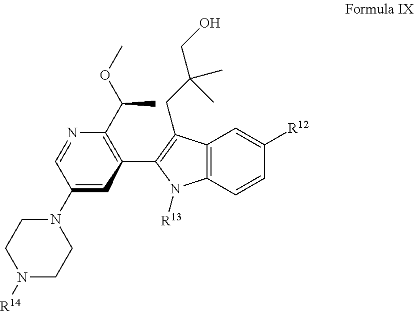

the method including the steps of:
a) borylating a compound of Formula VIII to form a compound Formula X:

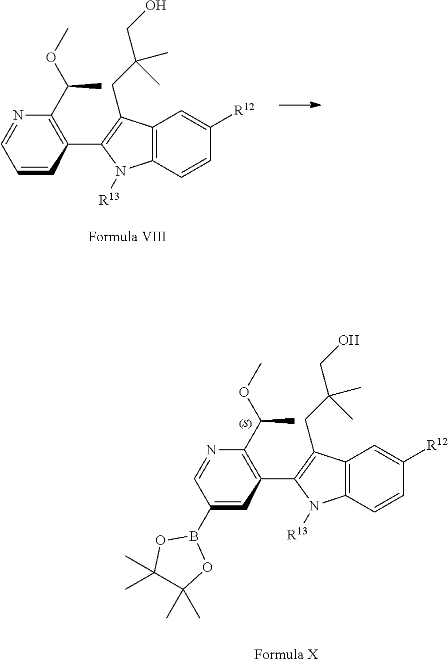

and
b) coupling the compound of Formula X with the compound of Formula XI to form a compound of Formula XII:

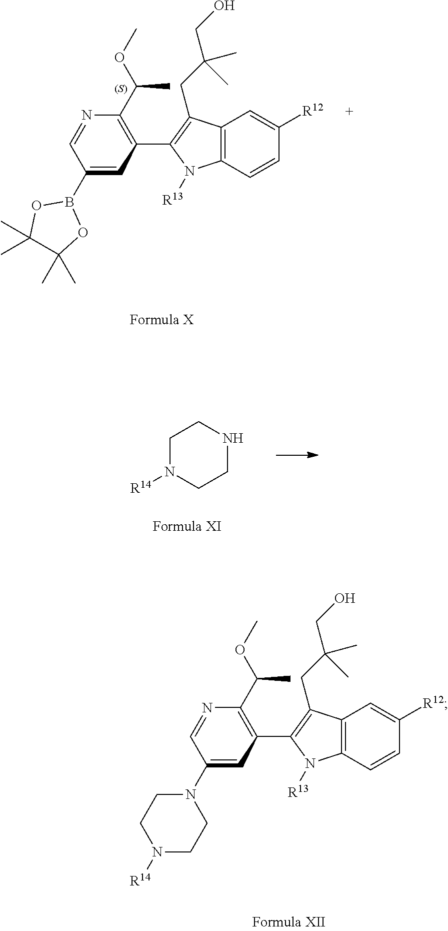

wherein R¹² is halogen;
R¹³ is optionally substituted C₁-C₆ alkyl; and
R¹⁴ is optionally substituted 3- to 6-membered cycloalkyl.

In some embodiments, the borylating step (a) includes contacting the compound of Formula VIII with a borylating agent. In some embodiments, the borylating agent is bis(pinacolato)diboron (B₂Pin₂). In some embodiments, the borylating step (a) further includes contacting the compound of Formula VIII with 4,4′-di-tert-butyl-2,2′-dipyridyl (dtbpy). In some embodiments, the borylating step (a) further includes contacting the compound of Formula VIII with an iridium catalyst. In some embodiments, the iridium catalyst is [Ir(OMe)(COD)]2.

In some embodiments, the borylating step (a) is carried out according to the following scheme:

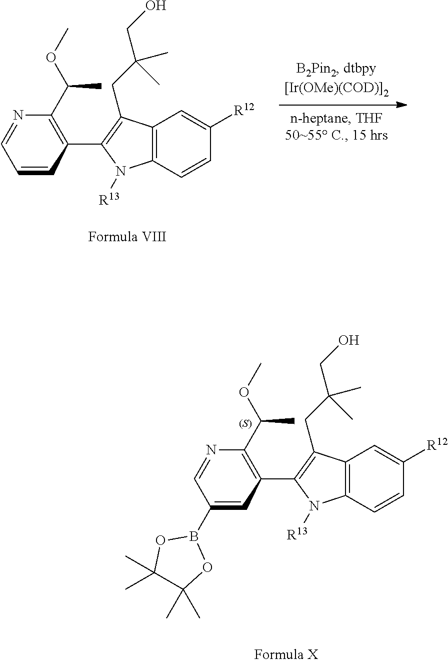

In some embodiments, the coupling step (b) includes contacting the compound of Formula X and the compound of Formula XI with a copper catalyst. In some embodiments, the copper catalyst is Cu(OAc)₂. In some embodiments, the coupling step (b) includes contacting the compound of Formula X and the compound of Formula XI with a base. In some embodiments, the base is an amine base. In some embodiments, the base is triethylamine or tetramethylpiperidine. In some embodiments, the coupling step (b) is carried out according to the following scheme:

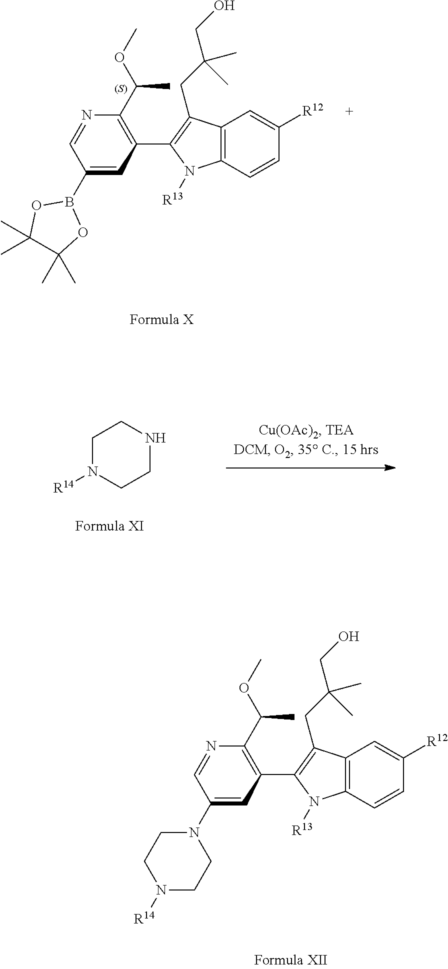

In some embodiments, the compound of Formula IX is Compound H:

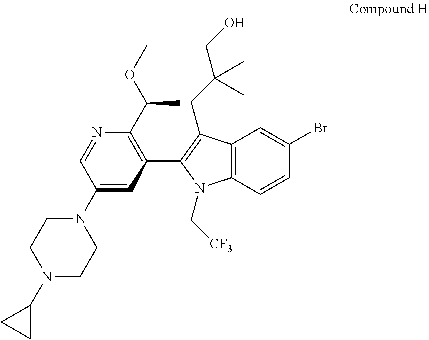

the method includes the steps of:
a) borylating a compound of Formula VIII to form a compound Formula X having the following structure:

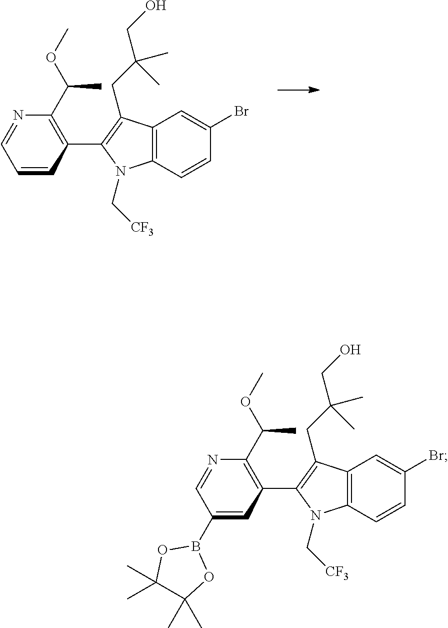

b) coupling the compound of Formula X with the compound of Formula XI to form Compound H having the following structure:

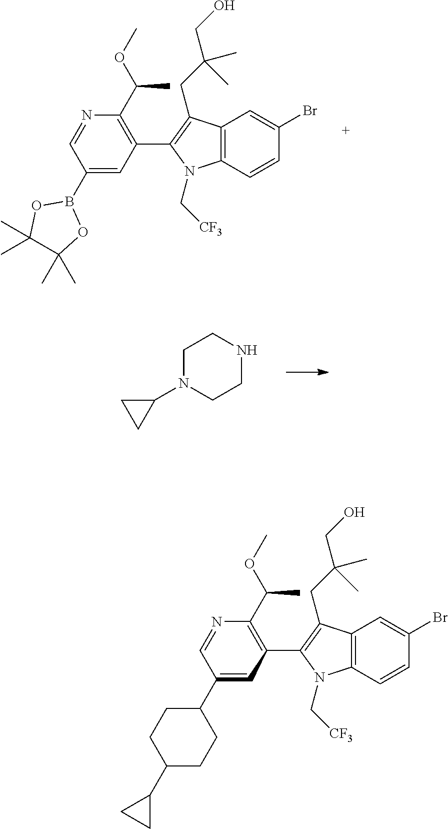

In some embodiments, the borylating step (a) includes contacting the compound of Formula VIII with a borylating agent. In some embodiments, the borylating agent is bis(pinacolato)diboron (B₂Pin₂). In some embodiments, the borylating step (a) further includes contacting the compound of Formula VIII with 4,4′-di-tert-butyl-2,2′-dipyridyl (dtbpy). In some embodiments, the borylating step (a) further includes contacting the compound of Formula VIII with an iridium catalyst. In some embodiments, the iridium catalyst is [Ir(OMe)(COD)]₂. In some embodiments, the borylating step (a) is carried out according to the following scheme:

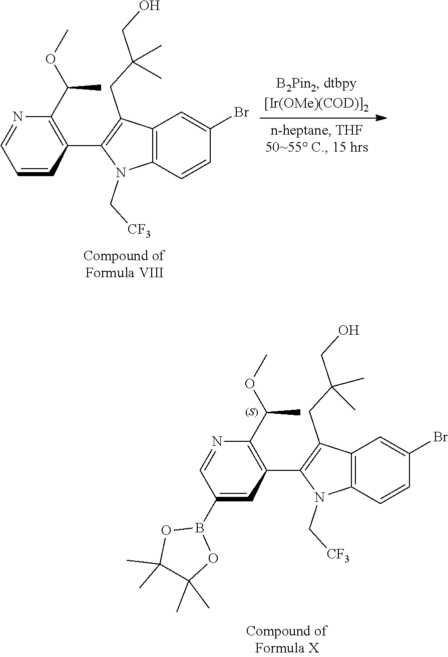

In some embodiments, the coupling step (b) includes contacting the compound of Formula X and the compound of Formula XI with a copper catalyst. In some embodiments, the copper catalyst is Cu(OAc)₂. In some embodiments, the coupling step (b) includes contacting the compound of Formula X and the compound of Formula XI with a base. In some embodiments, the base is an amine base. In some embodiments, the base is triethylamine or tetramethylpiperidine. In some embodiments, the coupling step (b) is carried out according to the following scheme:

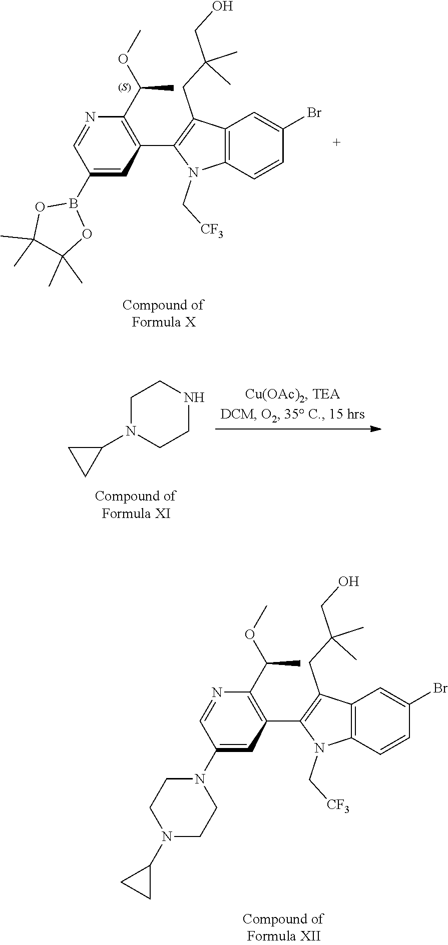

In some embodiments, the compound, or a salt thereof, of Formula X:

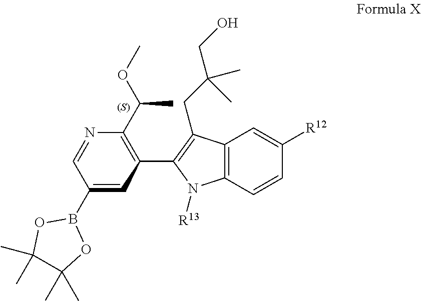

is further prepared by:
a) borylating the compound of Formula VIII to form a compound of Formula XIII:

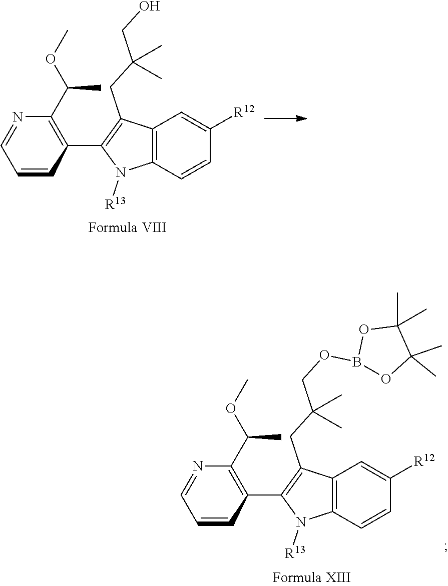

and
b) hydrolyzing and borylating the compound of Formula XIII in one step to form Formula X:

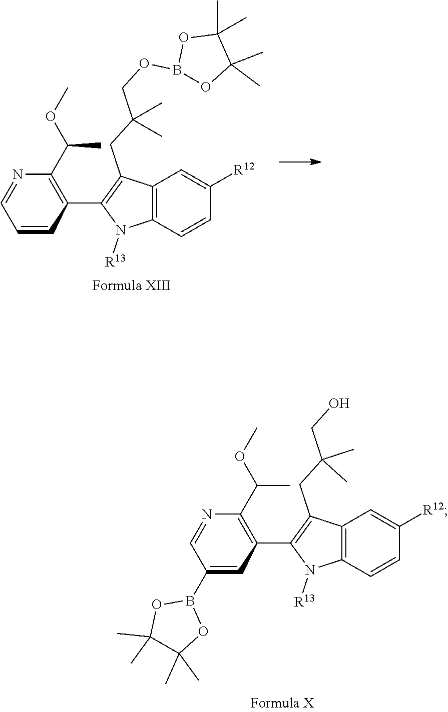

wherein R¹² is halogen; and
R¹³ is optionally substituted C₁-C₆ alkyl.

In some embodiments, the borylating step (a) includes contacting the compound of Formula VIII with a borylating agent. In some embodiments, the borylating agent is pinacolborane (HBPin). In some embodiments, the borylating step (a) is carried out according to the following scheme:

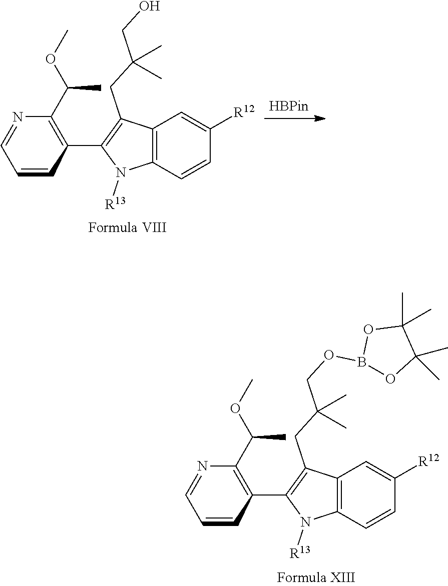

In some embodiments, the hydrolyzing and borylating step (b) includes contacting the compound of Formula XIII with a borylating agent. In some embodiments, the borylating agent is bis(pinacolato)diboron (B₂Pin₂). In some embodiments, the hydrolyzing and borylating step (b) further includes contacting the compound of Formula XIII with 4,4′-di-tert-butyl-2,2′-dipyridyl (dtbpy). In some embodiments, the hydrolyzing and borylating step (b) further includes contacting the compound of Formula XIII with an iridium catalyst. In some embodiments, the iridium catalyst is [Ir(OMe)(COD)]₂. In some embodiments, the hydrolyzing and borylating step (b) is carried out according to the following scheme:

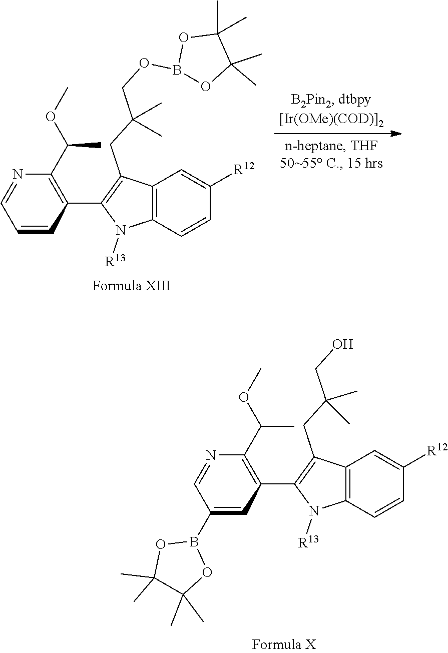

In yet another aspect, the disclosure provides a method of preparing a compound, or a salt thereof, of Formula XIV:

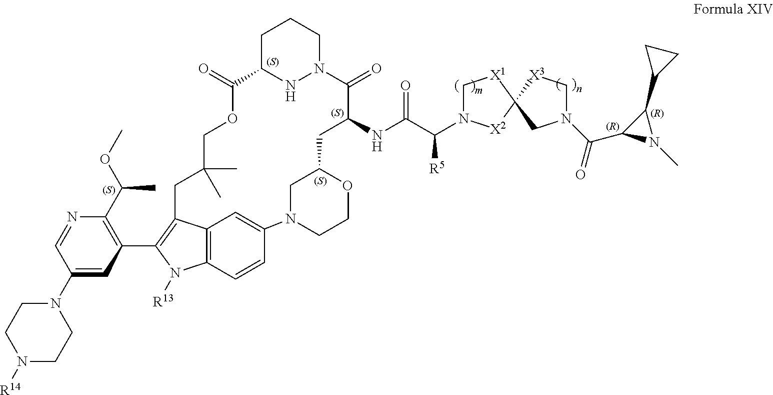

the method including the steps of:
a) reacting (e.g., esterifying) a compound of Formula XV with a compound of Formula XII to form a compound of Formula XVI:

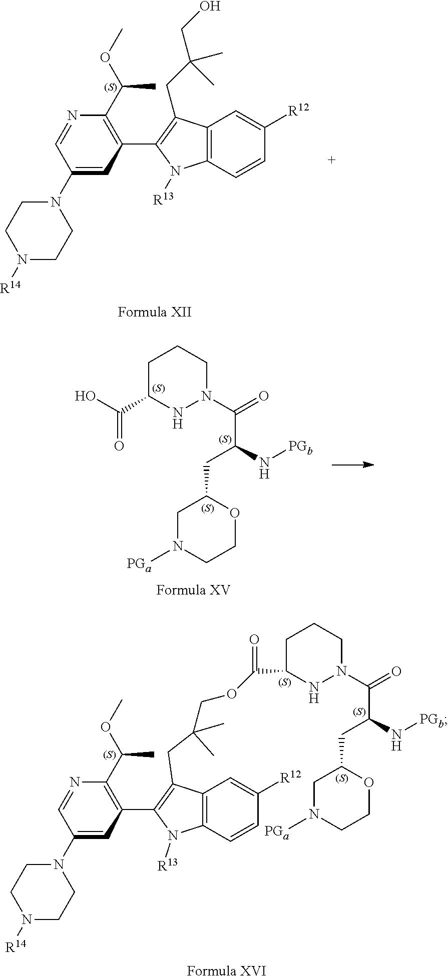

b) removing (e.g., deprotecting) PGₐ of the compound of Formula XVI to form a compound of Formula XVII:

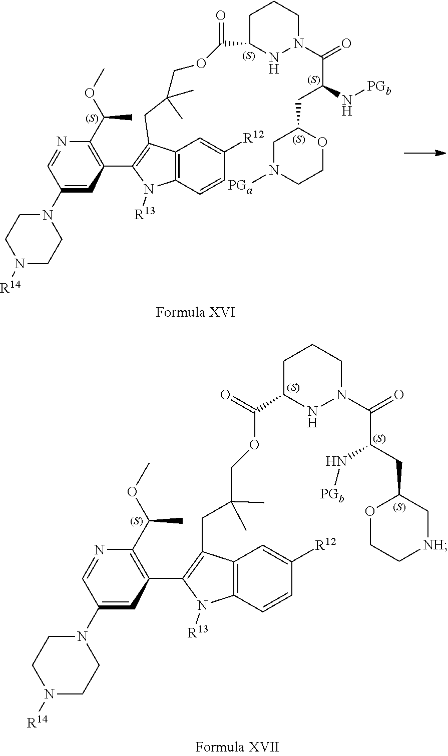

c) cyclizing the compound of Formula XVII with a transition metal catalyst to form a compound of Formula XVII:

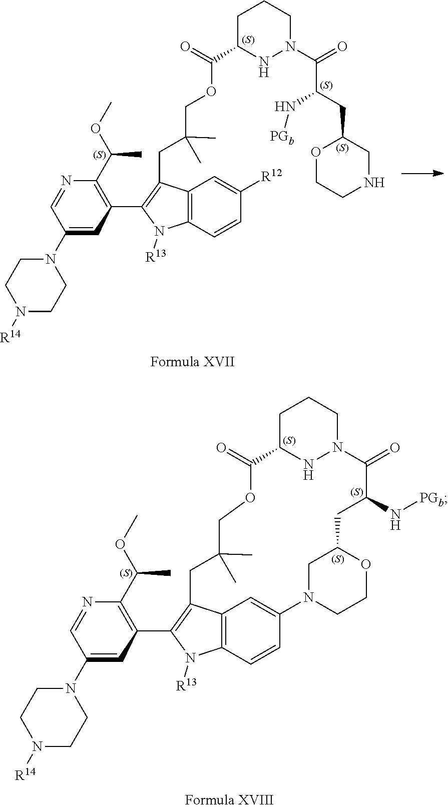

d) removing (e.g., deprotecting) PG_{b} of the compound of Formula XVIII to form a compound of Formula XIX:

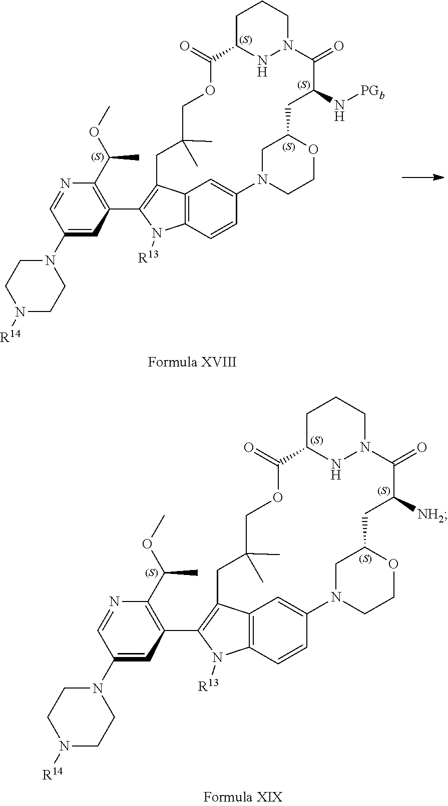

and
e) coupling the compound of Formula XIX with the compound of Formula IV to form a compound of Formula XIV:

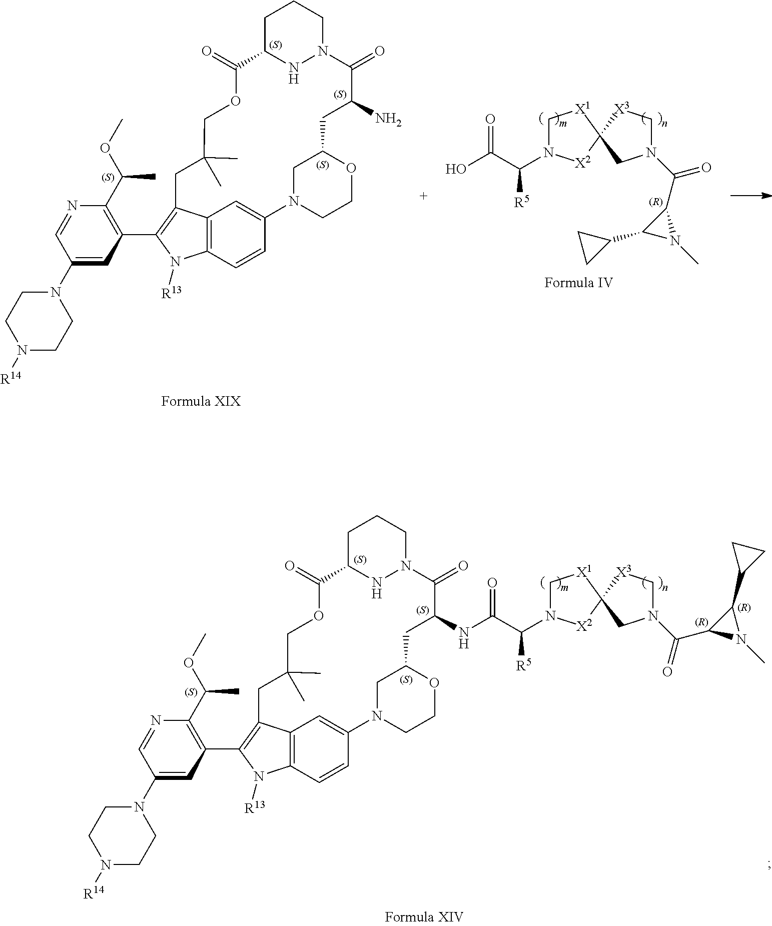

wherein n and m are each, independently, 0, 1, 2, 3, 4, or 5;
X¹, X², and X³ are each, independently, —CH₂—, —CHF—, —CF₂—, —C(O)—, or —O—;
R¹² is halogen;
R¹³ is optionally substituted C₁-C₆ alkyl;
R¹⁴ is optionally substituted 3- to 6-membered cycloalkyl;
PGₐ is an acid-labile protecting group; and
PG_{b} is an acid-stable protecting group.

In some embodiments, step (a) includes contacting the compound of Formula XV and the compound of Formula XII with a carbodiimide coupling reagent, an anti-racemization agent, and a base. In some embodiments, the carbodiimide coupling reagent is EDCl, the anti-racemization agent is HOBt, and the base is N,N-diisopropylethylamine.

In some embodiments, step (a) is carried out according to the following scheme:

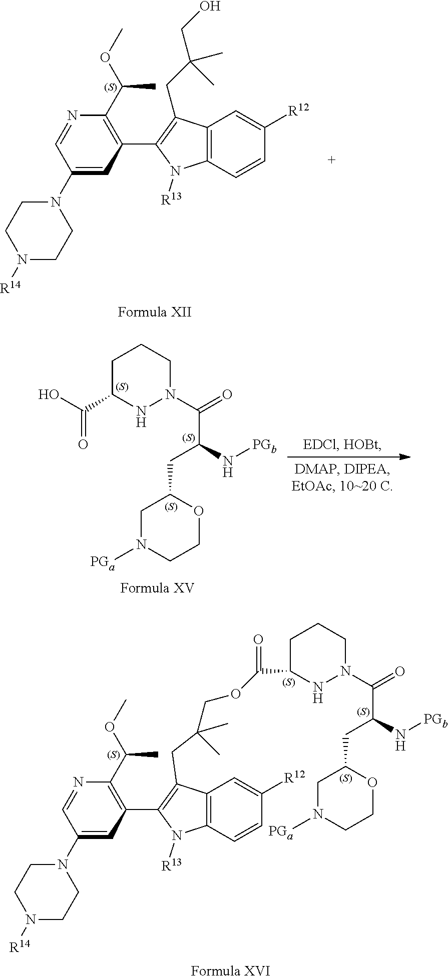

In some embodiments, the deprotecting step (b) includes contacting the compound of Formula XVI with an acid. In some embodiments, the acid is trifluoroacetic acid. In some embodiments, the deprotecting step (b) is carried out according to the following scheme:

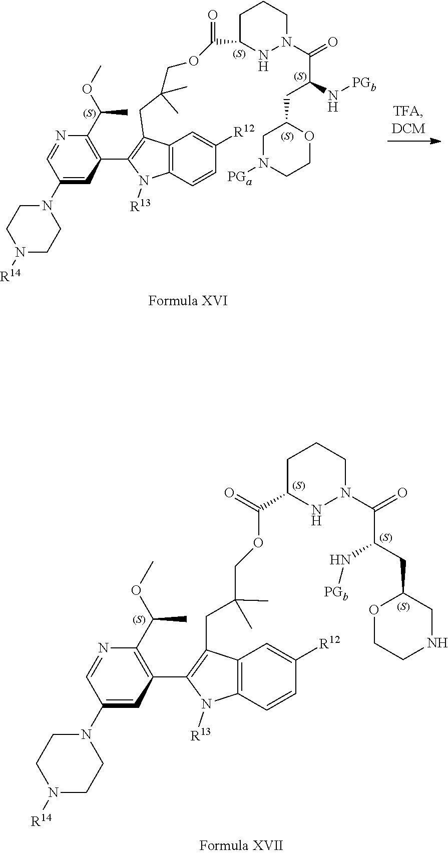

In some embodiments, the cyclizing step (c) includes contacting the compound of Formula XVII with a transition metal catalyst, a phosphine ligand, and a base. In some embodiments, the transition metal catalyst includes palladium, platinum, nickel, copper, and iron catalysts. In some embodiments, the transition metal catalyst is a palladium catalyst. In some embodiments, the palladium catalyst is P(tBu)₃ Pd G3 or Pd₂(dba)₃. In some embodiments, the phosphine ligand is Q-Phos or [(tBu)₃PH]BF₄. In some embodiments, the base is K₃PO₄. In some embodiments, the cyclizing step (c) is carried out according to the following scheme:

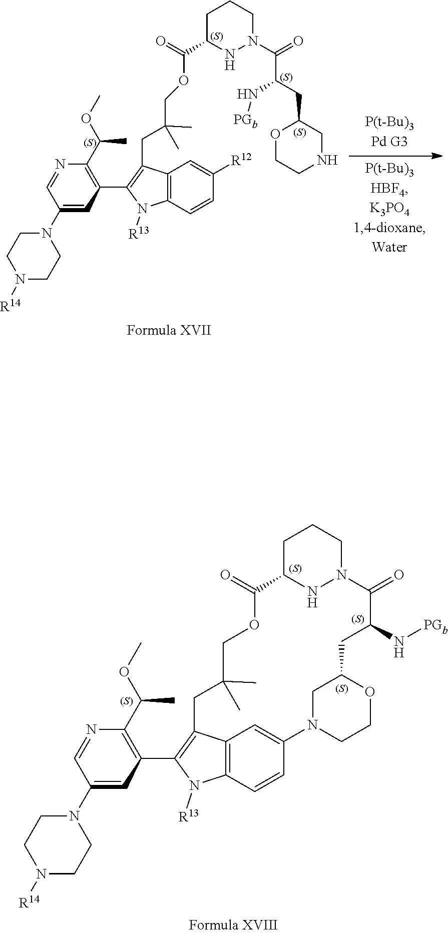

In some embodiments, the cyclizing step (c) is carried out according to the following scheme:

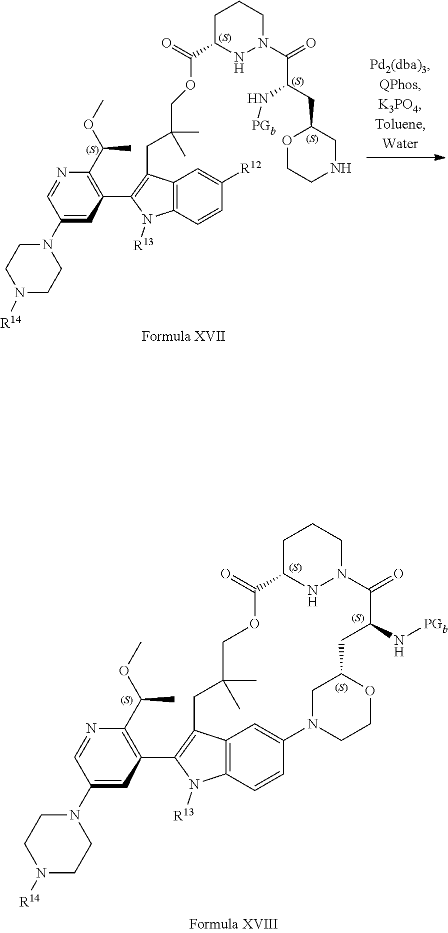

In some embodiments, the cyclizing step (c) further includes contacting the compound of Formula XVIII with hydrochloric acid to form a hydrochloride salt of the compound of Formula XVIII.

In some embodiments, the deprotecting step (d) includes contacting the compound of Formula XVIII with a palladium catalyst and hydrogen gas in an organic solvent. In some embodiments, the palladium catalyst is Pd/C. In some embodiments, the organic solvent is methyl tert-butyl ether. In some embodiments, the deprotecting step (d) is carried out according to the following scheme:

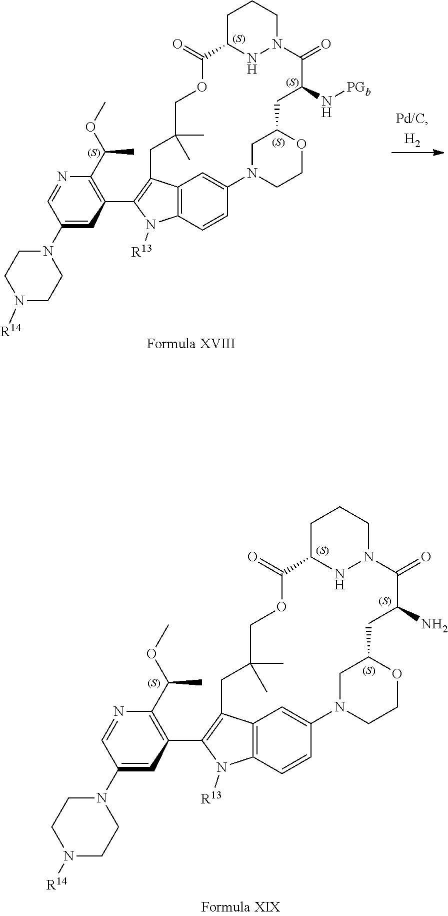

In some embodiments, the deprotecting step (d) further includes washing the compound of Formula XIX with N-acetyl cysteine.

In some embodiments, the deprotecting step (e) includes contacting the compound of Formula XIX and the compound of Formula IV with a coupling reagent, an anti-racemization reagent, and a base. In some embodiments, the coupling reagent is PyBOP or EDCl. In some embodiments, the anti-racemization agent is selected from the group consisting of Oxyma, HOBt, or HOPO. In some embodiments, the base is N,N-diisopropylethylamine.

In some embodiments, the coupling step (e) is carried out according to the following scheme:

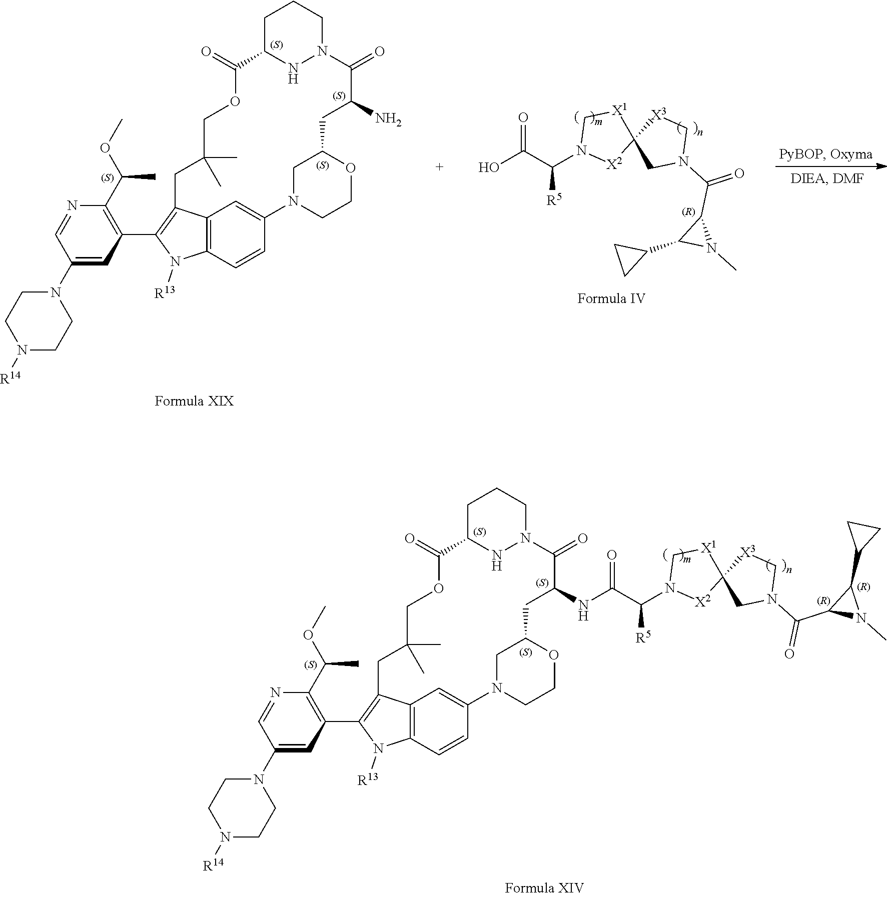

In some embodiments, the coupling step (e) is carried out according to the following scheme:

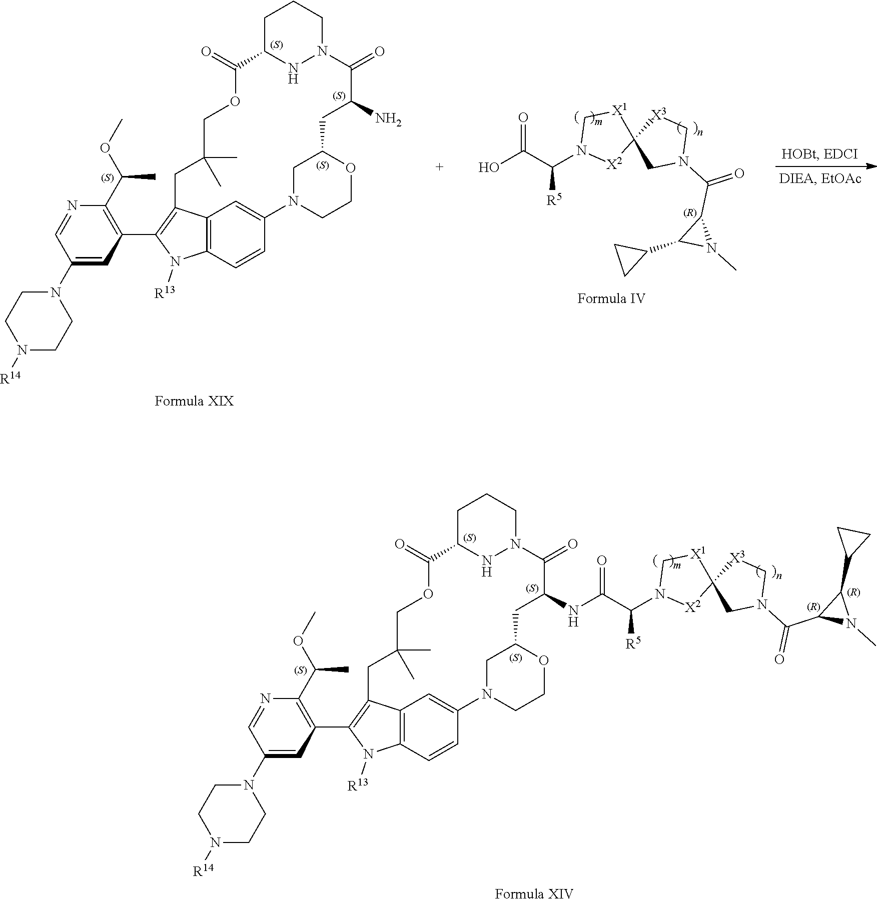

In some embodiments, the coupling step (e) is carried out according to the following scheme:

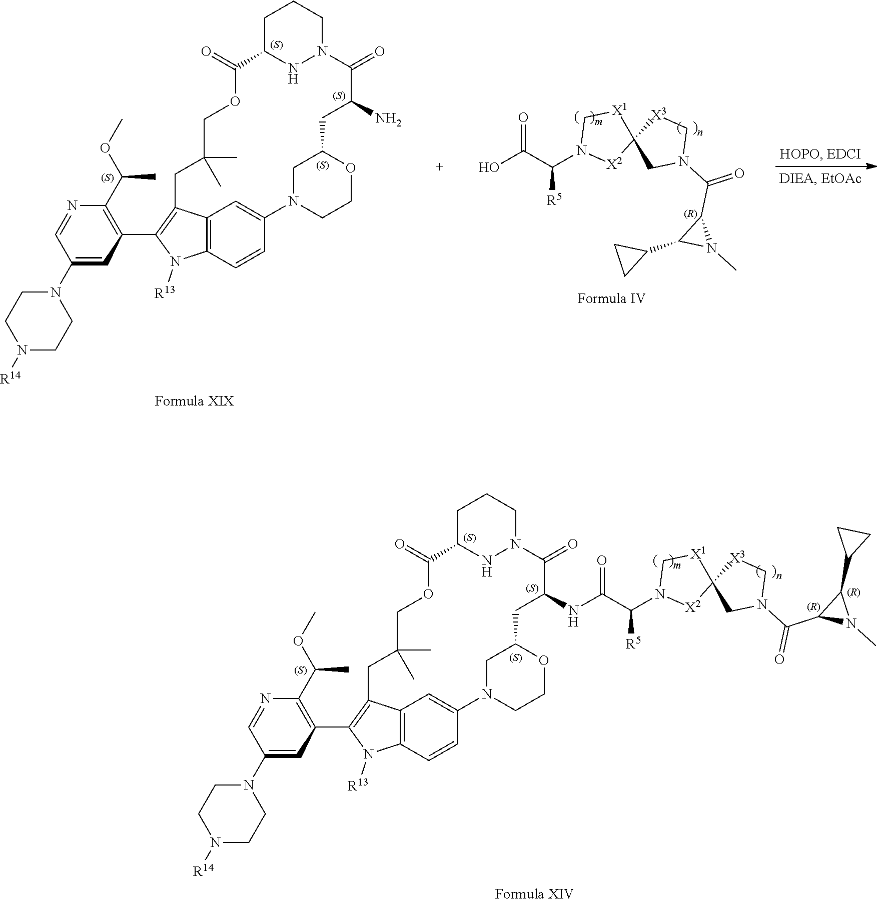

In some embodiments, the method further includes the step of purifying the compound of Formula XIV. In some embodiments, the purifying includes recrystallizing the compound of Formula XIV. In some embodiments, the recrystallizing includes adding a first solvent, followed by adding a second solvent. In some embodiments, the first solvent is dioxane or 2-methyltetrahydrofuran. In some embodiments, the second solvent is diisopropyl ether or isopropyl alcohol. In some embodiments, the recrystallizing further includes adding a third solvent. In some embodiments, the third solvent is heptane. In some embodiments, R¹² is —Br. In some embodiments, R¹³ is —CH₂CF₃. In some embodiments, PGₐ is Boc. In some embodiments, PG_{b} is CBz.

In some embodiments, the compound of Formula XIV is Compound 1:

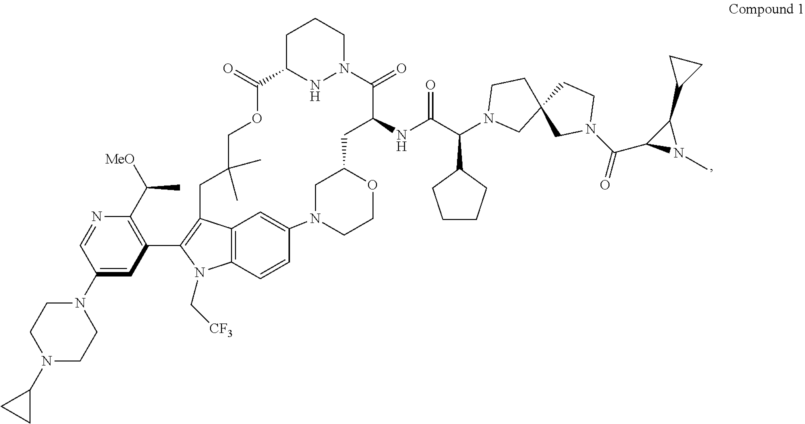

and the method includes the steps of:
a) reacting (e.g., esterifying) a compound of Formula XV with a compound of Formula XII to form a compound of Formula XVI having the following structure:

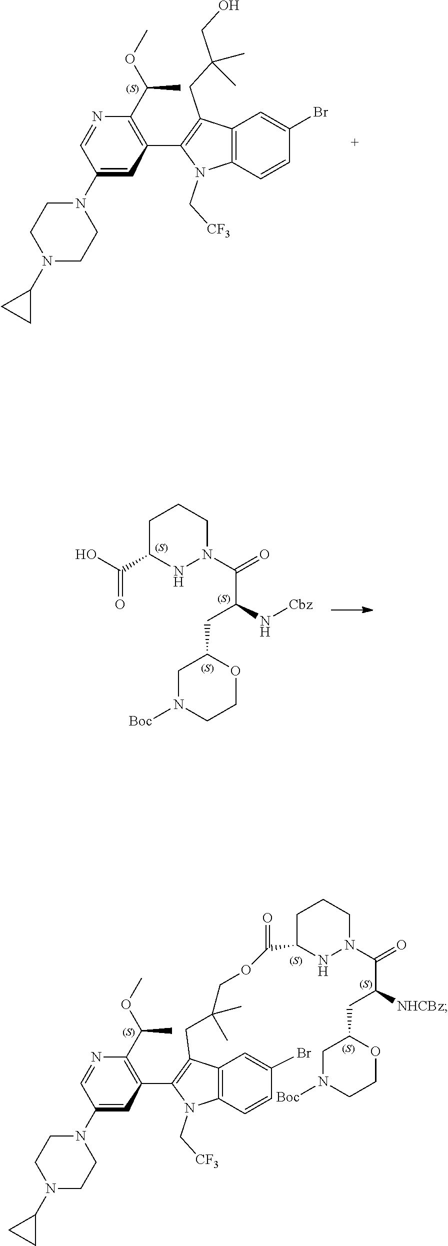

b) deprotecting the Boc of the compound of Formula XVI to form a compound of Formula XVII having the following structure:

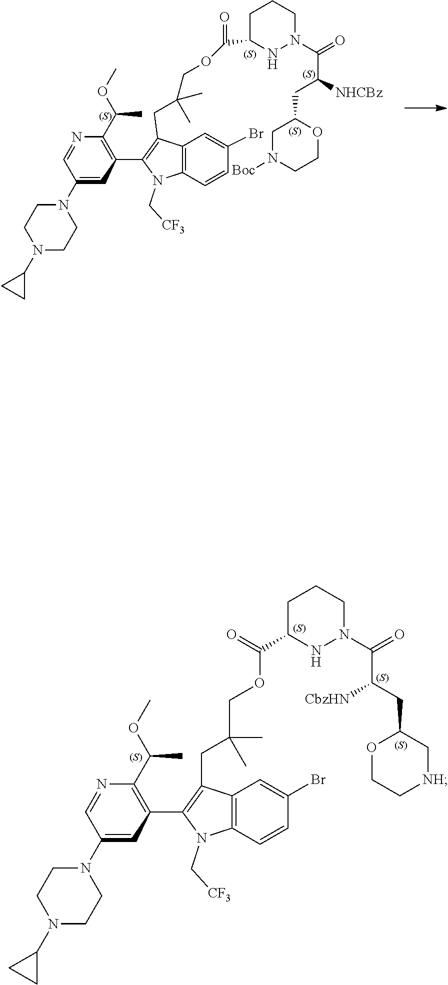

c) cyclizing the compound of Formula XVII with a transition metal catalyst to form a compound of Formula XVII having the following structure:

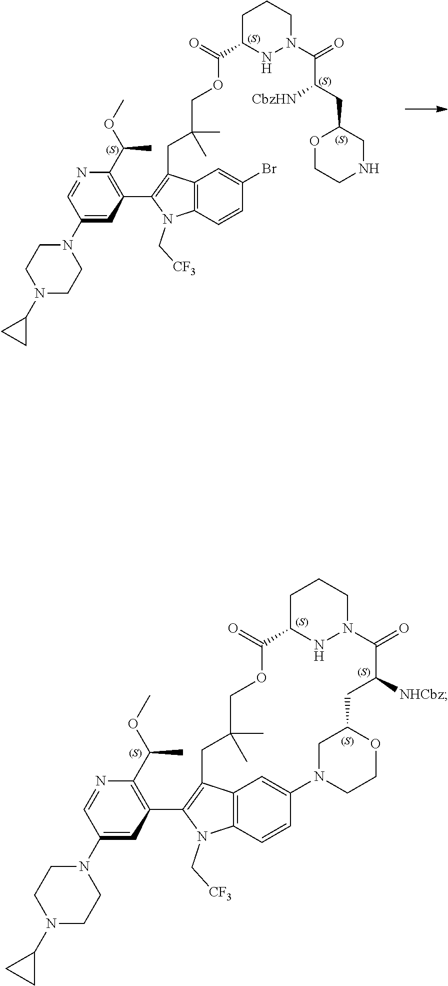

d) removing (e.g., deprotecting) PG_{b} of the compound of Formula XVIII to form a compound of Formula XIX having the following structure:

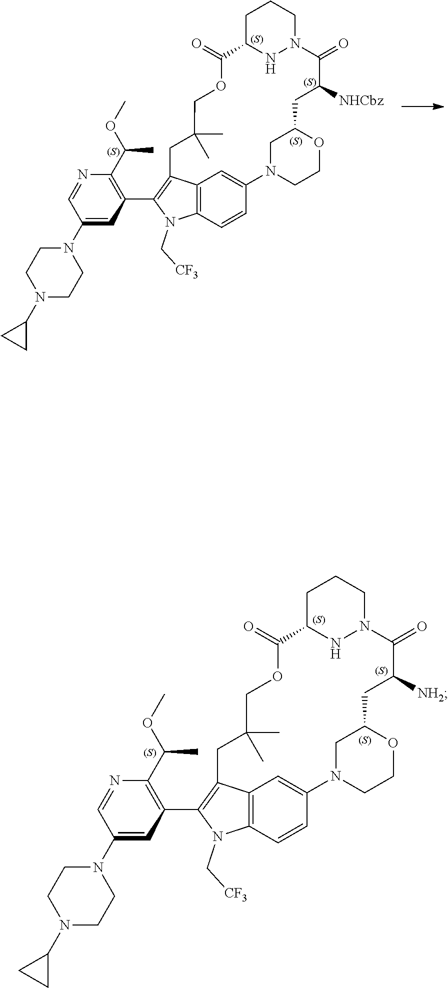

and
e) coupling the compound of Formula XIX with the compound of Formula IV to form Compound 1 having the following structure:

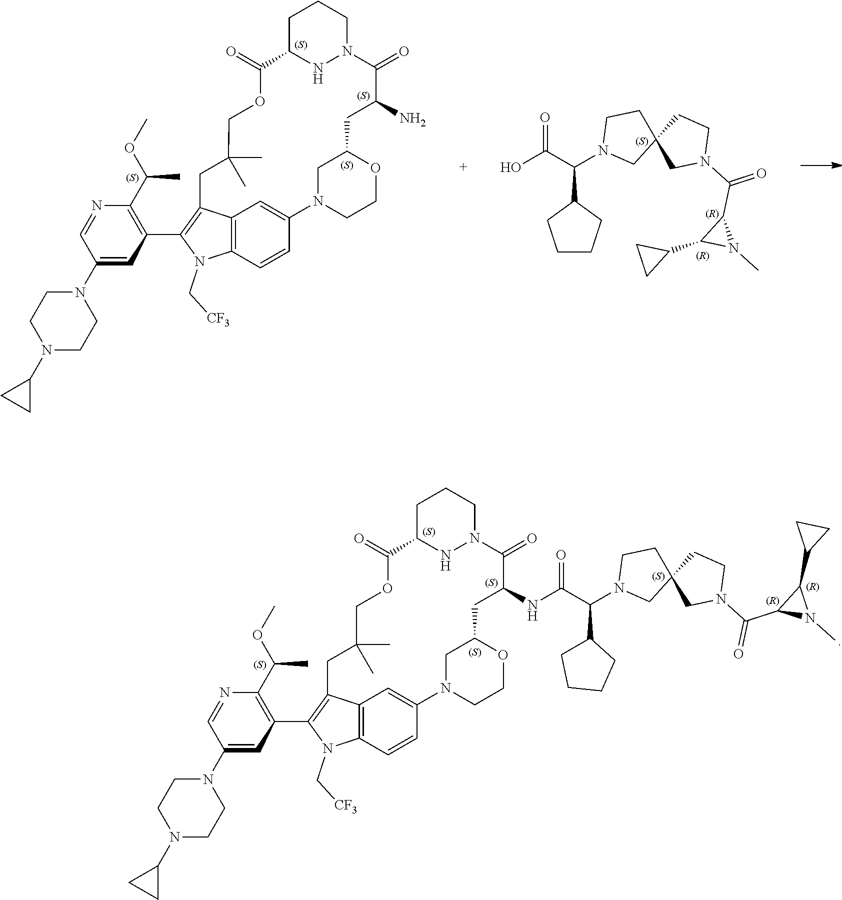

In some embodiments, step (a) includes contacting the compound of Formula XV and the compound of Formula XII with a carbodiimide coupling reagent, an anti-racemization agent, and a base. In some embodiments, the carbodiimide coupling reagent is EDCl, the anti-racemization agent is HOBt, and the base is N,N-diisopropylethylamine. In some embodiments, step (a) is carried out according to the following scheme:

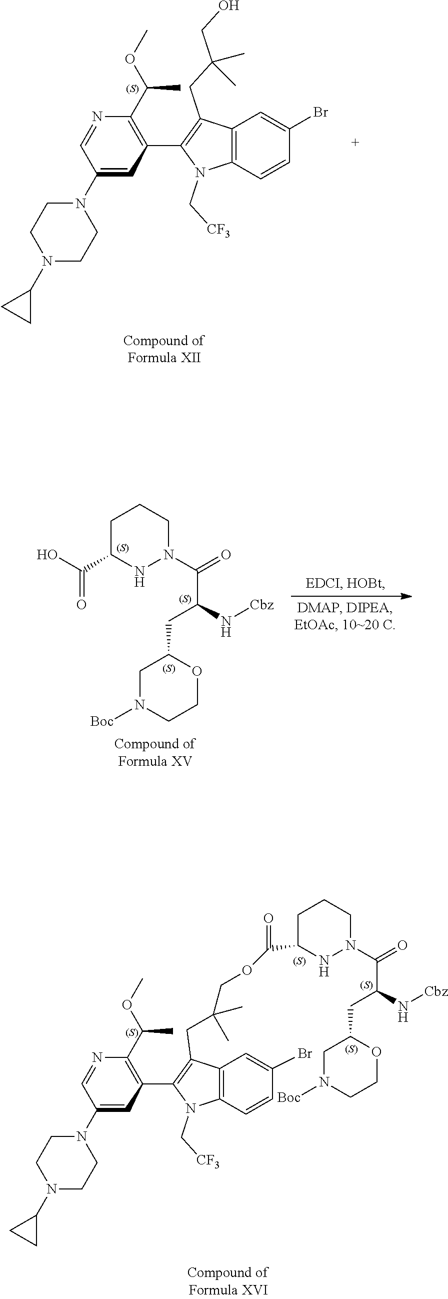

In some embodiments, the deprotecting step (b) includes contacting the compound of Formula XVI with an acid. In some embodiments, the acid is trifluoroacetic acid. In some embodiments, the deprotecting step (b) is carried out according to the following scheme:

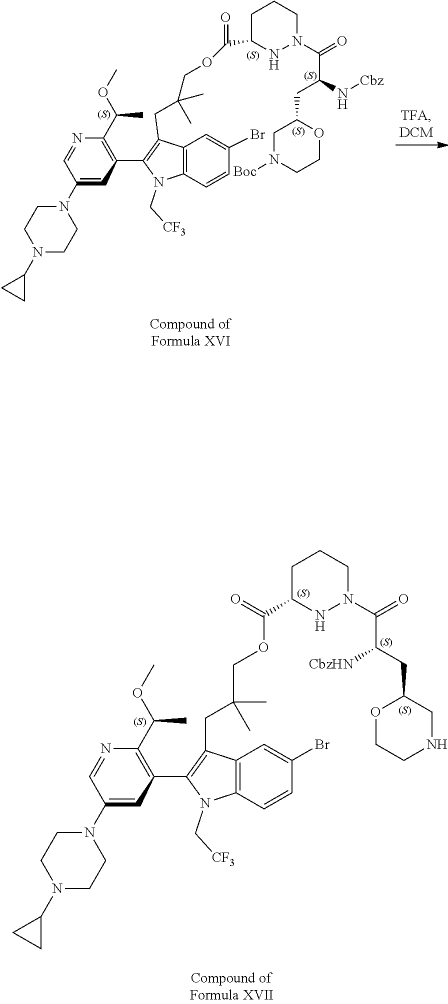

In some embodiments, the cyclizing step (c) includes contacting the compound of Formula XVII with a transition metal catalyst, a phosphine ligand, and a base. In some embodiments, the transition metal catalyst includes palladium, platinum, nickel, copper, and iron catalysts. In some embodiments, the transition metal catalyst is a palladium catalyst. In some embodiments, the palladium catalyst is P(tBu)₃ Pd G3 or Pd₂(dba)₃. In some embodiments, the phosphine ligand is Q-Phos or [(tBu)₃PH]BF₄. In some embodiments, the base is K₃PO₄.

In some embodiments, the cyclizing step (c) is carried out according to the following scheme:

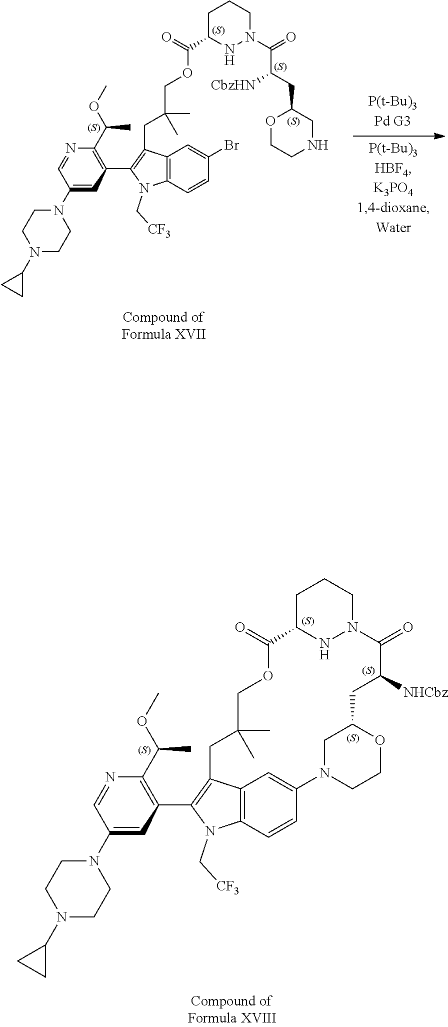

In some embodiments, the cyclizing step (c) is carried out according to the following scheme:

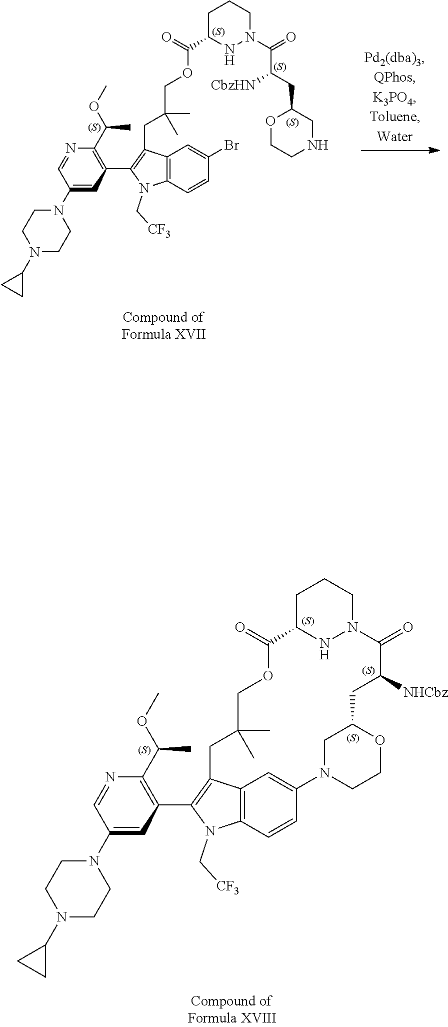

In some embodiments, the cyclizing step (c) further includes contacting the compound of Formula XVIII with hydrochloric acid to form a hydrochloride salt of the compound of Formula XVIII.

In some embodiments, the deprotecting step (d) includes contacting the compound of Formula XVIII with a palladium catalyst and hydrogen gas in an organic solvent. In some embodiments, the palladium catalyst is Pd/C. In some embodiments, the organic solvent is methyl tert-butyl ether. In some embodiments, the deprotecting step (d) is carried out according to the following scheme:

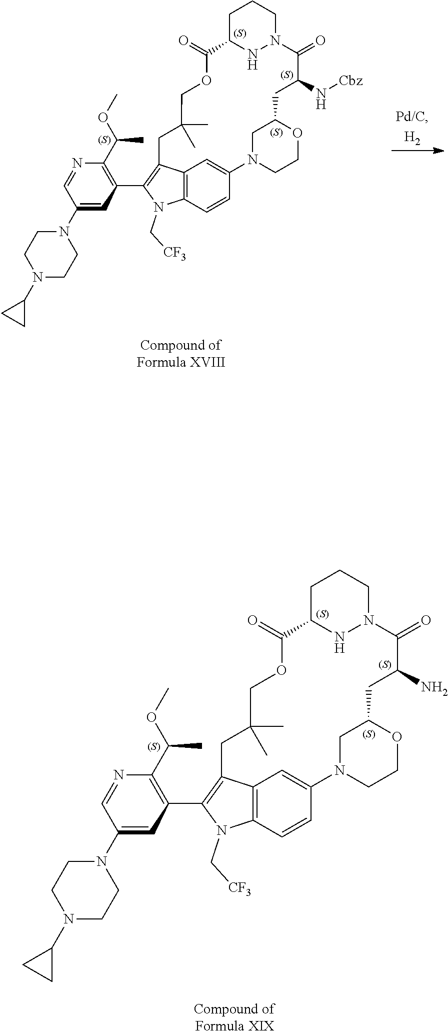

In some embodiments, the deprotecting step (d) further includes washing the compound of Formula XIX with N-acetyl cysteine.

In some embodiments, the deprotecting step (e) includes contacting the compound of Formula XIX and the compound of Formula IV with a coupling reagent, an anti-racemization reagent, and a base.

In some embodiments, the coupling reagent is PyBOP or EDCl. In some embodiments, the anti-racemization agent is selected from the group consisting of Oxyma, HOBt, or HOPO. In some embodiments, the base is N,N-diisopropylethylamine.

In some embodiments, the coupling step (e) is carried out according to the following scheme:

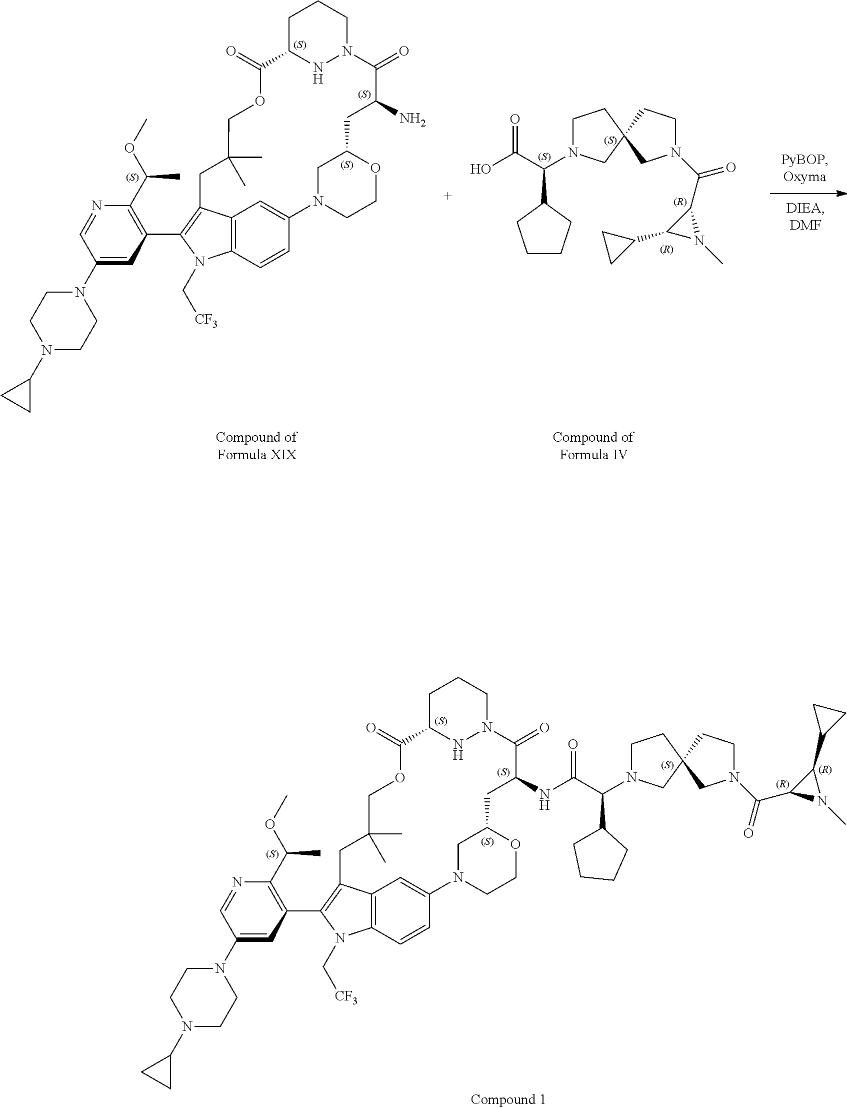

In some embodiments, the coupling step (e) is carried out according to the following scheme:

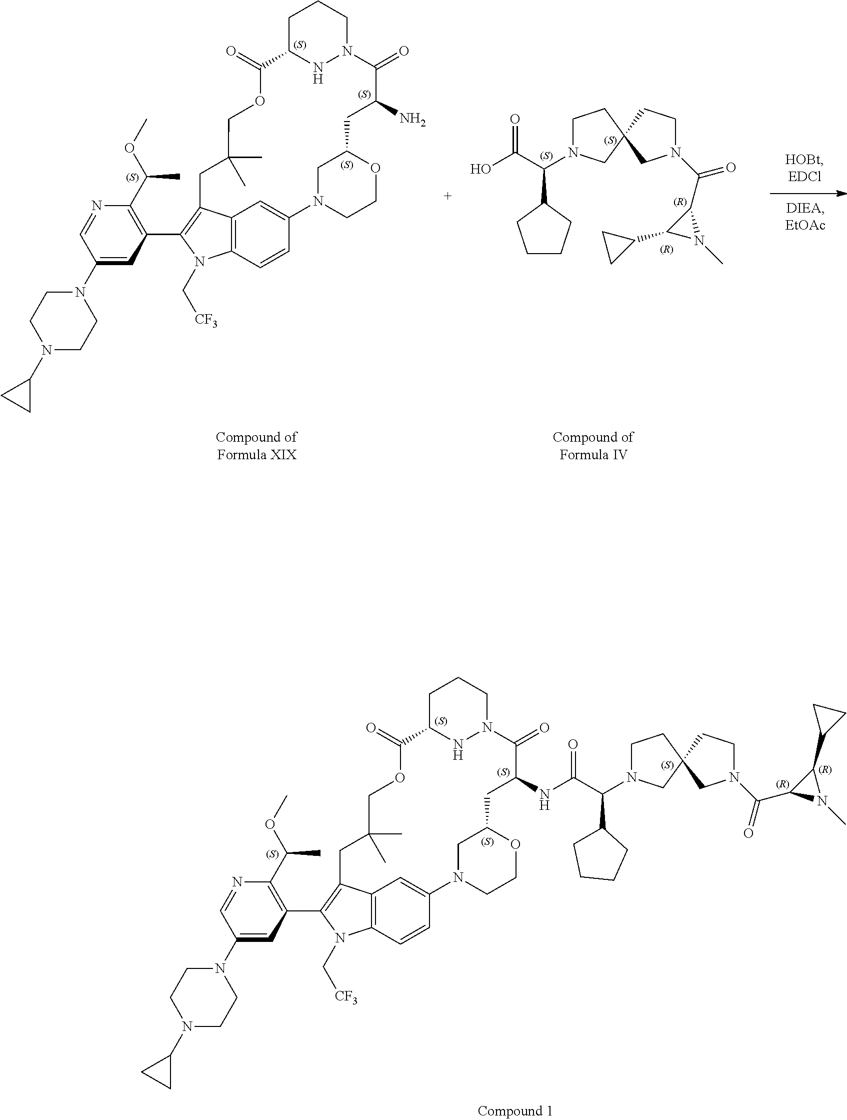

In some embodiments, the coupling step (e) is carried out according to the following scheme:

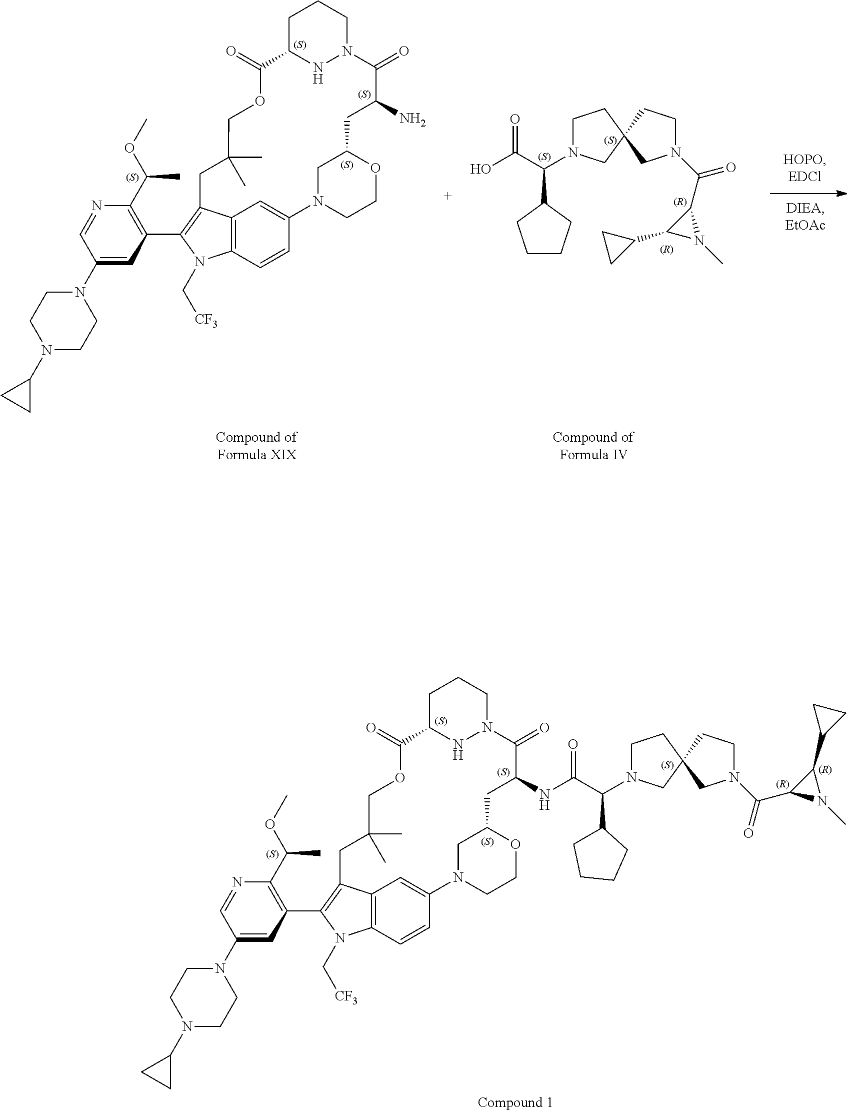

In some embodiments, the method further includes the step of purifying Compound 1. In some embodiments, the purifying includes recrystallizing Compound 1. In some embodiments, the recrystallizing includes adding a first solvent, followed by adding a second solvent. In some embodiments, the first solvent is dioxane or 2-methyltetrahydrofuran. In some embodiments, the second solvent is diisopropyl ether or isopropyl alcohol. In some embodiments, the recrystallizing further includes adding a third solvent. In some embodiments, the third solvent is heptane.

### Aziridine-containing Compounds

The compounds prepared by the methods described herein may be useful as cross-linking groups.

For example, a compound of Formula IV contains an aziridine moiety that may act as an

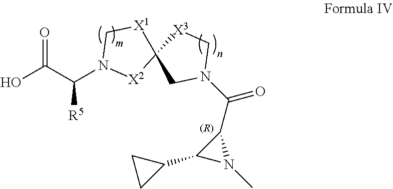

Persons of skill in the art will be familiar with nucleophiles that can react with an aziridine electrophile. For example, a nucleophilic amino acid (e.g., cysteine, aspartic acid, glutamic acid, tyrosine, arginine, histidine, or lysine) may react with the aziridine in the Compound of Formula IV.

The compound of Formula IV may be combined with a monovalent organic moiety. Those of skill in the art are familiar with organic moieties. The monovalent organic moiety may be, for example, a small molecule (e.g., a macrocyclic small molecule), a polymer, a nucleic acid (e.g., a DNA or RNA oligonucleotide), a polypeptide, an oligosaccharide, an organometallic, or a protein, such as a mutated protein. The organic moiety may be bound to the Compound of Formula IV as disclosed herein in a variety of ways, and persons of skill in the art are familiar with methodologies of installing a synthetic intermediate of Formula IV as described herein to a monovalent organic moiety. Exemplary methods are disclosed in WO 2021/091967, WO 2022/235870, and WO 2023/060253, the disclosure of each of which is incorporated herein by reference.

Persons of skill in the art will be familiar with methods of combining carboxylic acid-containing moieties with other compounds. For example, in some embodiments, a compound of Formula IV may be reacted with an alcohol in an esterification reaction. In some embodiments, a compound of Formula IV may be reacted with an amine in a peptide coupling reaction. Those of skill in the art will be familiar with peptide coupling reactions. Nonlimiting examples of peptide coupling reagents that may be used to react the carboxylic acid moiety of Formula IV with an amine include carbodiimide coupling reagents (e.g., DCC, DIC, and EDC), uronium coupling reagents (e.g., COMU, HATU, HBTU, HCTU, TATU, TOTU, TBTU), carbonyl diimidazole (CDI), and phosphonium coupling reagents (e.g., BOP, PyBOP, BOP—CI, PyAOP, PyBOP). In some embodiments, 1-hydroxybenzotriazole (HOBt) may be added to the peptide coupling reaction.

In some embodiments, the carboxylic acid may first be converted to an ester that can react directly with a nucleophile.

For example, the compound of Formula IV may be converted to a compound of Formula V:

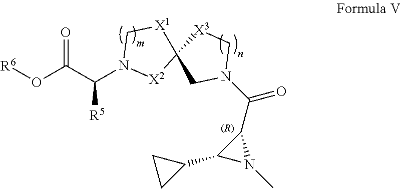

wherein R⁶ is

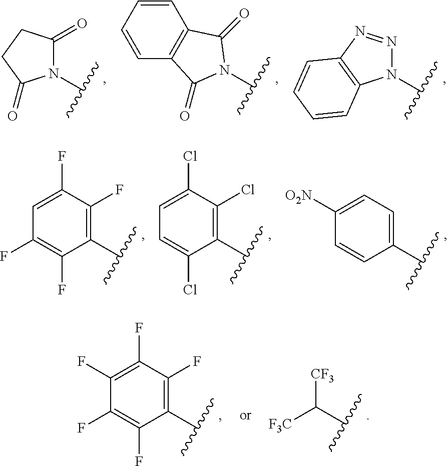

In some embodiments, the compound of Formula IV or Formula V is reacted with an amine of Formula VI, or a salt thereof:

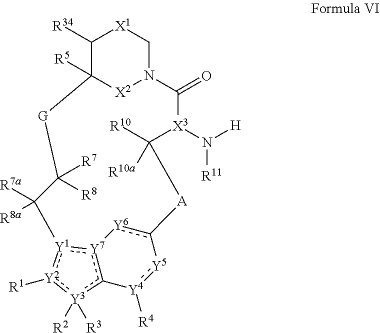

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CR^{x}, CH₂, or N;
Y⁶ is C(O), CR^{z}, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{y} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

In some embodiments, the compound of Formula IV or Formula V is reacted with an amine of Formula VII, or a salt thereof:

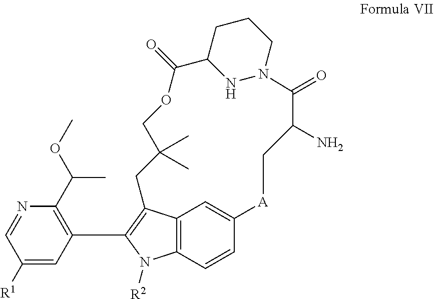

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene; R¹ is hydrogen or optionally substituted 3 to 10-membered heterocycloalkyl; and R² is optionally substituted C₁-C₆ alkyl, optionally substituted C₃₋₆ cycloalkyl, or optionally substituted C₆₋₁₀ aryl.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

All variables described in the Examples below have the same definitions as defined in the Summary, above.

### Example 1—Synthesis of a Compound of Formula I

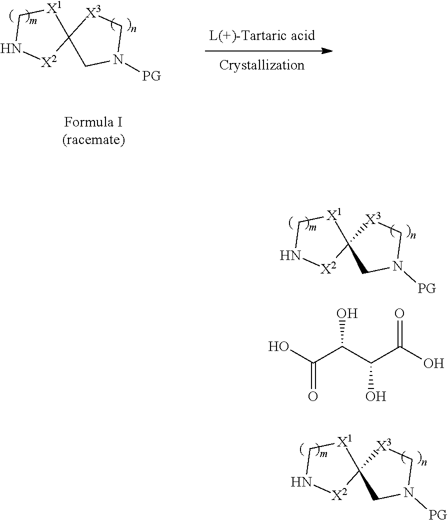

To a reactor was charged a compound of Formula I racemate (8.40 mol, 1.0 equiv), EtOH (9.6 V), L(+)-Tartaric acid (0.5 equiv) and H₂O (0.35 V). The reaction mixture was purged with N₂ gas for three times and then stirred at 80° C. for 3 h. Then the reaction was cooled down to 25° C. slowly in 12 h. The reaction mixture was stirred at 20-25° C. for 4 h. The slurry was filtered, and the cake was washed with EtOH (1 V). The wet cake was charged with EtOH (3 V) and H₂O (0.105 V) into a reactor. The mixture was purged with N₂ gas for three times and then stirred at 80° C. for 3 h. Then the reaction was cooled down to 25° C. slowly in 12 h. The reaction mixture was stirred at 20-25° C. for 4 h. The slurry was filtered, and the cake was washed with EtOH (1 V) and MTBE (1 V) respectively. The cake was dried under reduced pressure (40° C., 50-80 mbar) to afford a compound of Formula I tartrate as off-white solids (100% a/a purity, 93.3% ee, 39% yield). Table 1 summarizes the HPLC method for this synthesis.

TABLE 1
HPLC Method for Example 1
Achiral HPLC method:

| Column: | Agilent Poroshell 120 EC-C18 (4.6 × 100 mm, 2.7 μm) | | |
|---|---|---|---|
| Mobile Phase: | A: 0.05% TFA in water | | |
| | B: 0.05% TFA in MeCN | | |
| | Time (min) | A % | B % |
| Gradient: | 0.0 | 95 | 5 |
| | 10.0 | 5 | 95 |
| | 12.0 | 5 | 95 |
| | 12.1 | 95 | 5 |
| | 17.0 | 95 | 5 |

| | |
|---|---|
| Flow Rate: | 1.2 mL/min |
| UV Detector Wavelength: | 210 nm |
| Column Temperature: | 40° C. |

Chiral HPLC method:

| Column: | CHIRALCEL IG-3 (4.6 × 250 mm, 3 μm) | | |
|---|---|---|---|
| Mobile Phase: | A: hexane (0.1% DEA) | | |
| | B: IPA (0.1% DEA) | | |
| | Time (min) | A % | B % |
| Gradient: | 0.0 | 75 | 15 |
| | 60.0 | 75 | 15 |

| | |
|---|---|
| Flow Rate: | 0.8 mL/min |
| UV Detector Wavelength: | 250 nm |
| Column Temperature: | 40° C. |

### Example 2—Synthetic Procedure for benzyl (2R,3R)-3-cyclopropyl-1-methylaziridine-2-carboxylate and (2R,3R)-3-cyclopropyl-1-methylaziridine-2-carboxylic acid (Compound A barium salt)

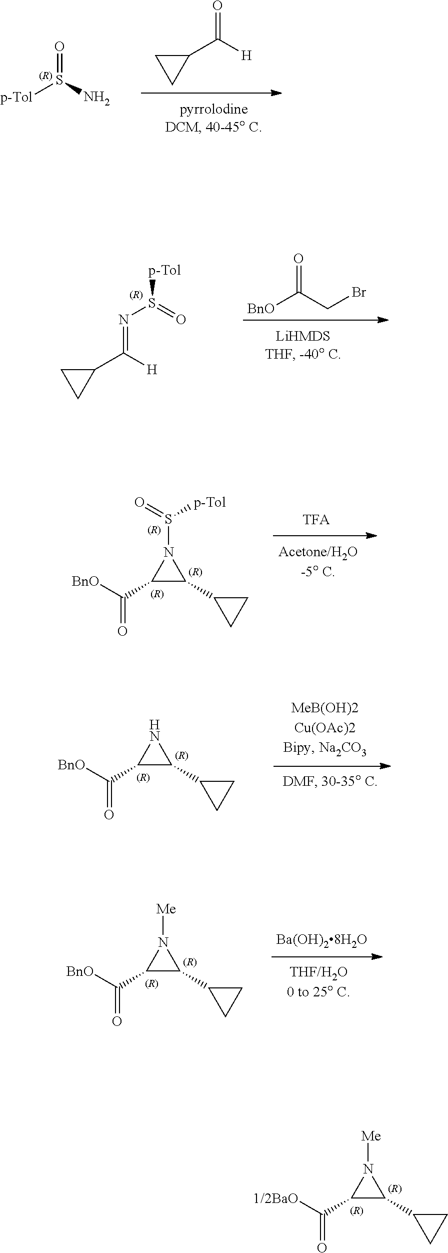

### Part 1—Synthesis of (R,E)-N-(cyclopropylmethylene)-4-methylbenzenesulfinamide

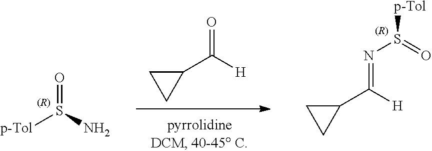

To a reactor were charged cyclopropanecarbaldehyde (5.78 kg, 82.46 mol, 2.0 equiv), pyrrolidine (14.66 g, 0.41 mol, 0.5 mol %) and DCM (128.00 L, 20 V). The reaction mixture was stirred at 20-25° C. for 30 min. The cyclopropanecarbaldehyde solution (64.00 L, 41.2 mol, 10 V) was transferred to a jacketed reactor with distillation device, followed by adding the starting material (6.4 kg, 1.0 equiv). The reaction mixture was distilled at 40-45° C. to remove H₂O. When the reaction solution was less than 7 V, another portion of cyclopropanecarbaldehyde solution (19.20 L, 3 V) was added. The cyclopropanecarbaldehyde solution was added completely within 53 h. The reaction was monitored by HPLC (IPC≤1% a/a). After completion, the reaction was cooled to 20-25° C. and the reaction solution was filtered. The filtrate was concentrated to 4 V under reduced pressure (35-40° C.) and n-heptane (10.00 L, 1.5 V) was charged. The mixture was concentrated to 4 V under reduced pressure (45-49° C.) and n-heptane (10.00 L, 1.5 V) was charged. The process was repeated 4 times until the solvent was swapped to heptane. The mixture was stirred at 15-20° C. for 14 h. The slurry was filtered, and the wet cake was dried under reduced pressure (35-40° C., 100-200 mbar) for 16 h. The product was obtained as light-yellow solids (95.8% a/a, 93.7% w/w, 84% yield).

LCMS (ESI+): Calculated for C₁₁H₁₃NOS [M+H]⁺: 208.1; found: 208.1.

¹H NMR (400 MHz, DMSO-d₆) δ 7.58-7.50 (m, 3H), 7.38 (d, J=8.0 Hz, 2H), 2.37 (s, 3H), 2.00-1.87 (m, 1H), 1.13-0.92 (m, 4H).

### Part 2—Synthesis of benzyl (2R,3R)-3-cyclopropyl-1-((R)-p-tolylsulfinyl)aziridine-2-carboxylate

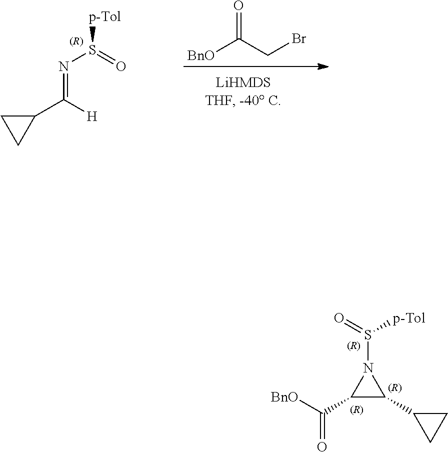

To a reactor was charged (R,E)-N-(cyclopropylmethylene)-4-methylbenzenesulfinamide (1.0 kg, 4.82 mol, 1.0 equiv), benzyl bromoacetate (1.44 kg, 6.29 mol, 1.3 equiv), THF (10.00 L, 10 V) under the protection of N₂. The reaction mixture was cooled to −50° C. to −40° C. and stirred for 30 min. LiHMDS (1.0 M in THF, 4.82 mol, 4.82 L, 1.0 equiv) was added dropwise to the reaction mixture in 2.5 h at −45±5° C. After completion the reaction was quenched by pouring the reaction mixture into ice water (20.00 L, 20 V). MTBE (10.00 L, 10 V) was added, and the mixture was stirred at 10-20° C. for 10 min. The mixture was settled for 30 min before organic phase was separated. The aqueous phase was extracted with MTBE (10.00 L, 10 V). The combined organic phase was washed with aq. NaCl solution (10 wt %, 10.00 L, 10 V). The organic phase was concentrated under reduced pressure (25-35° C., 50-100 mbar). The residue was dissolved in MTBE (10.00 L, 10 V) and charged DABCO (81.10 g, 0.72 mol, 0.15 equiv). The mixture was stirred for 9 h and was sampled for HPLC analysis (IPC: benzyl bromoacetate=0% a/a). The mixture was washed with aq. NaCl solution (10 wt %, 10.00 L, 10 V). The organic phase was concentrated under reduced pressure (25-35° C., 50-100 mbar) to afford the crude product as brown oil (80.4% a/a purity).

The crude product combined with different batches (7.5 kg, 1.0 equiv) and isopropyl ether (5.25 L, 0.7 V) were added to a reactor. The mixture was stirred at 20-25° C. for 30 min before it was cooled to 0±5° C. Product seed (1.0 g) was added. The mixture was cooled to −15° C. to −20° C. and stirred at this temperature for 2 h. The slurry was filtered, and the cake was rinsed with isopropyl ether (pre-cooled to −20° C., 0.75 L, 0.1 V). The wet cake was dried to afford light yellow solids (3.1 kg, 89.1% a/a purity). The solids were dissolved in isopropyl ether (2.70 L, 0.9 V). The solution was cooled to −15° C. to −20° C. and stirred at this temperature for 1 h. The slurry was filtered, and the cake was rinsed with isopropyl ether (pre-cooled to −20° C., 0.60 L, 0.2 V). The wet cake was dried to afford light yellow solids (2.48 kg, 98.3% a/a purity) of the product.

LCMS (ESI+): Calculated for C₂₀H₂₁NO₃S [M+H]⁺: 356.1; found: 356.3.

¹H NMR (400 MHz, DMSO-d₆) δ 7.63-7.56 (m, 2H), 7.40-7.29 (m, 5H), 7.19-7.10 (m, 2H), 5.14-5.02 (m, 2H), 3.15 (d, J=7.1 Hz, 1H), 2.38 (s, 3H), 2.35 (d, J=7.5 Hz, 1H), 1.05-0.92 (m, 1H), 0.61-0.44 (m, 3H), 0.44-0.34 (m, 1H).

### Part 3—Synthesis of benzyl (2R,3R)-3-cyclopropylaziridine-2-carboxylate

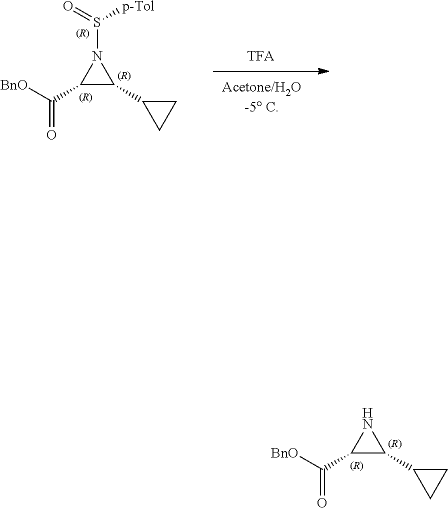

To a reactor were charged benzyl (2R,3R)-3-cyclopropyl-1-((R)-p-tolylsulfinyl)aziridine-2-carboxylate (3.00 kg, 8.44 mol, 1.0 equiv), acetone (30.00 L, 10 V) and H₂O (15.00 L, 5 V). The reaction mixture was cooled to −10° C. to −5° C. TFA (4.81 kg, 42.20 mol, 5.0 equiv) was added to the reaction dropwise in 90 min at −15° C. to −5° C. The reaction mixture was stirred at −10° C. to 0° C. for 3 h.

The reaction was quenched with aq. ammonia solution (25% w/w, 1 V, 7.0 equiv). MTBE (45.00 L, 15 V) was added, and the phases were separated. The aqueous phase was extracted with MTBE (1×30.00 L, 1×10 V; 3×15.00 L, 3×5 V). The combined organic phase was washed with aq. NaCl solution (13% w/w, 2×30.00 L, 2×10 V). The organic phase was concentrated and swapped with MTBE (10.00 L×3) to remove acetone. The MTBE solution was washed with water (30.00 L, 10 V). The organic phase was concentrated to dryness to afford oil (25-35° C., 50-100 mbar). The residue was dissolved in isopropyl ether (2.50 L, 0.42 V) and n-heptane (2.00 L, 0.33 V) was added to the solution. The solution was cooled to 10° C. and product seed (20 g) was added. The mixture was stirred for 1 h and then cooled to 5-10° C. n-heptane (2.00 L, 0.33 V) was added to the solution and the mixture was stirred at 5-10° C. for 2 h. The slurry was filtered at 5-10° C. and the cake was rinsed with isopropyl ether/n-heptane (1:5 v/v, 0.2 V). The cake was dried under reduced pressure (100-200 mbar, 25° C.) to afford the product (1.50 kg, 92.1% a/a, 89.6% w/w assay, 100% ee, 99.4% de).

LCMS (ESI+): Calculated for C₁₃H₁₅NO₂ [M+H]⁺: 218.1; found: 218.2.

¹H NMR (400 MHz, CDCl₃) δ 7.43-7.31 (m, 5H), 5.32-5.20 (m, 2H), 2.73 (d, J=6.0 Hz, 1H), 1.68 (dd, J=8.2, 6.0 Hz, 1H), 0.83-0.70 (m, 1H), 0.65-0.56 (m, 1H), 0.54-0.40 (m, 2H), 0.36-0.25 (m, 1H).

### Part 4—Synthesis of benzyl (2R,3R)-3-cyclopropyl-1-methylaziridine-2-carboxylate

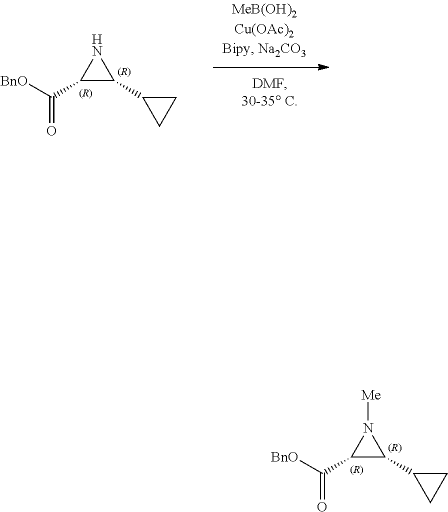

To a reactor were charged benzyl (2R,3R)-3-cyclopropylaziridine-2-carboxylate (10.90 kg, 50.17 mol, 1.0 equiv), DMF (109.00 L, 10 V), MeB(OH)₂ (9.00 kg, 150.35 mol, 3.0 equiv), Cu(OAc)₂ (10.10 kg, 50.17 mol, 1.0 equiv), 2,2′-dipyridine (7.90 kg, 50.17 mol, 1.0 equiv), anhydrous Na₂CO₃ (16.00 kg, 150.96 mol, 3.0 equiv) and 4 Å molecular sieves (43.60 kg, 4 w/w). The reaction mixture was bubbled with 5% O₂/95% N₂ (10-14 L/min) while maintaining the temperature at 20±10° C. The reaction was heated to 30-35° C. and stirred at the temperature for 15 h under 5% O₂/95% N₂. A sample was taken for HPLC analysis.

The reaction was cooled to 20° C. The reaction mixture was filtered, and the cake was rinsed with MTBE (109 L, 10 V). The filtrate was transferred to a reactor, MTBE (109 L, 10 V) and H₂O (109 L, 10V) were added. The phases were separated, and the aqueous phase was extracted with MTBE (2×109 L, 2×10 V). The combined organic phase was washed with aq. NaCl solution (5×109 L, 5×1.0V) and H₂O (163.50 L, 15 V) respectively. The organic phase was concentrated to 6-7 V under reduced pressure (s 45° C.). The concentrated solution of the product was used for the next step (92.49% a/a, 15.67% HPLC assay, 9.9 kg of product, 78.8% yield, 99.6% ee, 100% de).

### Part 4b—Alternative Synthesis of benzyl (2R,3R)-3-cyclopropyl-1-methylaziridine-2-carboxylate

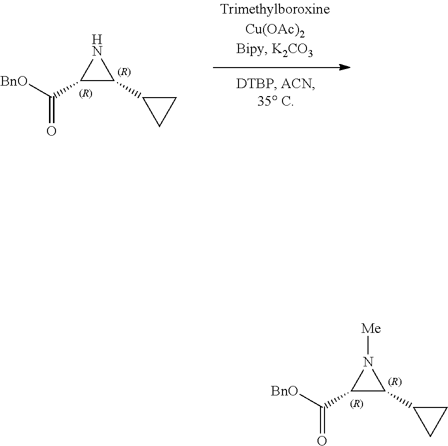

To a reactor were charged benzyl (2R,3R)-3-cyclopropylaziridine-2-carboxylate (10 g, 0.046 mole, 1.0 equiv), acetonitrile (118.5 g, 12×), trimethylboroxine (8.09 g 50% THE solution, 0.7 equiv, 0.032 mol), Cu(OAc)₂ (8.36 kg, 0.046 mol, 1.0 equiv), 2,2′-dipyridine (7.2 g, 0.046 mol, 1.0 equiv), anhydrous K₂CO₃ (12.72 g, 0.092 mol, 2.0 equiv), and di-tert-butyl peroxide (6.73 g, 0.046 mol, 1 equiv). The reaction was heated to 35° C. and stirred at the temperature for 23 h A sample was taken for HPLC analysis. The reaction was cooled to 20° C. The reaction mixture was filtered, and the cake was rinsed with MTBE (37 g, 3.7×). The filtrate was transferred to a reactor and cooled to 0° C., after which 30% sodium thiosulfate (145.43 g, 14.5×) was added dropwise. The mixture was concentrated to 10 V at 35° C. under vacuum. The residual mixture was extracted with MTBE (111 g, 11.1×). The organic phase was concentrated to 2 V under vacuum. The concentrated solution of the product was used for the next step (94.0% purity, 94.0% yield).

LCMS (ESI+): Calculated for C₁₄H₁₇NO₂ [M+H]⁺: 232.1; found: 232.1.

¹H NMR (400 MHz, DMSO-d₆) δ 7.41-7.30 (m, 5H), 5.22-5.07 (m, 2H), 2.26 (s, 3H), 2.20 (d, J=6.6 Hz, 1H), 1.39 (t, J=6.9 Hz, 1H), 0.82-0.69 (m, 1H), 0.49-0.28 (m, 3H), 0.22-0.11 (m, 1H).

### Part 5—Synthesis of (2R,3R)-3-cyclopropyl-1-methylaziridine-2-carboxylic acid, % Ba salt

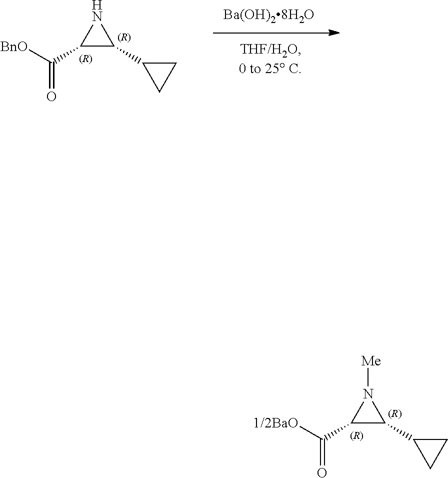

To a reactor were charged benzyl (2R,3R)-3-cyclopropyl-1-methylaziridine-2-carboxylate (1.50 kg, 6.48 mol, 1.00 equiv), H₂O (1.50 L, 1 V) and THE (15.00 L, 10 V) at 25-30° C. The reaction was cooled to 0-5° C. Ba(OH)₂·8H₂O (1.02 kg, 3.24 mol, 0.5 equiv) was added to the reaction mixture and the reaction was warmed to 25-30° C. The reaction was stirred at 25-30° C. for 16 h. A sample was taken for HPLC analysis.

The reaction was concentrated under reduced pressure (25-30° C., 50-100 mbar) and the solvent was swapped with MeCN (2×15.00 L, 2×10 V). MeCN (15.00 L, 10 V) was charged, and the mixture was stirred at 25-30° C. for 16 h. The reaction mixture was concentrated under reduced pressure (25-30° C., 50-100 mbar) and the solvent was swapped with MTBE (3×15.00 L, 3×10 V). MTBE (15.00 L, 10 V) was charged, and the mixture was stirred at 25-30° C. for 16 h. The slurry was filtered, and the cake was rinsed with MTBE (2×3.00 L, 2×2 V). The wet cake was dried under reduced pressure (25-30° C., 50-100 mbar) to afford crude (2R,3R)-3-cyclopropyl-1-methylaziridine-2-carboxylic acid, M Ba salt (1.38 kg, 58.9% w/w assay). Table 2 summarizes the HPLC method for this synthesis.

LCMS (ESI+): Calculated for C₇H₁₁NO₂ [M+H]⁺: 142.1; found: 142.1.

¹H NMR (400 MHz, Methanol-d₄) δ 2.38 (s, 3H), 2.11 (d, J=7.3 Hz, 1H), 1.23-1.12 (m, 1H), 1.00-0.86 (m, 1H), 0.67-0.53 (m, 1H), 0.52-0.40 (m, 2H), 0.37-0.24 (m, 1H).

TABLE 2
HPLC Method for Example 2
IPC method:

| Column: | XBridge C18 (4.6 × 150 mm, 3.5 μm) | | |
|---|---|---|---|
| Mobile Phase: | A: 10 mM NH₄OAc in water | | |
| | B: MeCN | | |
| | Time (min) | A % | B % |
| Gradient: | 0.0 | 65 | 35 |
| | 10.0 | 5 | 95 |
| | 12.0 | 5 | 95 |
| | 13.0 | 65 | 35 |

| | |
|---|---|
| Flow Rate: | 1.0 mL/min |
| UV Detector Wavelength: | 210 nm |
| Column Temperature: | 40° C. |

### Example 3—Alternate Synthetic Route for benzyl (2R,3R)-3-cyclopropylaziridine-2-carboxylate

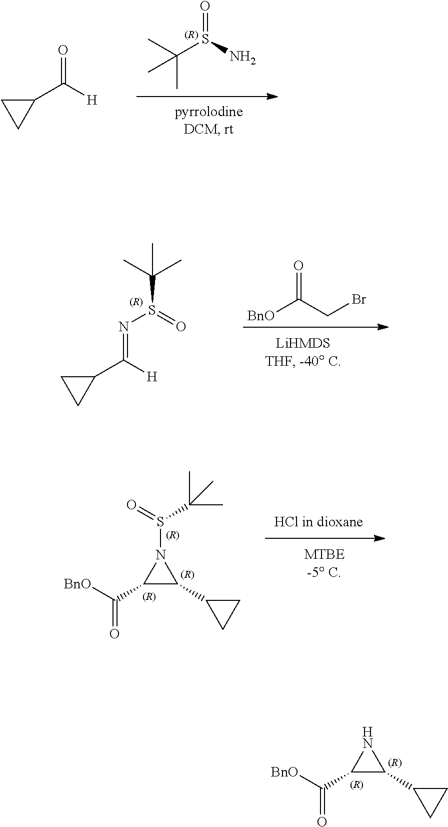

### Part 1—Synthesis of (R,E)-N-(cyclopropylmethylene)-2-methylpropane-2-sulfinamide

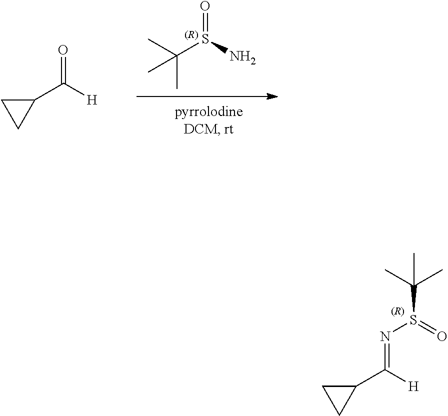

To a reactor were charged (R)-t-butyl-sulfinylamine (2.00 kg, 16.50 mol, 1.0 equiv), cyclopropanecarbaldehyde (1.20 kg, 17.12 mol, 1.05 equiv), 4 Å MS (1.60 kg, 0.8 w/w), DCM (10.00 L, 5 V) and pyrrolidine (5.87 g, 0.08 mmol). The reaction mixture was stirred at rt for 16 h at which point HPLC analysis showed reaction completion. After completion, the mixture was filtered through celite pad and rinsed with DCM (3.00 L, 1.5 V). The filtrate was concentrated to afford crude product (2.89 kg, 99.6% a/a, 94.7% assay, 95.8% yield).

LCMS (ESI+): Calculated for C₈H₁₅NOS [M+H]⁺: 174.1; found: 174.1.

¹H NMR (400 MHz, CDCl₃) δ 7.42 (d, J=7.9 Hz, 1H), 1.94 (qt, J=8.0, 4.6 Hz, 1H), 1.13 (s, 9H), 1.08-1.01 (m, 2H), 0.95-0.86 (m, 2H).

### Part 2—Synthesis of benzyl (2R,3R)-1-((R)-tert-butylsulfinyl)-3-cyclopropylaziridine-2-carboxylate

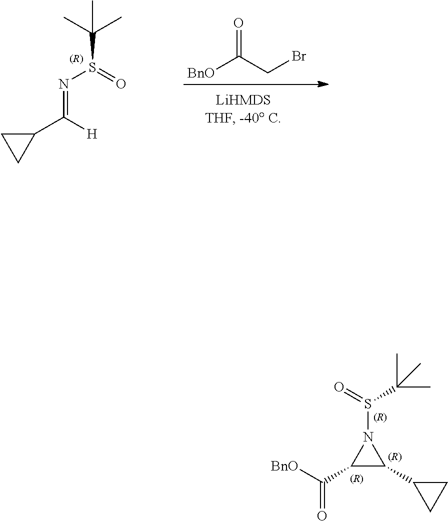

To a reactor were added the crude product from part 1 (1.10 kg, 6.35 mol, 1.0 equiv), benzyl bromoacetate (1.89 kg, 8.25 mol, 1.3 equiv) and THF (11.00 L, 10 V). The reaction was cooled to −70° C.-75° C. LiHMDS (1.0 M in THF, 6.35 mol, 1.0 equiv) was added dropwise in 2 h at −70° C.˜-75° C. and the reaction mixture was stirred for 1 h. The reaction was warmed gradually to rt and stirred for overnight at which point HPLC analysis showed reaction completion (IPC: starting material s 1% a/a, 210 nm). After completion, the reaction mixture was poured into ice water (6.05 L, 5.5 V). MTBE (11.00 L, 10 V) was added, and the mixture was stirred for 10 min at 20-25° C. The mixture was settled for 30 min and organic phase was separated. The aqueous phase was extracted with MTBE (2×11.00 L, 2×10 V). The combined organic phase was washed with aq. NaCl solution (10 wt %, 11.00 L, 10 V). The organic phase was concentrated under reduced pressure (25-35° C., 50-100 mbar). The residue was dissolved in MTBE (11.00 L, 10 V) and charged DABCO (0.21 kg, 1.90 mol, 0.3 equiv). The mixture was stirred for 2 h at 20-25° C. and was sampled for HPLC analysis (IPC: benzyl bromoacetate=0% a/a). The mixture was washed with H₂O (11.00 L, 10 V). The organic phase was added aq. NaOH solution (0.1 M, 11.00 L, 10 V) and the mixture was stirred at 20-25° C. for 2 h. The reaction mixture was sampled for HPLC analysis. The organic phase was separated, and the aqueous phase was extracted with MTBE (10 V). The combined organic phase was washed with aq. NaCl solution (10 wt %, 11.00 L, 10 V) and H₂O (11.00 L, 10 V) respectively. The organic solvent was concentrated under reduced pressure and MTBE (1.10 L, 1 V) was added to the residue. The mixture was cooled to 10° C. and the product seed was charged, followed by adding n-heptane (8.80 L, 8 V) in 2 h. The mixture was cooled to −5° C. and stirred for 16 h at −5° C. The slurry was filtered, and the cake was dried to afford the product (1.25 kg, 96.4% LCAP, 94.1% assay, 57.7% yield).

LCMS (ESI+): Calculated for C₁₇H₂₃NO₃S [M+H]⁺: 322.2; found: 322.2.

¹H NMR (400 MHz, CDCl₃) δ 7.43-7.33 (m, 5H), 5.24 (q, J=12.3 Hz, 2H), 3.41 (d, J=7.1 Hz, 1H), 2.07 (t, J=6.9 Hz, 1H), 1.24 (s, 9H), 0.55-0.47 (m, 3H), 0.35-0.26 (m, 1H).

### Part 3—Synthesis of benzyl (2R,3R)-3-cyclopropylaziridine-2-carboxylate

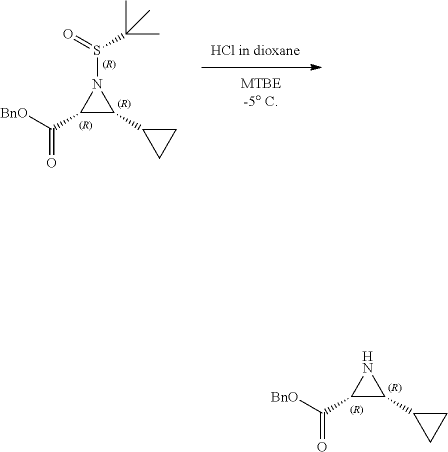

To a reactor were added benzyl (2R,3R)-1-((R)-tert-butylsulfinyl)-3-cyclopropylaziridine-2-carboxylate (0.10 kg, 0.31 mol, 1.0), MTBE (1.50 L, 15 V). The reaction mixture was cooled to −10° C.˜−5° C. HCl in dioxane (4.0 M, 1.56 mol, 5.0 equiv) was added dropwise in 35 min at −10° C. to −5° C. The reaction was stirred at −10° C. to −5° C. for 30 min and the reaction was sampled for HPLC analysis. The reaction mixture was filtered at −10° C. to −5° C. and the cake was rinsed with pre-cooled MTBE (−20° C., 0.50 L, 5 V) to give the crude product as an HCl salt.

To a reactor were added MTBE (1.00 L, 10 V) and triethylamine (62.90 g, 0.62 mol, 2.0 equiv). The solution was cooled to −10° C.˜−5° C. The crude product HCl salt was added to the reaction in 10 min at −10° C. to −5° C. The reaction was stirred at −10° C. to −5° C. for 3 h and then warmed to 0-5° C. H₂O (0.5 L, 5 V) was added to the reaction and stirred for 10 min. The organic phase was separated, and the aqueous phase was extracted with MTBE (0.50 L×2, 5 V×2). The combined organic phase was concentrated under reduced pressure at 30° C. to give crude product. The crude was dissolved in MTBE (0.10 L, 1 V) and the solution was cooled to 15° C. n-Heptane (0.70 L, 7 V) was added dropwise over 2 h at 15° C. The mixture was cooled to −5° C. and stirred at −5° C. overnight. The slurry was filtered and dried under vacuum to afford benzyl (2R,3R)-3-cyclopropylaziridine-2-carboxylate as off-white solids (53.30 g, 99.8% LCAP, 78.8% yield).

LCMS (ESI+): Calculated for C₁₃H₁₅NO₂ [M+H]⁺: 218.1; found: 218.2.

¹H NMR (400 MHz, CDCl₃) δ 7.43-7.31 (m, 5H), 5.32-5.20 (m, 2H), 2.73 (d, J=6.0 Hz, 1H), 1.68 (dd, J=8.2, 6.0 Hz, 1H), 0.83-0.70 (m, 1H), 0.65-0.56 (m, 1H), 0.54-0.40 (m, 2H), 0.36-0.25 (m, 1H).

### Example 4—Synthetic Procedure for benzyl (S)-2-cyclopentyl-2-((S)-2,7-diazaspiro[4.4]nonan-2-yl)acetate (a compound of Formula IIIe-1)

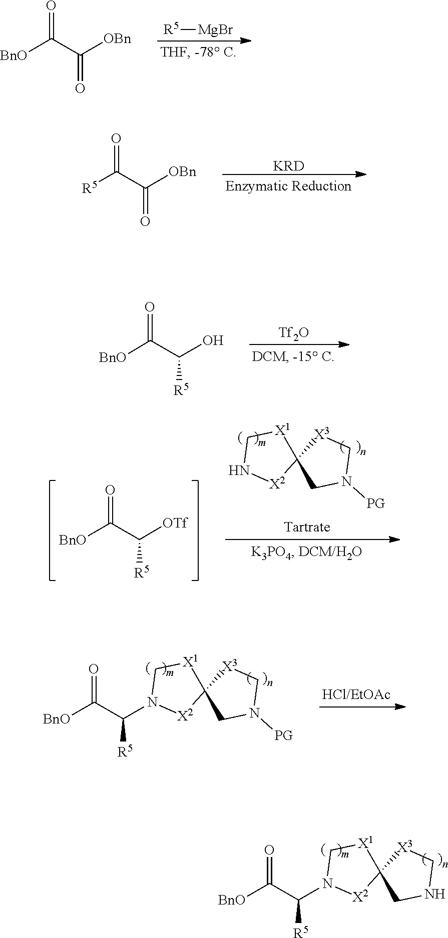

### Part 1—Synthesis of a Compound of Formula IIIc-1

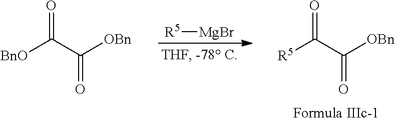

To a reactor were charged dibenzyl oxalate (1.00 kg, 3.70 mol, 1.0 equiv) and THF (30.00 L, 30.0 V) at 20-25° C. as solution A. To another reactor was charged R⁵—MgBr (0.22 M in THF, 1.3 equiv) at 20-25° C. under N₂ flow as solution B. Solution A and solution B were mixed with a flat-push flow reactor (flow rate was A: 250 mL/min, B: 182.2 mL/min, temperature was −75 to −70° C., retention time in reactor was 15 s). The reaction was monitored by HPLC at which point HPLC analysis showed reaction completion. The reaction was quenched by sat. NH₄Cl (10.00 L, 10 V) aqueous solution. The mixture was stirred at 20-25° C. for 30 min. The phases were separated, and the organic phase was washed with aq. citric acid (0.5 wt %, 10.00 L, 10 V). The organic phase was concentrated under vacuum to dryness and isopropyl alcohol (2.00 L, 2 V) was added to the residue to give the product as an IPA solution. To a reactor was charged aq. NaHSO₃ solution (40 wt %, 1.50 L, 1.5 V), followed by adding the product IPA solution. The mixture was stirred at 20-25° C. for 2 h. IPA (13.5 V) was charged to the mixture in 2 h. The mixture was continued to stir at 20-25° C. for 10 h. The slurry was filtered, and the cake was washed with IPA (3 V). The cake was dissolved in H₂O (7 V) and aq. Na₂CO₃ (20 wt %, 2-2.5 V) was added dropwise to the solution to adjust pH≥9 while stirring at −5° C.-5° C. MTBE (5 V) was added to the solution and the mixture was stirred at rt for 30 min. The organic phase was separated and washed with aq. citric acid (0.3 V) and brine (5 V) respectively. The organic phase was concentrated under vacuum to dryness to the compound of Formula IIIc-1 as a colorless oil (99.4% LCAP, 96.8% assay, 58% yield).

### Part 2—Synthesis of a Compound of Formula IIId-1

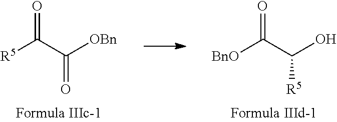

To a reactor were charged H₂O (2.50 L, 10 V), Na₂HPO₄ (84.80 g, 33.9% w/w), NaH₂PO₄ (2.00 g, 0.82% w/w) at 20-25° C. The reaction was warmed to 30-35° C. Glucose (0.58 kg, 3.23 mol, 3.0 equiv) and NADP (6.25 g, 2.5% w/w) were added to the reaction mixture. EW-KRED-R122 (5.00 g, 2% w/w) and GDH (12.50 g, 5% w/w) were added to the reaction mixture. The reaction mixture was stirred at 30-35° C. for at least 10 min. A compound of Formula IIIc-1 (1.08 mol, 1.0 equiv) in n-heptane (0.25 L, 1 V) was added dropwise to the reaction mixture over 10 min and the reaction mixture was stirred at 30-35° C. for 20 h during which pH was checked after 8 h (the pH was controlled at 7.3-8.2 with 1M aq. NaOH solution). The reaction was monitored by HPLC at which point HPLC analysis showed reaction completion. After completion of reaction, MTBE (5.00 L, 20 V) was added, and the mixture was kept stirring for 10 min. The mixture was filtered′ and the cake was washed with MTBE (0.50 L, 2 V). The filtrate was settled for 10 min and the organic phase was separated. The aqueous phase was washed with MTBE (5.00 L, 20 V). The combined organic phase was washed with aq. NaCl (20 wt %, 5.00 L, 20 V). The organic phase was concentrated under vacuum to dryness to afford colorless oil as a compound of Formula IIId-1 (97.8% LCAP, 97.2% assay, 100% ee, 97.5% yield).

### Part 3—Synthesis of a Compound of Formula IIIe-1

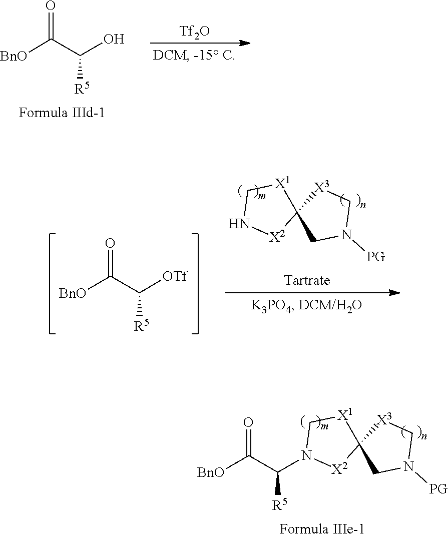

To a reactor were charged a compound of Formula IIId-1 (83.18 mol, 1.0 equiv) and DCM (234.00 L, 12 V) at 15-25° C., followed by adding DIPEA (21.50 kg, 166.36 mol, 2.0 equiv). The reaction mixture was cooled to −10-0° C. under N₂ flow. Tf₂O (31.85 kg, 116.45 mol, 1.4 equiv) was added dropwise to the reaction mixture over 1 h. The reaction mixture was kept stirring for 1 h at −10-0° C. and a sample was taken for HPLC analysis. H₂O (19.50 L, 1 V) was added to the reaction at 0-5° C., followed by adding a compound of Formula I (½ tartaric salt, 91.92 mol, 1.1 equiv) and K₃PO₄ (45.90 kg, 216.23 mol, 2.6 equiv). The reaction was warmed to 20-25° C. over 1 h and was continued to stir for 6 h. After completion, aq. HCl (0.5 M, 293.00 L, 15 V) was added to the reaction at 10±5° C. The mixture was stirred for 30 min and the organic phase was separated. To the organic phase was added H₂O (195.00 L, 10 V) at 10±5° C. The mixture was stirred for 30 min and the organic phase was separated. The organic phase was concentrated to dryness to afford the product as yellow oil (93.2% LCAP, 91.6% yield). Table 3 summarizes the HPLC method for this synthesis.

TABLE 3
HPLC Method
IPC method:

| Column: | XBridge C18 (4.6 × 150 mm, 3.5 μm) | | |
|---|---|---|---|
| Mobile Phase: | A: 0.05% TFA in water (v/v) | | |
| | B: 0.05% TFA in MeCN (v/v) | | |
| | Time (min) | A % | B % |
| Gradient: | 0.0 | 60 | 40 |
| | 12.0 | 5 | 95 |
| | 15.0 | 5 | 95 |
| | 16.0 | 60 | 40 |

| | |
|---|---|
| Flow Rate: | 1.0 mL/min |
| UV Detector Wavelength: | 210 nm |
| Column Temperature: | 40° C. |

### Part 4—Synthesis of a Compound of Formula III-1

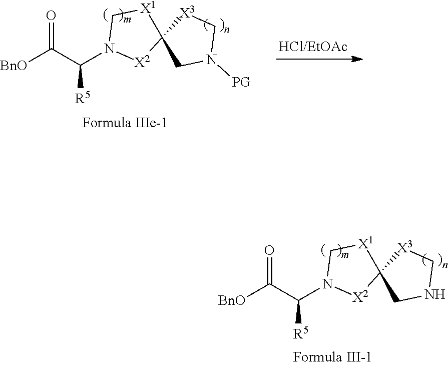

To a reactor were charged a compound of Formula IIIe-1 (68.00 mol, 1.0 equiv) and EtOAc (150.50 L, 5 V). The solution was cooled to 0-5° C. HCl in EtOAc solution (4 M, 150.50 L, 5 V) was added dropwise to the reaction. The reaction was warmed to 20-30° C. and was stirred at 20-30° C. for 6 h. The reaction was monitored by HPLC at which point HPLC analysis showed reaction completion. After completion, the reaction was cooled to 0-10° C., aq. HCl (0.5 M, 301.00 L, 10 V) was added to the reaction. The mixture was stirred at 20-30° C. for 20 min, settled and phases are separated. The organic phase was extracted with aq. HCl (0.5 M, 301.00 L, 10 V) at 0-20° C. The combined aqueous phase was washed with MTBE (2×301.00 L, 2×10 V). The pH of aqueous phase was adjusted to 11-12 at 5-10° C. using aq. NaOH solution (20 wt %). The aqueous phase was extracted with MTBE (2×301.00 L, 2×10 V). The combined organic phase was washed with aq. NaCl solution (26 wt %, 301.00 L, 10 V). The organic phase was concentrated to dryness under vacuum to afford a Compound of Formula III-1 crude as colorless oil (assay 90.1% yield).

To a reactor were added the compound of Formula III-1 crude (61.29 mol, 1.0 equiv) and IPA (210.00 L, 10 V) at 20±10° C. In another reactor, a solution of D-(+)-2,3-dibenzoyl tartaric acid (21.96 kg, 61.29 mol, 1.0 equiv) in IPA solution (210.00 L, 10 V) was prepared. ⅕ of the IPA solution of D-(+)-2,3-dibenzoyl tartaric acid (42.00 L, 2 V) was added to the solution dropwise over 1.5 h at 40-45° C. A seed of the compound of Formula III-1 (0.01 w/w) was charged to the reaction at 40-45° C. The mixture was stirred at 40-45° C. for 1.5 h. The rest of the portion (⅘) of D-(+)-2,3-dibenzoyl tartaric acid in IPA solution (168.00 L, 8 V) was added dropwise to the reaction over 6 h. The mixture was stirred at 40-45° C. for 3 h and then was cooled to 0-5° C. The mixture was continued to stir at 0-5° C. for 6 h. The slurry was filtered, and the cake was washed with IPA (42.00 L, 2 V) and MTBE (42.00 L, 2 V). The cake was dissolved in H₂O (210.00 L, 10 V) and MTBE (420.00 L, 20 V) was added to the solution. The pH of aqueous phase was adjusted to 11-12 with aq. NaOH solution (10 wt %). The mixture was stirred for 45 min, then settled and the phase was separated. The organic phase was washed with aq. NaCl solution (26 wt %, 210.00 L, 10 V). The organic phase was concentrated to dryness under vacuum to afford the product as colorless oil (99.96% LCAP, 99.01% ee, 85.8% yield). Table 4 summarizes the HPLC method for this synthesis.

TABLE 4
HPLC Method
IPC method:

| Column: | XBridge C18 (4.6 × 150 mm, 3.5 μm) | | |
|---|---|---|---|
| Mobile Phase: | A: 0.05% TFA in water (v/v) | | |
| | B: 0.05% TFA in MeCN (v/v) | | |
| | Time (min) | A % | B % |
| Gradient: | 0.0 | 60 | 40 |
| | 12.0 | 5 | 95 |
| | 15.0 | 5 | 95 |
| | 16.0 | 60 | 40 |

| | |
|---|---|
| Flow Rate: | 0.8 mL/min |
| UV Detector Wavelength: | 210 nm |
| Column Temperature: | 40° C. |

### Example 5—Alternate Synthesis for a Compound of Formula IIId-1

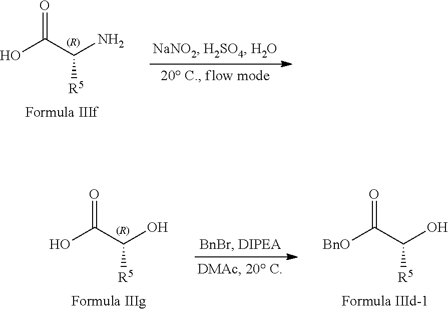

### Part 1—Synthesis of a Compound of Formula IIIg

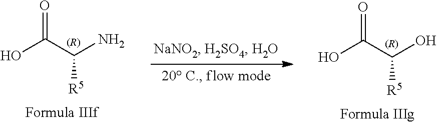

To a reactor were charged H₂SO₄ (56.76 kg, 578.78 mol, 2.5 equiv) and H₂O (660.00 L, 20 V) at 20-25° C., followed by a compound of Formula IIIf (231.51 mol, 1.0 equiv). The reaction mixture was stirred at 20-25° C. for 30 min to form a homogeneous solution. The solution was filtered via a microporous filter (0.22 μm) to tank A as solution A. To another reactor were charged NaNO₂ (102.45 kg, 1484.88 mol, 6.4 equiv) and H₂O (800.00 L, 24.1 V) at 20-25° C. The reaction mixture was stirred at 20-25° C. for 30 min and transferred to tank B as solution B. Solution A and solution B were mixed with a continuous micro reactor. Flow rate was A: 37.7 L/h, B: 27.7 L/h, temperature was 50° C., retention time in micro reactor was 2 min. The solution was purged with N₂ gas for 12 h to push most of the N₀₂ gas into the exhaust gas absorption reactor. NaCl (198.9 kg, 6 w/w) was charged to the reactor and was kept agitating to dissolve the solids. The solution was extracted with MTBE (2×994.50 L, 2×30 V). The organic phase was concentrated to 2-3 V under reduced pressure (0.08 MPa, 20-30° C.). The MTBE residue was swapped with n-heptane to 2-3 V under reduced pressure (0.08 Mpa, 20-30° C.) until MTBE residue is less than 3 wt % and KF is less than 0.5 wt %. EtOAc (19.90 L, 0.6 V) was added to the n-heptane residue and the mixture was heated to 45±2° C. The reaction mixture was stirred at 45±2° C. for at least 8 h and then gradually cooled to 30° C. by the speed of 1.5° C./h. The slurry was filtered, and the cake was washed with EtOAc/n-heptane (1:5 v/v, 33.00 L, 1 V). The wet cake was dried under vacuum (0.09 Mpa, 45±5° C.) to afford a compound of Formula IIIg as off-white solids (91.4% LCAP, 44.9% yield). Table 5 summarizes the HPLC method for this synthesis.

TABLE 5
HPLC Method
IPC method:

| Column: | XBridge C18 (4.6 × 150 mm, 3.5 μm) | | |
|---|---|---|---|
| Mobile Phase: | A: 0.05% TFA in water | | |
| | B: 0.0% TFA in MeCN | | |
| | Time (min) | A % | B % |
| Gradient: | 0.0 | 90 | 10 |
| | 10.0 | 50 | 50 |
| | 13.0 | 5 | 95 |
| | 16.0 | 5 | 95 |
| | 17.0 | 90 | 10 |

| | |
|---|---|
| Flow Rate: | 0.8 mL/min |
| UV Detector Wavelength: | 210 nm |
| Column Temperature: | 40° C. |

### Part 2—Synthesis of a Compound of Formula IIId-1

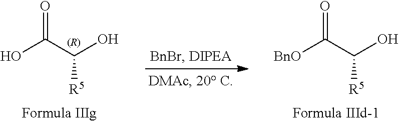

A reactor was charged with dimethylacetamide (128.00 L, 5 V), DIPEA (34.44 kg, 266.46 mol, 1.5 equiv) and a compound of Formula IIIg (177.57 mol, 1.0 equiv) at 20-25° C. under N₂ atmosphere. The reaction mixture was cooled to 0-10° C. and BnBr (36.45 kg, 213.11 mol, 1.2 equiv) was added dropwise. The reaction mixture was warmed to 15-25° C. and was continued to stir at 15-25° C. for 6 h. The reaction was monitored by GC at which point GC analysis showed reaction completion. DMAP (13.01 kg, 106.49 mol, 0.6 equiv) was added and the mixture was stirred for 18 h at 20-25° C. and a sample was taken for GC analysis to make sure BnBr is not detected. H₂O (256.00 L, 10V) was added to the solution and the resulting solution was extracted with MTBE (2×128.00 L, 2×5 V). The combined organic phase was washed with aq. citric acid (2×128.00 L, 2×5 V) and H₂O (128.00 L, 5 V) respectively. The organic phase was concentrated to 2-3 V under reduced pressure (0.08 Mpa, 20-35° C.). The MTBE residue was swapped with DCM (256.00 L, 10 V) to 2-3 V under reduced pressure (0.08 Mpa, 20-35° C.) until MTBE residue was less than 3% LCAP by GC analysis. A compound of Formula IIId-1 was obtained as solution in DCM (95.6% LCAP, 105% yield). Table 6 summarizes the HPLC method for this synthesis.

TABLE 6
HPLC Method
IPC method:

| Column: | Rtx-35 30 m*0.32 mm, 1 μm | |
|---|---|---|
| Flow | 2 | mL/min |
| Inject temperature | 230° | C. |
| Oven temperature | 40° C. for 0 min, then 20° C./min to | |
| | 280° C. for 3 min | |
| Detection temperature | 280° | C. |
| Split | 30:1 | |
| Air flow | 300 | mL/min |
| N₂ flow | 30 | mL/min |
| H₂ flow | 30 | mL/min |

### Example 6—Synthesis Procedure for a Compound of Formula IV

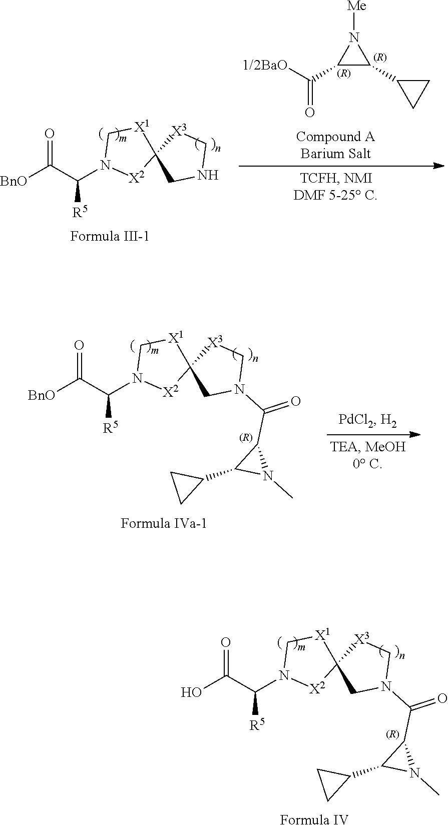

### Part 1—Synthesis of a Compound of Formula IV-1

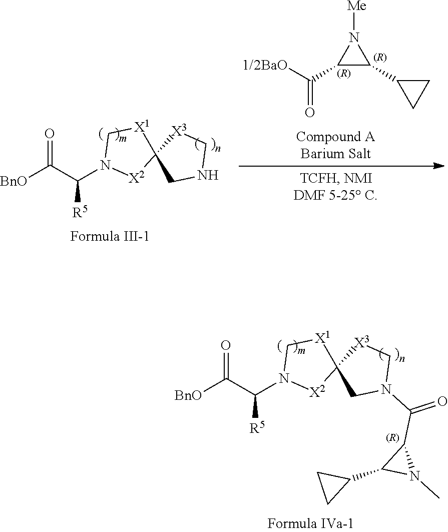

In a reactor Compound A barium salt (8.5 kg, 40.88 mol, 1.0 equiv) was dissolved in DMF (56.00 L, 4 V) at 15±10° C. NMI (13.4 kg, 163.22 mol, 4.0 equiv) and a compound of Formula III-1 (40.88 mol, 1.0 equiv) were added to the solution, followed by adding TCFH (13.80 kg, 49.18 mol, 1.2 equiv) solution in DMF (4.00 L, 1 V) dropwise over 1 h at 15±10° C. The reaction mixture was stirred at 15±10° C. for at least 30 min. The reaction was monitored by HPLC at which point HPLC analysis showed reaction completion.

The reaction mixture was cooled to 5±5° C. and quenched with H₂O (28.00 L, 2 V). The mixture was warmed to 20±5° C. and stirred for 10 min. MTBE (280.00 L, 20 V), H₂O (182.00 L, 13 V) and Na₂CO₃ (1.12 kg, 0.08 w/w) were added to the mixture and stirred for 20 min. The organic phase was separated, and the aqueous phase was extracted with MTBE (140.00 L, 10 V). The combined organic phase was washed with aq. N₂CO₃ solution (15 wt %, 2×164.60 kg, 2×11.76 w/w). The organic phase was concentrated under reduced pressure (0.08 MPa, 20-40° C.) to 4-5 V. The MTBE was swapped with EtOAc by adding EtOAc (280.00 L, 20 V) and concentrated under reduced pressure (0.08 MPa, 20-40° C.) to 4-5 V, the process was repeated twice. A portion of EtOAc (210.00 L, 15 V) was added to the residue and the mixture was stirred for 40 min. The solution was cooled to −5±5° C. and maleic acid (2.37 kg, 20.42 mol, 0.5 equiv) was added portion-wise to the solution. A compound of Formula IVa-1 maleate seed was added to the reaction and stirred for 15 min. The solution of maleic acid (6.17 kg, 1.3 equiv) in EtOAc (280.00 L, 20 V) was added dropwise to the reaction over 7.5 h and continued to stir at −5±5° C. for 7.5 h. The slurry was filtered, and the cake was washed with pre-cooled EtOAc (42.00 L, 3 V) at −5±5° C. The wet cake was collected.

To a reactor were added MTBE (140.00 L, 10 V) and the wet cake at 0±5° C. aq. Na₂CO₃ solution (15 wt %) was added at 0±5° C. until the pH of aqueous layer is 9-11. The mixture was warmed to 25±5° C. and stirred for 30 min. The organic phase was separated, and the aqueous phase was extracted with MTBE (140.00 L, 10 V). The combined organic phase was washed with aq. Na₂CO₃ solution (15% wt %, 140.00 L, 10 V). The organic phase was concentrated under reduced pressure (0.08 MPa, 20-40° C.) to 4-5 V. The MTBE was swapped with MeOH by adding MeOH (280.00 L, 20 V) and concentrated under reduced pressure (0.08 MPa, 20-40° C.) to 4-5 V, the process was repeated twice. A portion of MeOH (112.00 L, 8 V) was added to the residue and the mixture was stirred for 30 min at 20±10° C. A sample was taken for GC analysis (MTBE residue s 3 wt %). A compound of Formula IVa-1 was obtained as MeOH solution (97.8% LCAP, 50.7% yield).

### Part 2—Synthesis of a Compound of Formula IV

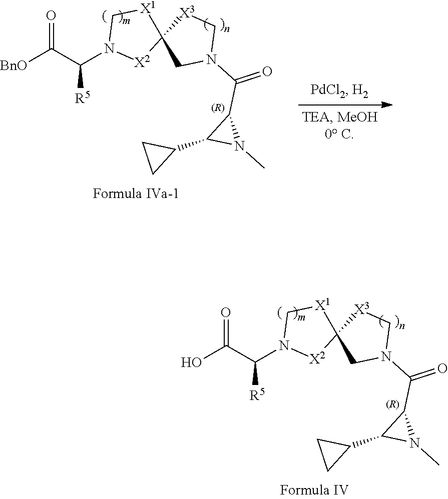

MeOH solution of a compound of Formula IVa-1 (13.00 mol, 1.0 equiv) and TEA (4.00 kg, 39.00 mol, 3.0 equiv) were added into a reactor. The mixture was cooled to 0±5° C. and PdC12 (60.50 g, 1% w/w) was added to the reaction. The reaction mixture was stirred for at least 3 h at 0±5° C. under pressure of 0-0.15 MPa with H₂. A sample was taken for HPLC analysis. The reaction mixture was filtered via diatomite and washed the filter cake with MeOH (42.35 L, 7 V). The filtrate was concentrated to 8-10 V under reduced pressure (20-30° C., 0.08 Mpa). The solvent was swapped with THE (60.50 L, 10 V) to 8-10 V under reduced pressure (20-30° C., 0.08 Mpa) twice. To the residue was added THE (60.50 L, 10 V) and TEA (2.00 kg, 19.76 mol, 1.5 equiv). The resulting solution was concentrated to 8-10 V under reduced pressure (20-30° C., 0.08 Mpa) and THE (18.15 L, 3 V) was added to the residue. The solution was stirred for at least 30 min at 20±10° C. and slurry formed. MTBE (60.50 L, 10 V) was added to the slurry over 1.2 h at 20±10° C. The mixture was concentrated to 10-13 V under reduced pressure (20-30° C., 0.08 Mpa). The solvent was swapped with MTBE (48.40 L, 8 V) to 10-13 V for 6 times by charging MTBE dropwise over 1 h and then concentration under reduced pressure (20-30° C., 0.08 Mpa). MTBE (48.40 L, 8 V) was added to the suspension dropwise over 1 h at 20±10° C. and a sample was taken for analysis (THF residue≤7% w/w). The slurry was filtered, and the cake was washed with (18.15 L, 3 V). The cake was dried under vacuum (30±5° C., 0.08 Mpa) for at least 8 h until LOD≤10% w/w. The compound of Formula IV was obtained as off-white solids (95.9% LCAP, 90.2% w/w assay, 76.4% yield).

### Compound of Formula IV Na Salt

MeOH solution of a compound of Formula IV (1.0 equiv) and TEA (3.0 equiv) were added into a reactor. The mixture was cooled to 0±5° C. and PdCl₂ (1% w/w) was added to the reaction. The reaction mixture was stirred for at least 3 h at 0±5° C. under pressure of 0-0.15 MPa with H₂. A sample was taken for HPLC analysis. The reaction mixture was filtered via diatomite and washed the filter cake with MeOH (7 V). The filtrate was concentrated to 3-4 V under reduced pressure (20-30° C., 0.08 Mpa). MeOH (7 V) was added to the residue and the solution was stirred for 10 min at rt. NaOMe (1.03 equiv, 30 wt % MeOH solution) was added to the solution at rt and the resulting mixture was stirred for at least 3 h at rt. The reaction solution was concentrated to 3-4 V under reduced pressure (20-30° C., 0.08 Mpa) to afford solution A. In another reactor was charged MTBE (60 V) and the reactor was cooled to −5-5° C. The seeds of the Compound of Formula IV Na salt (2 wt %) was added to MTBE solution and the suspension was stirred for 30 min. Solution A of the compound of Formula IV was added to MTBE over 5 h at −5-5° C. and the suspension was stirred for 2 h. The slurry was filtered, and the wet cake was washed with MTBE (2 V). The wet cake was dried at 30-40° C. for 5 h under vacuum to afford the compound of Formula IV Na salt. Table 7 summarizes the HPLC method for this synthesis.

TABLE 7
HPLC Method
IPC method:

| Column: | XBridge C18 (4.6 × 150 mm, 3.5 μm) | | |
|---|---|---|---|
| Mobile Phase: | A: 10 mM NH₄OAc in water | | |
| | B: MeOH | | |
| | Time (min) | A % | B % |
| Gradient: | 0.0 | 90 | 10 |
| | 13.0 | 5 | 95 |
| | 16.0 | 5 | 95 |
| | 17.0 | 90 | 10 |

| | |
|---|---|
| Flow Rate: | 1.0 mL/min |
| UV Detector Wavelength: | 210 nm |
| Column Temperature: | 40° C. |

### Example 7—Alternate Synthesis Route for a Compound of Formula IVa-1

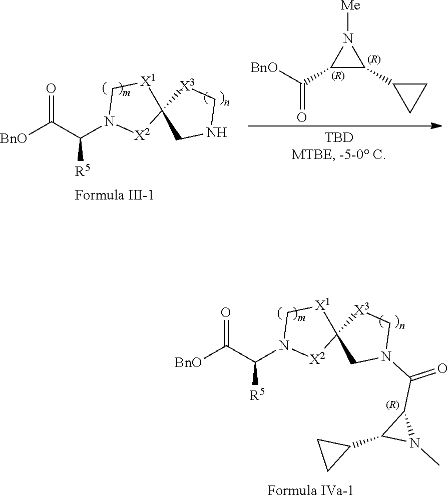

To a reactor were charged MTBE (90.50 mL, 5 V), a compound of Formula III-1 (52.85 mmol, 1.0 equiv), benzyl (2R,3R)-3-cyclopropyl-1-methylaziridine-2-carboxylate (12.84 g, 55.49 mmol, 1.05 equiv) and 1,5,7-triazobicyclo[4,4,0]dec-5-ene (TBD) (2.21 g, 15.86 mmol, 0.3 equiv). The reaction mixture was cooled to −5-0° C. The reaction mixture was stirred at −5-0° C. for 24 hours at which point HPLC analysis showed reaction completion. Aq. Na₂CO₃ solution (5 wt %, 181.00 mL, 10 V) was added to quench the reaction and the mixture was stirred at 15-25° C. for at least 10 min. The phases were separated, and the organic phase was washed with aq. Na₂CO₃ solution (5 wt %, 181.00 mL, 10 V). The organic phase was concentrated to dryness (T s 40° C.). The residue was dissolved in EtOAc (145.00 mL, 8 V) and the solution was cooled to −10-0° C. To the solution was added maleic acid (3.07 g, 26.42 mmol, 0.5 equiv), followed by adding a compound of Formula IVa-1 maleate seed (1 wt %) at −10-0° C. The mixture was stirred for at least 10 min before the solution of maleic acid in MTBE/EtOAc (v/v=3:2, 181.00 mL, 10 V) was added dropwise over 3 h. The suspension was stirred for 12 h at −10-0° C. The slurry was filtered, and the cake was washed with pre-cooled MTBE/EtOAc (v/v=1:2, 54.30 mL, 3 V). The filter cake was transferred to a reactor where MTBE (181.00 mL, 10 V) was cooled to −5-0° C. Aq. Na₂CO₃ solution (15 wt %, 181.00 mL, 10 V) was added to the solution to adjust the pH=8-10. The mixture was stirred for at least 10 min at 15-25° C. The phases were separated, and the aqueous phase was extracted with MTBE (90.50 mL, 5 V). The combined organic phase was washed with aq. Na₂CO₃ solution (5 wt %, 181.00 mL, 10 V). The organic phase was concentrated to dryness to afford the compound of Formula IVa-1 maleate (97.2% LCAP, 92.7% QNMR assay). Table 8 summarizes the HPLC method for this synthesis.

TABLE 8
HPLC Method
IPC method:

| Column: | XBridge C18 (4.6 × 150 mm, 3.5 μm) | | |
|---|---|---|---|
| Mobile Phase: | A: 0.05% TFA in water (v/v) | | |
| | B: 0.05% TFA in MeCN (v/v) | | |
| | Time (min) | A % | B % |
| Gradient: | 0.0 | 90 | 10 |
| | 12.0 | 45 | 55 |
| | 15.0 | 5 | 95 |
| | 17.0 | 5 | 95 |
| | 18.0 | 90 | 10 |

| | |
|---|---|
| Flow Rate: | 0.8 mL/min |
| UV Detector Wavelength: | 210 nm |
| Column Temperature: | 40° C. |

### Example 8—Synthetic Procedure for Compound B—tert-butyl (S)-2,7-diazaspiro[4.4]nonane-2-carboxylate hemi((2R,3R)-2,3-dihydroxysuccinate)

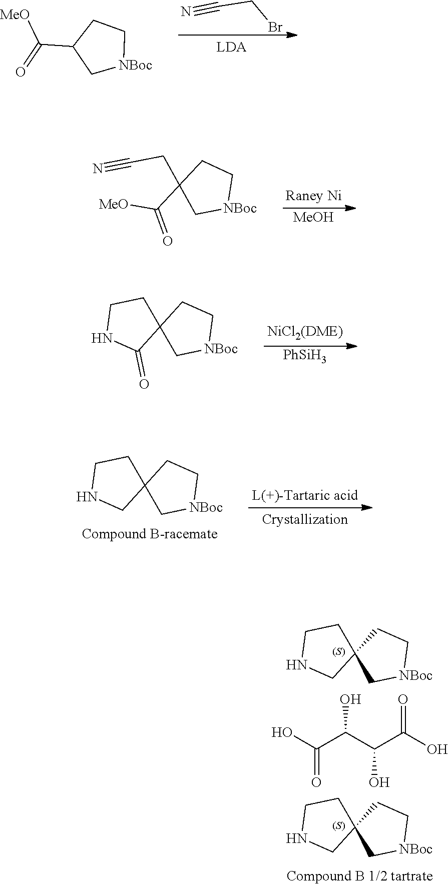

### Part 1—Synthesis of 1-(tert-butyl) 3-methyl 3-(cyanomethyl)pyrrolidine-1,3-dicarboxylate

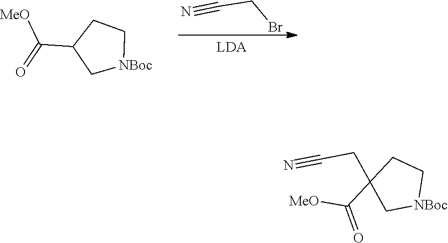

To a reactor was charged THE (16.00 L, 8 V). The reactor was cooled to −20 to −15° C. n-BuLi (2.5 M in hexane, 4.54 L, 1.3 equiv) was added to the reactor dropwise while maintaining the inner temperature between −20 to −15° C. Then di-isopropylethylamine (DIPEA, 1.24 kg, 12.25 mol, 1.4 equiv) was added dropwise at −20 to −15° C. The reaction mixture was stirred at −20 to −15° C. for 30 min under N₂. The reaction mixture was cooled to −70 to −80° C. 1-(tert-butyl) 3-methyl pyrrolidine-1,3-dicarboxylate (2.00 kg, 8.72 mol, 1.0 equiv) in THE (2.00 L, 1 V) was added dropwise to the reaction mixture at −70 to −80° C. The reaction mixture was stirred at −70 to −80° C. for 30 min under N₂. 2-bromoacetonitrile (1.36 kg, 11.34 mol, 1.3 equiv) in THE (2.00 L, 1 V) was added dropwise to the reaction mixture at −70 to −80 The reaction mixture was warmed to 20-30° C. The reaction was monitored by HPLC. The reaction mixture was quenched by adding 15% w/w aq. NH₄Cl (16.00 L, 8V) dropwise while maintaining temperature at 20±10 00 The mixture was extracted with EtOAc (20.00 L, 1 BV) and EtOAc (10.00 L, 5V). The combined organic layer was washed with 15% w/w aq. NaCl (8.00 L×3, 4V). The organic layer was then concentrated (40-50 00, 100±50 mbar) to afford 1-(tert-butyl) 3-methyl 3-(cyanomethyl)pyrrolidine-1,3-dicarboxylate as dark red oil (7.70 kg, 99.66% a/a purity, 51.3% w/w assay, 60% yield). LCMS (ESI): Calculated for C₁₃H₂₀N₂O₄ [M+H₂O]⁺: 286.2; found: [M+H₂O]: 286.2. ¹H NMR (300 MHz, DMSO-d₆, 25° C.) δ 3.71 (s, 3H), 3.68-3.64 (d, J=12 Hz, 1H), 3.38-3.25 (m, 3H), 3.00 (t, J=2.1 Hz, 2H), 2.31-2.21 (m, 1H), 2.02-1.90 (m, 1H), 1.90 (s, 9H). Table 9 summarizes the HPLC method for this synthesis.

TABLE 9
HPLC Method for Part 1
HPLC method:

| Column: | Agilent Poroshell 120 EC-C18 | | |
|---|---|---|---|
| | (3 × 50 mm, 1.9 μm) | | |
| Mobile Phase: | A: 0.1% H₃PO₄ in water | | |
| | B: MeCN | | |
| | Time (min) | A % | B % |
| Gradient: | 0.0 | 95 | 5 |
| | 6.0 | 5 | 95 |
| | 8.0 | 5 | 95 |
| | 8.1 | 95 | 5 |
| | 10.0 | 95 | 5 |

| | |
|---|---|
| Flow Rate: | 0.8 mL/min |
| UV Detector | 210 nm |
| Wavelength: | |
| Column Temperature: | 40° C. |
| Retention Times: | 1-(tert-butyl) 3-methyl pyrrolidine-1,3- |
| | dicarboxylate: 3.781 min |
| | 1-(tert-butyl) 3-methyl 3-(cyanomethyl)pyrrol- |
| | idine-1,3-dicarboxylate: 3.689 min |

### Part 2—Synthesis of tert-butyl 6-oxo-2, 7-diazaspiro[4.4]nonane-2-carboxylate

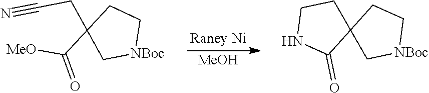 [SMILES: "MeOH" ; COC(=O)C1(CC#N)CCN(C(=O)OC(C)(C)C)C1 ; CC(C)(C)OC(=O)N1CCC2(CCNC2=O)C1]

To a reactor were charged MeOH (59.00 L, 10 V), 1-(tert-butyl) 3-methyl 3-(cyanomethyl)pyrrolidine-1,3-dicarboxylate (2) (5.90 kg, 21.99 mol, 1.0 equiv) and Raney Ni (5.90 kg, 100% w/w). The reaction mixture was purged with N₂ gas three times, followed by purging with H₂ for three times to 1 atm. The reaction mixture was stirred at 20-30° C. for 16 hours at which point HPLC analysis showed reaction completion (IPC: 2/(2+3)≤1% a/a, 210 nm). The resulting mixture was filtered through diatomite (5.90 kg, 100% w/w). The cake was washed with MCOH (23.60 L, 4 V) and H₂O (23.60 L, 4 V) respectively. The filtrate was concentrated (40-50° C., 80-150 mbar) to a volume of 4-5 V. DCM (47.20 L, 8 V) was added to the residue. The organic layer was separated, and the aqueous layer was extracted with CM (23.60 L, 4 V). The combined organic layers were dried over anhydrous Na₂SO₄ (11.80 kg) for 30 min at 20-30° C. The organic phase was filtered and concentrated under reduced pressure to 2 V (40-50° C., 80-150 mbar). MTBE (11.80 L, 2 V) was added to the residue and the mixture was concentrated to obtain solid (40-50° C., 80-150 mbar). MTBE (35.40 L, 6 V) was added to the residue and the mixture was stirred at 25±5 00 for 1 h. n-Heptane (11.80 L, 2 V) was added dropwise to the mixture and the mixture was stirred at 25±5° C. for 3-5 h. The resulting slurry was filtered and the cake was washed with n-heptane/MTBE (v/v, 1:1, 5.90 L, 1 V). The wet cake was dried under reduced pressure (40-50° C., 80-150 mbar) for 16 h. tert-butyl 6-oxo-2,7-diazaspiro[4.4]nonane-2-carboxylate was obtained as yellow solids (2.25 kg, 98.9% a/a purity, 97% w/w assay, 61% yield). LCMS (ESI): Calculated for C₁₂H₂₀N₂O₃ [M-^{t}Bu+H]: 185.1; found: [M-^{t}Bu+H]: 185.2. ¹H NMR (300 MHz, DMSO-d₆, 25° C.) δ 6.38 (s, 1H), 3.66-3.25 (m, 6H), 2.23-2.12 (m, 3H), 1.80-1.77 (m, 1H), 1.45 (s, 9H). Table 10 summarizes the HPLC method for this synthesis.

TABLE 10
HPLC Method for Part 2
HPLC method:

| Column: | Agilent Poroshell 120 EC-C18 | | |
|---|---|---|---|
| | (3 × 50 mm, 1.9 μm) | | |
| Mobile Phase: | A: 0.1% H₃PO₄ in water | | |
| | B: MeCN | | |
| | Time (min) | A % | B % |
| Gradient: | 0.0 | 95 | 5 |
| | 6.0 | 5 | 95 |
| | 8.0 | 5 | 95 |
| | 8.1 | 95 | 5 |
| | 10.0 | 95 | 5 |

| | |
|---|---|
| Flow Rate: | 0.8 mL/min |
| UV Detector | 210 nm |
| Wavelength: | |
| Column Temperature: | 40° C. |
| Retention Times: | 1-(tert-butyl) 3-methyl 3-(cyanomethyl)pyrrol- |
| | idine-1,3-dicarboxylate: 3.698 min |
| | tert-butyl 6-oxo-2,7-diazaspiro[4.4]nonane-2- |
| | carboxylate: 2.906 min |

### Part 3—Synthesis of tert-butyl 2,7-diazaspiro[4.4]nonane-2-carboxylate

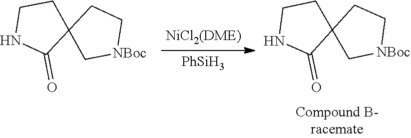 [SMILES: CC(C)(C)OC(=O)N1CCC2(CCNC2=O)C1 ; "PhSiH3" ; CC(C)(C)OC(=O)N1CCC2(CCNC2=O)C1]

To a sealed autoclave reactor were charged toluene (10.00 L, 5 V), tert-butyl 6-oxo-2,7-diazaspiro[4.4]nonane-2-carboxylate (2.00 kg, 8.32 mol, 1.0 equiv), NiCl₂(DME) (0.013 kg, 0.058 mol, 0.007 equiv) and PhSiH₃ (1.98 kg, 18.30 mol, 2.2 equiv). The reaction mixture was purged with N₂ gas three times. The reaction was stirred at 115-120° C. for 24 h at which point HPLC analysis showed reaction completion (IPC: 3/(3+4) s 2% a/a, 210 nm). The reaction mixture was cooled to room temperature. MTBE (20.00 L, 10 V) and aq. citric acid (20% w/w, 12.00 L, 6 V) were added. The mixture was stirred at 20-30° C. for 2-3 h. The aqueous layer was separated, and the organic layer was extracted with aq. citric acid (20% w/w, 2.00 L, 1 V). To the combined aqueous phase was added MTBE (10.00 L, 5 V). The aqueous layer was separated and adjusted to pH=13-14 with aq. KOH (20% w/w, 14.00 L, 7 V). The aqueous phase was extracted with MTBE (30.00 L×4, 15 V). The combined organic phases were dried over anhydrous Na₂SO₄, and filtered. The filtrate was concentrated under reduced pressure (40-50° C., 50-300 mbar) to afford tert-butyl 2,7-diazaspiro[4.4]nonane-2-carboxylate (1.95 kg, 97.7% a/a purity, 80.8% w/w assay, 86% yield) as a brown oil. LCMS (ESI+): Calculated for C₁₂H₂₂N₂O₂ [M+H]⁺: 227.2; found: 227.1. ¹H NMR (400 MHz, CDCl₃) δ 3.52-3.31 (m, 2H), 3.31-3.16 (m, 2H), 3.01 (t, J=7.1 Hz, 2H), 2.85 (dd, J=11.0, 6.0 Hz, 1H), 2.77 (dd, J=11.0, 4.5 Hz, 1H), 1.99 (s, 1H), 1.87-1.64 (m, 4H), 1.46 (s, 9H). Table 11 summarizes the HPLC method for this synthesis.

TABLE 11
HPLC Method for Part 3
HPLC method:

| Column: | Agilent Poroshell 120 EC-C18 | | |
|---|---|---|---|
| | (3 × 50 mm, 1.9 μm) | | |
| Mobile Phase: | A: 0.1% H₃PO₄ in water | | |
| | B: MeCN | | |
| | Time (min) | A % | B % |
| Gradient: | 0.0 | 95 | 5 |
| | 6.0 | 5 | 95 |
| | 8.0 | 5 | 95 |
| | 8.1 | 95 | 5 |
| | 10.0 | 95 | 5 |

| | |
|---|---|
| Flow Rate: | 0.8 mL/min |
| UV Detector | 210 nm |
| Wavelength: | |
| Column Temperature: | 40° C. |
| Retention Times: | tert-butyl 6-oxo-2,7-diazaspiro[4.4]nonane- |
| | 2-carboxylate: 2.906 min |
| | Compound B racemate: 2.121 min |

### Part 4—tert-butyl (S)-2,7-diazaspiro[4.4]nonane-2-carboxylate % tartaric acid (Compound B % tartaric acid salt)

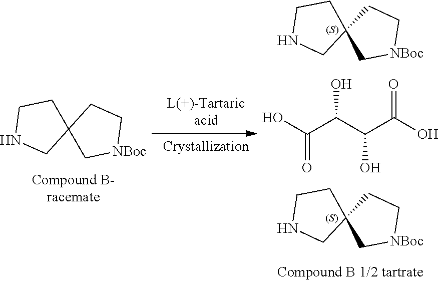

A reactor was charged Compound B-racemate (1.90 kg, 8.40 mol, 1.0 equiv), EtOH (18.24 L, 9.6 V), L(+)-Tartaric acid (0.63 kg, 4.20 mol, 0.5 equiv) and H₂O (0.66 L, 0.35 V). The reaction mixture was purged with N₂ gas for three times and then stirred at 80° C. for 3 h. Then the reaction was cooled down to 25° C. slowly in 12 h. The reaction mixture was stirred at 20-25° C. for 4 h. The slurry was filtered, and the cake was washed with EtOH (1.90 L, 1 V). The wet cake was charged with EtOH (5.70 L, 3 V) and H₂O (0.20 L, 0.105 V) into a reactor. The mixture was purged with N₂ gas for three times and then stirred at 80° C. for 3 h. Then the reaction was cooled down to 25° C. slowly in 12 h. The reaction mixture was stirred at 20-25° C. for 4 h. The slurry was filtered, and the cake was washed with EtOH (1.90 L, 1 V) and MTBE (1.90 L, 1 V) respectively. The cake was dried under reduced pressure (40° C., 50-80 mbar) to afford Compound B % tartrate as off-white solids (0.979 kg, 100% a/a purity, 93.3% ee, 39% yield). LCMS (ESI+): Calculated for C₁₂H₂₂N₂O₂ [M+H]⁺: 227.2; found: 227.1. ¹H NMR (400 MHz, D₂O) δ 4.28 (s, 1H), 3.44 (dt, J=12.1, 7.4 Hz, 3H), 3.40-3.30 (m, 3H), 3.25 (s, 2H), 2.02 (dt, J=11.0, 7.0 Hz, 2H), 1.96 (td, J=7.0, 3.0 Hz, 2H), 1.42 (s, 9H). Tables 12 and 13 summarize the HPLC method for this synthesis.

TABLE 12
Achiral HPLC Method for Part 4

| Column: | Agilent Poroshell 120 EC-C18 | | |
|---|---|---|---|
| | (4.6 × 100 mm, 2.7 μm) | | |
| Mobile Phase: | A: 0.05% TFA in water | | |
| | B: 0.05% TFA in MeCN | | |
| | Time (min) | A % | B % |
| Gradient: | 0.0 | 95 | 5 |
| | 10.0 | 5 | 95 |
| | 12.0 | 5 | 95 |
| | 12.1 | 95 | 5 |
| | 17.0 | 95 | 5 |

| | |
|---|---|
| Flow Rate: | 1.2 mL/min |
| UV Detector Wavelength: | 210 nm |
| Column Temperature: | 40° C. |
| Retention Times: | Compound B: 4.253 min |

TABLE 13
Chiral HPLC Method for Part 4

| Column: | CHIRALCEL IG-3 (4.6 × 250 mm, 3 μm) | | |
|---|---|---|---|
| Mobile Phase: | A: hexane (0.1% DEA) | | |
| | B: IPA (0.1% DEA) | | |
| | Time (min) | A % | B % |
| Gradient: | 0.0 | 75 | 15 |
| | 60.0 | 75 | 15 |

| | |
|---|---|
| Flow Rate: | 0.8 mL/min |
| UV Detector Wavelength: | 250 nm |
| Column Temperature: | 40° C. |
| Retention Times: | Compound B enantiomer: 32.026 min |
| | Compound B: 41.782 min |

### Example 9—Alternative Synthesis of Compound B—tert-butyl (S)-2,7-diazaspiro[4.4]nonane-2-carboxylate hemi((2R,3R)-2,3-dihydroxysuccinate)

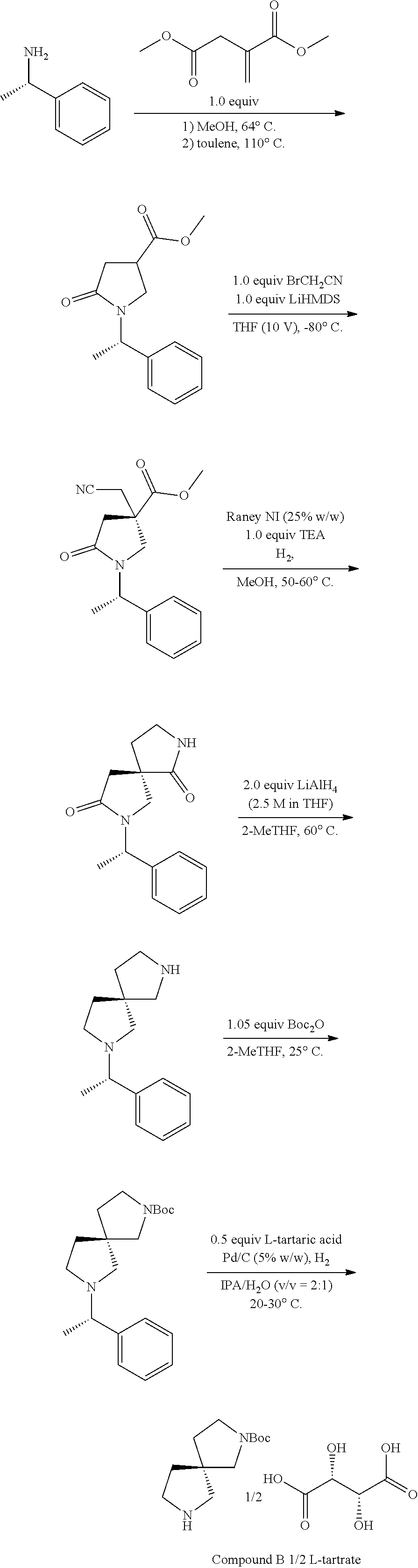

### Part 1—Synthesis of methyl 5-oxo-1-((S)-1-phenylethyl)pyrrolidine-3-carboxylate

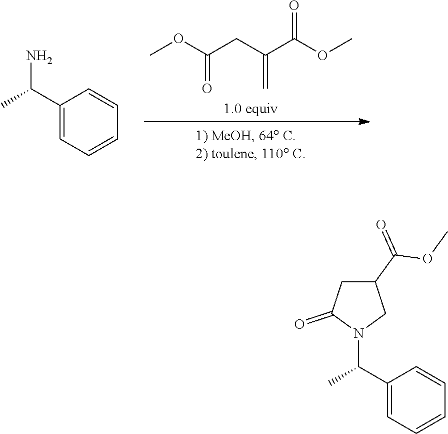

To a reactor was charged MeOH (10 L, 4 V), (S)-(−)-1-phenylethylamine (2.50 kg, 20.63 mol, 1.0 equiv), and dimethyl itaconate (3.26 kg, 20.61 mol, 1.0 equiv). The reaction mixture was stirred at 60-70° C. for 22 h. After completion, the reaction mixture was concentrated under vacuum to remove MeOH. Toluene (12.5 L, 5 V) was added to the residue and the mixture was stirred at 100-110° C. for 20 h. The mixture was cooled to 20-25° C. and aq. citric acid solution (20% w/w, 1.5 L) was added. The mixture was stirred at 20-25° C. for 3-5 h. The organic layer was separated and concentrated under vacuum to afford methyl 5-oxo-1-((S)-1-phenylethyl)pyrrolidine-3-carboxylate as orange oil (5.1 kg).

### Part 2—Synthesis of methyl (R)-3-(cyanomethyl)-5-oxo-1-((S)-1-phenylethyl)pyrrolidine-3-carboxylate

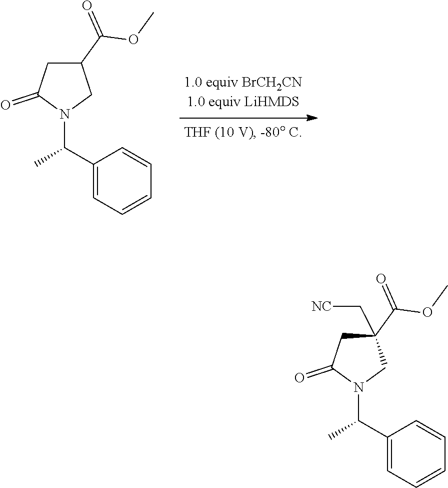 [SMILES: COC(=O)C1CC(=O)N([C@@H](C)c2ccccc2)C1 ; COC(=O)[C@]1(CC#N)CC(=O)N([C@@H](C)c2ccccc2)C1]

To a reactor was charged THE (16 L, 10 V), and methyl 5-oxo-1-((S)-1-phenylethyl)pyrrolidine-3-carboxylate (1.70 kg, 6.87 mol, 1.0 equiv). The mixture was cooled to −75 to −85° C. LiHMDS (1.0 M, 1.15 kg, 1.0 equiv) was added and the reaction mixture was stirred at −75 to −85° C. for 2 h. BrCH₂CN (0.82 kg, 6.84 mol, 1.0 equiv) was added and the reaction mixture was stirred at −75 to −85° C. for 2 h. After completion, HOAc (0.82 kg, 13.65 mol, 2.0 equiv) was added at −15-20° C. The mixture was concentrated and EtOAc (5.1 L, 3 V) was added. To the mixture was added aq. NaCl solution (25% w/w, 2.5 L, 1.5 V) and the mixture was stirred for 0.5 h. The organic phase was separated and concentrated. MTBE (3.5 L, 2 V) was charged to the residue and the mixture was stirred for 2-4 h. The resulting slurry was filtered and the wet cake was collected. The wet cake was dried to afford methyl (R)-3-(cyanomethyl)-5-oxo-1-((S)-1-phenylethyl)pyrrolidine-3-carboxylate as orange powder (0.63 kg).

### Part 3—Synthesis of (R)-7-((S)-1-phenylethyl)-2,7-diazaspiro[4.4]nonane-1,8-dione

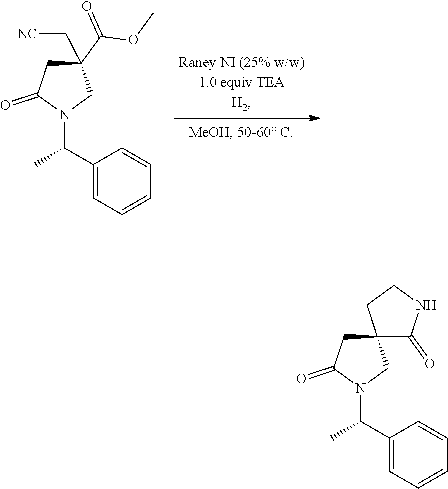 [SMILES: COC(=O)[C@]1(CC#N)CC(=O)N([C@@H](C)c2ccccc2)C1 ; C[C@@H](c1ccccc1)N1C[C@@]2(CCNC2=O)CC1=O]

To a reactor was charged MeOH (4.5 L, 5 V) and Raney Ni (0.225 kg, 25% w/w) at 15-25° C. To the mixture was added methyl (R)-3-(cyanomethyl)-5-oxo-1-((S)-1-phenylethyl)pyrrolidine-3-carboxylate (0.90 kg, 3.14 mol, 1.0 equiv) and TEA (0.32 kg, 3.16 mol, 1.0 equiv). The mixture was degassed with N₂ gas and then H₂ (1.0 MPa) was charged to the reactor. The reaction was stirred at 50-60° C. for 24 h under the atmosphere of H₂. After completion, the mixture was filtered and the filtrate was concentrated. To the concentrated residue was added 2-MeTHF (0.8 L, 1 V) and n-heptane (0.4 L, 0.5 V). The mixture was stirred at 60-70° C. for 2-4 h. The mixture was cooled and the slurry was filtered. The wet cake was dried to give (R)-7-((S)-1-phenylethyl)-2,7-diazaspiro[4.4]nonane-1,8-dione as yellow solids (0.62 kg).

### Part 4—Synthesis of (S)-2-((S)-1-phenylethyl)-2,7-diazaspiro[4.4]nonane

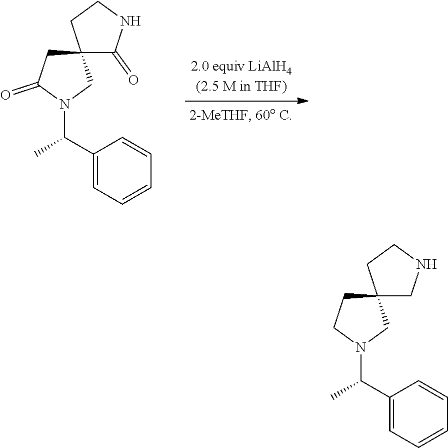 [SMILES: C[C@@H](c1ccccc1)N1C[C@@]2(CCNC2=O)CC1=O ; C[C@@H](c1ccccc1)N1CC[C@]2(CCNC2)C1]

To a reactor was charged 2-MeTHF (6.0 L, 5 V) and LiAlH₄ (2.5 M in THF, 3.72 L, 9.29 mol, 2.0 equiv). (R)-7-((S)-1-phenylethyl)-2,7-diazaspiro[4.4]nonane-1,8-dione (1.2 kg, 4.64 mol, 1.0 equiv) was charged to the reactor dropwise at 50-60° C. The reaction mixture was stirred at 50-60° C. for 12 h. The reaction mixture was cooled to 15-25° C. and diluted with 2-MeTHF (3.6 L, 3 V). To the mixture was added H₂O (0.35 L, 0.3 V), aq. NaOH solution (0.35 L, 0.3 V) and H₂O (1.06 L, 0.9 V) sequentially at 15-25° C. The mixture was further stirred for 1 h. The resulting slurry was filtered and the filtrate was collected as (S)-2-((S)-1-phenylethyl)-2,7-diazaspiro[4.4]nonane in 2-MeTHF solution, which was used directly for next step (0.86 kg assay).

### Part 5—Synthesis of tert-butyl (S)-7-((S)-1-phenylethyl)-2,7-diazaspiro[4.4]nonane-2-carboxylate

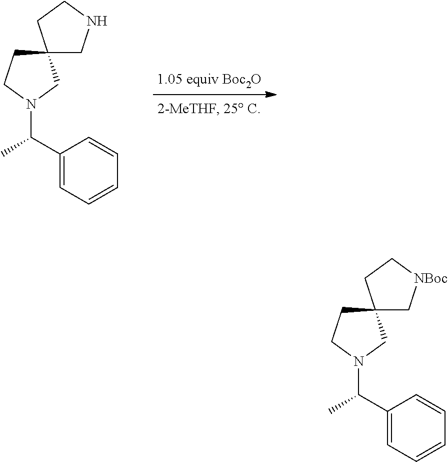 [SMILES: C[C@@H](c1ccccc1)N1CC[C@]2(CCNC2)C1 ; C[C@@H](c1ccccc1)N1CC[C@]2(CCN(C(=O)OC(C)(C)C)C2)C1]

To a reactor was added 2-MeTHF solution of (S)-2-((S)-1-phenylethyl)-2,7-diazaspiro[4.4]nonane (0.86 kg assay, 3.73 mol, 1.0 equiv) and Boc₂O (0.85 kg, 3.9 mol, 1.05 equiv). The resulting mixture was stirred at 15-25° C. for 1 h and then stirred at 45-55° C. for 12 h. The organic solution was washed with aq. NaCl solution (25% w/w, 2.4 L, 2.8 V). The organic layer was concentrated to afford tert-butyl (S)-7-((S)-1-phenylethyl)-2,7-diazaspiro[4.4]nonane-2-carboxylate as orange oil (1.24 kg).

### Part 6—Synthesis of Compound B ½ L-tartrate—tert-butyl (S)-2,7-diazaspiro[4.4]nonane-2-carboxylate ½ L-tartrate

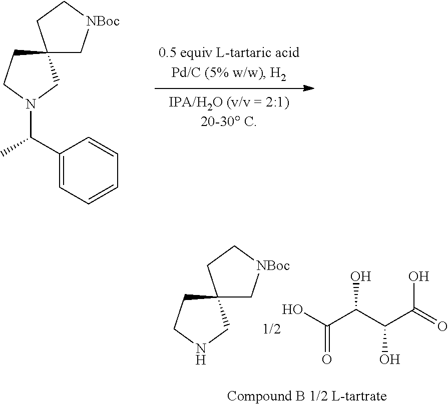 [SMILES: C[C@@H](c1ccccc1)N1CC[C@]2(CCN(C(=O)OC(C)(C)C)C2)C1 ; CC(C)(C)OC(=O)N1CC[C@]2(CCNC2)C1 ; O=C(O)[C@H](O)[C@@H](O)C(=O)O]

To a reactor was added IPA (4.2 L, 3.4 V) and H₂O (2.1 L), tert-butyl (S)-7-((S)-1-phenylethyl)-2,7-diazaspiro[4.4]nonane-2-carboxylate (1.23 kg, 3.72 mol, 1.0 equiv), and L-tartaric acid (0.28 kg, 1.86 mol, 0.5 equiv) were added to the reactor at 15-25° C. Pd/C (0.06 kg, 5% w/w) was added to the reaction mixture under N₂. The mixture was degassed with H₂ (0.5 MPa) and stirred at 15-25° C. for 12 h. The mixture was filtered and the filtrate was collected. The filtrate was concentrated to 1-2 V under vacuum at 40-50° C. To the concentrated residue was added IPA (3.6 L, 3 V). The resulting mixture was stirred at 40-50° C. for 2 h. The mixture was cooled to 15 to 25° C. and stirred for 16 h. The slurry was filtered and the wet cake was collected. The wet cake was dried to afford Compound B % tartrate as white solids (0.90 kg).

### Example 10—Synthetic Procedure for Compound D—benzyl (S)-2-cyclopentyl-2-((S)-2,7-diazaspiro[4.4]nonan-2-yl)acetate

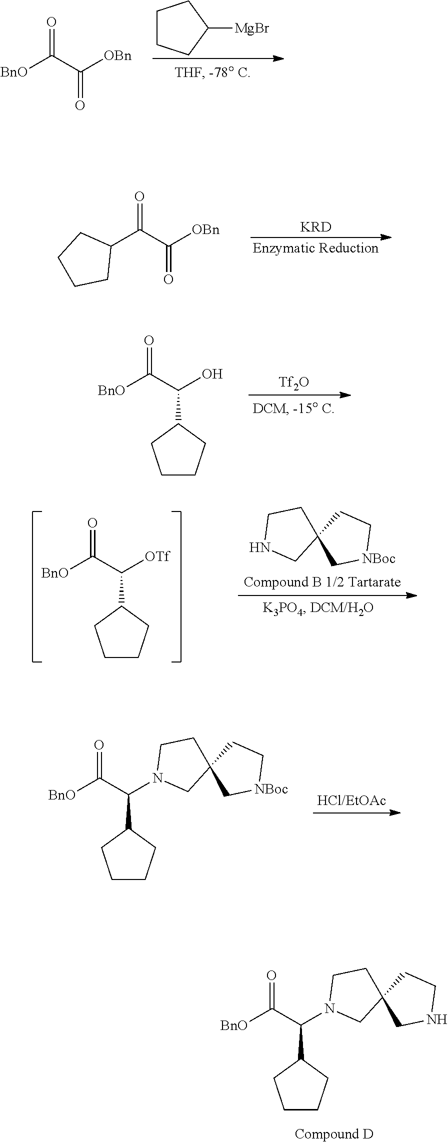

### Part 1—Synthesis of benzyl 2-cyclopentyl-2-oxoacetate

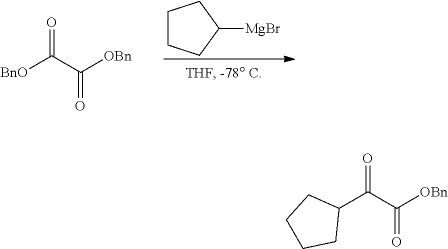

To a reactor were charged dibenzyl oxalate (1.00 kg, 3.70 mol, 1.0 equiv) and THF (30.00 L, 30.0 V) at 20-25° C. as solution A. To another reactor was charged cyclopentyl magnesium bromide (0.22 M in THF, 4.81 mol, 1.3 equiv) at 20-25° C. under N₂ flow as solution B. Solution A and solution B were mixed with a flat-push flow reactor (flow rate was A: 250 mL/min, B: 182.2 mL/min, temperature was −75 to −70° C., retention time in reactor was 15 s). The reaction was monitored by HPLC at which point HPLC analysis showed reaction completion. The reaction was quenched by sat. NH₄Cl (10.00 L, 10 V) aqueous solution. The mixture was stirred at 20-25° C. for 30 min. The phases were separated, and the organic phase was washed with aq. citric acid (0.5 wt %, 10.00 L, 10 V). The organic phase was concentrated under vacuum to dryness and isopropyl alcohol (2.00 L, 2 V) was added to the residue to give benzyl 2-cyclopentyl-2-oxoacetate IPA solution. To a reactor was charged aq. NaHSO₃ solution (40 wt %, 1.50 L, 1.5 V), followed by adding the benzyl 2-cyclopentyl-2-oxoacetate IPA solution. The mixture was stirred at 20-25° C. for 2 h. IPA (13.50 L, 13.5 V) was charged to the mixture in 2 h. The mixture was continued to stir at 20-25° C. for 10 h. The slurry was filtered, and the cake was washed with IPA (3.00 L, 3 V). The cake was dissolved in H₂O (7.00 L, 7 V) and aq. Na₂CO₃ (20 wt %, 2.00-2.50 L, 2-2.5 V) was added dropwise to the solution to adjust pH≥9 while stirring at −5° C.-5° C. MTBE (5.00 L, 5 V) was added to the solution and the mixture was stirred at rt for 30 min. The organic phase was separated and washed with aq. citric acid (0.5 wt %, 3.00 L, 3 V) and brine (5.00 L, 5 V) respectively. The organic phase was concentrated under vacuum to dryness to afford benzyl 2-cyclopentyl-2-oxoacetate as a colorless oil (543 g, 99.4% LCAP, 96.8% assay, 58% yield).

LCMS (ESI+): Calculated for C₁₄H₁₆O₃ [M+NH₄]⁺: 250.1; found: 250.0.

¹H NMR (400 MHz, DMSO-d₆) δ 7.59-7.26 (m, 5H), 5.28 (s, 2H), 3.47 (tt, J=8.9, 6.8 Hz, 1H), 1.97-1.77 (m, 2H), 1.77-1.63 (m, 2H), 1.63-1.43 (m, 4H).

### Part 2—Synthesis of benzyl (R)-2-cyclopentyl-2-hydroxyacetate

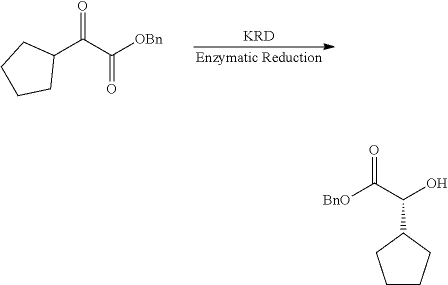 [SMILES: O=C(OCc1ccccc1)C(=O)C1CCCC1 ; "KRD" ; O=C(OCc1ccccc1)[C@H](O)C1CCCC1]

To a reactor were charged H₂O (2.50 L, 10 V), Na₂HPO₄ (84.80 g, 33.9% w/w), NaH₂PO₄ (2.00 g, 0.82% w/w) at 20-25° C. The reaction was warmed to 30-35° C. Glucose (0.58 kg, 3.23 mol, 3.0 equiv) and NADP (6.25 g, 2.5% w/w) were added to the reaction mixture. EW-KRED-R122 (5.00 g, 2% w/w) and GDH (12.50 g, 5% w/w) were added to the reaction mixture. The reaction mixture was stirred at 30-35° C. for at least 10 min. Benzyl 2-cyclopentyl-2-oxoacetate (0.25 kg, 1.08 mol, 1.0 equiv) in n-heptane (0.25 L, 1 V) was added dropwise to the reaction mixture over 10 min and the reaction mixture was stirred at 30-35° C. for 20 h during which pH was checked after 8 h (the pH was controlled at 7.3-8.2 with 1M aq. NaOH solution). The reaction was monitored by HPLC at which point HPLC analysis showed reaction completion. After completion of reaction, MTBE (5.00 L, 20 V) was added, and the mixture was kept stirring for 10 min. The mixture was filtered′ and the cake was washed with MTBE (0.50 L, 2 V). The filtrate was settled for 10 min and the organic phase was separated. The aqueous phase was washed with MTBE (5.00 L, 20 V). The combined organic phase was washed with aq. NaCl (20 wt %, 5.00 L, 20 V). The organic phase was concentrated under vacuum to dryness to afford benzyl (R)-2-cyclopentyl-2-hydroxyacetate as a colorless oil (245 g, 97.8% LCAP, 97.2% assay, 100% ee, 97.5% yield).

LCMS (ESI+): Calculated for C₁₄H₁₈O₃ [M+Na]⁺: 257.1; found: 257.2.

¹H NMR (400 MHz, DMSO-d₆) δ 7.47-7.27 (m, 5H), 5.38 (d, J=6.1 Hz, 1H), 5.13 (d, J=1.2 Hz, 2H), 3.93 (t, J=6.2 Hz, 1H), 2.28-2.05 (m, 1H), 1.65-1.49 (m, 4H), 1.49-1.27 (m, 4H).

### Part 3—Synthesis of tert-butyl (S)-7-((S)-2-(benzyloxy)-1-cyclopentyl-2-oxoethyl)-2,7-diazaspiro[4.4]nonane-2-carboxylate

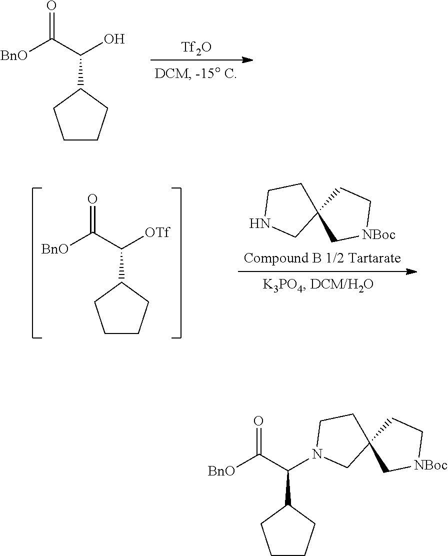

A reactor was charged with benzyl (R)-2-cyclopentyl-2-hydroxyacetate (19.45 kg, 83.18 mol, 1.0 equiv) and DCM (234.00 L, 12 V) at 15-25° C., followed by adding DIPEA (21.50 kg, 166.36 mol, 2.0 equiv). The reaction mixture was cooled to −10-0° C. under N₂ flow. Tf₂O (31.85 kg, 116.45 mol, 1.4 equiv) was added dropwise to the reaction mixture over 1 h. The reaction mixture was kept stirring for 1 h at −10-0° C. and a sample was taken for HPLC analysis. H₂O (19.50 L, 1 V) was added to the reaction at 0-5° C., followed by adding Compound B (½ tartaric salt, 27.70 kg, 91.92 mol, 1.1 equiv) and K₃PO₄ (45.90 kg, 216.23 mol, 2.6 equiv). The reaction was warmed to 20-25° C. over 1 h and was continued to stir for 6 h. After completion, aq. HCl (0.5 M, 293.00 L, 15 V) was added to the reaction at 10±5° C. The mixture was stirred for 30 min and the organic phase was separated. To the organic phase was added H₂O (195.00 L, 10 V) at 10±5° C. The mixture was stirred for 30 min and the organic phase was separated. The organic phase was concentrated to dryness to afford tert-butyl (S)-7-((S)-2-(benzyloxy)-1-cyclopentyl-2-oxoethyl)-2,7-diazaspiro[4.4]nonane-2-carboxylate as yellow oil (33.76 kg, 93.2% LCAP, 91.6% yield). Table 14 summarizes the HPLC method for this synthesis.

LCMS (ESI+): Calculated for C₂₆H₃₈N₂O₄ [M+H]⁺: 443.3; found: 443.4.

¹H NMR (400 MHz, DMSO-d₆) δ 7.63-7.15 (m, 5H), 5.13 (d, J=1.3 Hz, 2H), 3.24 (dt, J=11.5, 5.1 Hz, 1H), 3.19 (d, J=8.0 Hz, 1H), 3.14 (d, J=10.4 Hz, 1H), 3.09 (d, J=10.6 Hz, 1H), 3.04 (d, J=10.4 Hz, 1H), 2.79 (dt, J=12.8, 5.4 Hz, 1H), 2.68 (t, J=7.7 Hz, 1H), 2.61 (dd, J=8.8, 3.1 Hz, 1H), 2.52 (d, J=1.8 Hz, 1H), 2.29-2.11 (m, 1H), 1.84-1.64 (m, 3H), 1.60 (t, J=7.0 Hz, 2H), 1.57-1.44 (m, 5H), 1.39 (s, 10H), 1.16-1.03 (m, 1H).

TABLE 14
HPLC Method for Parts 2 and 3
IPC method:

| Column: | XBridge C18 (4.6 × 150 mm, 3.5 μm) | | |
|---|---|---|---|
| Mobile Phase: | A: 0.05% TFA in water (v/v) | | |
| | B: 0.05% TFA in MeCN (v/v) | | |
| | Time (min) | A % | B % |
| Gradient: | 0.0 | 60 | 40 |
| | 12.0 | 5 | 95 |
| | 15.0 | 5 | 95 |
| | 16.0 | 60 | 40 |

| | |
|---|---|
| Flow Rate: | 1.0 mL/min |
| UV Detector Wavelength: | 210 nm |
| Column Temperature: | 40° C. |
| Retention Times: | benzyl (R)-2-cyclopentyl-2-hydroxyacetate: 6.79 min |
| | benzyl (R)-2-cyclopentyl-2-trifylacetate: 11.18 min |
| | tert-butyl (S)-7-((S)-2-(benzyloxy)-1-cyclopentyl-2-oxoethyl)- |
| | 2,7-diazaspiro[4.4]nonane-2-carboxylate: 5.62 min |

### Part 4—Synthesis of benzyl (S)-2-cyclopentyl-2-((S)-2,7-diazaspiro[4.4]nonan-2-yl)acetate

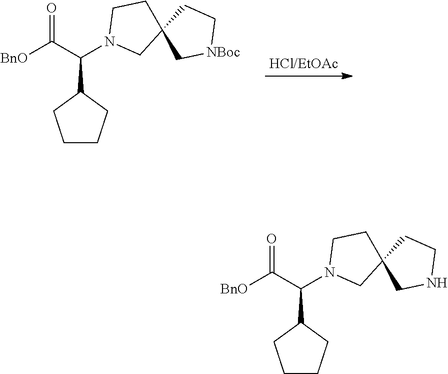 [SMILES: CC(C)(C)OC(=O)N1CC[C@]2(CCN([C@H](C(=O)OCc3ccccc3)C3CCCC3)C2)C1 ; O=C(OCc1ccccc1)[C@H](C1CCCC1)N1CC[C@]2(CCNC2)C1]

To a reactor were charged tert-butyl (S)-7-((S)-2-(benzyloxy)-1-cyclopentyl-2-oxoethyl)-2,7-diazaspiro[4.4]nonane-2-carboxylate (30.1 kg, 68.00 mol, 1.0 equiv) and EtOAc (150.50 L, 5 V). The solution was cooled to 0-5° C. HCl in EtOAc solution (4 M, 150.50 L, 5 V) was added dropwise to the reaction. The reaction was warmed to 20-30° C. and was stirred at 20-30° C. for 6 h. The reaction was monitored by HPLC at which point HPLC analysis showed reaction completion. After completion, the reaction was cooled to 0-10° C., aq. HCl (0.5 M, 301.00 L, 10 V) was added to the reaction. The mixture was stirred at 20-30° C. for 20 min, settled and phases are separated. The organic phase was extracted with aq. HCl (0.5 M, 301.00 L, 10 V) at 0-20° C. The combined aqueous phase was washed with MTBE (2×301.00 L, 2×10 V). The pH of aqueous phase was adjusted to 11-12 at 5-10° C. using aq. NaOH solution (20 wt %). The aqueous phase was extracted with MTBE (2×301.00 L, 2×10 V). The combined organic phase was washed with aq. NaCl solution (26 wt %, 301.00 L, 10 V). The organic phase was concentrated to dryness under vacuum to afford crude benzyl (S)-2-cyclopentyl-2-((S)-2,7-diazaspiro[4.4]nonan-2-yl)acetate as colorless oil (assay 20.99 kg, 90.1% yield).

To a reactor were added crude benzyl (S)-2-cyclopentyl-2-((S)-2,7-diazaspiro[4.4]nonan-2-yl)acetate (20.99 kg, 61.29 mol, 1.0 equiv) and IPA (210.00 L, 10 V) at 20±10° C. In another reactor, a solution of D-(+)-2,3-dibenzoyl tartaric acid (21.96 kg, 61.29 mol, 1.0 equiv) in IPA solution (210.00 L, 10 V) was prepared. One fifth of the IPA solution of D-(+)-2,3-dibenzoyl tartaric acid (42.00 L, 2 V) was added to the benzyl (S)-2-cyclopentyl-2-((S)-2,7-diazaspiro[4.4]nonan-2-yl)acetate solution dropwise over 1.5 h at 40-45° C. A seed of benzyl (S)-2-cyclopentyl-2-((S)-2,7-diazaspiro[4.4]nonan-2-yl)acetate (0.21 kg, 0.01 w/w) was charged to the reaction at 40-45° C. then. The mixture was stirred at 40-45° C. for 1.5 h. The rest of portion (⅘) of D-(+)-2,3-dibenzoyl tartaric acid in IPA solution (168.00 L, 8 V) was added dropwise to the reaction over 6 h. The mixture was stirred at 40-45° C. for 3 h and then was cooled to 0-5° C. The mixture was continued to stir at 0-5° C. for 6 h. The slurry was filtered, and the cake was washed with IPA (42.00 L, 2 V) and MTBE (42.00 L, 2 V). The cake was dissolved in H₂O (210.00 L, 10 V) and MTBE (420.00 L, 20 V) was added to the solution. The pH of aqueous phase was adjusted to 11-12 with aq. NaOH solution (10 wt %). The mixture was stirred for 45 min, then settled and the phase was separated. The organic phase was washed with aq. NaCl solution (26 wt %, 210.00 L, 10 V). The organic phase was concentrated to dryness under vacuum to afford benzyl (S)-2-cyclopentyl-2-((S)-2,7-diazaspiro[4.4]nonan-2-yl)acetate as colorless oil (19.98 kg, 99.96% LCAP, 99.01% ee, 85.8% yield). Table 15 summarizes the HPLC method for this synthesis.

LCMS (ESI+): Calculated for C₂₁H₃₀N₂O₂ [M+H]⁺: 343.2; found: 343.3.

¹H NMR (400 MHz, CDCl₃) δ 7.82-7.30 (m, 4H), 5.15 (s, 2H), 3.13 (d, J=10.6 Hz, 1H), 2.92 (dtd, J=16.7, 9.4, 5.1 Hz, 3H), 2.82 (d, J=10.8 Hz, 1H), 2.79-2.65 (m, 3H), 2.60 (d, J=8.6 Hz, 1H), 2.27 (dq, J=10.4, 7.8 Hz, 1H), 1.86-1.74 (m, 1H), 1.69 (tdd, J=10.0, 6.2, 4.2 Hz, 4H), 1.63-1.48 (m, 4H), 1.48-1.38 (m, 1H), 1.20 (s, 2H).

TABLE 15
HPLC Method for Part 4
IPC method:

| Column: | XBridge C18 (4.6 × 150 mm, 3.5 μm) | | |
|---|---|---|---|
| Mobile Phase: | A: 0.05% TFA in water (v/v) | | |
| | B: 0.05% TFA in MeCN (v/v) | | |
| | Time (min) | A % | B % |
| Gradient: | 0.0 | 60 | 40 |
| | 12.0 | 5 | 95 |
| | 15.0 | 5 | 95 |
| | 16.0 | 60 | 40 |

| | |
|---|---|
| Flow Rate: | 0.8 mL/min |
| UV Detector Wavelength: | 210 nm |
| Column Temperature: | 40° C. |
| Retention Times: | tert-butyl (S)-7-((S)-2-(benzyloxy)-1-cyclopentyl-2-oxoethyl)- |
| | 2,7-diazaspiro[4.4]nonane-2-carboxylate: 10.65 min |
| | benzyl (S)-2-cyclopentyl-2-((S)-2,7-diazaspiro[4.4]nonan-2- |
| | yl)acetate: 7.44 min |

### Example 11—Alternative Synthetic Route for benzyl (R)-2-cyclopentyl-2-hydroxyacetate

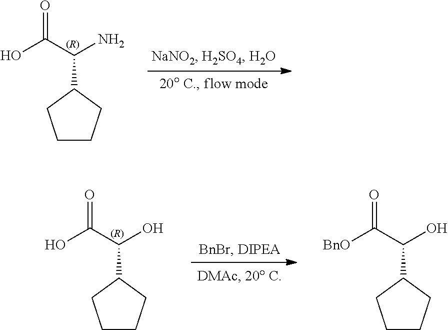

### Part 1—Synthesis of (R)-2-cyclopentyl-2-hydroxyacetic acid

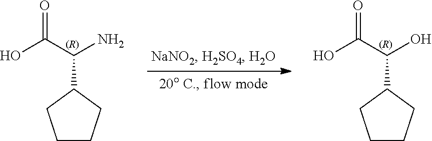

A reactor was charged with H₂SO₄ (56.76 kg, 578.78 mol, 2.5 equiv) and H₂O (660.00 L, 20 V) at 20-25° C., followed by (R)-2-amino-2-cyclopentylacetic acid (33.15 kg, 231.51 mol, 1.0 equiv). The reaction mixture was stirred at 20-25° C. for 30 min to form a homogeneous solution. The solution was filtered via a microporous filter (0.22 μm) to tank A as solution A. To another reactor were charged NaNO₂ (102.45 kg, 1484.88 mol, 6.4 equiv) and H₂O (800.00 L, 24.1 V) at 20-25° C. The reaction mixture was stirred at 20-25° C. for 30 min and transferred to tank B as solution B. Solution A and solution B were mixed with a continuous micro reactor. Flow rate was A: 37.7 L/h, B: 27.7 L/h, temperature was 50° C., retention time in micro reactor was 2 min. The solution was purged with N₂ gas for 12 h to push most of the NO₂ gas into the exhaust gas absorption reactor. NaCl (198.9 kg, 6 w/w) was charged to the reactor and was kept agitating to dissolve the solids. The solution was extracted with MTBE (2×994.50 L, 2×30 V). The organic phase was concentrated to 2-3 V under reduced pressure (0.08 MPa, 20-30° C.). The MTBE residue was swapped with n-heptane to 2-3 V under reduced pressure (0.08 Mpa, 20-30° C.) until MTBE residue is less than 3 wt % and KF is less than 0.5 wt %. EtOAc (19.90 L, 0.6 V) was added to the n-heptane residue and the mixture was heated to 45±2° C. The reaction mixture was stirred at 45±2° C. for at least 8 h and then gradually cooled to 30° C. by the speed of 1.5° C./h. The slurry was filtered, and the cake was washed with EtOAc/n-heptane (1:5 v/v, 33.00 L, 1 V). The wet cake was dried under vacuum (0.09 Mpa, 45±5° C.) to afford (R)-2-cyclopentyl-2-hydroxyacetic acid as off-white solids (13.3 kg, 91.4% LCAP, 44.9% yield). Table 16 summarizes the HPLC method for this synthesis.

LCMS (ESI+): Calculated for C₇H₁₂O₃ [M−H₂O]⁺: 127.1; found: 127.3.

¹H NMR (400 MHz, DMSO-d₆) δ 3.80 (d, J=5.9 Hz, 1H), 2.13 (td, J=7.9, 5.9 Hz, 1H), 1.71-1.50 (m, 4H), 1.50-1.30 (m, 4H).

TABLE 16
HPLC Method for Part 1
IPC method:

| Column: | XBridge C18 (4.6 × 150 mm, 3.5 μm) | | |
|---|---|---|---|
| Mobile Phase: | A: 0.05% TFA in water | | |
| | B: 0.0% TFA in MeCN | | |
| | Time (min) | A % | B % |
| Gradient: | 0.0 | 90 | 10 |
| | 10.0 | 50 | 50 |
| | 13.0 | 5 | 95 |
| | 16.0 | 5 | 95 |
| | 17.0 | 90 | 10 |

| | |
|---|---|
| Flow Rate: | 0.8 mL/min |
| UV Detector | 210 nm |
| Wavelength: | |
| Column Temperature: | 40° C. |
| Retention Times: | (R)-2-amino-2-cyclopentylacetic acid: 3.63 min |
| | (R)-2-cyclopentyl-2-hydroxyacetic acid: 7.32 min |

### Part 2—Synthesis of benzyl (R)-2-cyclopentyl-2-hydroxyacetate

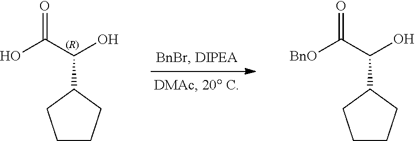

A reactor was charged with dimethylacetamide (128.00 L, 5 V), DIPEA (34.44 kg, 266.46 mol, 1.5 equiv) and (R)-2-cyclopentyl-2-hydroxyacetic acid (25.6 kg, 177.57 mol, 1.0 equiv) at 20-25° C. under N₂ atmosphere. The reaction mixture was cooled to 0-10° C. and BnBr (36.45 kg, 213.11 mol, 1.2 equiv) was added dropwise. The reaction mixture was warmed to 15-25° C. and was continued to stir at 15-25° C. for 6 h. The reaction was monitored by GC at which point GC analysis showed reaction completion. DMAP (13.01 kg, 106.49 mol, 0.6 equiv) was added and the mixture was stirred for 18 h at 20-25° C. and a sample was taken for GC analysis to make sure BnBr is not detected. H₂O (256.00 L, 10V) was added to the solution and the resulting solution was extracted with MTBE (2×128.00 L, 2×5 V). The combined organic phase was washed with aq. citric acid (2×128.00 L, 2×5 V) and H₂O (128.00 L, 5 V) respectively. The organic phase was concentrated to 2-3 V under reduced pressure (0.08 Mpa, 20-35° C.). The MTBE residue was swapped with DCM (256.00 L, 10 V) to 2-3 V under reduced pressure (0.08 Mpa, 20-35° C.) until MTBE residue was less than 3% LCAP by GC analysis. Benzyl (R)-2-cyclopentyl-2-hydroxyacetate was obtained as solution in DCM (44.02 kg assay, 95.6% LCAP, 105% yield). Table 17 summarizes the HPLC method for this synthesis.

LCMS (ESI+): Calculated for C₁₄H₁₈O₃ [M+Na]⁺: 257.1; found: 257.2.

¹H NMR (400 MHz, DMSO-d₆) δ 7.47-7.27 (m, 5H), 5.38 (d, J=6.1 Hz, 1H), 5.13 (d, J=1.2 Hz, 2H), 3.93 (t, J=6.2 Hz, 1H), 2.28-2.05 (m, 1H), 1.65-1.49 (m, 4H), 1.49-1.27 (m, 4H).

TABLE 17
HPLC Method for Parts 1 and 2
IPC method:

| Column: | Rtx-35 30 m*0.32 mm, 1 μm | |
|---|---|---|
| Flow | 2 | mL/min |
| Inject temperature | 230° | C. |
| Oven temperature | 40° C. for 0 min, then 20° | |
| | C./min to 280° C. for 3 min | |
| Detection temperature | 280° | C. |
| Split | 30:1 | |
| Air flow | 300 | mL/min |
| N₂ flow | 30 | mL/min |
| H₂ flow | 30 | mL/min |

| | |
|---|---|
| Retention times | (R)-2-cyclopentyl-2-hydroxyacetic |
| | acid: 8.48 min |
| | benzyl (R)-2-cyclopentyl-2-hydroxy- |
| | acetate: 12.02 min |

### Example 12—Synthetic Procedure for (S)-2-cyclopentyl-2-((S)-7-((2R,3R)-3-cyclopropyl-1-methylaziridine-2-carbonyl)-2,7-diazaspiro[4.4]nonan-2-yl)acetic acid (Compound E) and the sodium salt thereof

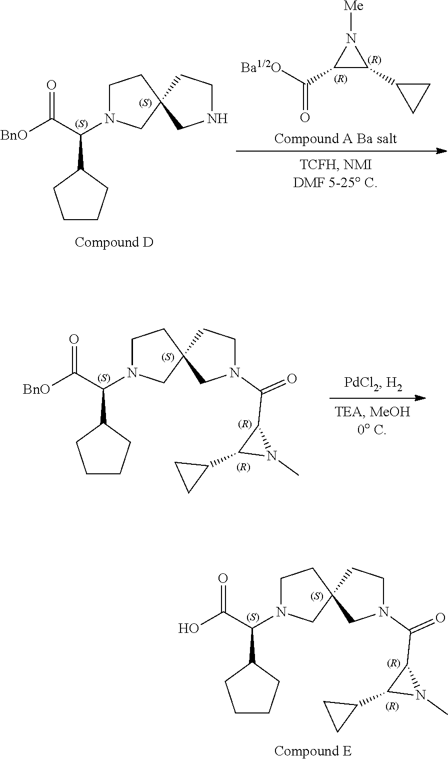

### Part 1—Synthesis of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate

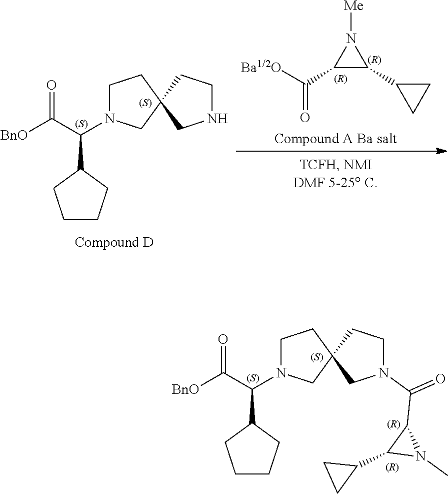

In a reactor, Compound A Ba salt (8.5 kg, 40.88 mol, 1.0 equiv) was dissolved in DMF (56.00 L, 4 V) at 15±10° C. NMI (13.4 kg, 163.22 mol, 4.0 equiv) and Compound D (14.00 kg, 40.88 mol, 1.0 equiv) were added to the solution, followed by adding TCFH (13.80 kg, 49.18 mol, 1.2 equiv) solution in DMF (4.00 L, 1 V) dropwise over 1 h at 15±10° C. The reaction mixture was stirred at 15±10° C. for at least 30 min. The reaction was monitored by HPLC at which point HPLC analysis showed reaction completion. The reaction mixture was cooled to 5±5° C. and quenched with H₂O (28.00 L, 2 V). The mixture was warmed to 20±5° C. and stirred for 10 min. MTBE (280.00 L, 20 V), H₂O (182.00 L, 13 V) and Na₂CO₃ (1.12 kg, 0.08 w/w) were added to the mixture and stirred for 20 min. The organic phase was separated, and the aqueous phase was extracted with MTBE (140.00 L, 10 V). The combined organic phase was washed with aq. N₂CO₃ solution (15 wt %, 2×164.60 kg, 2×11.76 w/w). The organic phase was concentrated under reduced pressure (0.08 MPa, 20-40° C.) to 4-5 V. The MTBE was swapped with EtOAc by adding EtOAc (280.00 L, 20 V) and concentrated under reduced pressure (0.08 MPa, 20-40° C.) to 4-5 V, the process was repeated twice. A portion of EtOAc (210.00 L, 15 V) was added to the residue and the mixture was stirred for 40 min. The solution was cooled to −5±5° C. and maleic acid (2.37 kg, 20.42 mol, 0.5 equiv) was added portion-wise to the solution. A seed of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate as the maleate salt was added to the reaction and stirred for 15 min. The solution of maleic acid (6.17 kg, 1.3 equiv) in EtOAc (280.00 L, 20 V) was added dropwise to the reaction over 7.5 h and continued to stir at −5±5° C. for 7.5 h. The slurry was filtered, and the cake was washed with pre-cooled EtOAc (42.00 L, 3 V) at −5±5° C. The wet cake was collected. To a reactor were added MTBE (140.00 L, 10 V) and the wet cake at 0±5° C. aq. Na₂CO₃ solution (15 wt %) was added at 0±5° C. until the pH of aqueous layer is 9-11. The mixture was warmed to 25±5° C. and stirred for 30 min. The organic phase was separated, and the aqueous phase was extracted with MTBE (140.00 L, 10 V). The combined organic phase was washed with aq. Na₂CO₃ solution (15% wt %, 140.00 L, 10 V). The organic phase was concentrated under reduced pressure (0.08 MPa, 20-40° C.) to 4-5 V. The MTBE was swapped with MeOH by adding MeOH (280.00 L, 20 V) and concentrated under reduced pressure (0.08 MPa, 20-40° C.) to 4-5 V, the process was repeated twice. A portion of MeOH (112.00 L, 8 V) was added to the residue and the mixture was stirred for 30 min at 20±10° C. A sample was taken for GC analysis (MTBE residue s 3 wt %). Benzyl (S)-2-cyclopentyl-2-((S)-7-((2R,3R)-3-cyclopropyl-1-methylaziridine-2-carbonyl)-2,7-diazaspiro[4.4]nonan-2-yl)acetate was obtained as MeOH solution (128.7 kg solution, 9.65 kg benzyl (S)-2-cyclopentyl-2-((S)-7-((2R,3R)-3-cyclopropyl-1-methylaziridine-2-carbonyl)-2,7-diazaspiro[4.4]nonan-2-yl)acetate by assay, 97.8% LCAP, 50.7% yield).

LCMS (ESI+): Calculated for C₂₈H₃₉N₃O₃ [M+H]⁺: 466.3; found: 466.6.

¹H NMR (400 MHz, Methanol-d₄) δ 7.49-7.30 (m, 5H), 6.27 (s, 4H), 5.41-5.20 (m, 2H), 3.87-3.69 (m, 2H), 3.69-3.32 (m, 7H), 2.83 (d, J=2.4 Hz, 3H), 2.55 (dt, J=12.0, 8.2 Hz, 1H), 2.45 (t, J=7.5 Hz, 1H), 2.27-1.96 (m, 4H), 1.96-1.84 (m, 1H), 1.77-1.63 (m, 1H), 1.53 (d, J=4.8 Hz, 5H), 1.37-1.23 (m, 1H), 0.85-0.61 (m, 4H), 0.53 (tq, J=6.3, 4.3 Hz, 1H).

### Part 2—Synthesis of (S)-2-cyclopentyl-2-((S)-7-((2R,3R)-3-cyclopropyl-1-methylaziridine-2-carbonyl)-2,7-diazaspiro[4.4]nonan-2-yl)acetic acid (Compound E)

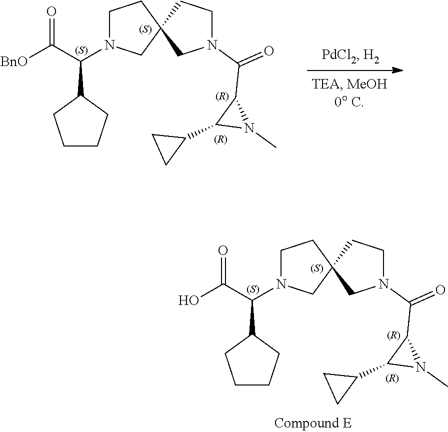

A MeOH solution of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate (6.05 kg assay, 13.00 mol, 1.0 equiv) and TEA (4.00 kg, 39.00 mol, 3.0 equiv) were added into a reactor. The mixture was cooled to 0±5° C. and PdCl₂ (60.50 g, 1% w/w) was added to the reaction. The reaction mixture was stirred for at least 3 h at 0±5° C. under pressure of 0-0.15 MPa with H₂. A sample was taken for HPLC analysis. The reaction mixture was filtered via diatomite and washed the filter cake with MeOH (42.35 L, 7 V). The filtrate was concentrated to 8-10 V under reduced pressure (20-30° C., 0.08 Mpa). The solvent was swapped with THE (60.50 L, 10 V) to 8-10 V under reduced pressure (20-30° C., 0.08 Mpa) twice. To the residue was added THE (60.50 L, 10 V) and TEA (2.00 kg, 19.76 mol, 1.5 equiv). The resulting solution was concentrated to 8-10 V under reduced pressure (20-30° C., 0.08 Mpa) and THE (18.15 L, 3 V) was added to the residue. The solution was stirred for at least 30 min at 20±10° C. and slurry formed. MTBE (60.50 L, 10 V) was added to the slurry over 1.2 h at 20±10° C. The mixture was concentrated to 10-13 V under reduced pressure (20-30° C., 0.08 Mpa). The solvent was swapped with MTBE (48.40 L, 8 V) to 10-13 V for 6 times by charging MTBE dropwise over 1 h and then concentration under reduced pressure (20-30° C., 0.08 Mpa). MTBE (48.40 L, 8 V) was added to the suspension dropwise over 1 h at 20±10° C. and a sample was taken for analysis (THF residue≤7% w/w). The slurry was filtered, and the cake was washed (18.15 L, 3 V). The cake was dried under vacuum (30±5° C., 0.08 Mpa) for at least 8 h until LOD≤10% w/w. Compound E was obtained as off-white solids (4.13 kg, 95.9% LCAP, 90.2% w/w assay, 76.4% yield).

LCMS (ESI+): Calculated for C₂₁H₃₃N₃O₃ [M+H]⁺: 376.3; found: 376.4.

¹H NMR (400 MHz, Methanol-d₄) δ 3.87-3.76 (m, 1H), 3.73-3.63 (m, 1H), 3.63-3.38 (m, 7H), 2.44-2.25 (m, 5H), 2.19 (dq, J=13.1, 6.7, 6.2 Hz, 1H), 2.14-1.95 (m, 3H), 1.90 (qt, J=6.9, 2.9 Hz, 1H), 1.82-1.51 (m, 7H), 1.46 (dt, J=9.0, 7.0 Hz, 1H), 0.73-0.56 (m, 1H), 0.46 (dddd, J=17.8, 7.5, 5.7, 4.3 Hz, 3H), 0.28 (dtt, J=11.5, 6.2, 2.7 Hz, 1H).

### Sodium Salt of Compound E

A MeOH solution of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate (1.0 equiv) and TEA (3.0 equiv) were added into a reactor. The mixture was cooled to 0±5° C. and PdCl₂ (1% w/w) was added to the reaction. The reaction mixture was stirred for at least 3 h at 0±5° C. under pressure of 0-0.15 MPa with H₂. A sample was taken for HPLC analysis. The reaction mixture was filtered via diatomite and washed the filter cake with MeOH (7 V). The filtrate was concentrated to 3-4 V under reduced pressure (20-30° C., 0.08 Mpa). MeOH (7 V) was added to the residue and the solution was stirred for 10 min at rt. NaOMe (1.03 equiv, 30 wt % MeOH solution) was added to the solution at rt and the resulting mixture was stirred for at least 3 h at rt. The reaction solution was concentrated to 3-4 V under reduced pressure (20-30° C., 0.08 Mpa) to afford solution A. In another reactor was charged MTBE (60 V) and the reactor was cooled to −5-5° C. The seeds of Compound E Na salt (2 wt %) was added to MTBE solution and the suspension was stirred for 30 min. Solution A of Compound E was added to MTBE over 5 h at −5-5° C. and the suspension was stirred for 2 h. The slurry was filtered, and the wet cake was washed with MTBE (2 V). The wet cake was dried at 30-40° C. for 5 h under vacuum to afford Compound E Na salt. Table 18 summarizes the HPLC method for this synthesis.

LCMS (ESI+): Calculated for C₂₁H₃₃N₃O₃ [M+H]⁺: 376.26; found: 376.28.

¹H NMR (400 MHz, Methanol-d₄) δ 3.70-3.65 (m, 1H), 3.57-3.35 (m, 3H), 3.01-2.79 (m, 4H), 2.39 (s, 3H), 2.34 (t, J=7.2 Hz, 1H), 2.29-2.16 (m, 1H), 2.10-1.70 (m, 6H), 1.70-1.47 (m, 5H), 1.42 (p, J=7.2 Hz, 2H), 0.69-0.21 (m, 5H).

TABLE 18
HPLC Method for Parts 1 and 2
IPC method:

| Column: | XBridge C18 (4.6 × 150 mm, 3.5 μm) | | |
|---|---|---|---|
| Mobile Phase: | A: 10 mM NH₄OAc in water | | |
| | B: MeOH | | |
| | Time (min) | A % | B % |
| Gradient: | 0.0 | 90 | 10 |
| | 13.0 | 5 | 95 |
| | 16.0 | 5 | 95 |
| | 17.0 | 90 | 10 |

| | |
|---|---|
| Flow Rate: | 1.0 mL/min |
| UV Detector | 210 nm |
| Wavelength: | |
| Column Temperature: | 40° C. |
| Retention Times: | Compound E: 6.46 min |
| | benzyl (S)-2-cyclopentyl-2-((S)-7-((2R,3R)-3- |
| | cyclopropyl-1-methylaziridine-2-carbonyl)-2,7- |
| | diazaspiro[4.4]nonan-2-yl)acetate: 14.05 min |

### Example 13—Alternative Synthetic Route for benzyl (S)-2-cyclopentyl-2-((S)-7-((2R,3R)-3-cyclopropyl-1-methylaziridine-2-carbonyl)-2,7-diazaspiro[4.4]nonan-2-yl)acetate

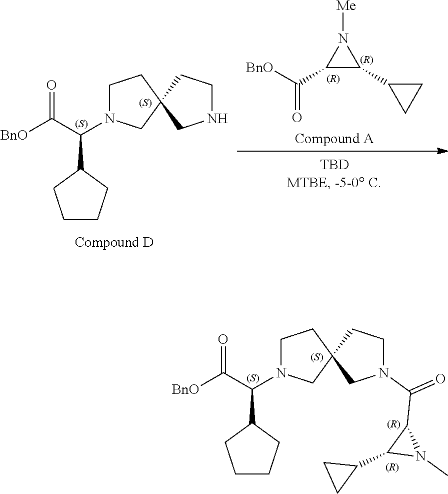

To a reactor were charged MTBE (90.50 mL, 5 V), Compound D (18.10 g, 52.85 mmol, 1.0 equiv), Compound A (12.84 g, 55.49 mmol, 1.05 equiv) and 1,5,7-triazobicyclo[4,4,0]dec-5-ene(TBD) (2.21 g, 15.86 mmol, 0.3 equiv). The reaction mixture was cooled to −5-0° C. The reaction mixture was stirred at −5-0° C. for 24 hours at which point HPLC analysis showed reaction completion. Aq. Na₂CO₃ solution (5 wt %, 181.00 mL, 10 V) was added to quench the reaction and the mixture was stirred at 15-25° C. for at least 10 min. The phases were separated, and the organic phase was washed with aq. Na₂CO₃ solution (5 wt %, 181.00 mL, 10 V). The organic phase was concentrated to dryness (T s 40° C.). The residue was dissolved in EtOAc (145.00 mL, 8 V) and the solution was cooled to −10-0° C. To the solution was added maleic acid (3.07 g, 26.42 mmol, 0.5 equiv), followed by adding seeds of the maleate salt of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate (181.00 mg, 1 wt %) at −10-0° C. The mixture was stirred for at least 10 min before the solution of maleic acid in MTBE/EtOAc (v/v=3:2, 181.00 mL, 10 V) was added dropwise over 3 h. The suspension was stirred for 12 h at −10-0° C. The slurry was filtered and the cake was washed with pre-cooled MTBE/EtOAc (v/v=1:2, 54.30 mL, 3 V). The filter cake was transferred to a reactor where MTBE (181.00 mL, 10 V) was cooled to −5-0° C. Aq. Na₂CO₃ solution (15 wt %, 181.00 mL, 10 V) was added to the solution to adjust the pH=8-10. The mixture was stirred for at least 10 min at 15-25° C. The phases were separated, and the aqueous phase was extracted with MTBE (90.50 mL, 5 V). The combined organic phase was washed with aq. Na₂CO₃ solution (5 wt %, 181.00 mL, 10 V). The organic phase was concentrated to dryness to afford the maleate salt of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate (19.3 g, 97.2% LCAP, 92.7% QNMR assay).

### Besylate salt of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate

A reactor was charged with MTBE (5 V), Compound D (1.0 equiv), Compound A (1.05 equiv), and 1,5,7-triazobicyclo[4,4,0]dec-5-ene(TBD) (0.3 equiv). The reaction mixture was cooled to −5-0° C. The reaction mixture was stirred at −5-0° C. for 24 hours at which point HPLC analysis showed reaction completion. Aq. Na₂CO₃ solution (5 wt %, 10 V) was added to quench the reaction and the mixture was stirred at 15-25° C. for at least 10 min. The phases were separated, and the organic phase was washed with aq. Na₂CO₃ solution (5 wt %, 10 V). The organic phase was concentrated to dryness (T s 40° C.). Benzenesulfonic acid (2.1 equiv) was dissolved in THE (4.53 V) and set aside for later use. The concentrated benzyl (S)-2-cyclopentyl-2-((S)-7-((2R,3R)-3-cyclopropyl-1-methylaziridine-2-carbonyl)-2,7-diazaspiro[4.4]nonan-2-yl)acetate residue was diluted with acetone (10 V). The solution was cooled to 7±3° C. The premade benzenesulfonic acid solution in THE (3.8/21) was added portion-wise to benzyl (S)-2-cyclopentyl-2-((S)-7-((2R,3R)-3-cyclopropyl-1-methylaziridine-2-carbonyl)-2,7-diazaspiro[4.4]nonan-2-yl)acetate and a seed of the besylate salt of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate (0.5% w/w) was then added to the mixture. The mixture was stirred at 7±3° C. for 1 h before another portion of benzenesulfonic acid THE solution (15.2/21) was added. The resulting mixture was stirred at 7±3° C. for 16 h. Then the rest of benzenesulfonic acid THE solution (0.5/21) was added to the mixture and continued to stir for 6 h. The slurry was filtered and the wet cake was washed with THE/acetone (v/v=1:2, 3 V). The wet cake was added in MTBE (10 V) and aq. Na₂CO₃ solution (15% w/w) was added to adjust pH to 7.5-9.0 at 0±5° C. To the mixture was added H₂O (5 V) and the biphasic solution was stirred for 0.5 h before the organic phase was separated. The aqueous phase was washed with MTBE (5 V). The organic phases were combined and washed with aq. Na₂CO₃ solution (5% w/w, 10 V). The organic phase was concentrated under vacuum to 4-5 V at NMT 40° C. The concentrated residue was diluted with acetone (10 V) and the resulting mixture was concentrated under vacuum to 4-5 V at NMT 40° C. The solvent wash with acetone was repeated once. The solution of benzyl (S)-2-cyclopentyl-2-((S)-7-((2R,3R)-3-cyclopropyl-1-methylaziridine-2-carbonyl)-2,7-diazaspiro[4.4]nonan-2-yl)acetate in acetone was used for next step. Table 19 summarizes the HPLC method for this synthesis.

LCMS (ESI+): Calculated for C₂₈H₃₉N₃O₃ [M+H]⁺: 466.3; found: 466.6.

¹H NMR (400 MHz, Methanol-d₄) δ 7.49-7.30 (m, 5H), 6.27 (s, 4H), 5.41-5.20 (m, 2H), 3.87-3.69 (m, 2H), 3.69-3.32 (m, 7H), 2.83 (d, J=2.4 Hz, 3H), 2.55 (dt, J=12.0, 8.2 Hz, 1H), 2.45 (t, J=7.5 Hz, 1H), 2.27-1.96 (m, 4H), 1.96-1.84 (m, 1H), 1.77-1.63 (m, 1H), 1.53 (d, J=4.8 Hz, 5H), 1.37-1.23 (m, 1H), 0.85-0.61 (m, 4H), 0.53 (tq, J=6.3, 4.3 Hz, 1H).

TABLE 19
HPLC Method for besylate salt formation
IPC method:

| Column: | XBridge C18 (4.6 × 150 mm, 3.5 μm) | | |
|---|---|---|---|
| Mobile Phase: | A: 0.05% TFA in water (v/v) | | |
| | B: 0.05% TFA in MeCN (v/v) | | |
| | Time (min) | A % | B % |
| Gradient: | 0.0 | 90 | 10 |
| | 12.0 | 45 | 55 |
| | 15.0 | 5 | 95 |
| | 17.0 | 5 | 95 |
| | 18.0 | 90 | 10 |

| | |
|---|---|
| Flow Rate: | 0.8 mL/min |
| UV Detector | 210 nm |
| Wavelength: | |
| Column Temperature: | 40° C. |
| Retention Times: | Compound D: 9.35 min |
| | benzyl (S)-2-cyclopentyl-2-((S)-7-((2R,3R)-3- |
| | cyclopropyl-1-methylaziridine-2-carbonyl)-2,7- |
| | diazaspiro[4.4]nonan-2-yl)acetate: 10.04 min |

### Example 14—Synthesis of (S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethyipropan-1-ol (Compound H)

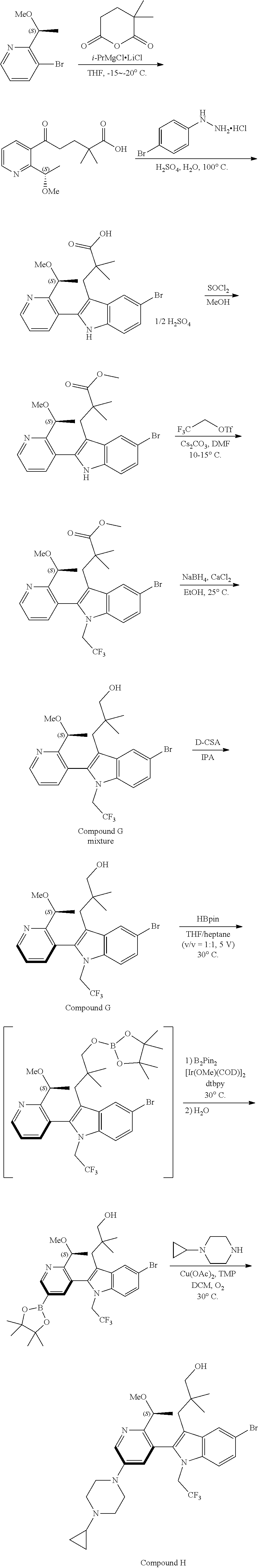

### Part 1—Synthesis of (S)-5-(2-(1-methoxyethyl)pyridin-3-yl)-2,2-dimethyl-5-oxopentanoic acid

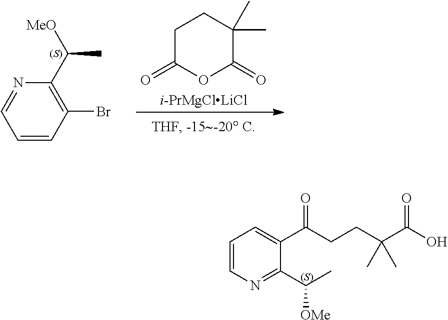

Reactor A was charged with THE (230.5 kg, 2 V) and i-PrMgCl·LiCl (465.0 kg, 1.3 mol/L, 1.03 equiv) under nitrogen protection with stirring. The reactor was cooled to −20±5° C. A solution of 3-bromo-2-[(1S)-1-methoxyethyl]pyridine (129.7 kg, 600.2 mol, 1.0 equiv) in THE (350.4 kg, 3 V) was added to the reactor dropwise (21 kg/min) while maintaining the inner temperature at −20±5° C. The reaction mixture was stirred for 2 h at −20±5° C. and sampled for IPC. Reactor B was charged with THE (577.4 kg, 5 V) and 2,2-dimethylglutaric anhydride (89.2 kg, 627.5 mol, 1.05 equiv) with stirring at −10±5° C. The solution of reactor A was charged to reactor B dropwise at −10±5° C. (3.9 kg/min). The reaction mixture was sampled for HPLC analysis. A solution of 2,2-dimethylglutaric anhydride (0.1 equiv) in THE (0.3 V) was added to the reaction mixture and stirred for additional 5 h at −10±5° C. The reaction mixture was sampled for HPLC analysis. The reaction mixture was quenched by adding H₂O (3 V) at −5±5° C. The mixture temperature was adjusted to 20±5° C. and stirred for at least 0.5 h. n-heptane (3 V) and 4% NaOH aq. solution (3 V) were added to the organic phase. After stirring for 0.5 h, the aqueous phase was separated. The combined aqueous layer was adjusted to pH=7.5±0.3 with KHSO₄ at 20±5° C. The aqueous phase was washed with n-heptane (5 V×1, 2 V×1). The aqueous phase was filtered and the filtrate was adjusted to pH=5.7-7.0 with KHSO₄. The crystal seed of (S)-5-(2-(1-methoxyethyl)pyridin-3-yl)-2,2-dimethyl-5-oxopentanoic acid (14.76% w/w) was added to the solution. The solution was acidified to pH=5.5±0.2 with KHSO₄. The slurry was stirred for at least 5 h at 15-25° C. and filtered. The filter cake of the product was obtained as light yellow solids.

LCMS (ESI+): Calculated for C₁₅H₂₂NO₄ (M+H): 280.1; Found: 280.1

¹H NMR (400 MHz, DMSO-d₆, 25° C.) δ 12.2 (s, 1H), 8.59 (dd, J=4.8, 5.2 Hz, 1H), 7.89 (dd, J=7.6, 8.0 Hz, 1H), 7.40-7.37 (m, 1H), 4.58-4.53 (m, 1H), 3.11 (s, 3H), 2.81-2.77 (m, 2H), 1.82-1.78 (m, 2H), 1.42 (d, J=6.4 Hz, 3H), 1.12 (s, 6H).

### Part 2—Synthesis of (S)-3-(5-bromo-2-(2-(1-methoxyethyl)pyridin-3-yl)-1H-indol-3-yl)-2,2-dimethylpropanoic acid

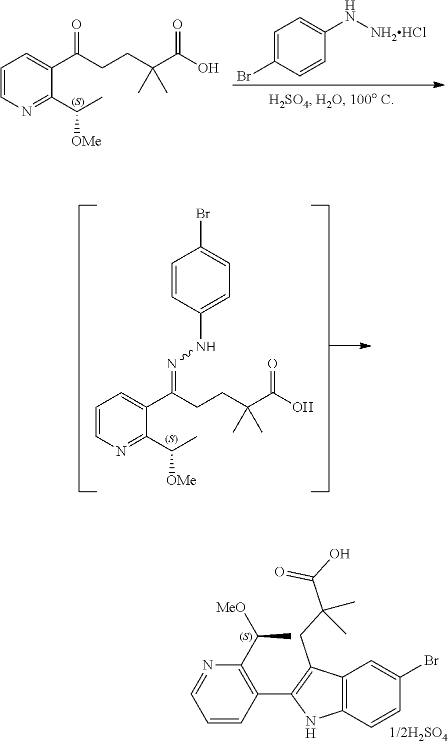

To a reactor was charged H₂O (522 L, 2.5 V). H₂SO₄ (150.7 kg, 1537 mol, 2.0 equiv) was added dropwise to the reactor at 20±20° C. (S)-5-(2-(1-methoxyethyl)pyridin-3-yl)-2,2-dimethyl-5-oxopentanoic acid (215.6 kg, 771.8 mol, 1.0 equiv) and 4-bromophenylhydrazine hydrochloride (189.5 kg, 847.9 mol, 1.1 equiv) were added to the reactor at 20±10° C. The reaction mixture was heated to 70-75° C. and stirred for at least 0.5 h. The reaction mixture was then heated at 95±5° C. for 32 h. The mixture was sampled for HPLC analysis. After completion, the reaction was cooled to 60-65° C. Water (1185.8 kg, 5.5 V) and 30% NaOH aq. solution (189.3 kg, 1.9 equiv) were added, and the mixture was stirred at 60-65° C. for 5 h. The mixture was cooled to 25±5° C. and stirred for 2 h. The mixture was filtered and washed with H₂O (800 L, 3 V). The wet cake was added to H₂O (1022.5 kg) and H₂SO₄ (53.9 kg) at 25±5° C. The mixture was warmed to 80-85° C. and stirred for 5 h. The mixture was then cooled to 25-30° C. and stirred for 16 h. The slurry was filtered and washed with H₂O (1020 L, 3 V). The filter cake was dried under 50±5° C. for 36 h to afford the product as yellow solids.

LCMS (ESI+): Calculated for C₂₁H₂₄BrN₂O₃ (M+H): 433.1; Found: 433.1

¹H NMR (400 MHz, DMSO-d₆, 25° C.) δ 12.1 (s, 1H), 11.4 (s, 1H), 8.70 (s, 1H), 7.82-7.77 (m, 2H), 7.45-7.20 (m, 3H), 4.19 (d, J=5.6 Hz 1H), 2.93 (s, 4H), 2.78 (s, 1H), 1.36 (d, J=4.0 Hz, 3H), 0.88 (d, J=5.2 Hz, 6H).

### Part 3—methyl (S)-3-(5-bromo-2-(2-(1-methoxyethyl)pyridin-3-yl)-1H-indol-3-yl)-2,2-dimethylpropanoate

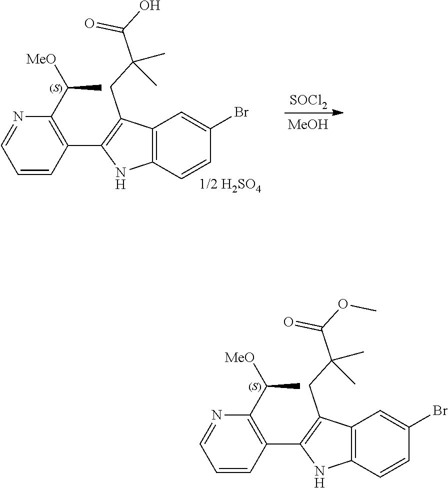

To a reactor were charged MeOH (5.45 L, 5 V) and (S)-3-(5-bromo-2-(2-(1-methoxyethyl)pyridin-3-yl)-1H-indol-3-yl)-2,2-dimethylpropanoic acid·½H₂SO₄ (1.09 kg, 2.27 mol, 1.0 equiv). SOCl₂ (331 g, 2.78 mol, 1.2 equiv) was then added at 50-60° C. The reaction mixture was stirred at 65° C. for 12 h. After completion, the reaction mixture was cooled to 10° C. The mixture was adjusted to pH=7-8 with 5% Na₂CO₃ at 10-15° C. The mixture was filtered and the filter cake was washed with H₂O (2.2 L, 2 V). The wet cake was triturated with H₂O (5.4 L, 5 V) at 25° C. for 6 h. The mixture was filtered and the filter cake was washed with H₂O (2.2 L, 2 V). The filter cake was dried in blast air oven (N₂, 45-50° C.) for 24 h to afford the product.

LCMS (ESI+): Calculated for C₂₂H₂₆BrN₂O₃ (M+H+): 447.1; Found: 446.9

1H NMR (300 MHz, DMSO-d6) δ 11.41 (s, 1H), 8.72 (dd, J=4.7, 1.8 Hz, 1H), 7.81 (dd, J=7.7, 1.8 Hz, 1H), 7.65 (d, J=1.9 Hz, 1H), 7.47 (dd, J=7.8, 4.7 Hz, 1H), 7.32 (d, J=8.6 Hz, 1H), 7.22 (dd, J=8.6, 1.9 Hz, 1H), 4.23 (q, J=6.3 Hz, 1H), 3.32 (s, 9H), 2.95 (s, 4H), 1.39 (d, J=6.3 Hz, 3H), 0.94 (s, 6H).

### Part 4—Synthesis of methyl (S)-3-(5-bromo-2-(2-(1-methoxyethyl)pyridin-3-yl)-1-(2,2,2-trifluoroethyl)-1H-indol-3-yl)-2,2-dimethylpropanoate

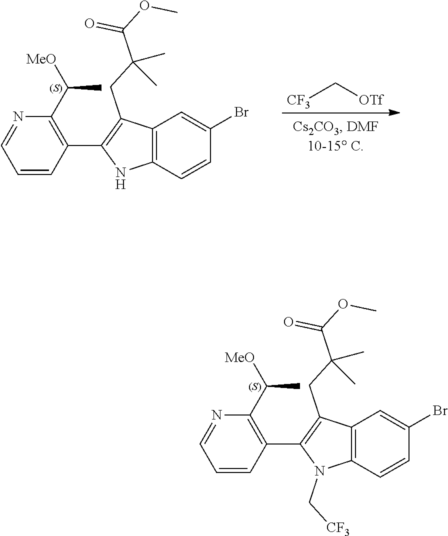

To a reactor were charged DMF (1163.1 kg, 5 V), methyl (S)-3-(5-bromo-2-(2-(1-methoxyethyl)pyridin-3-yl)-1H-indol-3-yl)-2,2-dimethylpropanoate (239.04 kg, 536.7 mol, 1.0 equiv), and Cs₂CO₃ (245.94, 754.8 mol, 1.4 equiv). The reactor was rinsed with DMF (454.6 kg, 2 V). The reaction mixture was cooled to 15-20° C. and 2,2,2-trifluoroethyl trifluoromethanesulfonate (170 kg, 732.4 mol, 1.35 equiv) was added. The reaction mixture was stirred at 15-20° C. for 16 h. The reaction mixture was sampled for HPLC analysis. After completion the reaction was quenched with AcOH (64.4 kg, 2.0 equiv) and H₂O (1435.0 kg, 6 V) at 15-20° C. The resulting mixture was extracted with MTBE two times (5 V, 2 V). The combined organic layers were concentrated to 3-4 V at 35-45° C. The concentrated mixture was diluted with EtOH (4 V) and further concentrated to about 3-4 V under reduced pressure at 35-45° C. The previous unit operation was repeated until the MTBE content in EtOH solution was below 0.5% w/w. The resulting EtOH solution of methyl (S)-3-(5-bromo-2-(2-(1-methoxyethyl)pyridin-3-yl)-1-(2,2,2-trifluoroethyl)-1H-indol-3-yl)-2,2-dimethylpropanoate, as a 1.4:1 mixture of diastereomers, was used directly in the next step.

LCMS (ESI+): Calculated for C₂₄H₂₇BrF₃N₂O₃ (M+H+): 529.1; Found: 529.9

1H NMR (300 MHz, DMSO-d₆) δ 8.77 (dt, J=4.8, 1.4 Hz, 1H), 7.86-7.75 (m, 1H), 7.73-7.62 (m, 2H), 7.55 (ddd, J=7.8, 6.0, 4.7 Hz, 1H), 7.40 (ddd, J=8.8, 4.0, 1.9 Hz, 1H), 5.37 (dd, J=16.5, 8.6 Hz, 1H), 4.82 (dd, J=17.9, 9.0 Hz, 1H), 4.46 (dd, J=16.4, 9.5 Hz, OH), 3.93 (dt, J=18.4, 6.2 Hz, 1H), 3.66-3.56 (m, 2H), 3.46 (d, J=13.2 Hz, 3H), 3.06 (s, 1H), 2.94 (s, 2H), 1.84-1.70 (m, 2H), 1.32 (dd, J=37.8, 6.2 Hz, 3H), 1.05-0.87 (m, 7H).

### Part 5—Synthesis of Mixture of Atropisomers of Compound G—(S)-3-(5-bromo-2-(2-(1-methoxyethyl)pyridin-3-yl)-1-(2,2,2-trifluoroethyl)-1H-indol-3-yl)-2,2-dimethylpropan-1-ol

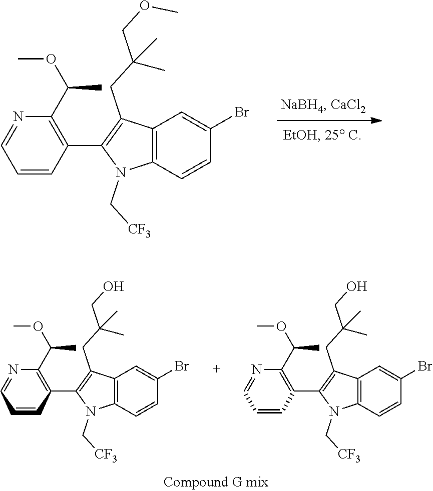

To a reactor was charged the EtOH solution of methyl (S)-3-(5-bromo-2-(2-(1-methoxyethyl)pyridin-3-yl)-1-(2,2,2-trifluoroethyl)-1H-indol-3-yl)-2,2-dimethylpropanoate (1.0 equiv, 5 V) at 15-20° C. under N₂. To the mixture was charged CaCl₂) (60.04 kg, 541.0 mol, 1.0 equiv) at 20-30° C. batchwise, followed by addition of NaBH₄ (51.04 kg, 1349.2 mol, 2.5 equiv) at 20-30° C. The reaction mixture was agitated at 20-30° C. under for 14 h. The reaction mixture was sampled for HPLC analysis. The reaction was quenched by dropwise addition of aq. HCl (3 M) to the point of pH=1-2. Aq. NaOH (30% w/w) was added to the mixture to adjust the pH=4-4.5. The resulting mixture was concentrated 3-5 V. The concentrated mixture was further diluted with MTBE (891.2 kg, 5 V) and H₂O (1197.5 kg, 5 V). The organic phase was separated. The organic layer was washed with H₂O (718.5 kg, 3 V), aq. NaOH (484.0 kg, 2 V) and H₂O (718.5 kg, 3 V) sequentially. The organic layer was concentrated to 3-5 V at 45-60° C. The concentrated mixture was diluted with IPA (756.3 kg, 4 V) and further concentrated to about 3-4 V under reduced pressure at 45-60° C. The previous unit operation was repeated until the MTBE content in IPA solution was below 2% w/w. The resulting IPA solution of Compound G mixture, as a 1.46:1 mixture of diastereomers (247.6 kg, 98.8% a/a, 92% isolated yield) was purified by column purification or by chemical resolution.

### Part 6—Purification of Compound G Mixture

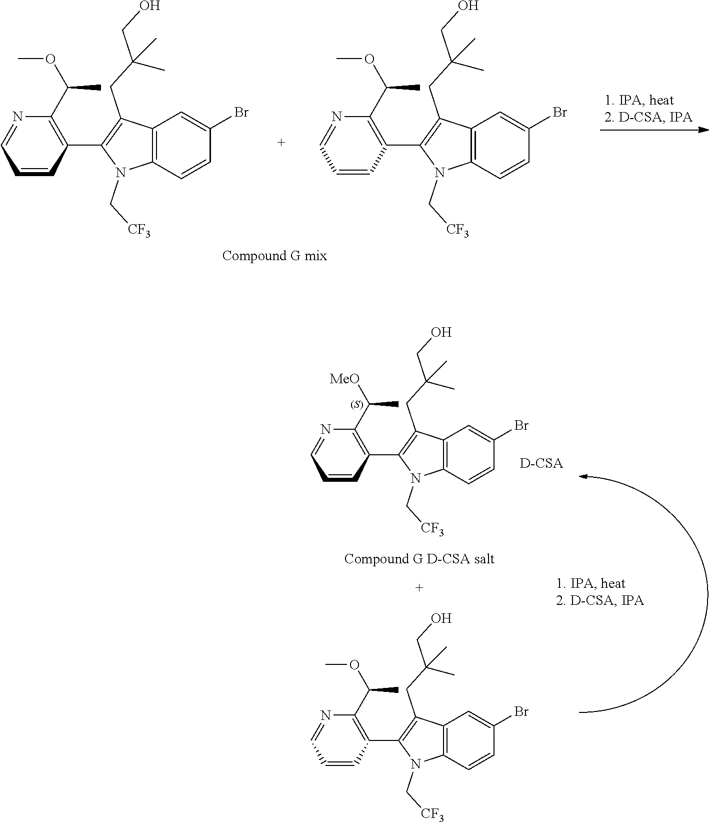

### Condition 1: Column Chromatography

The mixture of Compound G (19.0 kg) was treated with SiO₂ (30.0 kg). The silica gel was loaded on column chromatography (240 kg SiO₂, n-heptane/EtOAc=1/0 to 0/1) to obtain the pure fractions containing Compound G (˜1000 L). The fractions were concentrated to −25 L. To the concentrated residue was added n-heptane (100 L) and the resulting mixture was concentrated to −25 L. The n-heptane wash was repeated once. The residue was diluted with n-heptane (100 L) and the mixture was stirred at 15-20° C. for 12 h. The slurry was filtered and the wet cake was dried in the oven under vacuum to obtain Compound G (18.0 kg, 98.9% a/a) as white solids.

### Condition 2: Resolution by CSA

The IPA solution of the Compound G mixture was added to a reactor, to which was charged IPA (1314.0 kg, 1.2 V). The IPA solution of Compound G mixture (assay weight 247.6 kg, 495.8 mol, 1.0 equiv) was then pumped through the continuous reactor& quenching reactor (the continuous reactor: 220±10° C., quenching reactor: 0-20° C., back pressure value: 4.00±0.20 MPa) with flow rate of pump 80.0 L/h. The equilibrated IPA solution of Compound G was charged to reactor while remaining agitated at 20±10° C. The mixture was sampled for HPLC analysis (criterion: diastereomers ratio≥1.77). D-camphor-10-sulfonic acid (CSA, 150.0 kg, 645.7 mol, 1.3 equiv) was added to the reaction mixture followed by adding IPA (21.5 kg, 0.1 V) to rinse the charging port. The reaction mixture was stirred at 25±5° C. for 2 h and then stirred at 53±3° C. for 2 h. Compound G CSA seed (0.1% w/w) was charged to the reaction mixture at 43±3° C. and then stirred at 43±3° C. for 2 h. The solution was further cooled to −6±3° C. and stirred at the temperature for 4 h. The resulting suspension was filtered and washed with IPA (212.0 kg, 1.0 V). To further control the atropisomer impurity of Compound G, the wet cake is re-suspended in IPA (10 V) and agitated at 67-70° C. for 3-4 h. The mixture was cooled to 5° C. and stirred at 5° C. for 2 h. The resulting wet cake (assay weight 200.0 kg, 1.0 equiv) was suspended in MTBE (3.5 V) at 20±5° C., followed by adding H₂O (4 V). The pH was adjusted to pH=8-10 by slowly charging aq. NaOH (30% w/w). The mixture was agitated at 20±5° C. for 0.5 h. The organic phase was separated and washed with H₂O (3×3 V). The organic layer was concentrated to 1-2 V. The concentrated mixture was diluted with n-heptane (3 V) and further concentrated to about 2-3 V under reduced pressure. The solvent swap with n-heptane was repeated two times. The concentrated mixture was diluted with n-heptane (3 V) and the mixture was agitated at 55±5° C. for 3 h. The mixture was cooled to 20±5° C. and stirred for 1 h at 20±5° C. The slurry was filtered and washed with n-heptane (1.2 V). The wet cake was dried at 45-55° C. to afford the product as off-white solids (143.6 kg, 99.05% a/a, 58% yield). To further control the atropisomer impurity of Compound G, the solid was re-suspended in THF/n-heptane (v/v=1:4, 5 V) and agitated at 67-70° C. for 3-4 h. The mixture was cooled to 5° C. and stirred at 5° C. for 2 h. The slurry was filtered and washed with n-heptane (1.5V). The wet cake was dried at 45-55° C. to afford Compound G as off-white solids. To recover Compound G, the mother liquor from resolution process was charged into a reactor. The solution was concentrated to 4-5 V under vacuum at NMT 50° C. 30% w/w aq. NaOH solution was added to the concentrated residue to adjust the pH to 8-10 at 15-30° C. The mixture was concentrated to 0.8-1.2 V under vacuum at NMT 50° C. MTBE (5 V) and H₂O (5 V) were added to the residue. The biphasic system was stirred at 25±5° C. for 0.5 h. The organic phase was separated and washed with H₂O (5 V). The organic phase was then concentrated to 1.2-1.6 V at NMT 50° C. The concentrated residue was diluted with IPA (1.6 V) and the mixture was then concentrated to 1.2-1.6 V at NMT 50° C. The solvent swap with IPA was repeated once and IPA (3 V) was added to the concentrated residue. The IPA solution of the Compound G mixture was subjected to the equilibrium process and CSA salt formation again. The IPA solution of the Compound G mixture was pumped through the continuous reactor and quenching reactor (the continuous reactor: 220±10° C.; quenching reactor: 0-20° C.; back pressure: 4.00±0.20 MPa) with flow rate of pump 80.0 L/h. The equilibrated IPA solution of Compound G was charged to reactor while remaining agitated at 20±10° C. The mixture was sampled for HPLC analysis (criterion: diastereomers ratio≥1.77). D-camphor-10-sulfonic acid (1.3 equiv) was added to the reaction mixture followed by adding IPA (0.1 V) to rinse the charging port. The reaction mixture was stirred at 25±5° C. for 2 h and then stirred at 53±3° C. for 2 h. A CSA seed of Compound G (0.1% w/w) was charged to the reaction mixture at 43±3° C. and then stirred at 43±3° C. for 2 h. The solution was further cooled to −6±3° C. and stirred at the temperature for 4 h. The resulting suspension was filtered and washed with IPA (1.0 V). The resulting wet cake (1.0 equiv) was suspended in MTBE (3.5 V) at 20±5° C., followed by adding H₂O (4 V). The pH was adjusted to pH=8-10 by slowly charging aq. NaOH (30% w/w). The mixture was agitated at 20±5° C. for 0.5 h. The organic phase was separated and washed with H₂O (3×3 V). The organic layer was concentrated to 1-2 V. The concentrated mixture was diluted with n-heptane (3 V) and further concentrated to about 2-3 V under reduced pressure. The solvent swap with n-heptane was repeated two times. The concentrated mixture was diluted with n-heptane (3 V) and the mixture was agitated at 55±5° C. for 3 h. The mixture was cooled to 20±5° C. and stirred for 1 h at 20±5° C. The slurry was filtered and washed with n-heptane (1.2 V). The wet cake was dried at 45-80° C. to afford atropisomerically pure Compound G as off-white solids.

### Condition 3: Resolution by Methanesulfonic Acid (MsOH)

To the toluene solution of the Compound G mixture was charged xylene (3 V). The mixture was concentrated to 2.5-3.5 V. The residue was agitated at 137±5° C. for 24 h. The mixture was cooled to 60-70° C. and a sample was taken for HPLC analysis. The residue was diluted with 2-MeTHF (5 V) at 20±5° C. The solution was washed with aq. HCl (0.2 M, 3 V) twice. The organic phase was separated and extracted with aq. HCl (3 M, 3 V) twice. The combined aqueous phase was washed with MTBE (3 V). The aqueous phase was adjusted to pH=8-10 with 30% aq. NaOH. The aqueous phase was extracted with IPAc (5 V). The organic phase was extracted and washed with H₂O (3 V). The organic phase was concentrated to 2-3 V under reduced pressure. The solvent swap with IPAc was repeated twice. The concentrated residue was diluted with IPAc (5 V). To the IPAc solution was added methanesulfonic acid (MSA, 0.35 equiv) at 20±10° C. The mixture was stirred at 30±5° C. for 4 h. The slurry was filtered and the cake was washed with IPAc (2V). The cake containing the undesired atropisomer of Compound G as a mesylate salt is collected and subject to recovery. The filtrate was added 5% aq. NaHCO₃ (3 V) and the pH of aqueous phase should be 8. To the biphasic system was added H₂O (3 V). The organic phase was separated and concentrated to 1.8-2.3 V under vacuum at NMT 45° C. The concentrated residue was diluted with MeOH (7 V). The resulting solution was concentrated to 1.8-2.3 V under vacuum at NMT 45° C. To the MeOH solution was added MSA (1.0 equiv) at 20±10° C. The mixture was stirred at 40° C. for 0.5 h until all solids are dissolved. To the solution was added H₂O (2.4 V) over 2 h followed by adding Compound G seed (10% w/w). The mixture was stirred at 40° C. for 4 h before H₂O (1.8 V) was added dropwise. Another portion of H₂O (6 V) was added dropwise and the mixture was stirred for 1 h. The mixture was cooled to 20° C. and the slurry was stirred for 4 h. The slurry was filtered and the wet cake was washed with MeOH/H₂O (v/v=1:3.5, 1.8 V). The wet cake was dried in the oven at 50-55° C. for 16 h to afford Compound G as white solids. To recover Compound G, the filtrate was concentrated under vacuum. The residue was combined with the wet cake of the undesired atropisomer of Compound G as the mesylate salt. Aq. NaOH (30% w/w) was added to adjust the pH to 8-10. MTBE (5 V) was added to the reaction mixture. The mixture was stirred for 1 h at 20±5° C. The organic phase was separated and concentrated to 1.5-3 V. The concentrated residue was diluted with MTBE (5 V). The resulting mixture was concentrated to 1.5-3 V. The solvent swap with MTBE was repeated twice. The Compound G MTBE solution was concentrated to 1.5-2.5 V and to the residue was added xylene (3 V). The mixture was stirred at 138-143° C. for 24 h before it was cooled to 60° C. To the mixture was added IPA (6.3 V) and the resulting solution was concentrated to 2-3 V under vacuum. The solvent swap with IPA was repeated twice. To the IPA solution was added D-camphor-10-sulfonic acid (1.3 equiv) at 15±5° C. The mixture was cooled to −5-0° C. and stirred for 14 h at −5-0° C. The slurry was filtered and washed with IPA (2 V). The wet cake was dried at 45-80° C. to afford Compound G as off-white solids.

LCMS (ESI+): Calculated for C₂₃H₂₇BrF₃N₂O₂ (M+H⁺): 499.1; Found: 499.1

1H NMR (400 MHz, DMSO-d6) δ 8.75 (dd, J=4.8, 5.2 Hz, 1H), 7.98 (d, J=1.6 Hz, 1H), 7.78-7.76 (m, 1H), 7.66-7.63 (m, 1H), 7.54-7.53 (m, 1H), 7.40-7.38 (m, 1H), 5.39-5.30 (m, 1H), 4.60-4.56 (m, 1H), 4.51-4.42 (m, 1H), 3.98-3.93 (m, 1H), 3.33 (s, 2H), 3.03-2.94 (m, 3H), 2.60-2.50 (m, 1H), 2.29-2.26 (m, 1H), 1.36 (d, J=6.4 Hz, 2H), 0.60 (s, 6H).

### Part 7a—Two-step synthesis of (S)-3-(5-bromo-2-(2-(1-methoxyethyl)-5-(4,4,5,5-tetramethyl-1,3,2-dioxaborolan-2-yl)pyridin-3-yl)-1-(2, 2, 2-trifluoroethyl)-1H-indol-3-yl)-2, 2-dimethylpropan-1-ol

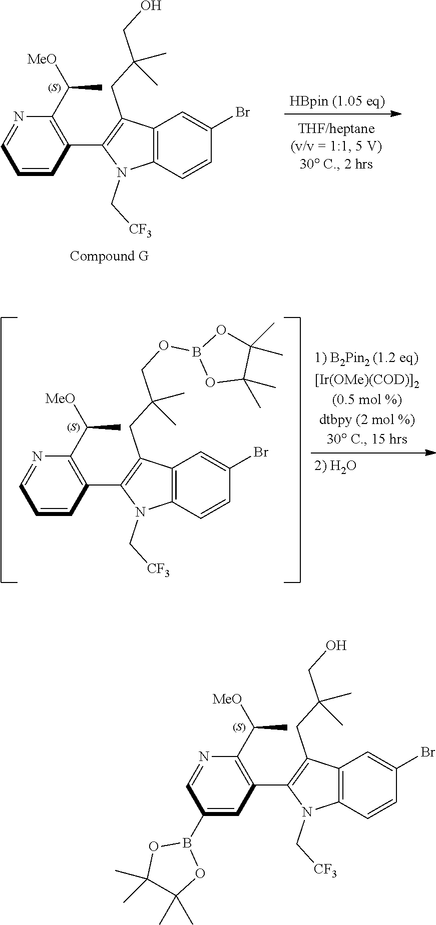

Compound G (200.0 kg, 400.5 mol, 1.0 equiv) was dissolved in THE (500.0 L, 2.5 V) and heptane (500.0 L, 2.5 V) at 20±5 c. HBPin (53.8 kg, 420.4 mol, 1.05 equiv) was added to the reaction mixture dropwise at 20±5° C. and the mixture was stirred at 30-35 for 2 h. The reaction mixture was sampled for ¹HNMR to indicate that Compound G was consumed completely. The reaction mixture was cooled to 20° C. B₂Pin₂ (119.0 kg, 468.6 mol, 1.2 equiv), dtbpy (2.1 kg, 8.01 mol, 0.02 equiv) and [Ir(OMe)(COD)]2 (1.3 kg, 2.0 mol, 0.005 equiv) were added to the reaction mixture sequentially. The resulting mixture was stirred at 30-35 for 12 h. A sample was taken for HPLC analysis. The reaction was quenched by charging H₂O (0.1 V) and THE (0.5 V) at 0-5° C., followed by charging MTBE (4 V) and H₂O (4 V). The mixture was filtered and the cake was washed with MTBE (3 V). The filtrate was separated and the organic layer was washed with H₂O (3 V). The organic layer was concentrated to 1-2 V at 40-45° C. The concentrated mixture was diluted with DCM (4 V) and further concentrated to 1-2 V. The previous unit operation was repeated twice. The resulting DCM solution of (S)-3-(5-bromo-2-(2-(1-methoxyethyl)-5-(4,4,5,5-tetramethyl-1,3,2-dioxaborolan-2-yl)pyridin-3-yl)-1-(2,2,2-trifluoroethyl)-1H-indol-3-yl)-2,2-dimethylpropan-1-ol was used directly for next step (479 kg, 86% assay yield, 43.1% w/w, 85.1% a/a).

### Part 7b—One-Step Synthesis of (S)-3-(5-bromo-2-(2-(1-methoxyethyl)-5-(4,4,5,5-tetramethyl-1,3,2-dioxaborolan-2-yl)pyridin-3-yl)-1-(2,2,2-trifluoroethyl)-1H-indol-3-yl)-2,2-dimethylpropan-1-ol

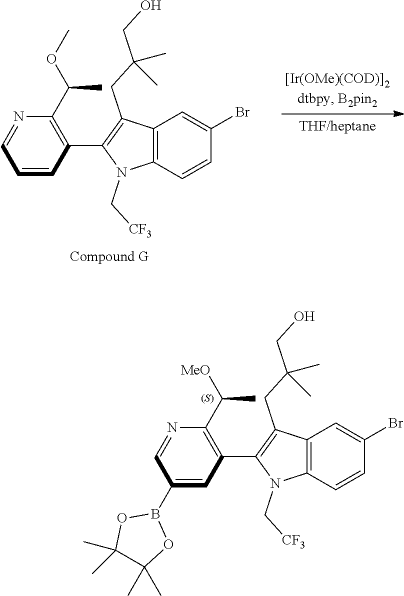

To a reactor was charged n-heptane (4 V), dtbpy (1.6 mol %) at 25° C., followed by adding Compound G (1.0 equiv) and B₂Pin₂ (2.0 equiv). N₂ was bubbled under surface of reaction mixture for 1 h. [Ir(OMe)(COD)]2 (0.8 mol %) was added to the suspension under the atmosphere of N₂. The reaction mixture was stirred under N₂ gas until all the solids were dissolved. The reaction mixture was then stirred at 55 for 2 h. After completion, the reaction solution was washed with H₂O (3 V) twice. The organic phase was concentrated to dryness and used for next step.

LCMS (ESI+): Calculated for C₂₃H₂₇BBrF₃N₂O₄ (M-2,3-dimethylbutane): 544.1; Found: 544.0

1H NMR (300 MHz, chloroform-d) δ 9.11 (d, J=1.7 Hz, 1H), 8.10 (t, J=1.5 Hz, 1H), 7.92 (d, J=1.9 Hz, 1H), 7.40 (dd, J=8.7, 1.9 Hz, 1H), 7.29 (s, 1H), 4.73 (s, 1H), 4.47 (q, J=8.6 Hz, 2H), 4.03 (q, J=6.2 Hz, 1H), 3.82-3.72 (m, 1H), 3.58-3.45 (m, 2H), 3.04 (s, 3H), 2.74 (d, J=14.1 Hz, 1H), 2.23 (d, J=14.1 Hz, 1H), 1.91-1.83 (m, 1H), 1.46 (d, J=6.2 Hz, 4H), 1.36 (s, 12H), 1.28 (d, J=5.2 Hz, 35H), 0.95-0.85 (m, 5H), 0.76 (d, J=10.4 Hz, 6H).

### Part 8—Synthesis of Compound H—(S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropan-1-ol

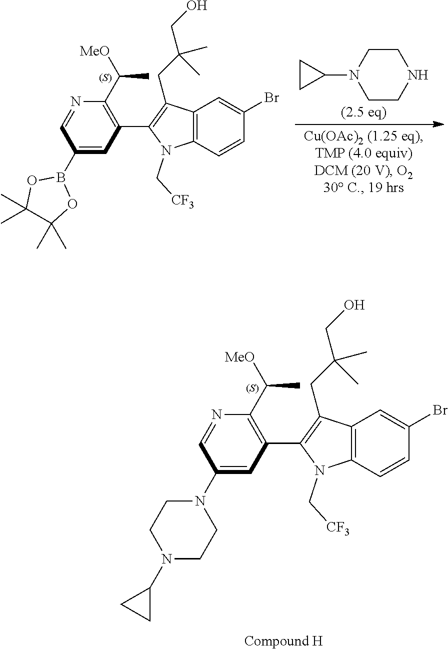

### Condition 1: TMP as Base

A reactor was charged with DCM (1173 L, 30 V), 2,2,6,6-tetramethylpiperidine (35.3 kg, 250.0 mol, 4.0 equiv), 1-cyclopropylpiperazine (19.7 kg, 156.1 mol, 2.5 equiv), and Cu(OAc)₂ (14.2 kg, 78.2 mol, 1.25 equiv) at 15-20° C. with agitation. 21% 02 was bubbled under the surface for 1 h at 20-25° C. (S)-3-(5-bromo-2-(2-(1-methoxyethyl)-5-(4,4,5,5-tetramethyl-1,3,2-dioxaborolan-2-yl)pyridin-3-yl)-1-(2,2,2-trifluoroethyl)-1H-indol-3-yl)-2,2-dimethylpropan-1-ol in DCM (39.1 kg assay weight, 62.5 mol, 1.0 equiv) was added to the reaction mixture at 10-20° C. The reaction mixture was heated at 25-35° C. during which 21% 02 was bubbled under surface and was stirred at this condition for 16 h. A sample was taken for HPLC analysis. The reaction was cooled to 20° C. The reaction mixture was quenched with a solution of NH₄₀H (1.5 V) and H₂O (3 V). The organic phase was separated and washed with a solution of NH₄₀H (1.5 V) and H₂O (3 V) for two additional times. The organic phase was washed with aq. EDTA (5% w/w, 3 V) and aq. NaCl (5% w/w, 4 V) sequentially. The organic phase was concentrated to about 1-2 V under reduced pressure at 40-45° C. The concentrated mixture was diluted by 2-MeTHF (6 V) and H₂O (3 V). The pH of the system was adjusted to pH=1.1-1.3 by aq. HCl (2 N). The water layer was washed by 2-MeTHF (4 V) two times (Compound G contained in the 2-MeTHF solution can be recycled, the detailed procedure is described below). The water phase pH was adjusted to 8-9 by adding aq. NaOH (30% w/w) slowly. The water phase was concentrated under reduced pressure until no fraction to obtain the Compound H crude. Compound H crude was dissolved in MeCN (2 V) and was stirred at 40-50° C. for 3 h. To the solution was added H₂O (1 V) at 35-45° C., followed by adding a seed of Compound H (0.5% w/w). The mixture was stirred at 35-45° C. for 2 h, to which was added H₂O (2 V). The mixture was cooled to 15-25° C. The slurry was filtered and the cake was washed with MeCN/H₂O (2 V, v/v=2:3). The wet cake was dried in blast air oven (N2, 45-50° C.) for 12 h to afford Compound H as brown solids (51.9% yield from Compound G, 98.8% a/a).

To recover Compound G, the 2-MeTHF solution containing Compound G is added to a reactor and was washed with aq. NaOH (0.1 N, 1 V) twice. The organic phase was separated, and H₂O (1 V) was added. The aqueous phase was adjusted to pH=1-1.2 with aq. HCl (3N). The organic phase was separated and concentrated under vacuum at 45° C. until dryness. The residue was dissolved in IPA (1 V) and concentrated to 1-2 V. The concentrated residue was diluted with IPA (8 V). D-camphor-10-sulfonic acid (CSA, 1.2 equiv) in IPA (2 V) was added to the reaction mixture dropwise at 15-20° C. The reaction mixture was stirred at 15-20° C. for 2 h and then stirred at 50-55° C. for 2 h. Compound G CSA seed (0.1% w/w) was charged to the reaction mixture at 43±3° C. and then stirred at 43±3° C. for 2 h. The solution was further cooled to −6±3° C. and stirred at the temperature for 4 h. The resulting suspension was filtered and washed with IPA (1.0 V). The wet cake was suspended in IPA (3 V) and stirred at 40° C. for 60 min. The mixture was cooled to 5° C. and stirred at 5° C. for 60 min. The slurry was filtered and washed with IPA (1 V) to afford wet cake as Compound G CSA salt. The resulting wet cake (1.0 equiv) was suspended in MTBE (3.5 V) at 20+5° C., followed by adding H₂O (4 V). The pH was adjusted to pH=8-10 by slowly charging aq. NaOH (30% w/w). The mixture was agitated at 20±5° C. for 0.5 h. The organic phase was separated and washed with H₂O (3×3 V). The organic layer was concentrated to 1-2 V. The concentrated mixture was diluted with n-heptane (3 V) and further concentrated to about 2-3 V under reduced pressure. The solvent swap with n-heptane was repeated twice. The concentrated mixture was diluted with n-heptane (3 V) and the mixture was agitated at 55±5° C. for 3 h. The mixture was cooled to 20±5° C. and stirred for 1 h at 20±5° C. The slurry was filtered and washed with n-heptane (1.2 V). The wet cake was dried in Cone dryer at 45-80° C. to afford the Compound G as off-white solids.

### Condition 2: TEA as Base

A reactor was charged with DCM (15 V), TEA (4.0 equiv), 1-cyclopropylpiperazine (2.5 equiv), and Cu(OAc)₂ (1.25 equiv) at 15-20° C. with agitation. 21% 02 was bubbled under surface for 1 h at 20-25° C. (S)-3-(5-bromo-2-(2-(1-methoxyethyl)-5-(4,4,5,5-tetramethyl-1,3,2-dioxaborolan-2-yl)pyridin-3-yl)-1-(2,2,2-trifluoroethyl)-1H-indol-3-yl)-2,2-dimethylpropan-1-ol in DCM (1.0 equiv) was added to the reaction mixture at 10-20° C. The reaction mixture was heated at 25-35° C. during which 21% 02 was bubbled under surface and was stirred at this condition for at least 5 h. A sample was taken for HPLC analysis. The reaction was cooled to 20° C. The reaction mixture was quenched with aq. solution of NH₄₀H (1.5 V) and H₂O (3 V). The organic phase was separated and washed with N-acetyl cysteine (1 N, 3 V) and H₂O (2 V) sequentially. The organic phase was concentrated to about 1-2 V under reduced pressure at 40-45° C. The concentrated mixture was diluted by 2-MeTHF (6 V) and H₂O (3 V). The pH of the system was adjusted to pH=1.1-1.3 by aq. HCl (2 N). The water layer was washed by 2-MeTHF (4 V) for two times. The water phase pH was adjusted to 8-9 by adding aq. NaOH (2 N) slowly. The water phase was concentrated under reduced pressure until no fraction to obtain crude Compound H. The crude Compound H was dissolved in MeCN (2 V) and was stirred at 40-50° C. for 3 h. To the solution was added H₂O (3 V) slowly at 35-45° C. The mixture was cooled to 15-25° C. and was further stirred for 3 h. The slurry was filtered and the cake was washed with MeCN/H₂O (2 V, v/v=2:3). The wet cake was dried under vacuum to afford Compound H as brown solids.

LCMS (ESI+): Calculated for C₃₀H₃₉BrF₃N₄O₂ (M+H): 623.2; Found: 622.9

1H NMR (400 MHz, DMSO-d6) δ 8.46 (d, J=2.0 Hz, 1H), 7.95 (s, 1H), 7.62 (d, J=4.4 Hz, 1H), 7.36 (d, J=2.0 Hz, 1H), 7.18 (s, 1H), 5.32-5.26 (m, 1H), 4.61-4.47 (m, 2H), 3.85-3.83 (m, 1H), 3.19 (s, 4H), 3.03-3.02 (m, 2H), 2.91 (s, 3H), 2.68 (s, 4H), 2.60 (s, 1H), 2.30-2.27 (m, 1H), 1.66 (s, 1H), 1.32 (d, J=2.0 Hz, 3H), 0.62 (s, 6H), 0.45-0.35 (m, 4H).

### Alternative Synthesis of the Mixture of Compound G Atropisomers—(S)-3-(5-bromo-2-(2-(1-methoxyethyl)pyridin-3-yl)-1-(2,2,2-trifluoroethyl)-1H-indol-3-yl)-2,2-dimethylpropan-1-ol

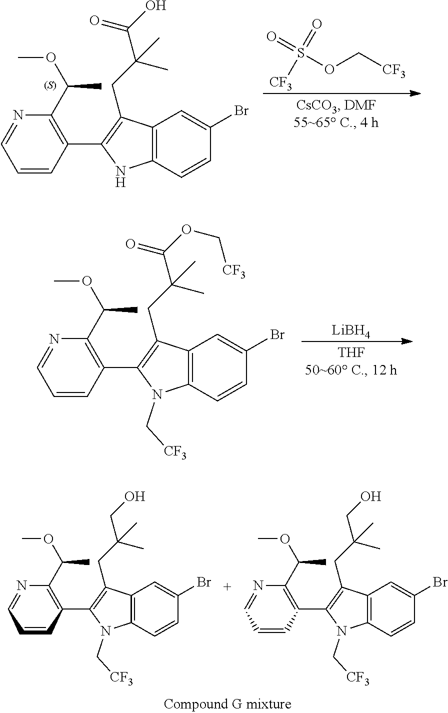

### Part 1—Synthesis of 2,2,2-trifluoroethyl (S)-3-(5-bromo-2-(2-(1-methoxyethyl)pyridin-3-yl)-1-(2,2,2-trifluoroethyl)-1H-indol-3-yl)-2,2-dimethylpropanoate

To a reactor were added DMF (770 L, 7 V) and (S)-3-(5-bromo-2-(2-(1-methoxyethyl)pyridin-3-yl)-1H-indol-3-yl)-2,2-dimethylpropanoic acid (110 kg, 255.0 mol, 1.0 equiv) at 10-20° C. The reaction mixture was cooled to 0-5° C., and Cs₂CO₃ (208.0 kg, 637.0 mol, 2.5 equiv) was added, followed by dropwise addition of 2,2,2-trifluoroethyl trifluoromethanesulfonate (148.0 kg, 637.0 mol, 2.5 equiv). The reaction mixture was stirred at 0-5° C. for 16 h. After completion, the reaction was quenched by adding H₂O at 0-20° C. The mixture was extracted with MTBE (550 L, 5 V). The organic layer was separated and the aqueous layer was extracted with MTBE (550 L, 5 V). The combined organic layers were washed with brine (330 L, 3 V). The organic layer was concentrated to 190-380 L under reduced pressure at 40-50° C. The concentrated residue was diluted with THE (220 L, 2 V) and further concentrated to about 1-2V under reduced pressure. The previous unit operation was repeated until the MTBE content in THE solution was below 1% w/w. The resulting THE solution of 2,2,2-trifluoroethyl (S)-3-(5-bromo-2-(2-(1-methoxyethyl)pyridin-3-yl)-1-(2,2,2-trifluoroethyl)-1H-indol-3-yl)-2,2-dimethylpropanoate was used directly for next step (123.6 kg assay weight, 97.5% yield).

LCMS (ESI+): Calculated for C₂₅H₂₆BrF₆N₂O₃ (M+H): 597.1; Found: 597

1H NMR (400 MHz, DMSO-d6) δ 8.76 (d, J=4.8 Hz, 1H), 7.83-7.77 (m, 2H), 7.68-7.64 (m, 1H), 7.57-7.52 (m, 1H), 7.43-7.39 (m, 1H), 4.50-4.43 (m, 2H), 3.59 (t, J=12 Hz, 1H), 3.04 (s, 1H), 2.92 (s, 2H), 2.88 (s, 2H), 2.73 (s, 2H), 1.76-7.73 (m, 1H), 1.37 (d, J=8.0 Hz, 1H), 1.23 (d, J=8.0 Hz, 1H), 0.98 (d, J=20 Hz, 6H).

### Part 2—Synthesis of the Mixture of Compound G Atropisomers—(S)-3-(5-bromo-2-(2-(1-methoxyethyl)pyridin-3-yl)-1-(2,2,2-trifluoroethyl)-1H-indol-3-yl)-2,2-dimethylpropan-1-ol

To a reactor was added THE solution of 2,2,2-trifluoroethyl (S)-3-(5-bromo-2-(2-(1-methoxyethyl)pyridin-3-yl)-1-(2,2,2-trifluoroethyl)-1H-indol-3-yl)-2,2-dimethylpropanoate (123.6 kg assay weight, 207.0 mol, 1.0 equiv) at 15-20° C. The reaction mixture was warmed to 50-60° C. and LiBH₄ (2M in THF, 155.7 L, 311.0 mol, 1.5 equiv) was added. The reaction mixture was stirred at 60-65° C. for 24 h. A sample was taken for HPLC analysis. After completion, the reaction was quenched with aq. HCl (0.5 M, 618.0 L, 5 V) at 10-20° C. Aq. HCl (0.5 M) was continued to add to adjust the pH to 4-5. MTBE (618 L, 5 V) was added to the mixture and stirred at 10-20° C. for 0.5 h. The organic layer was separated and the aqueous layer was washed with MTBE (370 L, 3 V). The combined organic layers were washed with aq. NaOH (247 L, 2 V) and brine (247 L, 2 V) sequentially. The organic layer was concentrated under reduced pressure at 40-45° C. to afford the mixture of Compound G atropisomers (93.5 kg, 90.1% a/a).

### Alternative Synthesis of the Mixture of Compound G Atropisomers—(S)-3-(5-bromo-2-(2-(1-m ethoxyethyl)pyridin-3-yl)-1-(2, 2, 2-trifluoroethyl)-1H-indol-3-yl)-2, 2-dim ethylpropan-1-ol

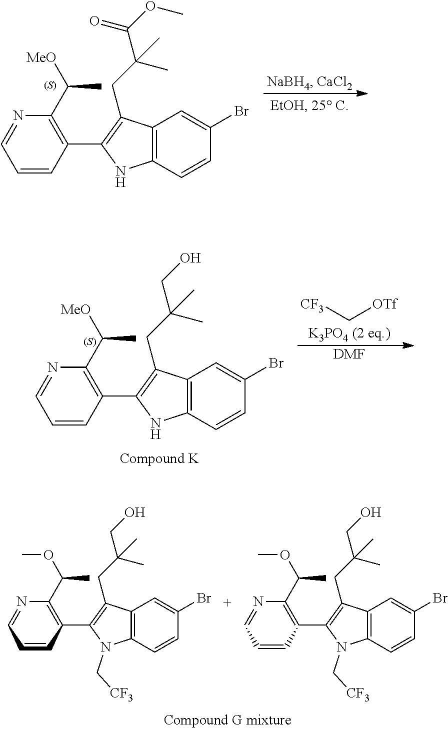

To a reactor was charged the EtOH solution of methyl (S)-3-(5-bromo-2-(2-(1-methoxyethyl)pyridin-3-yl)-1H-indol-3-yl)-2,2-dimethylpropanoate (1.0 equiv, 5 V) at 15-20° C. under N₂. To the mixture was charged CaCl₂ (1.25 equiv) at 5° C. batchwise, followed by addition of NaBH₄ (1.5 equiv) at 5° C. The reaction mixture was agitated at 20-30° C. under for 14 h. The reaction mixture was sampled for HPLC analysis. The reaction was quenched by dropwise addition of aq. HCl (3 M) until a pH of 1-2 was achieved. Aq. NaOH (30% w/w) was added to the mixture to adjust the pH between 4-5. The resulting mixture was concentrated and precipitated from water. The resulted cake was washed with water and dried at 45° C. under vacuum.

To a reactor were charged DMF (7 V), Compound K (1.0 equiv), and K₃PO₄ (1.4 equiv). The reactor was rinsed with DMF (454.6 kg, 7 V). The reaction mixture was cooled to 15-20° C. and 2,2,2-trifluoroethyl trifluoromethanesulfonate (2 equiv) was added. The reaction mixture was stirred at 15-20° C. for 16 h. The reaction mixture was sampled for HPLC analysis. After completion the reaction was quenched with AcOH (64.4 kg, 2.0 equiv) and H₂O (1435.0 kg, 6 V) at 15-20° C. The resulting mixture was extracted with MTBE two times (5 V, 2 V). The combined organic layers were concentrated to 3-4 V at 35-45° C. The concentrated mixture was diluted with IPA (4 V) and further concentrated to about 3-4 V under reduced pressure at 35-45° C. The resulting EtOH solution of methyl (S)-3-(5-bromo-2-(2-(1-methoxyethyl)pyridin-3-yl)-1-(2,2,2-trifluoroethyl)-1H-indol-3-yl)-2,2-dimethylpropanoate (Compound G), as a 1.4:1 mixture of diastereomers, was used directly in the next step.

### Example 15—Synthesis of Compound 1—(2S)-2-cyclopentyl-2-((S)-7-((2R,3R)-3-cyclopropyl-1-methylaziridine-2-carbonyl)-2,7-diazaspiro[4.4]nonan-2-yl)-N-((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)acetamide

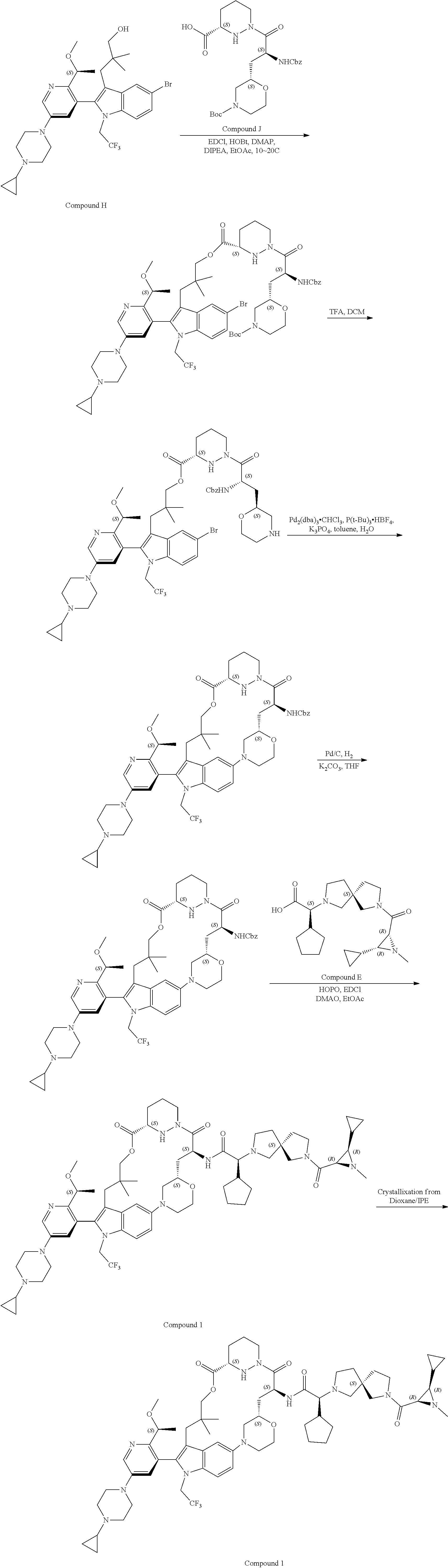

### Part 1—Synthesis of tert-butyl (S)-2-((S)-2-(((benzyloxy)carbonyl)amino)-3-((S)-3-((3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropoxy)carbonyl)tetrahydropyridazin-1(2H)-yl)-3-oxopropyl)morpholine-4-carboxylate

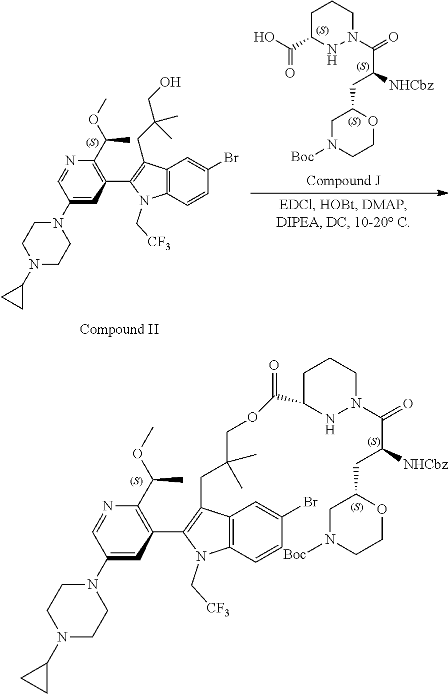

To a reactor was charged DCM (746.15 kg, 9 V), followed by addition of Compound H (67.35 kg, 108.0 mol, 1.0 equiv), Compound J (64.57 kg, 124.0 mol, 1.15 equiv) DCM solution (1 V) of 1-hydroxybenzotriazole (14.02 kg, 103.8 mol, 1.0 equiv), DMAP (6.50 kg, 53.2 mol, 0.5 equiv), DIPEA (27.00 kg, 208.9 mol, 2.0 equiv) and N-(3-Dimethylaminopropyl)-N′-ethylcarbodiimide hydrochloride (29.65 kg, 154.7 mol, 1.5 equiv) at 20±5° C. The reaction mixture was stirred at 15±5° C. for 16 h. Reaction monitoring by HPLC showed that the reaction is complete. The reaction mixture was quenched with aq. NaHCO₃ solution (7% w/w, 7.3% w/w of Compound H) at 15±5° C. and the organic layer was separated. The organic layer was washed with 5% w/w citric acid aqueous solution (731.64 kg). The crude organic solution was concentrated under reduced pressure at NMT 45° C. to 3.5-4.5 V. The concentrated residue was further diluted with DCM (511.85 kg, 6 V). The resulting solution was concentrated under reduced pressure at NMT 45° C. to 3.5-4.5 V. The DCM solution of tert-butyl (S)-2-((S)-2-(((benzyloxy)carbonyl)amino)-3-((S)-3-((3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropoxy)carbonyl)tetrahydropyridazin-1(2H)-yl)-3-oxopropyl)morpholine-4-carboxylate was used for next step without further purification (90.4% a/a).

LCMS (ESI+): Calculated for C₅₅H₇₂BrF₃N₈O₉ (M+H): 1125.5; Found: 1125.9

¹H NMR (400 MHz, DMSO-d₆, 25° C.) δ 8.48 (d, J=2.8 Hz, 1H), 7.87 (d, J=2.0 Hz, 1H), 7.65 (d, J=8.7 Hz, 1H), 7.39 (dd, J=8.8, 1.9 Hz, 1H), 7.33 (m, 5H), 7.22 (d, J=2.7 Hz, 1H), 7.12 (d, J=8.8 Hz, 1H), 5.33 (dd, J=17.0, 8.6 Hz, 1H), 5.07 (m, 2H), 4.98 (s, 2H), 4.51 (m, 1H), 3.97 (s, 1H), 2.88 (s, 3H), 1.37 (s, 9H), 1.32 (d, J=6.2 Hz, 3H), 0.78 (s, 3H), 0.73 (s, 3H), 0.42 (m, 2H), 0.34 (m, 2H).

### Part 2—Synthesis of 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate

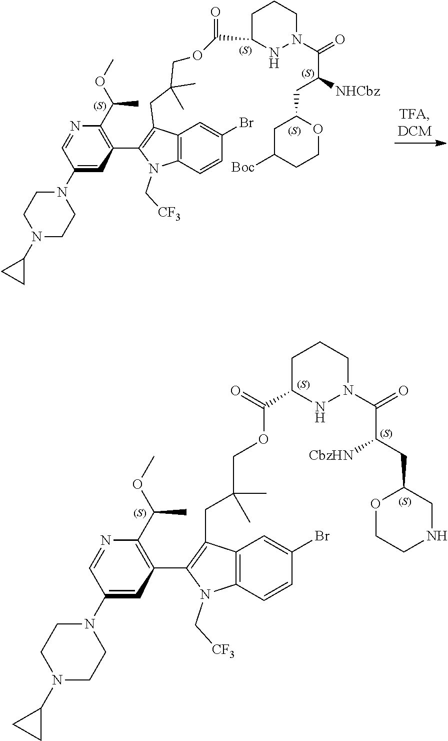

### Condition 1: Work-Up with K₂CO₃

To a reactor was charged with tert-butyl (S)-2-((S)-2-(((benzyloxy)carbonyl)amino)-3-((S)-3-((3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropoxy)carbonyl)tetrahydropyridazin-1(2H)-yl)-3-oxopropyl)morpholine-4-carboxylate DCM solution (64.1 kg, 1.0 eq) and it was cooled to 5±5° C. under nitrogen. TFA (237.10 kg, 2.4 V) was charged to the reactor dropwise while maintaining the inner temperature at 5±5° C. The reaction mixture was stirred at 10±5° C. for 1 h and then stirred at 15±5° C. for no less than 4 h. Reaction monitoring by HPLC showed reaction is complete. Aqueous solution of K₂CO₃ (731.65 kg, 40% w/w) was charged to reaction mixture to adjust the pH to the range of 7-10 while maintaining the temperature at 10±10° C. H₂O (57.50 kg, 4 V) was added to the mixture and stirred for 0.5 h. The organic layer was separated and was washed with H₂O (452.5 kg, 7 V). The solvent in the solution was exchanged with MTBE (470.9 kg, 10 V) by distillation. The resulting MTBE solution of crude product was added to n-heptane (872.05 kg, 20 V) in another reactor over 6 h at 15±5° C. The mixture was stirred at 15±5° C. for at least 3 h. The slurry was filtered, and the wet cake was washed with n-heptane (87 kg, 2V). The crude solid was dried at 25±5° C. under vacuum for 10 h to give 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate as a white solid (103.72 kg, 94.1% a/a, 90.1% yield over 2 steps).

### Condition 2: Work-Up with NaOH

To a reactor was charged tert-butyl (S)-2-((S)-2-(((benzyloxy)carbonyl)amino)-3-((S)-3-((3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropoxy)carbonyl)tetrahydropyridazin-1(2H)-yl)-3-oxopropyl)morpholine-4-carboxylate DCM solution (1.0 eq) and it was cooled to 5±5° C. under nitrogen. TFA (237.10 kg, 2.4 V) was charged to the reactor dropwise while maintaining the inner temperature at 5±5° C. The reaction mixture was stirred at 10±5° C. for 1 h and then stirred at 15±5° C. for no less than 4 h. Reaction monitoring by HPLC showed reaction is complete. Aqueous solution of NaOH (20% w/w, 5× of the starting material) was charged to reaction mixture and stirred for 1 h at 5° C. 5% aq. NaOH solution (4×f the starting material) was added to adjust the pH to the range of 8-10 while maintaining the temperature at 0-10° C. H₂O (4 V) was added to the mixture and stirred for 0.5 h. The organic layer was separated and was washed with H₂O (7 V). The solvent in the solution was exchanged with MTBE (470.9 kg, 10 V) by evaporation. The resulting MTBE solution of crude product was added to n-heptane (20 V) in another reactor over 6 h at 15±5° C. The mixture was stirred at 15±5° C. for at least 3 h. The slurry was filtered, and the wet cake was washed with n-heptane (2V). The crude solid was dried at 25±5° C. under vacuum for 10 h to give 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate as a white solid.

LCMS (ESI+): Calculated for C₅₀H₆₄BrF₃N₈O₇ (M+H): 1025.4; Found: 1025.8

¹H NMR (400 MHz, DMSO-d₆, 25° C.) δ 8.48 (d, J=2.9 Hz, 1H), 7.87 (d, J=2.0 Hz, 1H), 7.65 (d, J=8.7 Hz, 1H), 7.39 (dd, J=8.8, 1.9 Hz, 1H), 7.35 (m, 5H), 7.21 (d, J=2.8 Hz, 1H), 7.03 (d, J=8.8 Hz, 1H), 5.33 (dd, J=17.1, 8.6 Hz, 1H), 5.05 (m, 2H), 4.98 (s, 2H), 4.49 (dq, J=15.7, 9.3 Hz, 1H), 3.98 (s, 1H), 2.88 (s, 3H), 1.32 (d, J=6.2 Hz, 3H), 0.78 (s, 3H), 0.73 (s, 3H), 0.43 (m, 2H), 0.34 (m, 2H).

### Part 3—Synthesis of benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate

### Condition 1: P(t-Bu)₃·HBF₄ Ligand

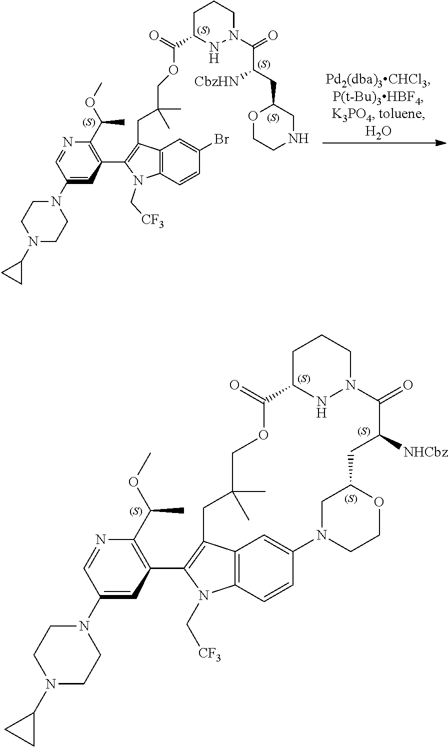

To a reactor was charged toluene (591.90 kg, 25 V), 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate (27.00 kg, 1.0 eq), anhydrous K₃PO₄ (27.90 kg, 5.0 eq). The mixture was degassed by bubbling nitrogen for 1 h under agitation at 20±5° C. (the content of 02 s 1000 ppm). Pd₂(dba)₃·CHCl₃ (1.41 kg, 0.047 eq), P(t-Bu)₃·HBF₄ (1.98 kg, 0.105 eq) and H₂O (4.35 kg, 9.0 equiv) were charged to the reactor and the reaction mixture was further degassed by bubbling nitrogen for 1 h. The reaction mixture was agitated at 90±5° C. for 11 h. Reaction monitoring by HPLC showed the reaction was complete. The crude reaction mixture was cooled to 20±10° C. and the mixture was filtered. To the filtrate EtOAc (449.90 kg, 10 V) was charged. The solvent in the resulting solution was concentrated to 3-5 V under reduced pressure at NMT 55° C. The solvent swap with EtOAc was repeated twice. The concentrated solution was washed with H₂O (99.50 kg, 2 V), aq. NaCl (10% w/w, 26.95 kg, 0.5 V), and aq. HCl (1% w/w, 17.50 kg, 0.4 V) successively at 5±10° C. The organic phase was separated and extracted with aq. HCl solution (1% w/w, 4 V and 3 V) twice. To the combined aqueous phases was charged EtOAc (183.25 kg, 4 V) and n-heptane (35.05 kg, 1 V) at 5±10° C. Aqueous NaCl solution (25% w/w, 33.45 kg, 0.6 V) was added dropwise to the mixture over 0.5 h. After stirring for 30 min, the aqueous phase was separated. The aqueous phase was concentrated under vacuum at NMT 35° C. to completely remove the organic solvent residue. The resulting suspension was stirred at 5±10° C. for at least 2 h. The slurry was filtered and the wet cake was washed with aq. NaCl solution (10% w/w, 10.35 kg, 0.2 V). The filter cake was collected as the HCl salt of the product. To a reactor was added EtOAc (468.10 kg, 10 V) and H₂O (262.00 kg, 5 V). The filter cake was added to the reactor at 5±10° C. with agitation. Aqueous K₂CO₃ solution (40% w/w, 20.95 kg) was added to the solution to adjust the pH to 8-10. The organic phase was separated, and the aqueous phase was washed with EtOAc (234.00 kg, 5 V). The combined organic phase was washed with aq. NaCl solution (10% w/w, 278.00 kg, 5 V). The organic phase was concentrated to 5.5-6.5 V under vacuum at NMT 45° C. The concentrated organic phase was extracted with aq. HCl (1% w/w, 103.70 kg, 2 V) two times and the aqueous phases were collected. The aqueous phase was concentrated at NMT 35° C. under reduced pressure (˜0.09 mpa) and solid precipitates crashed out. The residue was agitated for 1 h at 5±10° C. before aq. NaCl solution was added (25% w/w, 15.50 kg, 0.25 V). The resulting mixture was stirred for 2 h at 5±10° C. The slurry was filtered and the filter cake was washed with aq. NaCl solution (10% w/w, 16.10 kg, 0.3 V). The filter cake was collected as the HCl salt of the product. To a reactor was added EtOAc (234.00 kg, 5 V) and H₂O (130.50 kg, 2.5 V). The filter cake was added to the reactor at 5±10° C. with agitation. Aqueous K₂CO₃ solution (40% w/w) was added to the solution to adjust the pH to 8-10. The organic phase was separated, and the aqueous phase was washed with EtOAc (140.60 kg, 3 V). The combined organic phase was washed with aq. NaCl solution (10% w/w, 154.85 kg). The organic phase was concentrated to 1.5-2.5 V under vacuum at NMT 45° C. To a reactor with n-heptane (704.55 kg, 21 V) was charged the concentrated residue of the product at 20±5° C. over 2 h. The slurry was agitated for 2 h at 20±5° C. The slurry was filtered and the filter cake was washed with n-heptane (34.85 kg, 2 V). The wet cake was dried in oven at 30±5° C. for 10 h under vacuum to afford benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate (98.6% a/a, 20.60 kg, 44.6% yield) as white solid.

LCMS (ESI+): Calculated for C₅₀H₆₃F₃N₈O₇ (M+H): 945.5; Found: 945.9

¹H NMR (300 MHz, DMSO-d₆, 25° C.) 8.46 (dd, J=2.8 Hz, 1H), 7.57 (d, J=9.0 Hz, 1H), 7.35 (m, 5H), 7.17 (m, 2H), 7.08 (d, J=9.1 Hz, 1H), 6.98 (s, 1H), 5.28 (m, 2H), 4.97 (m, 2H), 4.71 (m, 1H), 3.33 (s, 3H), 1.33 (d, J=6.1 Hz, 3H), 0.83 (s, 3H), 0.43 (m, 7H), 0.34 (s, 2H).

### Condition 2: Qphos Ligand

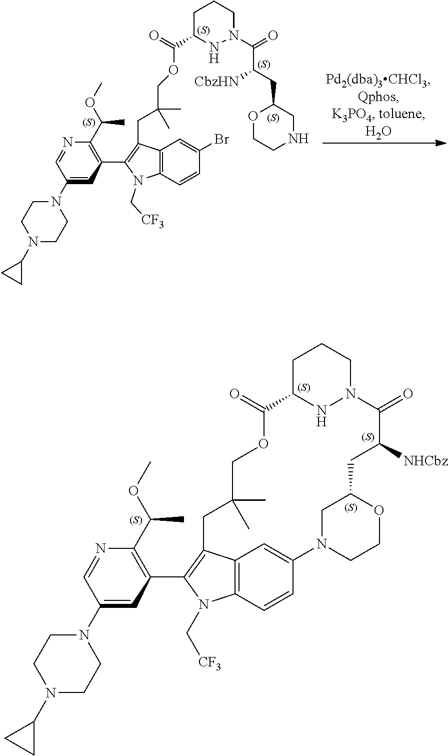

To a reactor was charged toluene (496.90 kg, 35 V), 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate (15.01 kg, 14.6 mol, 1.0 eq), anhydrous K₃PO₄ (16.21 kg, 76.4 mol, 5.0 eq). The mixture was degassed by bubbling nitrogen for 1 h under agitation at 20±5° C. (the content of 02 s 1000 ppm). Pd₂(dba)₃·CHCl₃ (745.60 g, 0.7 mol, 0.047 eq), 1,2,3,4,5-Pentaphenyl-1′-(di-tert-butylphosphino)ferrocene (1.09 kg, 1.5 mol, 0.105 eq) and H₂O (1.65 kg, 91.7 mol, 6.3 equiv) were charged to the reactor and the reaction mixture was further degassed by bubbling nitrogen for 1 h (the content of O₂≤1000 ppm). The reaction mixture was agitated at 90±5° C. for 10 h. Reaction monitoring by HPLC showed the reaction was complete. The crude reaction mixture was cooled to 20±10° C. and the mixture was filtered. The filtrate was concentrated to 3-5 V. To the concentrated residue EtOAc (358.00 kg, 10 V) was charged. The resulting solution was concentrated to 3-5 V under reduced pressure at NMT 55° C. The solvent swap with EtOAc was repeated twice. The concentrated solution was washed with H₂O (95.00 kg, 2.5 V) and aq. HCl (1% w/w, 25.00 kg, 0.5 V) successively at 0±5° C. The organic phase was separated and extracted with aq. HCl solution (1% w/w, 4 V and 3 V) twice. To the combined aqueous phases was charged 25% aq. NaCl (27.10 kg, 0.80 V), EtOAc (137.15 kg, 4 V) and n-heptane (25.85 kg, 1 V) at 5±10° C. After stirring for 30 min, the aqueous phase was separated. The aqueous phase was concentrated under vacuum at NMT 35° C. to completely remove the organic solvent residue. The resulting solution was stirred at 5±10° C. for at least 2 h. The mother liquor was sampled to get the concentration of the product. The slurry was filtered and the wet cake was washed with aq. NaCl solution (10% w/w, 10.00 kg, 0.3 V). The filter cake was collected as the HCl salt of the product. To a reactor was added EtOAc (340.45 kg, 10 V) and H₂O (185.50 kg, 5 V). The filter cake was added to the reactor at 5±10° C. with agitation. Aqueous K₂CO₃ solution (40% w/w, 16.00 kg) was added to the solution to adjust the pH to 8-10 range. The organic phase was separated and the aqueous phase was washed with EtOAc (174.00 kg, 5 V). The combined organic phases was washed with aq. NaCl solution (10% w/w, 190.50 kg, 5 V). The organic phase was concentrated to 2-3 V under vacuum at NMT 45° C. To a reactor with n-heptane (539.60 kg, 21 V) was charged the concentrated residue of the product at 20±5° C. over 2 h. The slurry was agitated for 2 h at 20±5° C. The slurry was filtered and the filter cake was washed with n-heptane (2 V). The wet cake was dried in oven at 30±5° C. for 10 h under reduced pressure to afford crude product. To a reactor with H₂O (91.50 kg, 6.8% w/w) and HCl (2.59 kg, 0.19% w/w) was charged crude product (13.38 kg, 14.2 mol, 1.0 equiv) at 5±5° C. The mixture was stirred at 5±5° C. for 2 h and then was charged 25% aq. NaCl (23.90 kg, 1 V) dropwise. The resulting slurry was filtered and the wet cake was washed with 10% aq. NaCl (31.40 kg, 2 V). The wet cake was suspended in H₂O (94.00 kg, 7 V) and EtOAc (96.45 kg, 8 V) at 5±10° C. The pH of aqeuous layer was adjusted to 8-10 with aq·K₂CO₃ solution (40% w/w, 10.65 kg). The organic layer was separated and the aqueous layer was washed with EtOAc (36.40 kg, 3 V). The organic layers were combined and washed with 10% w/w aq. NaCl solution (67.10 kg, 5% w/w). The organic phase was concentrated to 2-3 V under vacuum at NMT 45° C. The concentrated residue was diluted with EtOAc (60.75 kg, 5 V) and the resulting mixture was concentrated to 2-3 V under vacuum at NMT 45° C. The concentrated residue was added to n-heptane (208.10 kg, 21 V) at 20±5° C. dropwise and the mixture was stirred for 2 h. The slurry was filtered and wet cake was rinsed with n-heptane (18.65 kg, 2 V). The wet cake was dried in the oven under vacuum at 30±5° C. for 6 h to afford benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate (11.91 kg, 87.6% yield) as white solid.

### Condition 3: Using Dioxane as the Solvent

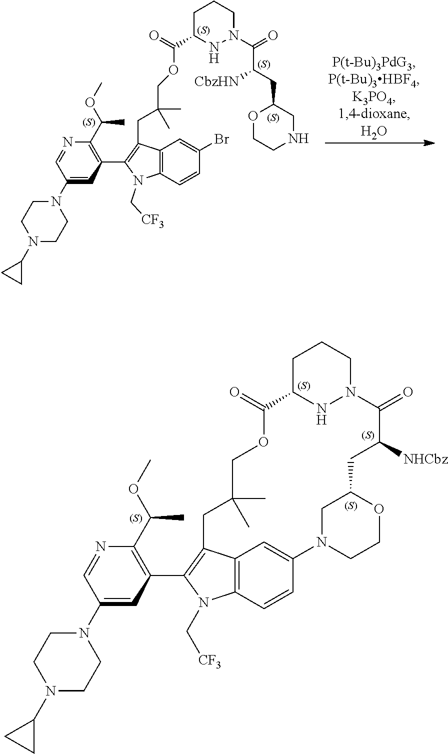

To a reactor was charged 1,4-dioxane (58.0 L, 35 V), 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate (1.50 kg, 1.5 mol, 1.0 eq), and anhydrous K₃PO₄ (1.03 kg, 4.8 mol, 3.3 eq). The mixture was degassed by bubbling nitrogen for 1 h under agitation at 20±5° C. (the content of 02 s 1000 ppm). P(t-Bu)₃PdG₃ (93 g, 0.15 mol, 0.11 eq), P(t-Bu)₃·HBF₄ (46.2 g, 0.15 mol, 0.11 eq) and H₂O (205 mL, 6.6 equiv) were charged to the reactor and the reaction mixture was further degassed by bubbling nitrogen for 1 h (the content of O₂≤1000 ppm). The reaction mixture was agitated at 90±5° C. for 10 h. Reaction monitoring by HPLC showed the reaction was complete. The crude reaction mixture was cooled to 20±10° C. and the mixture was filtered. The filter cake was washed with 1,4-dioxane (1.6 L, 1 V). The filtrate was concentrated to afford the crude product as yellow solids. The solids were dissolved in 2-MeTHF (13.8 L, 6 V) and aq. HCl (0.08 M, 5 V). The organic layer was separated and washed with aq. HCl (0.08 M, 4 V). The organic layer was extracted with aq. HCl (0.3 M, 5 V) for two times at −15° C. The combined aqueous layer was washed with 2-MeTHF (10.5 L, 5 V) at −15° C. twice. Na₂HCO₃ solids were added to the aqueous layer to adjust pH to 7 at 10-20° C. The resulting aqueous solution was extracted with 2-MeTHF (10.5 L, 10 V) two times. The combined organic layer was washed with aq. acetic acid (0.04 M, 10.5 L, 10 V) and 25% w/w aq. NaCl (10.5 L, 10 V) in sequence. The resulting organic layer was concentrated under reduced pressure to afford benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate as yellow solids (1.056 kg, 91.6% a/a).

### Condition 4: Pd(t-Bu₃P)₂ Catalyst and New Isolation Process

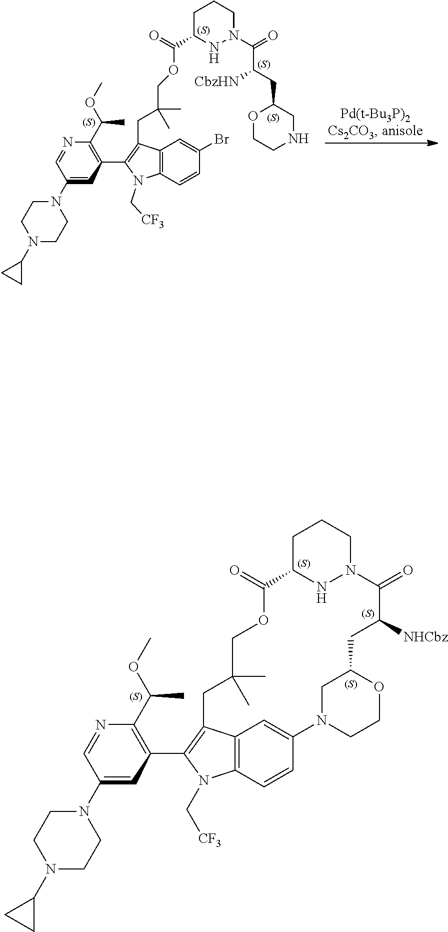

To a reactor was charged anisole (20 V) and 3-(5-bromo-2-(5-(4-cyclopropylpiperazin-1-yl)-2-((S)-1-methoxyethyl)pyridin-3-yl)-1-(2,2,2-trifluoroethyl)-1H-indol-3-yl)-2,2-dimethylpropyl (S)-1-((S)-2-(((benzyloxy)carbonyl)amino)-3-((S)-morpholin-2-yl)propanoyl)hexahydropyridazine-3-carboxylate (1.0 eq). The mixture was degassed by bubbling nitrogen for 1 h under agitation at 20±5° C. (the content of O₂≤1000 ppm). Cs₂CO₃ (3 equiv) and bis(tri-t-butylphosphine)palladium (0.1 equiv) were charged to the reactor and the reaction mixture was further degassed by bubbling nitrogen for 1 h (the content of 02 S 1000 ppm). The reaction mixture was agitated at 90±5° C. for 11 h. Reaction monitoring by HPLC showed the reaction was complete. The crude reaction mixture was cooled to 20±10° C. and the mixture was filtered. The filter cake was rinsed with anisole (3.5% w/w) and EtOAc (3.6% w/w). The filtrate was concentrated to 3-5 V under reduced pressure at NMT 55° C. The solvent swap with EtOAc was repeated twice. The EtOAc solution was extracted with aq. HCl solution (1% w/w, 4 V and 3 V) twice to form the HCl salt of the product. Alternatively, the EtOAc solution was extracted with p-toluenesulfonic acid or trichloroacetic acid to form the corresponding tosylate salt of the product or the corresponding trichloroacetate salt of the product. The combined aqueous phase was seeded with the HCl salt of the product (0.01% w/w) at 5° C. The mixture was stirred for 5 h at 5° C. before 25% aq. NaCl (0.6% w/w) was added. The resulting mixture was continued to stir for 5 h at 5° C. The slurry was filtered, and the wet cake was washed with 10% aq. NaCl solution (0.2% w/w). The wet cake was suspended in EtOAc (9% w/w) and H₂O (6.5% w/w). A 40% K₂CO₃ solution was added to adjust the pH to 8-10 at 0-10° C. After agitating for 1 h, the organic phase was separated, and the aqueous phase was extracted with EtOAc (4.5% w/w). The organic layers were combined and purified through a pad of silica gel (100-200 mesh, 0.7% w/w). The eluent after silica gel was concentrated to 2.5 V under reduced pressure NMT 40° C. The concentrated solution was added to n-heptane (1.43% w/w) dropwise at 20° C. The resulting mixture was stirred for 5 h at 20° C. The slurry was filtered and the filter cake was washed with n-heptane (1.4% w/w). The wet cake was dried in the oven at 30° C. for 8 h under reduced pressure to afford benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate as white solid.

### Part 4—Synthesis of (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione

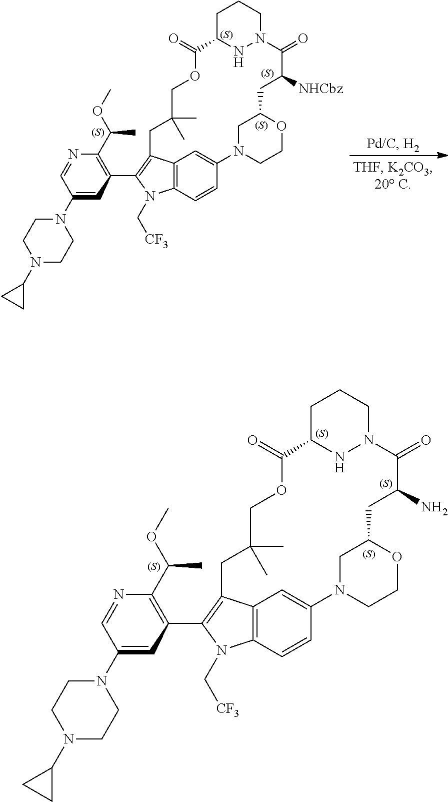

### Condition 1: Using K₂CO₃ Base

A pressure reactor was charged with THE (211.60 kg, 12 V), benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate (19.89 kg, 21 mol, 1.0 equiv), 5% w/w Pd/C (20% w/w, 4.25 kg), and K₂CO₃ (2.97 kg, 21 mol, 1 equiv). It was agitated at 0.2 mpa H₂, 20-25° C. The atmosphere was exchanged by venting and refilling with fresh H₂ gas several times during the course of the reaction. After reaction is complete as monitored by HPLC, the atmosphere was exchanged with N₂ gas and the reaction mixture was filtered through a pad of diatomite. The filtrates were then concentrated to 3-5 V under reduced pressure at NMT 40° C. and diluted with EtOAc (124.65 kg, 7 V). The mixture was further concentrated under reduced pressure at NMT 40° C. to 3-5 V. The concentrated residue was diluted with EtOAc (127.40 kg, 7 V) and the resulting solution was washed with 5% w/w aq. N-acetyl-L-cysteine solution (198.05 kg, 10% w/w). The mixture was filtered via fluid filter to remove the Pd complex. The filtrate was added 5% w/w aq. Na₂CO₃ solution to adjust the pH of aqueous layer to 7.4-8.0. The organic layer was separated and washed with 5% w/w aq. Na₂CO₃ (5% w/w) and 5% w/w aq. NaCl (5% w/w) in sequence. The organic layer was concentrated under reduced pressure at NMT 40° C. to 3-5 V. The concentrated residue was diluted with EtOAc (5 V) and the resulting solution was further concentrated under reduced pressure at NMT 40° C. to 3-5 V. The solvent swap process was repeated two times. The resulting crude product solution in EtOAc was added MTBE (58.65 kg, 4 V) over 2 h, followed by adding a seed of the product (0.01% w/w, 200 g) at 20±10° C. The mixture was agitated at 20±10° C. for 2 h. The mixture was diluted with MTBE (5 V) and concentrated to 4-6 V under reduced pressure at NMT 40° C. The solvent swap with MTBE was repeated three times. The concentrated solution was cooled to 0±5° C. and stirred for 2 h. The slurry was filtered and the wet cake was washed with MTBE (20.85 kg, 1 V). The filter cake was dried in the oven at 30±5° C. under vacuum to afford (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione (13.79 kg, 99.7% a/a, 84% yield) as white solid.

LCMS (ESI+): Calculated for C₄₂H₅₇F₃N₈O₅ (M+H): 811.4; Found: 811.5

¹H NMR (400 MHz, DMSO-d₆, 25° C.) δ 8.45 (d, J=2.8 Hz, 1H), 7.58 (d, J=8.9 Hz, 1H), 7.15 (d, J=2.7 Hz, 1H), 7.09 (d, J=8.9 Hz, 1H), 6.99 (s, 1H), 5.35 (dd, J=16.5, 8.5 Hz, 1H), 5.06 (d, J=12.2 Hz, 1H), 4.77 (dd, J=16.6, 9.1 Hz, 1H), 3.33 (s, 3H), 1.33 (d, J=6.1 Hz, 3H), 0.79 (s, 3H), 0.42 (m, 4H), 0.34 (s, 3H).

### Condition 2: No K₂CO₃ Base and Inclusion of Citric Acid Wash

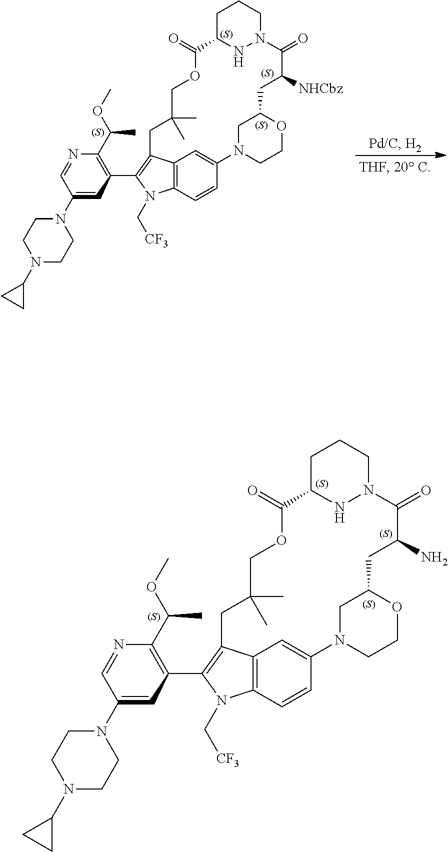

To a pressure reactor was charged and THE (12 V), benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate (1.0 equiv) and 5% w/w Pd/C (40% w/w, 4.25 kg). It was agitated at 0.2 mpa H₂, 20-25° C. The atmosphere was exchanged by venting and refilling with fresh H₂ gas several times during the course of the reaction. After reaction is complete as monitored by HPLC, the atmosphere was exchanged with N₂ gas and the reaction mixture was filtered through a pad of diatomite. MTBE (14 V) and 0.2% aq. citric acid solution (6 V) were added to the filtrate. The organic phase was separated and washed with 3% w/w aq. Na₂CO₃ solution (6 V). The organic layer was separated and washed with H₂O (3 V). The organic phase was separated and charged with MTBE (10 V). The resulting mixture was concentrated to 3-4 V. The solvent swap with MTBE was repeated two times. The concentrated residue was diluted with MTBE (1 V). The slurry was stirred for 3 h before it was filtered. The filter cake was washed with MTBE (1 V) and the wet cake was dried in the oven at 30-35° C. under reduced pressure to afford (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione as white solid.

### Condition 3: Using 2-MeTHF as Solvent, No Solvent Switch

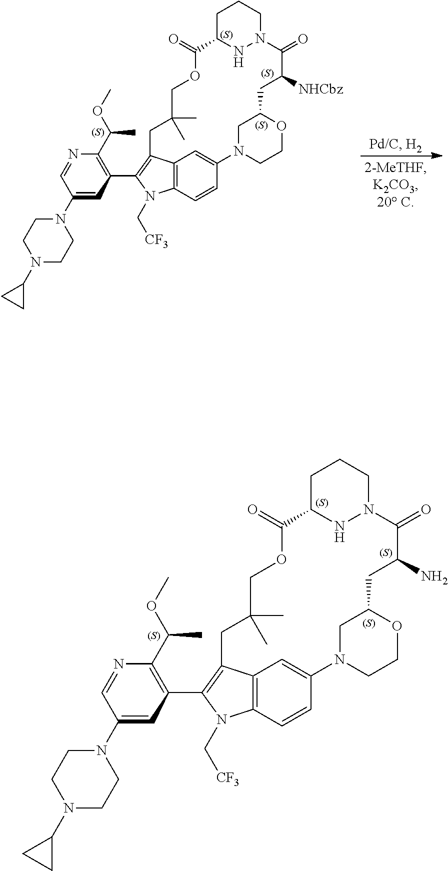

To an autoclave was charged 2-MeTHF (12 V), benzyl ((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)carbamate (1.0 equiv), 5% w/w Pd/C (20% w/w), and K₂CO₃ (1 equiv). It was agitated at 20-25° C. with the presence of 0.2 mpa H₂. The atmosphere was exchanged by venting and refilling with fresh H₂ gas several times during the course of the reaction. After reaction is complete as monitored by HPLC, the atmosphere was exchanged with N₂ gas and the reaction mixture was filtered through a pad of diatomite and the wet cake was washed with 2-MeTHF (5-7 times). The filtrate was washed with 5% w/w aq. N-acetyl-L-cysteine solution (10 V) at 10° C. The mixture was filtered. The filtrate was adjusted to pH=7.4-8.0 with 5% w/w aq. Na₂CO₃ solution. The organic phase was separated. The aqueous phase was extracted with 2-MeTHF (3 V). The organic phase was combined and washed with 5% w/w aq. Na₂CO₃ solution (5 V) and 5% w/w aq. NaCl solution (5 V) successively. The organic phase was concentrated to 7 V at NMT 35° C. and swapped with 2-MeTHF (10 V) twice. To the concentrated solution of product crude was added a seed of the product (0.01×) and then n-heptane (14 V) dropwise at 25° C. The resulting slurry was stirred at 5° C. for 9 h. The slurry was filtered and the wet cake was rinsed with 2-MeTHF/n-heptane (v/v=1:2, 3 V). The wet cake was dried in the oven at 30° C. for 39 h under vacuum. (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione was obtained as white solids (611.53 g, 97.1 assay, 85.5% yield).

### Part 5—Synthesis of Compound 1—(2S)-2-cyclopentyl-2-((S)-7-((2R,3R)-3-cyclopropyl-1-methylaziridine-2-carbonyl)-2,7-diazaspiro[4.4]nonan-2-yl)-N-((22S,63S,4S)-12-(5-(4-cyclopropylpiperazin-1-yl)-2-((S)-1-methoxyethyl)pyridin-3-yl)-10,10-dimethyl-5,7-dioxo-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-4-yl)acetamide

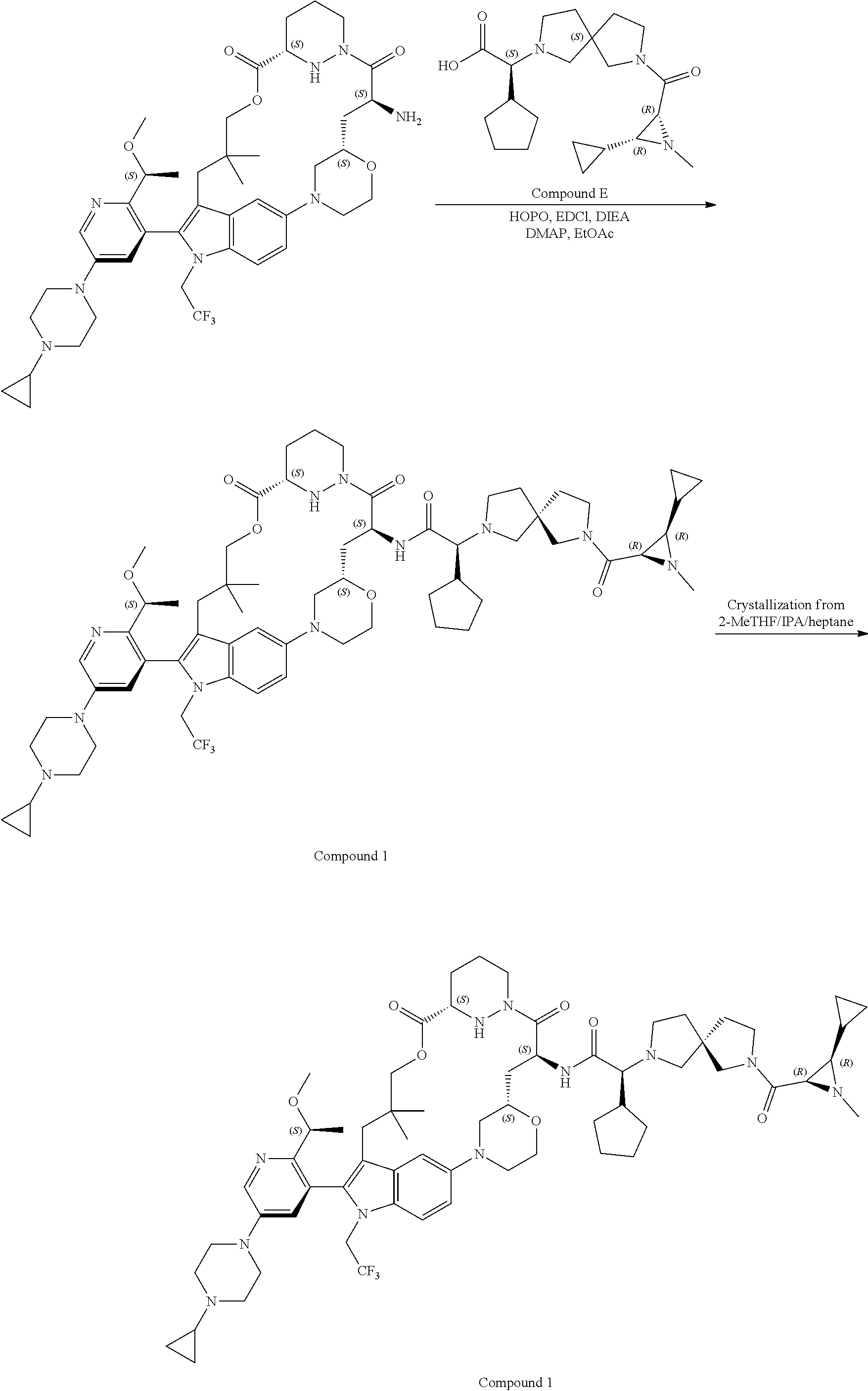

### Condition 1: HOPO/EDCl Coupling, 2-MeTHF/IPA/Heptane Crystallization

To a reactor was charged EtOAc (94.80 kg, 10 V), (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione (10.50 kg, 1.0 equiv), Compound E (5.30 kg, 1.1 equiv), DIPEA (6.60 kg, 4.0 equiv), DMAP (0.80 kg, 0.5 equiv), HOPO (0.23 kg, 0.15 equiv) and N-(3-Dimethylaminopropyl)-N′-ethylcarbodiimide hydrochloride (3.50 kg, 1.40 equiv) in sequence. The mixture was agitated at 20-30° C. for 9 h until reaction is complete. The crude reaction mixture was added to a reactor with H₂O (84.00 kg, 8 V) at 10±5° C. The biphasic mixture was agitated at 10±5° C. for 1 h and the organic phase was separated. The organic layer was washed with aq. NaHCO₃ (5 wt %, 105.20 kg), and aq. NaCl (2% w/w, 105.20 kg) in sequence. To the organic phase was charged H₂O (5 V) and the aqueous phase was adjusted to pH=5.5-6.1 with 5% w/w citric acid aq. solution (32.65 kg) at 5±5° C. The organic phase was separated. To the organic phase was added H₂O (10.05 kg, 1 V) and 1% w/w aq. Na₂CO₃ solution (7.70 kg) was added to adjust pH to 9-10 at 5±5° C. The organic phase was separated and washed with 2% w/w aq. NaCl solution (84.00 kg). The organic phase was concentrated to 3-5 V under vacuum at NMT 40° C. The concentrated residue was diluted with 2-MeTHF (10 V) and the mixture was concentrated to 3-5 V under vacuum at NMT 40° C. The solvent swap with 2-MeTHF was repeated three times. The crude product was obtained as 2-MeTHF solution which is further purified by recrystallization. To the 2-MeTHF solution of Compound 1 was added IPA (9.70 kg, 1.0 V) and n-heptane (20.85 kg, 2.5 V) at 5+5° C., followed by addition of Compound 1 seed crystal (5% w/w, 0.92 kg). The slurry was stirred at 5±5° C. for 12 h. The slurry was filtered and the wet cake was dried in the oven at 35±10° C. under vacuum to afford Compound 1 (10.84 kg, 71.6% yield).

LCMS (ESI+): Calculated for C₆₃H₈₈F₃N₁₁O₇ (M+H): 1168.7; Found: 1169.2

¹H NMR (400 MHz, CD3OD, 25° C.) δ 8.42 (d, J=2.4 Hz, 1H), 7.47 (d, J=9.2 Hz, 1H), 7.28 (d, J=1.2 Hz, 1H), 7.12 (m, 1H), 7.10 (m, 1H), 5.67 (d, J=9.2 Hz, 1H), 5.00 (m, 1H), 4.71 (m, 1H), 4.44 (d, J=12.8 Hz, 1H), 4.06 (d, J=2.0 Hz, 1H), 3.91-3.28 (m, 15H), 3.19 (s, 3H), 2.89-2.66 (m, 14H), 2.34 (d, J=14.0 Hz, 3H), 2.24-1.41 (m, 25H), 0.88 (s, 3H), 0.64 (m, 1H), 0.54 (s, 3H), 0.50-0.26 (m, 8H).

### Condition 2: PyBOP/Oxyma Coupling, 4-Dioxane/IPE Crystallization

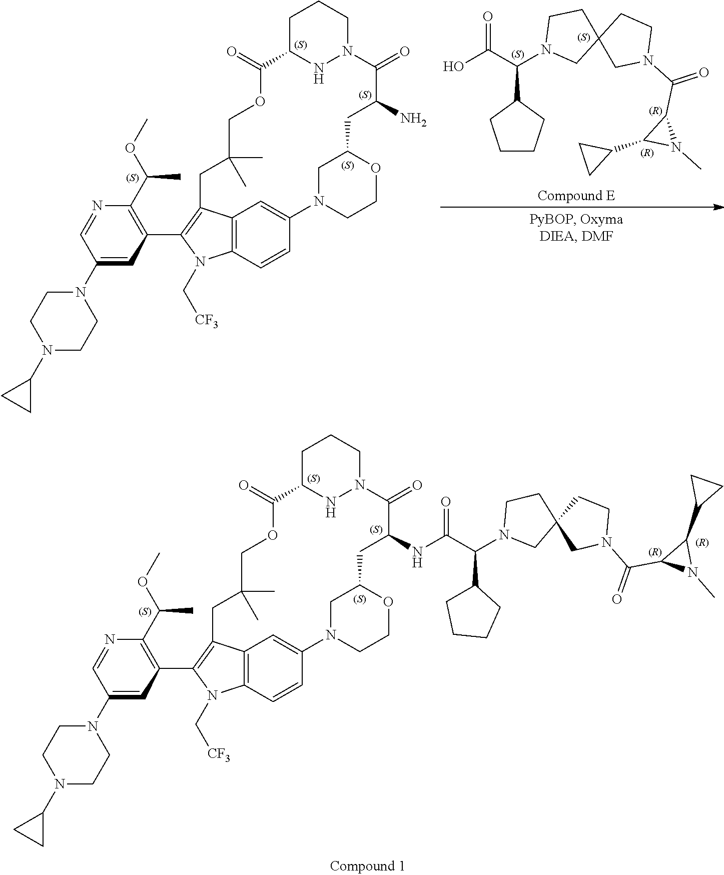

To a reactor was charged DMF (60 mL, 6 V), Compound E (5.10 g, 1.05 equiv), DIPEA (7.95 g, 5.0 equiv), (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione (10 g, 1.0 equiv) at 10-20° C. The mixture was cooled to 0±5° C. and oxyma (1.58 g, 0.9 equiv) was added. The reaction mixture was cooled to −10±5° C. and PyBOP (7.69 g, 1.2 equiv) in DMF (20 mL, 2 V) was added. The mixture was agitated at −10±5° C. for 1 h until reaction is complete. To the reaction mixture was added H₂O (35 mL, 3.5 V) and the mixture was filtered through microporous filter. Another portion of H₂O (54 V) was added to the filtrate at 20±5° C. and the mixture was stirred for 0.5 h. The slurry was filtered and the cake was washed with H₂O (5 V). The wet cake was dried under N₂ flow to afford pink solids. The pink solids were dissolved in IPOAc (7 V) and MTBE (5 V). The solution was washed with H₂O (12 V), 0.83% w/w aq. NaH₂PO₄ solution (12 V) and 3% w/w aq. NaHCO₃ solution (12 V) twice successively. The organic layer was concentrated to dryness. The residue was dissolved in IPOAc (15 V) and was slurried with 9% charcoal for 1 h at rt. The mixture was filtered. The filtrate was concentrated to dryness to afford crude Compound 1. The crude product was dissolved in 1,4-dioxane (2.5 V) and IPE (1.5 V) at 25° C. The solution was cooled to 10° C. and stirred for 2 h. IPE (1 V) was added dropwise to the solution over 8 h and then another portion of IPE (5 V) was added over 10 h. The resulting slurry was filtered and the filter cake was rinsed with 1,4-dioxane/IPE (v/v=2.5:7.5, 2 V). The solids were subjected to the second round of recrystallization by dissolving in 1,4-dioxane (2.5 V) and IPE (1.5 V) at 25° C. IPE (1 V) was dropped to the solution over 0.5 h at 25° C. Then the solution was cooled to 10° C. Compound 1 was seeded and the mixture was stirred for 3 h at 10° C. Another portion of IPE (3.5 V) was added to the mixture over 12 h and the slurry was stirred for 6 h. The slurry was filtered and the filter cake was rinsed with 1,4-dioxane/IPE (v/v=2.5:5.5, 2 V). The wet cake was dried under vacuum at 30° C. to afford Compound 1 as light yellow solids.

### Condition 3: HOBt/EDCl Coupling

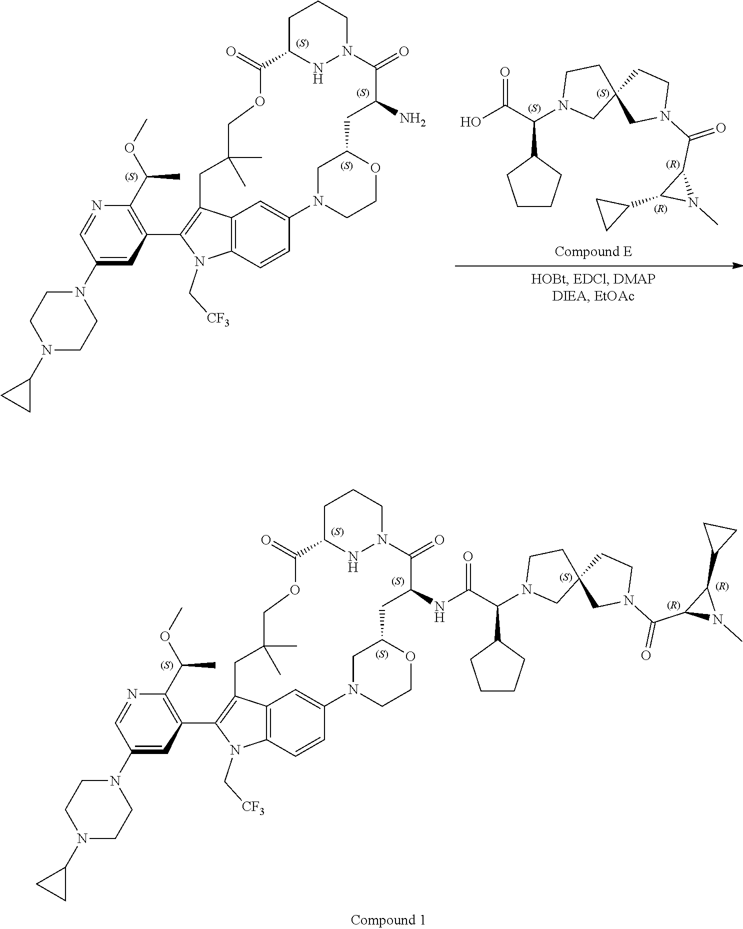

To a reactor was charged EtOAc (58.11 kg, 10 V), (22S,63S,4S)-4-amino-12-(5-(4-cyclopropylpiperazin-1-yl)-2-((S)-1-methoxyethyl)pyridin-3-yl)-10,10-dimethyl-11-(2,2,2-trifluoroethyl)-61,62,63,64,65,66-hexahydro-11H-8-oxa-2(4,2)-morpholina-1(5,3)-indola-6(1,3)-pyridazinacycloundecaphane-5,7-dione (6.40 kg, 1.0 equiv), Compound E (3.50 kg, 1.1 equiv), DIPEA (4.20 kg, 4.0 equiv), DMAP (481.9 g, 0.5 equiv), HOBt (74.9 g, 0.07 equiv) and N-(3-Dimethylaminopropyl)-N′-ethylcarbodiimide hydrochloride (2.15 kg, 1.40 equiv) in sequence. The mixture was agitated at 20-30° C. for 9 h until reaction is complete. The crude reaction mixture was added to a reactor with H₂O (52.60 kg, 8 V) at 10±5° C. The biphasic mixture was agitated at 10±5° C. for 1 h and the organic phase was separated. The organic layer was washed with aq. NaHCO₃ (5 wt %, 64.00 kg, 6.4% w/w) and aq. NaCl (2% w/w, 64.28 kg, 6.4% w/w) in sequence. To the organic phase was charged H₂O (5 V) and the aqueous phase was adjusted to pH=5.0-5.6 with 5% w/w citric acid aq. solution (1.35 kg) at 5±5° C. The organic phase was separated. To the organic phase was added H₂O (6.55 kg, 1 V) and 1% w/w aq. NaCO₃ solution (10.30 kg) was added to adjust pH to 9-10 at 5±5° C. The organic phase was separated and washed with 2% w/w aq. NaCl solution (51.20 kg, 8% w/w). The organic phase was concentrated to 3-5 V under vacuum at NMT 40° C. The concentrated residue was diluted with 2-MeTHF (10 V) and the mixture was concentrated to 3-5 V under vacuum at NMT 40° C. The solvent swap with 2-MeTHF was repeated three times. The crude product was obtained as a 2-MeTHF solution which is further purified by recrystallization. To the 2-MeTHF solution of Compound 1 was added n-heptane (15.00 kg, 2.5 V) at 5±5° C., followed by addition of a seed crystal of Compound 1 (5% w/w, 0.92 kg). The anti-solvent consisted of 2-MeTHF (7.90 kg, 1.33 V), IPA (3.70 kg, 0.67 V) and n-heptane (122.25 kg, 26 V) was added to the solution of Compound 1. The slurry was stirred at 5±5° C. for 12 h. The slurry was filtered and the wet cake was dried in the oven at 35±10° C. under vacuum to afford Compound 1 (6.24 kg, 98.4% a/a, 67.7% yield) as a white solid.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent, or patent application was specifically and individually indicated to be incorporated by reference in its entirety.

1. A method of separating enantiomers of a compound of Formula I 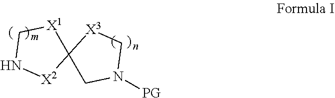
the method comprising the steps of:
a) contacting the compound of Formula I with a chiral acid to form a diastereomeric salt of the compound of Formula I; and
b) separating each diastereomer of the diastereomeric salt, thereby separating enantiomers of a compound of Formula I
wherein
n and m are each, independently, 0, 1, 2, 3, 4, or 5;
PG is a nitrogen protecting group; and
X¹, X², and X³ are each, independently, —CH₂—, —CHF—, —CF₂—, —C(O)—, or —O—.

2. The method of claim 1, wherein the chiral acid comprises a chiral carboxylic acid.

3. The method of claim 1 or 2, wherein the separating step (b) comprises recrystallization.

4. The method of any one of claims 1 to 3, wherein the compound of Formula I is further described by Formula Ia:

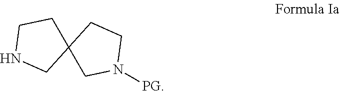

5. A barium salt of a compound of Compound A:

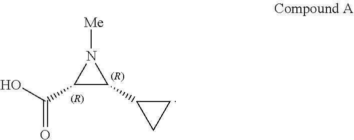

6. The barium salt of claim 5, wherein the barium salt is a 2:1 (compound A: barium) salt.

7. A method of preparing the barium salt of claim 5 or 6, the method comprising the steps of:
a) condensing a compound of Formula IIa and Compound C to form a compound of Formula IIb: 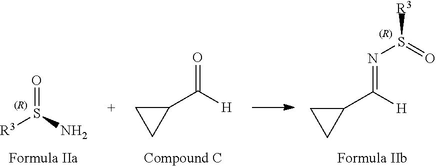
b) cyclizing the compound of Formula IIb and a compound of Formula IIc to form a compound of Formula IId: 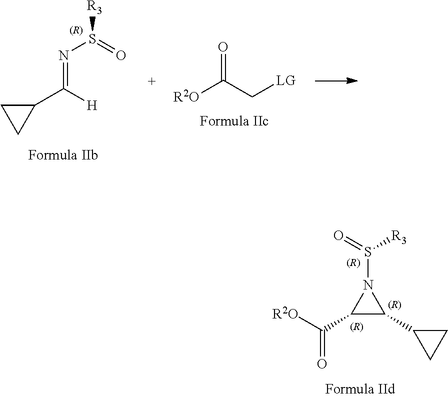
c) hydrolyzing the compound of Formula IId to form a compound of Formula IIe: 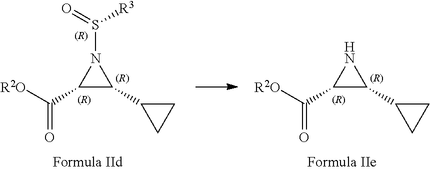
d) methylating the compound of Formula IIe to form a compound of Formula IIf: 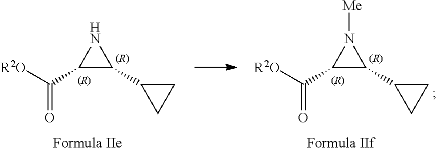
and
e) hydrolyzing the compound of Formula IIf to form the barium salt of Compound A: 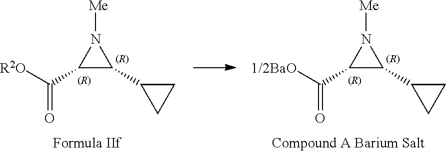
wherein
R² and R³ are each, independently, optionally substituted C₁-C₆ alkyl, optionally substituted C₃₋₆ cycloalkyl, or optionally substituted C₆-C₁₀ aryl, and
LG is a leaving group selected from the group consisting of halogen, triflate, mesylate, and tosylate.

8. The method of claim 7, wherein the methylating step (d) comprises contacting the compound of Formula IIe with MeB(OH)₂.

9. The method of claim 7 or 8, wherein the hydrolyzing step (e) comprises contacting the compound of Formula IIf with barium hydroxide.

10. A method of preparing a compound, or a salt thereof, of Formula III: 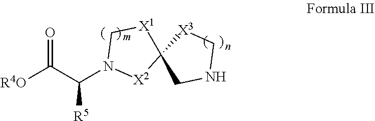
the method comprising the steps of:
a) coupling a compound of Formula IIId with a compound of Formula I to form a compound of Formula IIIe: 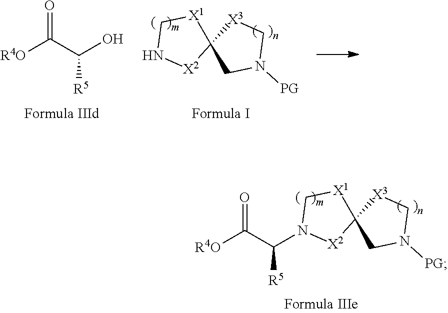
and
b) deprotecting the compound of Formula IIIe to form the compound of Formula III: 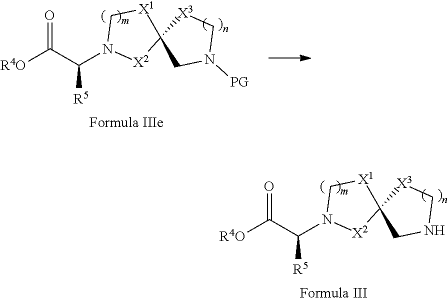
wherein
n and m are each, independently, 0, 1, 2, 3, 4, or 5;
PG is a nitrogen protecting group;
each R⁴ is independently, optionally substituted C₁-C₆ alkyl or optionally substituted C₆-C₁₀ aryl;
R⁵ is optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

11. The method of claim 10, wherein the compound of Formula IIId is prepared by a method comprising the steps of:
A1) combining a compound of Formula IIIa and a compound of Formula IIIb to form a compound of Formula IIIc: 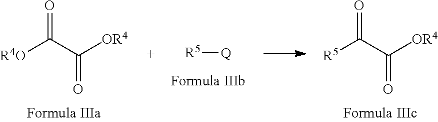
B1) reducing the compound of Formula IIIc to form the compound of Formula IId: 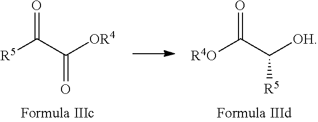
wherein Q is MgBr, MgCl, MgI, or Li.

12. The method of claim 11, wherein the reducing step (B1) comprises contacting the compound of Formula IIIc with a ketoreductase enzyme.

13. The method of claim 10, wherein the compound of Formula IIId is prepared by the steps of:
A2) converting a compound of Formula IIIf to form a compound of Formula IIIg: 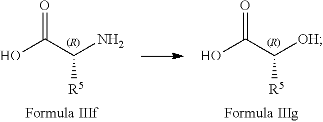
B2) reacting (e.g., esterifying) the compound of Formula IIIg to form the compound of Formula IIId:

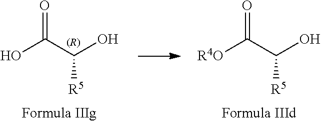

14. The method of claim 13, wherein the converting step (A2) comprises forming a diazonium salt of the compound of Formula IIIf.

15. A method of preparing a compound, or a salt thereof, of Formula IV: 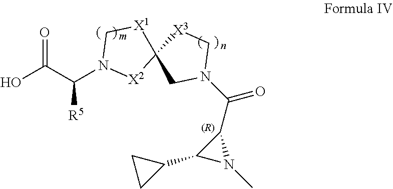
the method comprising the steps of:
a) coupling a compound of Formula III and a barium salt of compound A to form a compound of Formula IVa: 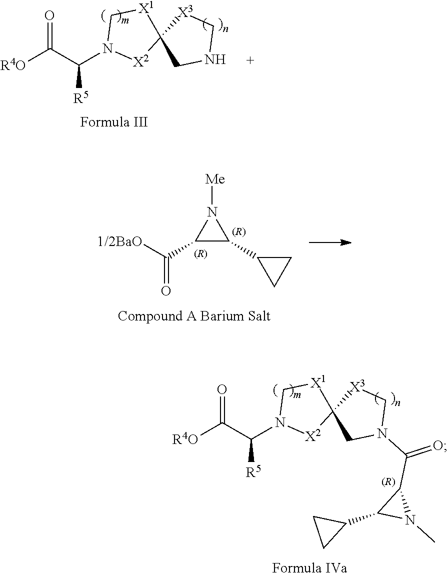
and
b) hydrolyzing the compound of Formula IVa to form the compound of Formula IV: 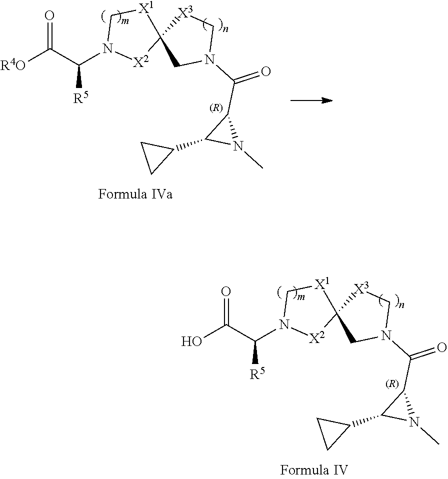
wherein
n and m are each, independently, 0, 1, 2, 3, 4, or 5;
R⁴ is optionally substituted C₁-C₆ alkyl or optionally substituted C₆-C₁₀ aryl;
R⁵ is optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

16. The method of claim 15, wherein the coupling step (a) comprises contacting the compound of Formula III and the Compound A barium salt with chloro-N,N,N′,N′-tetramethylformamidinium hexafluorophosphate (TCFH).

17. A method of preparing a compound, or a salt thereof, of Formula VIII: 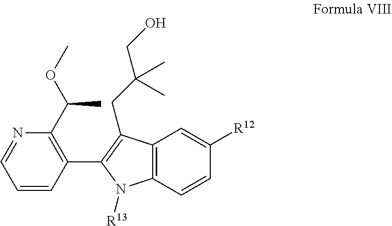
the method comprising the steps of:
a) esterifying and N-alkylating a compound of Formula VIIIa to form a compound of Formula VIIIb: 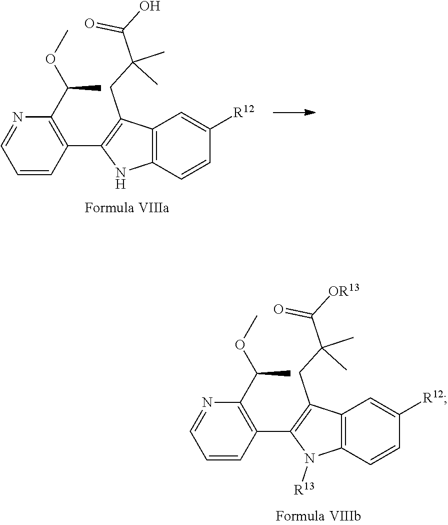
b) reducing the compound of Formula VIIIb to form a compound of Formula VIIIc as a mixture of atropisomers: 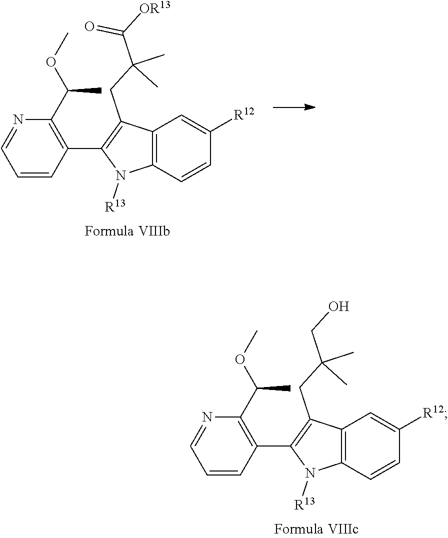
c) separating the mixture of atropisomers of Formula VIIIc to obtain a stereochemically pure compound of Formula VIII;
wherein R¹² is halogen; and
R¹³ is optionally substituted C₁-C₆ alkyl.

18. A method of preparing a compound, or a salt thereof, of Formula VIII: 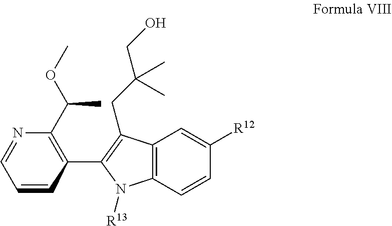
the method comprising the steps of:
a) esterifying a compound of Formula VIIIa to form a compound of Formula VIIId: 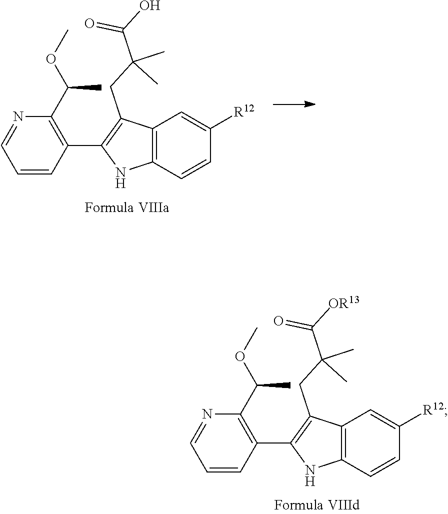
b) N-alkylating the compound of Formula VIIId to form the compound of Formula VIIIb: 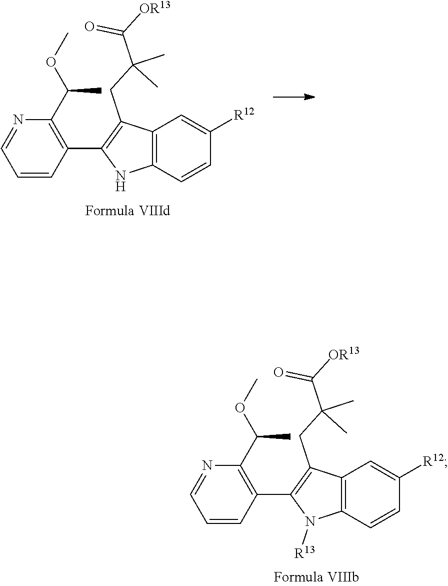
c) reducing the compound of Formula VIIIb to form a compound of Formula VIIIc as a mixture of atropisomers: 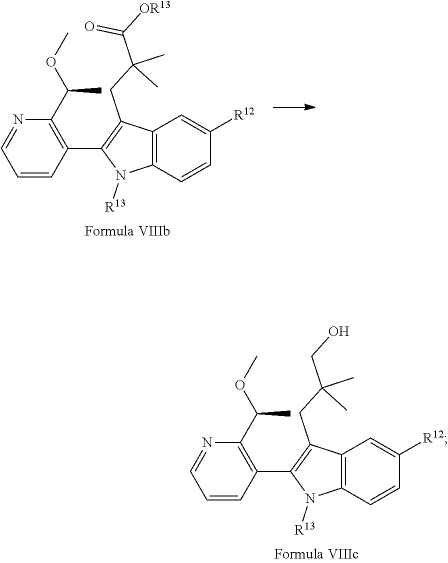
and
d) separating the mixture of atropisomers of Formula VIIIc to obtain the stereochemically pure compound of Formula VIII;
wherein R¹² is halogen; and
R¹³ is optionally substituted C₁-C₆ alkyl.

19. A method of preparing a compound, or a salt thereof, of Formula IX: 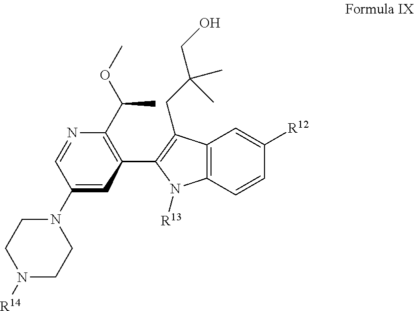
the method comprising the steps of:
a) borylating a compound of Formula VIII to form a compound Formula X: 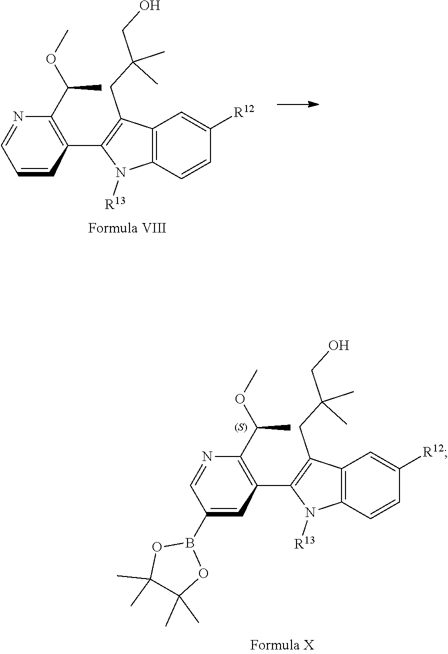
and
b) coupling the compound of Formula X with the compound of Formula XI to form a compound of Formula XII: 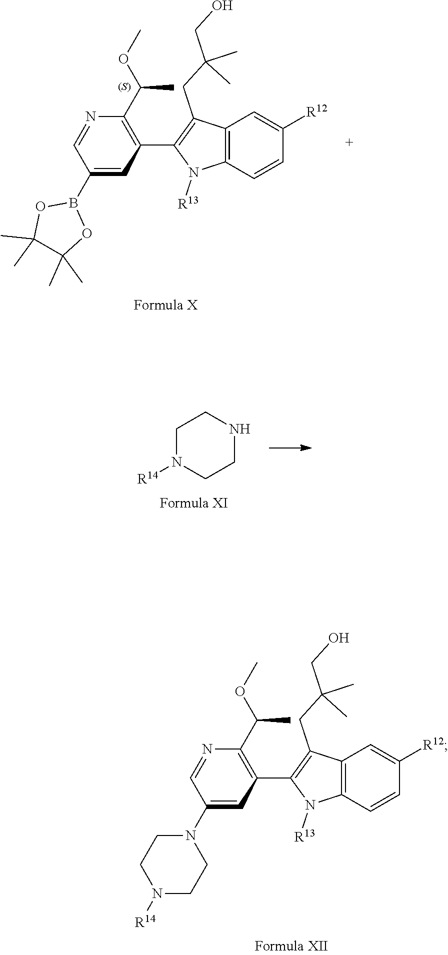
wherein R¹² is halogen;
R¹³ is optionally substituted C₁-C₆ alkyl; and
R¹⁴ is optionally substituted 3- to 6-membered cycloalkyl.

20. A method of preparing a compound, or a salt thereof, of Formula XIV: 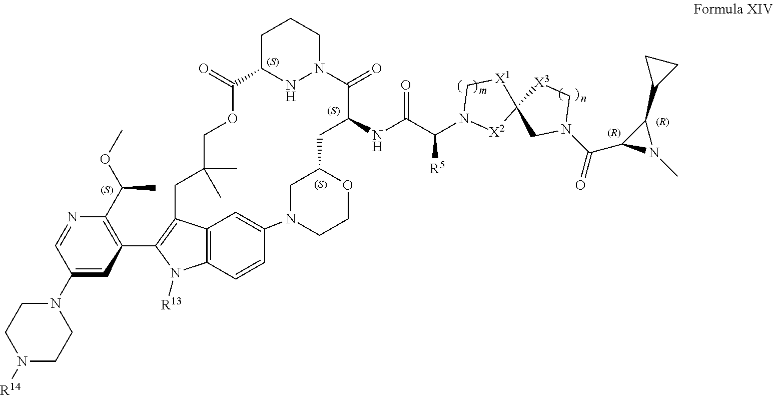
the method comprising the steps of:
a) reacting a compound of Formula XV with a compound of Formula XII to form a compound of Formula XVI: 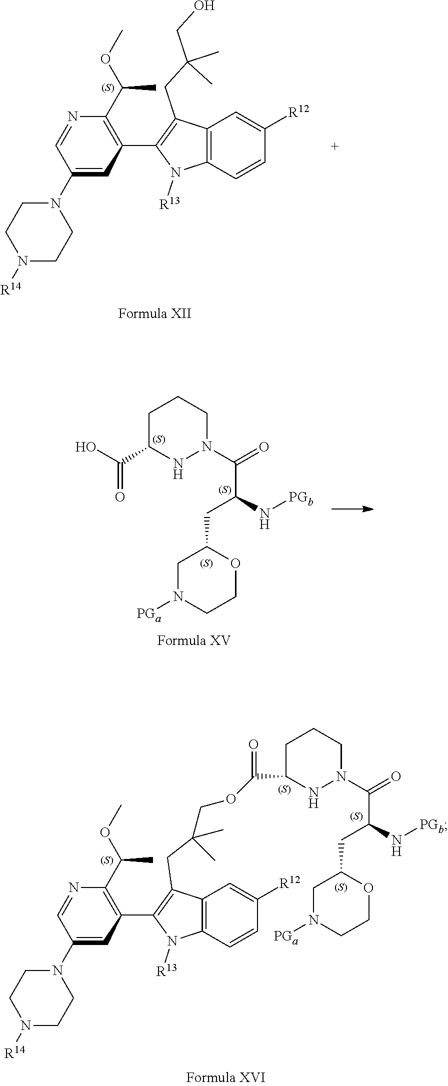
b) removing PGₐ of the compound of Formula XVI to form a compound of Formula XVII: 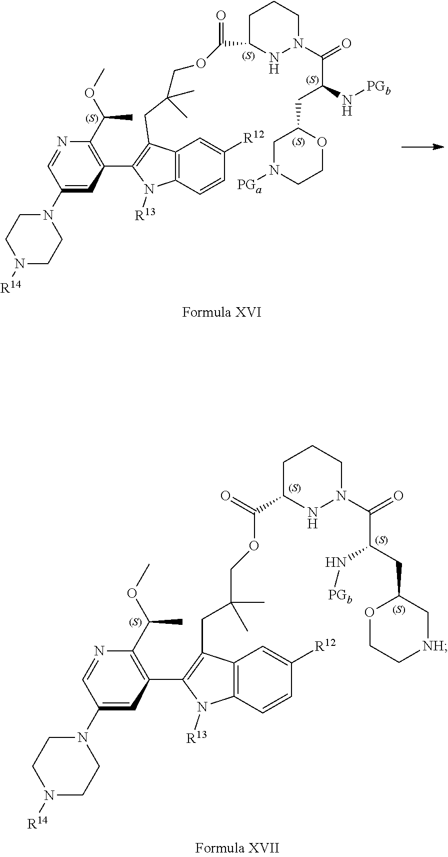
c) cyclizing the compound of Formula XVII with a transitional metal catalyst to form a compound of Formula XVIII: 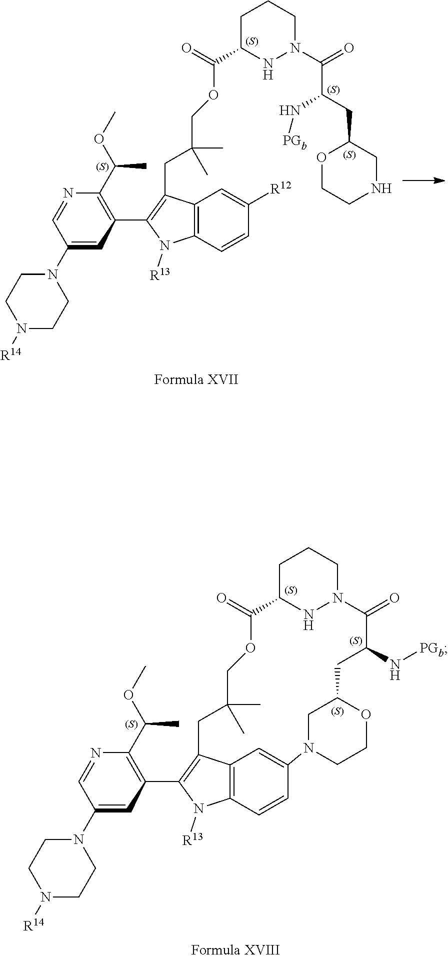
d) removing PG_{b} of the compound of Formula XVIII to form a compound of Formula XIX: 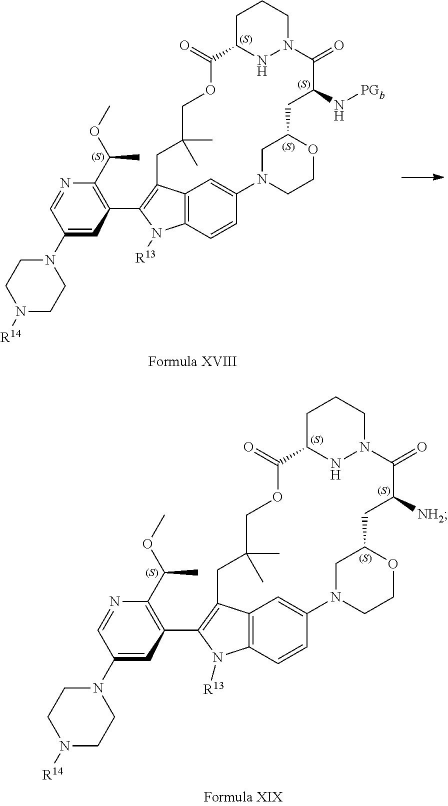
and
e) coupling the compound of Formula XIX with the compound of Formula IV to form a compound of Formula XIV: 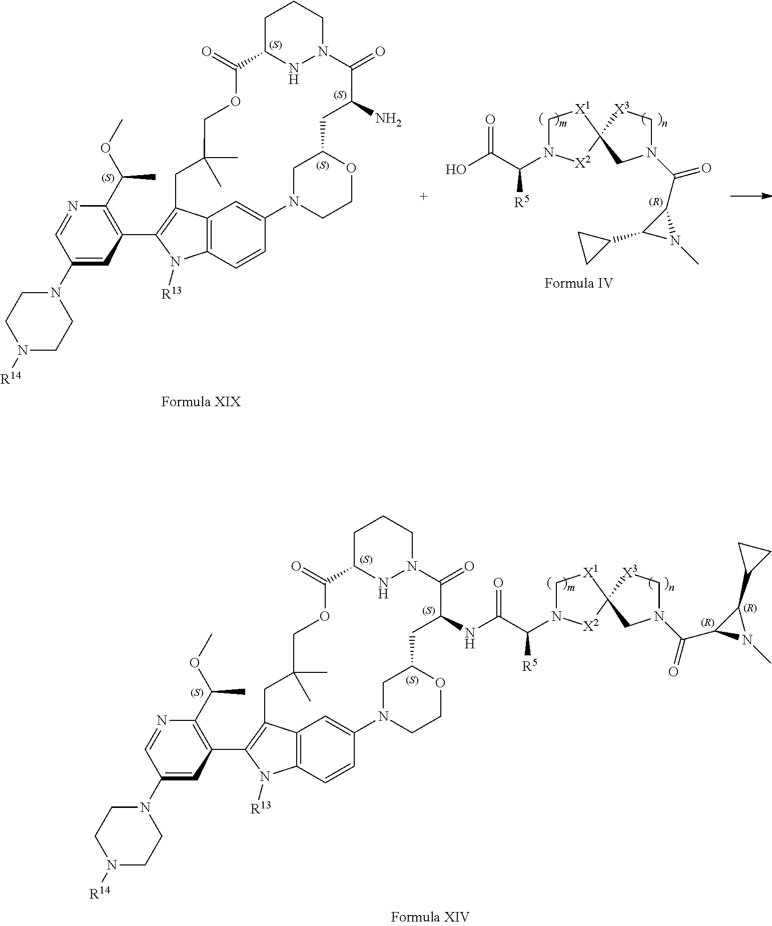
wherein n and m are each, independently, 0, 1, 2, 3, 4, or 5;
X¹, X², and X³ are each, independently, —CH₂—, —CHF—, —CF₂—, —C(O)—, or —O—;
R¹² is halogen;
R¹³ is optionally substituted C₁-C₆ alkyl;
R¹⁴ is optionally substituted 3- to 6-membered cycloalkyl;
PGₐ is an acid-labile protecting group; and
PG_{b} is an acid-stable protecting group.

21. The method of claim 20, wherein step (a) comprises reacting the compound of Formula XII and the compound of Formula XV in the presence of a carbodiimide coupling reagent, an anti-racemization agent, and a base.

22. The method of claim 20 or 21, wherein step (c) further comprises contacting the compound of Formula XVIII with hydrochloric acid to form a hydrochloride salt of the compound of Formula XVIII.

23. The method of any one of claims 20 to 22, wherein step (e) comprises coupling the compound of Formula XIX and the compound of Formula IV in the presence of a coupling reagent, an anti-racemization reagent, and a base.

24. A compound of Formula IIf-1: 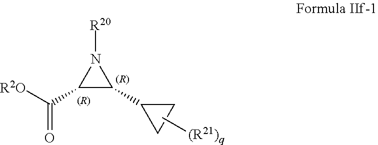
or a salt thereof, wherein
R² is optionally substituted C₁₋₆ alkyl, optionally substituted C₃₋₆ cycloalkyl, or optionally substituted C₆₋₁₀ aryl;
R²⁰ is hydrogen or C₁₋₆ alkyl optionally substituted with 1 or 2 substituents independently selected from CN, OH, C₁₋₆ alkoxy, and C₁₋₆ haloalkyl;
R²¹ is OH, halogen, CN, C₁₋₆ alkyl, C₁₋₆ alkoxy or C₁₋₆ haloalkyl; and
q is 0, 1, or 2.

25. A method of preparing a compound of Formula IIf: 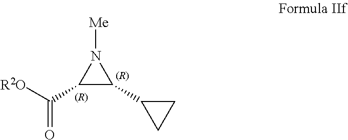
the method comprising alkylating a compound of Formula IIe with a methylating agent: 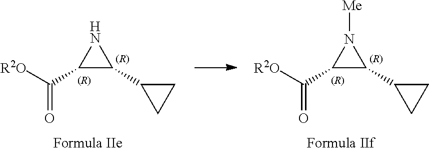
wherein R² is optionally substituted C₁₋₆ alkyl, optionally substituted C₃₋₆ cycloalkyl, or optionally substituted C₆₋₁₀ aryl.

26. The method of claim 25, wherein the methylating agent comprises trimethylboroxine.

27. A method of preparing a compound of Formula IVa: 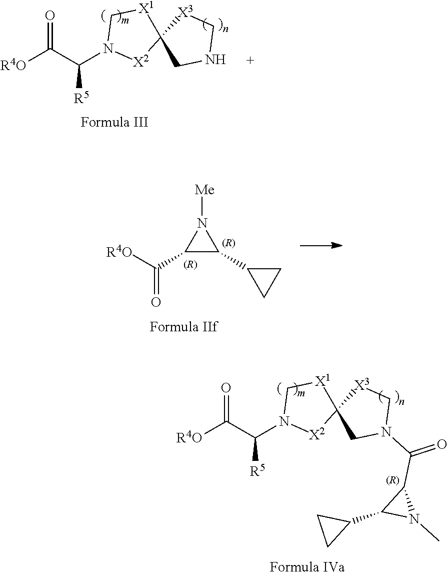
the method comprising coupling a compound of Formula III with a compound of Formula IIf
wherein n and m are each, independently, 0, 1, 2, 3, 4, or 5;
each R⁴ is independently optionally substituted C₁-C₆ alkyl or optionally substituted C₆-C₁₀ aryl;
each R⁵ is independently optionally substituted C₁-C₆ alkyl, optionally substituted C₁-C₆ heteroalkyl, optionally substituted 3- to 6-membered cycloalkyl, or optionally substituted 3- to 6-membered heterocycloalkyl; and
X¹, X², and X³ are each, independently, —CH₂—, —CHF—, —CF₂—, —C(O)—, or —O—.

28. The method of claim 27, wherein the coupling comprises contacting the compound of Formula III and the compound of Formula IIf with a base.

29. A method of preparing a compound of Formula VIIIc: 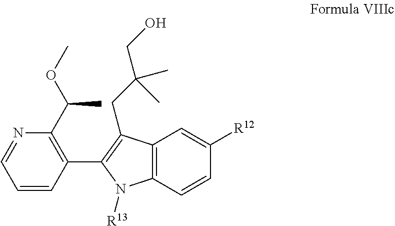
the method comprising N-alkylating a compound of Formula VIIIe with a compound having the structure of R¹³-LG¹: 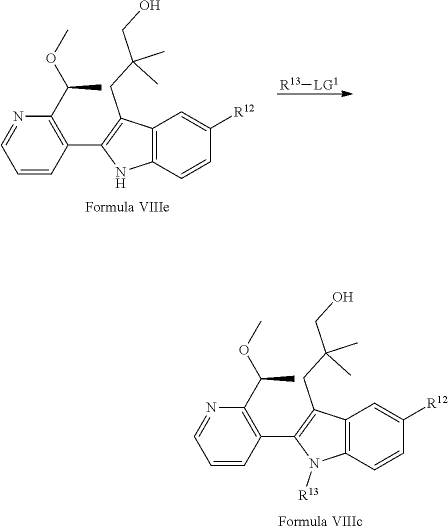
wherein each R¹² is halogen;
each R¹³ is optionally substituted C₁-C₆ alkyl; and
LG¹ is a leaving group.

30. A method of preparing a compound of Formula Ville: 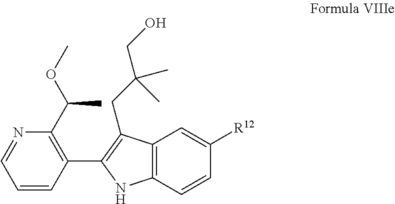
the method comprising reducing a compound of Formula VIIIf with a reducing agent: 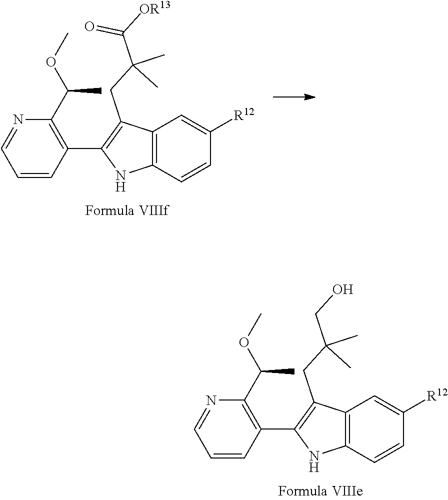
wherein R¹² is halogen; and
R¹³ is optionally substituted C₁-C₆ alkyl.

31. The method of claim 30, wherein the reducing agent is NaBH₄.
